SEQUENCE LISTING (196 sequences)

SEQ ID NO: 1 - protein, 511 aa, Human immunodeficiency virus
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKR

SEQ ID NO: 2 - protein, 470 aa, Human immunodeficiency virus
MRVKGILRNCQQWWIWGILGFWMLMICNVVGNLWVTVYYGVPVWKEAKTTLFCASDAKSYEREVHNVWATHACVPTDPDPQELVMANVTENFNMWKNDMVDQMHEDIISLWDQSLKPCVKLTPLCVTLNCTSPAAHNESETRVKHCSFNITTDVKDRKQKVNATFYDLDIVPLSSSDNSSNSSLYRLISCNTSTITQACPKVSFDPIPIHYCAPAGYAILKCNNKTFSGKGPCSNVSTVQCTHGIRPVVSTQLLLNGSLAEEEIVIRSENLTDNAKTIIVHLNKSVEIECIRPGNNTRKSIRLGPGQTFYATGDVIGDIRKAYCKINGSEWNETLTKVSEKLKEYFNKTIRFAQHSGGDLEVTTHSFNCRGEFFYCNTSELFNSNATESNITLPCRIKQIINMWQGVGRAMYAPPIRGEIKCTSNITGLLLTRDGGNNNNSTEEIFRPEGGNMRDNWRSELYKYKVVEIK

SEQ ID NO: 3 - protein, 473 aa, Human immunodeficiency virus
MRVRGILRNWPQWWIWSILGFWMLIICRVMGNLWVTVYYGVPVWKEAKATLFCASDARAYEKEVHNVWATHACVPTDPNPQEIYLGNVTENFNMWKNDMVDQMHEDIISLWDQSLKPCVKLTPLCVTLRCTNATINGSLTEEVKNCSFNITTELRDKKQKAYALFYRPDVVPLNKNSPSGNSSEYILINCNTSTITQACPKVSFDPIPIHYCAPAGYAILKCNNKTFNGTGPCNNVSTVQCTHGIKPVVSTQLLLNGSLAEEDIIIKSENLTNNIKTIIVHLNKSVEIVCRRPNNNTRKSIRIGPGQAFYATNDIIGDIRQAHCNINNSTWNRTLEQIKKKLREHFLNRTIEFEPPSGGDLEVTTHSFNCGGEFFYCNTTRLFKWSSNVTNDTITIPCRIKQFINMWQGAGRAMYAPPIEGNITCNSSITGLLLTRDGGKTDRNDTEIFRPGGGNMKDNWRNELYKYKVVEIK

SEQ ID NO: 4 - protein, 471 aa, Human immunodeficiency virus
MRVREIPRNYQQWWIWGILGFWMLMICSVVGNLWVTVYYGVPVWREAKTTLFCASDAKAYEREVHNVWATHACVPTDPNPQEMVLENVTENFNMWKNDMVDQMQEDIISLWDQSLKPCVKLTPLCVTLNCSKLNNATDGEMKNCSFNATTELRDKKKQVYALFYKLDIVPLDGRNNSSEYRLINCNTSTITQACPKVSFDPIPIHYCAPAGYAILKCNNKTFNGTGPCHNVSTVQCTHGIKPVISTQLLLNGSTAEEDIIIRSENLTNNAKTIIVHLNESIEIECTRPGNNTRKSIRIGPGQAFFATTNIIGDIRQAYCIINKANWTNTLHRVSKKLEEHFPNKTINFNSSSGGDLEITTHSFNCGGEFFYCNTSSLFNGTYNDTDIYNSTDIILLCRIKQIINMWQEVGRAMYAPPIEGNITCSSNITGLLLTRDGGLTNESKETFRPGGGDMRDNWRSELYKYKVVEIK

SEQ ID NO: 5 - protein, 864 aa, Human immunodeficiency virus
MRVKETQMNWPNLWKWGTLILGLVIICSASDNLWVTVYYGVPVWRDADTTLFCASDAKAHETEVHNVWATHACVPTDPNPQEIDLENVTENFNMWKNNMVEQMQEDVISLWDQSLKPCVKLTPLCVTLHCTNANLTKANLTNVNNRTNVSNIIGNITDEVRNCSFNMTTELRDKKQKVHALFYKLDIVPIEDNNDNSKYRLINCNTSVIKQACPKISFDPIPIHYCTPAGYAILKCNDKNFNGTGPCKNVSSVQCTHGIKPVVSTQLLLNGSLAEEEIIIRSEDLTNNAKTIIVHLNKSVVINCTRPSNNTRTSITIGPGQVFYRTGDIIGDIRKAYCEINGTEWNKALKQVTEKLKEHFNNKPIIFQPPSGGDLEITMHHFNCRGEFFYCNTTRLFNNTCIANGTIEGCNGNITLPCKIKQIINMWQGAGQAMYAPPISGTINCVSNITGILLTRDGGATNNTNNETFRPGGGNIKDNWRNELYKYKVVQIEPLGVAPTRAKRRVVEREKRAVGIGAMIFGFLGAAGSTMGAASITLTVQARQLLSGIVQQQSNLLRAIEAQQHLLQLTVWGIKQLQARVLAVERYLKDQKFLGLWGCSGKIICTTAVPWNSTWSNKSLEEIWNNMTWIEWEREISNYTNQIYEILTKSQDQQDRNEKDLLELDKWASLWTWFDITNWLWYIKIFIMIVGGLIGLRIIFAVLSIVNRVRQGYSPLSFQTPCHHQREPDRPERIEEEGGEQGRDRSVRLVSGFLALAWDDLRSLCLFSYHRLRDFILIAARTVELLGRSSLKGLRRGWEGLKYLGNLLLYWGQELKISAISLLDATAIAVAGWTDRVIEVAQGAWKAILHIPRRIRQGLERALQ

SEQ ID NO: 6 - protein, 487 aa, Human immunodeficiency virus
MRVRGILRNYQQWWIWGILGFWVLMICNGNLWVTVYYGVPVWKEAKTTLFCASDAKAYEKEVHNVWATHACVPTDPNPQEMVLENVTENFNMWKNDMVEQMHEDVISLWDQSLKPCVKLTPLCVTLECRQVNTTNATSSVNVTNGEEIKNCSFNATTEIRDKKQKVYALFYRLDIVPLEEERKGNSSKYRLINCNTSAITQACPKVTFDPIPIHYCAPAGYAILKCNNKTFNGTGPCNNVSTVQCTHGIKPVVSTQLLLNGSLAEGEIIIRSENLTNNVKTIIVHLNESVEIVCTRPNNNTRKSIRIGPGQTFYATGDIIGNIRQAYCNIKKDDWIRTLQRVGKKLAEHFPRRIINFTSPAGGDLEITTHSFNCRGEFFYCNTSSLFNSTYNPNDTNSNSSSSNSSLDITIPCRIKQIINMWQEVGRAMYAPPIEGNITCKSNITGLLLVRDGGVESNETEIFRPGGGDMRNNWRSELYKYKVVEIK

SEQ ID NO: 7 - protein, 493 aa, Human immunodeficiency virus
MRVMGIRKNYQHLWRWGTMGMMLLGILMICNATEKLWVTVYYGVPVWKEATTTLFCASDAKAYETEVHNVWATHACVPTDPNPQELVLENVTEYFDMWKNNMVEQMHEDIISLWDQSLKPCVKLTPLCVTLNCTDWTNGTDWNTTNSNNTTISKEETIEGGEMKNCSFNITTATGDKKKERAFFYKLDVAPIDNSNTSYRLISCNTSVITQACPKISFEPIPIHYCAPAGFAILKCNDKKFNGTGSCTNVSTVQCTHGIRPVVSTQLLLNGSLAEEEVVIRSKNFSDNAKIIIVQLNESVPINCTRPHNNTRKSIHIGPGRAWYATGDIIGDIRKAYCNISEAKWNNTLKQITEKLKEQFNKTIIVFNQPSGGDPEVTMHSFNCGGEFFYCNTSKLFNGTWNSTKRANNTEGIIILQCRIKQIINRWQEVGKAMYAPPIEGQIKCSSNITGLLLTRDGGKTANNTTEFFRPGGGNMKDNWRSELYKYKVVRIE

SEQ ID NO: 8 - protein, 471 aa, Human immunodeficiency virus
MRVRGIMRNWQQWWIWGSLGFWMLIICNVMGSLWVTVYYGVPVWREAKTTLFCASDAKAYETEAHSVWATHACVPTDPNPQEMVLENVTENFNMWKNDMVDQMHEDVISIWDQSLKPCVKLTPLCVTLDCSTYNNTHNISKEMKICSFNMTTELRDKKRKVNVLFYKLDLVPLTNSSNTTNYRLISCNTSTITQACPKVSFDPIPIHYCAPAGYAILKCNNKTFNGTGPCNNVSTVQCTHGIKPVVSTQLLLNGSLAEEEIIIRFENLTDNVKIIIVQLNETINITCTRPNNNTRKSIRIGPGQSFYATGEIVGNIREAHCNISASKWNKTLERVRTKLKEHFPNKTIEFEPSSGGDLEITTHSFNCGGEFFYCNTSGLFNSAINGTLTSNVTLPCRIKQIINMWQEVGRAMYAPPIAGNITCKSNITGLLLTRDGGENSSSTTETFRPTGGDMKNNWRSELYKYKVVEIK

SEQ ID NO: 9 - protein, 21 aa, Human immunodeficiency virus
RWCVYANVTIRGVLVRYRRCW

SEQ ID NO: 10 - protein, 23 aa, Human immunodeficiency virus
VCWFVYANVTIRGVLVRYNRTCY

SEQ ID NO: 11 - protein, 84 aa, Human immunodeficiency virus
HHMETPLDLLKLNLDERVYIKLRGARTLVGTLQAFDSHCNIVLSVKHCSFNITTDVKDRKQKVNATFYMVFIRGDTVTLISTPS

SEQ ID NO: 12 - protein, 84 aa, Human immunodeficiency virus
HHMETPLDLLKLNLDERVYIKLRGARTLVGTLQAFDSHCNIVLSDKHASFNITTDVKDRKQKVNATFEMVFIRGDTVTLISTPS

SEQ ID NO: 13 - protein, 84 aa, Human immunodeficiency virus
HHMETPLDLLKLNLDERVYIKLRGARTLVGTLQAFDSHCNIVLCDKHASFNITTDVKDRKQKVNATFECVFIRGDTVTLISTPS

SEQ ID NO: 14 - protein, 81 aa, Human immunodeficiency virus
PPVTHDLRVSLEEIYSGCTKVKHCSFNITTDVKDRKQKVNATFYIEVKKGWKEGTKITFPKEGDQTIPADIVFVLKDKPHN

SEQ ID NO: 15 - protein, 81 aa, Human immunodeficiency virus
PPVTHDLRVSTEEIYSGCTKVKHASFNITTDVKDRKQKVNATFYIEVKKGWKEGTKITFPKEGDQTIPADIVFVLKDKPHN

SEQ ID NO: 16 - protein, 81 aa, Human immunodeficiency virus
PPVTHDLRVSTEEIYSGCTKVKHASHNRTTDVKDRKQKVNATFYIEVKKGWKEGTKITFPKEGDQTIPADIVFVLKDKPHN

SEQ ID NO: 17 - protein, 93 aa, Human immunodeficiency virus
SHYDILQAPVISEKAYSAMERGVYSFWVSPGATKTEIKDAIQQAFGVRVIGISVKHCSFNITTDVKDRKQKVNATFAIVRLAEGQSIEALAGQ

SEQ ID NO: 18 - protein, 93 aa, Human immunodeficiency virus
SHYDILQAPVISEKAYSAMERGVYSFWVSPGATKTEIKDAIQQAFGVRVIGISTKHASFNITTDVKDRKQKVNATFAIVRLAEGQSIEALAGQ

SEQ ID NO: 19 - protein, 90 aa, Human immunodeficiency virus
SNVVLIGKKPVMNYVLAALTLLNQGVSEIVIKARGRAISKAVDTVEIVRNRFLPDKIEIKEVKHCSFNITTDVKDRKQKVNATFYAIRKK

SEQ ID NO: 20 - protein, 90 aa, Human immunodeficiency virus
SNVVLIGKKPVMNYVLAALTLLNQGVSEIVIKARGRAISKAVDTVEIVRNRFLPDKIEIKEVKHASFNITTDVKDRKQKVNATFIAIRKK

SEQ ID NO: 21 - protein, 79 aa, Human immunodeficiency virus
PKKVLTGVVVSDKMQKTVVVHCSFNITTDVKDRKQKVNATFYAHDPEEKYKLGDVVEIIESRPISKRKRFRVLRLVESG

SEQ ID NO: 22 - protein, 79 aa, Human immunodeficiency virus
PKKVLTGVVVSDKMQKTVVVHASFNITTDVKDRKQKVNATFYAHDPEEKYKLGDVVEIIESRPISKRKRFRVLRLVESG

SEQ ID NO: 23 - protein, 79 aa, Human immunodeficiency virus
PKKVLTGVVVSDKMQKTVVVHASRNITTDVKDRKQKVNATFYAHDPEEKYKLGDVVEIIESRPISKRKRFRVLRLVESG

SEQ ID NO: 24 - protein, 141 aa, Human immunodeficiency virus
TIGMVVIHKTGHIAAGTSTNGIKFKIHGRVGDSPIPGAGAYADDTAGAAAATGNGDILMRFLPSYQAVEYMRRGEDPTIACQKVISRIQKHFPEFFGAVICANVTGSYGAACNKLSTFTHFSFNITTDVKDRKQKVNATCI

SEQ ID NO: 25 - protein, 133 aa, Human immunodeficiency virus
PPTIQEIKQKIDSYNSREKHCLGMKLSEDGTYTGFIVVHLSLNRTTDVKDRKQKVNATFYMHISSTTTVSEVIQGLLDKFMVVDNPQKFALFKRIHKDGQVLFQKLSIADYPLYLRLLAGPDTDVLSFVLKEN

SEQ ID NO: 26 - protein, 162 aa, Human immunodeficiency virus
MKHISFNITTDVKDRKQKVNATPNKRLLDLLREDFGLTSVKEGCSEGECGACTVIFNGDPVTTCCMLAGQADESTIITLEGVAEDGKPSLLQQCFLEAGAVQCGYCTPGMILTAKALLDKNPDPTDEEITVAMSGNLCRCTGYIKIHAAVRYAVERCANAAA

SEQ ID NO: 27 - protein, 162 aa, Human immunodeficiency virus
MKHISFNITTDVKDRKRKINTTPNKRLLDLLREDFGLTSVKEGCSEGECGACTVIFNGDPVTTCCMLAGQADESTIITLEGVAEDGKPSLLQQCFLEAGAVQCGYCTPGMILTAKALLDKNPDPTDEEITVAMSGNLCRCTGYIKIHAAVRYAVERCANAAA

SEQ ID NO: 28 - protein, 98 aa, Human immunodeficiency virus
GSHVSFNITTDVKDRKQKVNATFYKNQNISYKDLEGKVKSVLESNRGITDVDLRLSKQAKYTVNFKNGTKKVIDLKSGIYTANLINSSDIKSININID

SEQ ID NO: 29 - protein, 105 aa, Human immunodeficiency virus
TNRLVLSGTVCRAPLRKVSPSGIPHCQFVLVHHCSFNITTDVKDRKQKVNATFYMPVIVSGHENQAITHSITVGSRITVQGFISCHKAKNGLSKMVLHAEQIELI

SEQ ID NO: 30 - protein, 105 aa, Human immunodeficiency virus
TNRLVLSGTVCRAPLRKVSPSGIPHCQFVLVHHASFNITTDVKDRKQKVNATFYMPVIVSGHENQAITHSITVGSRITVQGFISCHKAKNGLSKMVLHAEQIELI

SEQ ID NO: 31 - protein, 105 aa, Human immunodeficiency virus
TNRLVLSGTVCRAPLRKVSPSGIPHCQFVCVHHASFNITTDVKDRKQKVNATFYCPVIVSGHENQAITHSITVGSRITVQGFISCHKAKNGLSKMVLHAEQIELI

SEQ ID NO: 32 - protein, 88 aa, Human immunodeficiency virus
PVLENVQPNSAASKAGLQAGDRIVKVDGQPLTQWVTFVMLVRDNPGKHLSLNITTDVKDRKQKVNATPESKPGNGKAIGFVGIEPKVI

SEQ ID NO: 33 - protein, 85 aa, Human immunodeficiency virus
GDHCSINVTTDVKDRKQKVNATSYDKAPTVIRKAMDAHALDEDEPEDYELLQIISEDHKLKIPENANVFYAMNSAANYDFILKKR

SEQ ID NO: 34 - protein, 164 aa, Human immunodeficiency virus
SKHMSFNITTDVKDRKQKVNATPRMHLADALREVVGLTGTKIGCEQGVCGSCTILIDGAPMRSCLTLAVQAEGCSIETVEGLSQGEKLNALQDSFRRHHALQCGFCTAGMLATARSILAENPAPSRDEVREVMSGNLCRCTGYETIIDAITDPAVAEAARRGEV

SEQ ID NO: 35 - protein, 153 aa, Human immunodeficiency virus
TTPPARTAKQRIQDTLNRLELDVHASFNITTDVKDRKQKVNATYLWDGETFLVATPAASPTGRNLSETGRVRLGIGPTRDLVLVEGTALPLEPAGLPDGVGDTFAEKTGFDPRRLTTSYLYFRISPRRVQAWREANELSGRELMRDGEWLVTD

SEQ ID NO: 36 - protein, 162 aa, Human immunodeficiency virus
SDWDPVVKEWLVDTGYCCAGGIANAEDGVVFAAAADDDDGWSKLYKDDHSFNITTDVKDRKQKVNATEASTIKAAVDDGSAPNGVWIGGQKYKVVRPEKGFEYNDCTFDITMCARSKGGAHLIKTPNGSIVIALYDEEKEQDKGNSRTSALAFAEYLHQSGY

SEQ ID NO: 37 - protein, 83 aa, Human immunodeficiency virus
TNPKRSSDYYNRSTSPWNLHRNEDPERYPSVIWEAKCRHLGCINADGNVDYHMNSISQNITTDVKDRKQKVNATCTCVTPIVH

SEQ ID NO: 38 - protein, 125 aa, Human immunodeficiency virus
IVISMPQDFRPVSSIIDVDILPETHRRVRLCKYGTEKPLGFYIRHGSSNRTTDVKDRKQKVNATFISRLVPGGLAQSTGLLAVNDEVLEVNGIEVSGKSLDQVTDMMIANSRNLIITVRPANQRN

SEQ ID NO: 39 - protein, 109 aa, Human immunodeficiency virus
MLNRVFLEGEIESSCWSVKKTGFLVTIKKHCSFNITTDVKDRKQKVNATFYYVIYANGQLAYELEKHTKKYKTISIEGILRTYLERKSEIWKTTIEIVKIFNPKNEIVI

SEQ ID NO: 40 - protein, 109 aa, Human immunodeficiency virus
MLNRVFLEGEIESSCWSVKKTGFLVTIKKHASFNITTDVKDRKQKVNATFYYVIYANGQLAYELEKHTKKYKTISIEGILRTYLERKSEIWKTTIEIVKIFNPKNEIVI

SEQ ID NO: 41 - protein, 109 aa, Human immunodeficiency virus
MLNRVFLEGEIESSTWSVKKTGFLVTCKKHASFNITTDVKDRKQKVNATFYCVIYANGQLAYELEKHTKKYKTISIEGILRTYLERKSEIWKTTIEIVKIFNPKNEIVI

SEQ ID NO: 42 - protein, 79 aa, Human immunodeficiency virus
LTCVTKHCSFNITTDVKDRKQKVNATFYENCPDGQNLCFKRWQYISPRMYDFTRGCAATCPKAEYRDVINCCGTDKCNK

SEQ ID NO: 43 - protein, 79 aa, Human immunodeficiency virus
LTCVTKHASFNITTDVKDRKQKVNATFYENCPDGQNLCFKRWQYISPRMYDFTRGCAATCPKAEYRDVINCCGTDKCNK

SEQ ID NO: 44 - protein, 79 aa, Human immunodeficiency virus
LTCVTCHASFNITTDVKDRKQKVNATCYENCPDGQNLCFKRWQYISPRMYDFTRGCAATCPKAEYRDVINCCGTDKCNK

SEQ ID NO: 45 - protein, 88 aa, Human immunodeficiency virus
MIKVEIKPSQAQFTTRSGVSRQGKPYSLKHQSFNITTDVKDRKQKVNATLDEGQPAYAPGLYTVHLSSFKVGQFGSLMIDRLRLVPAK

SEQ ID NO: 46 - protein, 106 aa, Human immunodeficiency virus
AINRLQLVATLVEREVMRYTPAGVPIVNCLLSYVKHCSFNITTDVKDRKQKVNATFYFSIEALGAGKMASVLDRIAPGTVLECVGFLARKHGSGALVFHISGLEHH

SEQ ID NO: 47 - protein, 106 aa, Human immunodeficiency virus
AINRLQLVATLVEREVMRYTPAGVPIVNCLLSYVKHASFNITTDVKDRKQKVNATFYFSIEALGAGKMASVLDRIAPGTVLECVGFLARKHGSGALVFHISGLEHH

SEQ ID NO: 48 - protein, 106 aa, Human immunodeficiency virus
AINRLQLVATLVEREVMRYTPAGVPIVNCLLSCVKHASFNITTDVKDRKQKVNATFYCSIEALGAGKMASVLDRIAPGTVLECVGFLARKHGSGALVFHISGLEHH

SEQ ID NO: 49 - protein, 94 aa, Human immunodeficiency virus
SMYGVDLHKAKDLEGVDIILGVCSSGLLVYKDKLRINRFPWPKVLKISYKRSHFSINITTDVKDRKQKVNATLPSYRAAKKLWKVCVEHHTFFR

SEQ ID NO: 50 - protein, 113 aa, Human immunodeficiency virus
MDGRIKEVSVFTYHKKYNPDKHYHYSFNITTDVKDRKQKVNATFDEFQELHNKLSIIFPLWKLPGFPNRMVLGRTHIKDVAAKRKIELNSYLQSLMNASTDVAECDLVCTFFH

SEQ ID NO: 51 - protein, 182 aa, Human immunodeficiency virus
DYDYLIKLLALGDSGVGKTTFLYRYTDNKFNPKFITTVGIDVKHCSFNITTDVKDRKQKVNATFYDTAGQERFRSLTTAFFRDAMGFLLMFDLTSQQSFLNVRNWMSQLQANAYCENPDIVLIGNKADLPDQREVNERQARELADKYGIPYFETSAATGQNVEKAVETLLDLIMKRMEQCVE

SEQ ID NO: 52 - protein, 182 aa, Human immunodeficiency virus
DYDYLIKLLALGDSGVGKTTFLYRYTDNKFNPKFITTVGIDVKHASFNITTDVKDRKQKVNATFYDTAGQERFRSLTTAFFRDAMGFLLMFDLTSQQSFLNVRNWMSQLQANAYCENPDIVLIGNKADLPDQREVNERQARELADKYGIPYFETSAATGQNVEKAVETLLDLIMKRMEQCVE

SEQ ID NO: 53 - protein, 182 aa, Human immunodeficiency virus
DYDYLIKLLALGDSGVGKTTFLYRYTDNKFNPKFITTVGICVKHASFNITTDVKDRKQKVNATFYCTAGQERFRSLTTAFFRDAMGFLLMFDLTSQQSFLNVRNWMSQLQANAYCENPDIVLIGNKADLPDQREVNERQARELADKYGIPYFETSAATGQNVEKAVETLLDLIMKRMEQCVE

SEQ ID NO: 54 - protein, 181 aa, Human immunodeficiency virus
GQLTKQHVRALAISALAPKPHETLWDIGGGSGSIAIEWLRSTPQTTAVCFEISEERRERILSNAINLGVSDRIAVQQGAPRAFDDVPDNPDVIFIGGLTAPGVFAAAWKRLPVGGRLVANAVTVESEQMLWALRKQFGGTISSFAIVHHCSFNITTDVKDRKQKVNATFYALPVHQWTVVK

SEQ ID NO: 55 - protein, 181 aa, Human immunodeficiency virus
GQLTKQHVRALAISALAPKPHETLWDIGGGSGSIAIEWLRSTPQTTAVCFEISEERRERILSNAINLGVSDRIAVQQGAPRAFDDVPDNPDVIFIGGLTAPGVFAAAWKRLPVGGRLVANAVTVESEQMLWALRKQFGGTISSFAIVHHASFNITTDVKDRKQKVNATFYALPVHQWTVVK

SEQ ID NO: 56 - protein, 181 aa, Human immunodeficiency virus
GQLTKQHVRALAISALAPKPHETLWDIGGGSGSIAIEWLRSTPQTTAVCFEISEERRERILSNAINLGVSDRIAVQQGAPRAFDDVPDNPDVIFIGGLTAPGVFAAAWKRLPVGGRLVANAVTVESEQMLWALRKQFGGTISSFACVHHASFNITTDVKDRKQKVNATFYCLPVHQWTVVK

SEQ ID NO: 57 - protein, 92 aa, Human immunodeficiency virus
SKMLQHIDYRMRCILQDGRIFIGTFKAFDKHMNLILCDCDEFRVKHCSFNITTDVKDRKQKVNATFYEKRVLGLVLLRGENLVSMTVEGPPP

SEQ ID NO: 58 - protein, 92 aa, Human immunodeficiency virus
SKMLQHIDYRMRCILQDGRIFIGTFKAFDKHMNLILCDCDEFRVKHASFNITTDVKDRKQKVNATFYEKRVLGLVLLRGENLVSMTVEGPPP

SEQ ID NO: 59 - protein, 92 aa, Human immunodeficiency virus
SKMLQHIDYRMRCILQDGRIFIGTFKAFDKHMNLILCDCDEFCVKHASFNITTDVKDRKQKVNATFYCKRVLGLVLLRGENLVSMTVEGPPP

SEQ ID NO: 60 - protein, 196 aa, Human immunodeficiency virus
MIPDDEFIKNPSVPGPTAMEVRCLIMCLAEPGKNDVAVDVGCGTGGVTLELAGRVRRVYAIDRNPEAISTTEMNLQRHGLGDNVTLMEGDAPEALCKIPDIDIAVVGGSGGELQEILRIIKDKLKPGGRIIVTAILLETKFEAMECLRDLGFDVNITELNIVKHCSFNITTDVKDRKQKVNATFYRNPVALIYTGV

SEQ ID NO: 61 - protein, 196 aa, Human immunodeficiency virus
MIPDDEFIKNPSVPGPTAMEVRCLIMCLAEPGKNDVAVDVGCGTGGVTLELAGRVRRVYAIDRNPEAISTTEMNLQRHGLGDNVTLMEGDAPEALCKIPDIDIAVVGGSGGELQEILRIIKDKLKPGGRIIVTAILLETKFEAMECLRDLGFDVNITELNIVKHASFNITTDVKDRKQKVNATFYRNPVALIYTGV

SEQ ID NO: 62 - protein, 196 aa, Human immunodeficiency virus
MIPDDEFIKNPSVPGPTAMEVRCLIMCLAEPGKNDVAVDVGCGTGGVTLELAGRVRRVYAIDRNPEAISTTEMNLQRHGLGDNVTLMEGDAPEALCKIPDIDIAVVGGSGGELQEILRIIKDKLKPGGRIIVTAILLETKFEAMECLRDLGFDVNITELNCVKHASFNITTDVKDRKQKVNATFYCNPVALIYTGV

SEQ ID NO: 63 - protein, 159 aa, Human immunodeficiency virus
SLIRIGHGFDVHAFVKHCSFNITTDVKDRKQKVNATFYFIAHSDGDVALHALTDAILGAAALGDIGKLFPKNADSRGLLREAFRQVQEKGYKIGNVDITIIAQAPKMRPHIDAMRAKIAEDLQCDIEQVNVKATTTEKLGFTGRQEGIACEAVALLIRQ

SEQ ID NO: 64 - protein, 159 aa, Human immunodeficiency virus
SLIRIGHGFDVHAFVKHASFNITTDVKDRKQKVNATFYFIAHSDGDVALHALTDAILGAAALGDIGKLFPKNADSRGLLREAFRQVQEKGYKIGNVDITIIAQAPKMRPHIDAMRAKIAEDLQCDIEQVNVKATTTEKLGFTGRQEGIACEAVALLIRQ

SEQ ID NO: 65 - protein, 159 aa, Human immunodeficiency virus
SLIRIGHGFDVHAFGKHASFNITTDVKDRKQKVNATFGFIAHSDGDVALHALTDAILGAAALGDIGKLFPKNADSRGLLREAFRQVQEKGYKIGNVDITIIAQAPKMRPHIDAMRAKIAEDLQCDIEQVNVKATTTEKLGFTGRQEGIACEAVALLIRQ

SEQ ID NO: 66 - protein, 105 aa, Human immunodeficiency virus
GDTTITVVGNLTADPELRFTPSGAAVANFTVASTGSALFLRCNIWREAAENVAESLTRGSRVIVTGRLKVKHCSFNITTDVKDRKQKVNATFYEVEVDEIGPSLR

SEQ ID NO: 67 - protein, 105 aa, Human immunodeficiency virus
GDTTITVVGNLTADPELRFTPSGAAVANFTVASTGSALFLRCNIWREAAENVAESLTRGSRVIVTGRLKVKHASFNITTDVKDRKQKVNATFYEVEVDEIGPSLR

SEQ ID NO: 68 - protein, 105 aa, Human immunodeficiency virus
GDTTITVVGNLTADPELRFTPSGAAVANFTVASTGSALFLRCNIWREAAENVAESLTRGSRVIVTGRLCVKHASFNITTDVKDRKQKVNATFYCVEVDEIGPSLR

SEQ ID NO: 69 - protein, 96 aa, Human immunodeficiency virus
SGISEVRSDRDKFVIFLDVKHFSPEDLTVKVQEDFVEIHGVKHCSFNITTDVKDRKQKVNATFYFHRRYRLPSNVDQSALSCSLSADGMLTFSGPK

SEQ ID NO: 70 - protein, 96 aa, Human immunodeficiency virus
SGISEVRSDRDKFVIFLDVKHFSPEDLTVKVQEDFVEIHGVKHASFNITTDVKDRKQKVNATFYFHRRYRLPSNVDQSALSCSLSADGMLTFSGPK

SEQ ID NO: 71 - protein, 130 aa, Human immunodeficiency virus
MQDTIFLKGMRFYGYHGALSAENEIGQIFKVDVTLKVDLSEAGRTDNVIDTVHYGEVFEEVKSIMEGKAVNLLEHLAERIANRINSQYNRVMETKVRIVKHCSFNITTDVKDRKQKVNATFYIEIVRENK

SEQ ID NO: 72 - protein, 130 aa, Human immunodeficiency virus
MQDTIFLKGMRFYGYHGALSAENEIGQIFKVDVTLKVDLSEAGRTDNVIDTVHYGEVFEEVKSIMEGKAVNLLEHLAERIANRINSQYNRVMETKVRIVKHASFNITTDVKDRKQKVNATFYIEIVRENK

SEQ ID NO: 73 - protein, 123 aa, Human immunodeficiency virus
EEKRSSTGFLVKQRAFLKLYMITMTEQERLYGLKLLEVLRSEFKEIGFKPNHTEVYRSLHELLDDGIVKHCSFNITTDVKDRKQKVNATFYKDYEAAKLYKKQLKVELDRCKKLIEKALSDNF

SEQ ID NO: 74 - protein, 123 aa, Human immunodeficiency virus
EEKRSSTGFLVKQRAFLKLYMITMTEQERLYGLKLLEVLRSEFKEIGFKPNHTEVYRSLHELLDDGIVKHASFNITTDVKDRKQKVNATFFKDYEAAKLYKKQLKVELDRCKKLIEKALSDNF

SEQ ID NO: 75 - protein, 123 aa, Human immunodeficiency virus
EEKRSSTGFLVKQRAFLKLYMITMTEQERLYGGKLLEVLRSEFKEIGFKPNHTEVYRSLHELLDDGIVKHASFNITTDVKDRKQKVNATFYKDYEAAKLYKKQLKVELDRCKKLIEKALSDNF

SEQ ID NO: 76 - protein, 97 aa, Human immunodeficiency virus
MKTAYDVILAPVLSEKAYAGFAEGKYTFWVHPKATKTEIKNAVETAFKVKVVKVNTKHASFNITTDVKDRKQKVNATFAIVQVAPGQKIEALEGLIG

SEQ ID NO: 77 - protein, 165 aa, Human immunodeficiency virus
KIRIGHGFDVHKFGKHASFNITTDVKDRKQKVNATFGLVAHSDGDVVLHAISDAILGAMALGDIGKHFPDTDAAYKGADSRVLLRHCYALAKAKGFELGNLDVTIIAQAPKMAPHIEDMRQVLAADLNADVADINVKATTTEKLGFTGRKEGIAVEAVVLLSRQG

SEQ ID NO: 78 - protein, 21 aa, Human immunodeficiency virus
RWCVYAYVRIRGVLVRYRRCW

SEQ ID NO: 79 - protein, 96 aa, Human immunodeficiency virus
MHHHHHHMETPLDLLKLNLDERVYIKLRGARTLVGTLQAFDSHCNIVLSDAVETIYQLNNEELSESERRCEMVFIRGDTVTLISTPSEDDDGAVEI

SEQ ID NO: 80 - protein, 183 aa, Human immunodeficiency virus
KKQDPPVTHDLRVSLEEIYSGCTKKMKISHKRLNPDGKSIRNEDKILTIEVKKGWKEGTKITFPKEGDQTSNNIPADIVFVLKDKPHNIFKRDGSDVIYPARISLREALCGCTVNVPTLDGRTIPVVFKDVIRPGMRRKVPGEGLPLPKTPEKRGDLIIEFEVIFPERIPQTSRTVLEQVLPI

SEQ ID NO: 81 - protein, 95 aa, Human immunodeficiency virus
MSHYDILQAPVISEKAYSAMERGVYSFWVSPKATKTEIKDAIQQAFGVRVIGISTMNVPGKRKRVGRFIGQRNDRKKAIVRLAEGQSIEALAGQA

SEQ ID NO: 82 - protein, 97 aa, Human immunodeficiency virus
MSSGTPTPSNVVLIGKKPVMNYVLAALTLLNQGVSEIVIKARGRAISKAVDTVEIVRNRFLPDKIEIKEIRVGSQVVTSQDGRQSRVSTIEIAIRKK

SEQ ID NO: 83 - protein, 528 aa, Human immunodeficiency virus
PVSVDGETLTVEAVRRVAEERATVDVPAESIAKAQKSREIFEGIAEQNIPIYGVTTGYGEMIYMQVDKSKEVELQTNLVRSHSAGVGPLFAEDEARAIVAARLNTLAKGHSAVRPIILERLAQYLNEGITPAIPEIGSLGASGDLAPLSHVASTLIGEGYVLRDGRPVETAQVLAERGIEPLELRFKEGLALINGTSGMTGLGSLVVGRALEQAQQAEIVTALLIEAVRGSTSPFLAEGHDIARPHEGQIDTAANMRALMRGSGLTVEHADLRRELQKDKEAGKDVQRSEIYLQKAYSLRAIPQVVGAVRDTLYHARHKLRIELNSANDNPLFFEGKEIFHGANFHGQPIAFAMDFVTIALTQLGVLAERQINRVLNRHLSYGLPEFLVSGDPGLHSGFAGAQYPATALVAENRTIGPASTQSVPSNGDNQDVVSMGLISARNARRVLSNNNKILAVEYLAAAQAVDISGRFDGLSPAAKATYEAVRRLVPTLGVDRYMADDIELVADALSRGEFLRAIARETDIQLR

SEQ ID NO: 84 - protein, 141 aa, Human immunodeficiency virus
TIGMVVIHKTGHIAAGTSTNGIKFKIHGRVGDSPIPGAGAYADDTAGAAAATGNGDILMRFLPSYQAVEYMRRGEDPTIACQKVISRIQKHFPEFFGAVICANVTGSYGAACNKLSTFTQFSFMVYNSEKNQPTEEKVDCI

SEQ ID NO: 85 - protein, 163 aa, Human immunodeficiency virus
GSPEFPPTIQEIKQKIDSYNSREKHCLGMKLSEDGTYTGFIKVHLKLRRPVTVPAGIRPQSIYDAIKEVNPAATTDKRTSFYLPLDAIKQMHISSTTTVSEVIQGLLDKFMVVDNPQKFALFKRIHKDGQVLFQKLSIADYPLYLRLLAGPDTDVLSFVLKEN

SEQ ID NO: 86 - protein, 160 aa, Human immunodeficiency virus
MNKITINLNLNGEARSIVTEPNKRLLDLLREDFGLTSVKEGCSEGECGACTVIFNGDPVTTCCMLAGQADESTIITLEGVAEDGKPSLLQQCFLEAGAVQCGYCTPGMILTAKALLDKNPDPTDEEITVAMSGNLCRCTGYIKIHAAVRYAVERCANAAA

SEQ ID NO: 87 - protein, 98 aa, Human immunodeficiency virus
GSTVPYTITVNGTSQNILSNLTFNKNQNISYKDLEGKVKSVLESNRGITDVDLRLSKQAKYTVNFKNGTKKVIDLKSGIYTANLINSSDIKSININID

SEQ ID NO: 88 - protein, 103 aa, Human immunodeficiency virus
TNRLVLSGTVCRAPLRKVSPSGIPHCQFVLEHRSVQEEAGFHRQAWCQMPVIVSGHENQAITHSITVGSRITVQGFISCHKAKNGLSKMVLHAEQIELIDSGD

SEQ ID NO: 89 - protein, 91 aa, Human immunodeficiency virus
GIPIEPVLENVQPNSAASKAGLQAGDRIVKVDGQPLTQWVTFVMLVRDNPGKSLALEIERQGSPLSLTLIPESKPGNGKAIGFVGIEPKVI

SEQ ID NO: 90 - protein, 87 aa, Human immunodeficiency virus [sequence omitted]

SEQ ID NO: 91 - protein, 168 aa, Human immunodeficiency virus
MQAHEESQLMRISATINGKPRVFYVEPRMHLADALREVVGLTGTKIGCEQGVCGSCTILIDGAPMRSCLTLAVQAEGCSIETVEGLSQGEKLNALQDSFRRHHALQCGFCTAGMLATARSILAENPAPSRDEVREVMSGNLCRCTGYETIIDAITDPAVAEAARRGEV

SEQ ID NO: 92 - protein, 155 aa, Human immunodeficiency virus
GMTTPPARTAKQRIQDTLNRLELDVDAWVSTAGADGGAPYLVPLSYLWDGETFLVATPAASPTGRNLSETGRVRLGIGPTRDLVLVEGTALPLEPAGLPDGVGDTFAEKTGFDPRRLTTSYLYFRISPRRVQAWREANELSGRELMRDGEWLVTD

SEQ ID NO: 93 - protein, 166 aa, Human immunodeficiency virus
GSHMSDWDPVVKEWLVDTGYCCAGGIANAEDGVVFAAAADDDDGWSKLYKDDHEEDTIGEDGNACGKVSINEASTIKAAVDDGSAPNGVWIGGQKYKVVRPEKGFEYNDCTFDITMCARSKGGAHLIKTPNGSIVIALYDEEKEQDKGNSRTSALAFAEYLHQSGY

SEQ ID NO: 94 - protein, 137 aa, Human immunodeficiency virus
MIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 95 - protein, 128 aa, Human immunodeficiency virus
RKKPHIVISMPQDFRPVSSIIDVDILPETHRRVRLCKYGTEKPLGFYIRDGSSVRVTPHGLEKVPGIFISRLVPGGLAQSTGLLAVNDEVLEVNGIEVSGKSLDQVTDMMIANSRNLIITVRPANQRN

SEQ ID NO: 96 - protein, 110 aa, Human immunodeficiency virus
GGGGGGMLNRVFLEGEIESSCWSVKKTGFLVTIKQMRFFGERLFTDYYVIYANGQLAYELEKHTKKYKTISIEGILRTYLERKSEIWKTTIEIVKIFNPKNEIVIDYKEI

SEQ ID NO: 97 - protein, 65 aa, Human immunodeficiency virus
LTCVTSKSIFGITTENCPDGQNLCFKRWQYISPRMYDFTRGCAATCPKAEYRDVINCCGTDKCNK

SEQ ID NO: 98 - protein, 87 aa, Human immunodeficiency virus
MIKVEIKPSQAQFTTRSGVSRQGKPYSLNEQLCYVDLGNEYPVLVKITLDEGQPAYAPGLYTVHLSSFKVGQFGSLMIDRLRLVPAK

SEQ ID NO: 99 - protein, 101 aa, Human immunodeficiency virus
AINRLQLVATLVEREVMRYTPAGVPIVNCLLSYSGQAMEAQAARQVEFSIEALGAGKMASVLDRIAPGTVLECVGFLARKHRSSKALVFHISGLEHHHHHH

SEQ ID NO: 100 - protein, 279 aa, Human immunodeficiency virus
MHCKVSLLDDTVYECVVEKHAKGQDLLKRVCEHLNLLEEDYFGLAIWDNATSKTWLDSAKEIKKQVRGVPWNFTFNVKFYPPDPAQLTEDITRYYLCLQLRQDIVAGRLPCSFATLALLGSYTIQSELGDYDPELHGVDYVSDFKLAPNQTKELEEKVMELHKSYRSMTPAQADLEFLENAKKLSMYGVDLHKAKDLEGVDIILGVCSSGLLVYKDKLRINRFPWPKVLKISYKRSSFFIKIRPGEQEQYESTIGFKLPSYRAAKKLWKVCVEHHTFFR

SEQ ID NO: 101 - protein, 121 aa, Human immunodeficiency virus
MDGRIKEVSVFTYHKKYNPDKHYIYVVRILREGQIEPSFVFRTFDEFQELHNKLSIIFPLWKLPGFPNRMVLGRTHIKDVAAKRKIELNSYLQSLMNASTDVAECDLVCTFFHGSHHHHHH

SEQ ID NO: 102 - protein, 217 aa, Human immunodeficiency virus
MGSSHHHHHHSSGLVPRGSGDYDYLIKLLALGDSGVGKTTFLYRYTDNKFNPKFITTVGIDFREKRVVYNAQGPNGSSGKAFKVHLQLWDTAGQERFRSLTTAFFRDAMGFLLMFDLTSQQSFLNVRNWMSQLQANAYCENPDIVLIGNKADLPDQREVNERQARELADKYGIPYFETSAATGQNVEKAVETLLDLIMKRMEQCVEKTQIPDTVNGG

SEQ ID NO: 103 - protein, 178 aa, Human immunodeficiency virus
SNATDGQLTKQHVRALAISALAPKPHETLWDIGGGSGSIAIEWLRSTPQTTAVCFEISEERRERILSNAINLGVSDRIAVQQGAPRAFDDVPDNPDVIFIGGGLTAPGVFAAAWKRLPVGGRLVANAVTVESEQMLWALRKQFGGTISSFAISHEHTVGSFITMKPALPVHQWTVVKA

SEQ ID NO: 104 - protein, 91 aa, Human immunodeficiency virus
MTVGKSSKMLQHIDYRMRCILQDGRIFIGTFKAFDKHMNLILCDCDEFRKIKPKNSKQAEREEKRVLGLVLLRGENLVSMTVEGPPPKDTG

SEQ ID NO: 105 - protein, 192 aa, Human immunodeficiency virus
MIPDDEFIKNPSVPGPTAMEVRCLIMCLAEPGKNDVAVDVGCGTGGVTLELAGRVRRVYAIDRNPEAISTTEMNLQRHGLGDNVTLMEGDAPEALCKIPDIDIAVVGGSGGELQEILRIIKDKLKPGGRIIVTAILLETKFEAMECLRDLGFDVNITELNIARGRALDRGTMMVSRNPVALIYTGVSHENKD

SEQ ID NO: 106 - protein, 170 aa, Human immunodeficiency virus
MSLIRIGHGFDVHAFGEDRPLIIGGVEVPYHTGFIAHSDGDVALHALTDAILGAAALGDIGKLFPDTDMQYKNADSRGLLREAFRQVQEKGYKIGNVDITIIAQAPKMRPHIDAMRAKIAEDLQCDIEQVNVKATTTEKLGFTGRQEGIACEAVALLIRQEGGSHHHHHH

SEQ ID NO: 107 - protein, 165 aa, Human immunodeficiency virus
MAGDTTITVVGNLTADPELRFTPSGAAVANFTVASTPRMFDRQSGEWKDGEALFLRCNIWREAAENVAESLTRGSRVIVTGRLKQRSFETREGEKRTVVEVEVDEIGPSLRYATAKVNKASRSGGGGGGFGSGGGGSRQSEPKDDPWGSAPASGSFSGADDEPPF

SEQ ID NO: 108 - protein, 106 aa, Human immunodeficiency virus
GSGISEVRSDRDKFVIFLDVKHFSPEDLTVKVQEDFVEIHGKHNERQDDHGYISREFHRRYRLPSNVDQSALSCSLSADGMLTFSGPKIPSGVDAGHSERAIPVSR

SEQ ID NO: 109 - protein, 121 aa, Human immunodeficiency virus
MQDTIFLKGMRFYGYHGALSAENEIGQIFKVDVTLKVDLSEAGRTDNVIDTVHYGEVFEEVKSIMEGKAVNLLEHLAERIANRINSQYNRVMETKVRITKENPPIPGHYDGVGIEIVRENK

SEQ ID NO: 110 - protein, 122 aa, Human immunodeficiency virus
MKEEKRSSTGFLVKQRAFLKLYMITMTEQERLYGLKLLEVLRSEFKEIGFKPNHTEVYRSLHELLDDGILKQIKVKKEGAKLQEVVLYQFKDYEAAKLYKKQLKVELDRCKKLIEKALSDNF

SEQ ID NO: 111 - protein, 92 aa, Human immunodeficiency virus
TAYDVILAPVLSEKAYAGFAEGKYTFWVHPKATKTEIKNAVETAFKVKVVKVNTLHVRGKKKRLGRYLGKRPDRKKAIVQVAPGQKIEALEG

SEQ ID NO: 112 - protein, 159 aa, Human immunodeficiency virus
MKIRIGHGFDVHKFGEPRPLILCGVEVPYETGLVAHSDGDVVLHAISDAILGAMALGDIGKHFPDTDAAYKGADSRVLLRHCYALAKAKGFELGNLDVTIIAQAPKMAPHIEDMRQVLAADLNADVADINVKATTTEKLGFTGRKEGIAVEAVVLLSRQ

SEQ ID NO: 113 - protein, 142 aa, Human immunodeficiency virus
QCVTLRCTNATINGSLTEEVKNCSFNITTELRDKKQKAYALFYRPDVVPLNKNSPSGNSSEYILINCGGSGGSGGCVTLRCTNATINGSLTEEVKNCSFNITTELRDKKQKAYALFYRPDVVPLNKNSPSGNSSEYILINCL

SEQ ID NO: 114 - protein, 149 aa, Human immunodeficiency virus
QCVTLNCSDATYNNGTNSTDTMKICSFNATTELRDKKKKEYALFYRLDIVPLKNESESQNFSEYILINCGGSGGSGGSGGCVTLNCSDATYNNGTNSTDTMKICSFNATTELRDKKKKEYALFYRLDIVPLKNESESQNFSEYILINCL

SEQ ID NO: 115 - protein, 170 aa, Human immunodeficiency virus
QCVTLHCTNANLTKANLTNVNNRTNVSNIIGNITDEVNCSFNMTTELRDKKQKVHALFYKLDIVPIEDNNDNSKYRLINCGGSGGSGGSGGCVTLHCTNANLTKANLTNVNNRTNVSNIIGNITDEVNCSFNMTTELRDKKQKVHALFYKLDIVPIEDNNDNSKYRLINC

SEQ ID NO: 116 - protein, 139 aa, Human immunodeficiency virus
QLCVTLDCSTYNNTHNISKEMKICSFNMTTELRDKKRKVNVLFYKLDLVPLTNSSNTTNYRLISCGGSGGGSGGCVTLDCSTYNNTHNISKEMKICSFNMTTELRDKKRKVNVLFYKLDLVPLTNSSNTTNYRLISCNT

SEQ ID NO: 117 - protein, 127 aa, Human immunodeficiency virus
QCVTLHCTNAGGSGDEVNCSFNMTTELRDKKQKVHALFYKLDGGSGGSGGSKYRLINCGGSGGSGGSGGQCVTLHCTNAGGSGDEVNCSFNMTTELRDKKQKVHALFYKLDGGSGGSGGSKYRLINC

SEQ ID NO: 118 - protein, 127 aa, Human immunodeficiency virus
QLCVTLDCSTGGSGGEMKICSFNMTTELRDKKRKVNVLFYKLDGGSGGSGGTNYRLISCGGSGGGSGGCVTLDCSTGGSGGEMKICSFNMTTELRDKKRKVNVLFYKLDGGSGGSGGTNYRLISCNT

SEQ ID NO: 119 - protein, 167 aa, Helicobacter pylori
MLSKDIIKLLNEQVNKEMNSSNLYMSMSSWCYTHSLDGAGLFLFDHAAEEYEHAKKLIVFLNENNVPVQLTSISAPEHKFEGLTQIFQKAYEHEQHISESINNIVDHAIKGKDHATFNFLQWYVAEQHEEEVLFKDILDKIELIGNENHGLYLADQYVKGIAKSRKS

SEQ ID NO: 120 - protein, 191 aa, Human immunodeficiency virus
MLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIGVKHSSFNITTDVKDRKQKVNATFYGKDHATFNFLQWYVAEQHEEEVLFKDILDKIELIGNENHGLYLADQYVKGIAKSRKS

SEQ ID NO: 121 - protein, 191 aa, Human immunodeficiency virus
MLSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIGVKNSSFNITTELRDKKQKAYALFYGKDHATFNFLQWYVAEQHEEEVLFKDILDKIELIGNENHGLYLADQYVKGIAKSRKS

SEQ ID NO: 122 - protein, 220 aa, Human immunodeficiency virus
MDSKGSSQKGSRLLLLLVVSNLLLPQGVLALSKDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIGVRNSSFNMTTELRDKKQKVHALFYGKDHATFNFLQWYVAEQHEEEVLFKDILDKIELIGNENHGLYLADQYVKGIAKSRKS

SEQ ID NO: 123 - protein, 322 aa, Human immunodeficiency virus
QCVTLRCTNATINGSLTEEVKNCSFNITTELRDKKQKAYALFYRPDVVPLNKNSPSGNSSEYILINCGGSGGSGGCVTLRCTNATINGSLTEEVKNCSFNITTELRDKKQKAYALFYRPDVVPLNKNSPSGNSSEYILINCLGGGSGGGSGGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 124 - protein, 322 aa, Human immunodeficiency virus
QCVTLNCTSPAAHNESETRVKHCSFNITTDVKDRKQKVNATFYDLDIVPLSSSDNSSNSSLYRLISCGGSGGSGGCVTLNCTSPAAHNESETRVKHCSFNITTDVKDRKQKVNATFYDLDIVPLSSSDNSSNSSLYRLISCLGGGSGGGSGGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 125 - protein, 374 aa, Human immunodeficiency virus
MRPTWAWWLFLVLLLALWAPARGQCVTLHCTNANLTKANLTNVNNRTNVSNIIGNITDEVNCSFNMTTELRDKKQKVHALFYKLDIVPIEDNNDNSKYRLINCGGSGGSGGSGGCVTLHCTNANLTKANLTNVNNRTNVSNIIGNITDEVNCSFNMTTELRDKKQKVHALFYKLDIVPIEDNNDNSKYRLINCLGGGSGGGSGGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 126 - protein, 398 aa, Human immunodeficiency virus
QCVTLHCTNAGGSGDEVNCSFNMTTELRDKKQKVHALFYKLDGGSGGSGGSKYRLINCGGSGGSGGSGGQCVTLHCTNAGGSGDEVNCSFNMTTELRDKKQKVHALFYKLDGGSGGSGGSKYRLINCGGSGGSGGSGGCVTLHCTNANLTKANLTNVNNRTNVSNIIGNITDEVNCSFNMTTELRDKKQKVHALFYKLDIVPIEDNNDNSKYRLINCLGGGSGGGSGGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 127 - protein, 335 aa, Human immunodeficiency virus
QCVTLRCTNATINGSLTEEVKNCSFNITTELRDKKQKAYALFYRPDVVPLNKNSPSGNSSEYILINCGGSGGSGGCVTLHCTNANLTKANLTNVNNRTNVSNIIGNITDEVNCSFNMTTELRDKKQKVHALFYKLDIVPIEDNNDNSKYRLINCLGGGSGGGSGGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 128 - protein, 265 aa, Escherichia coli [sequence omitted]

SEQ ID NO: 129 - protein, 277 aa, Human immunodeficiency virus
MEFLKRSFAPLTEKQWQEIDNRAREIFKTQLYGRKFVDVEGPYGWEYAAHPLGEVEVVKHSSFNITTDVKDRKQKVNATFGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLK

SEQ ID NO: 130 - protein, 277 aa, Human immunodeficiency virus
MEFLKRSFAPLTEKQWQEIDNRAREIFKTQLYGRKFVDVEGPYGWEYAAHPLGEVEVVKNSSFNITTELRDKKQKAYALFGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLK

SEQ ID NO: 131 - protein, 277 aa, Human immunodeficiency virus
MEFLKRSFAPLTEKQWQEIDNRAREIFKTQLYGRKFVDVEGPYGWEYAAHPLGEVEVVRNSSFNMTTELRDKKQKVHALFGLRKSLPLIELRATFTLDLWELDNLERGKPNVDLSSLEETVRKVAEFEDEVIFRGCEKSGVKGLLSFEERKIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLK

SEQ ID NO: 132 - protein, 24 aa, Human immunodeficiency virus
  MISC_FEATURE (1)..(1): X is I, M, V, or A
XCNSXXNXXXXXXXXXXXXXXLCY

SEQ ID NO: 133 - protein, 24 aa, Human immunodeficiency virus
  MISC_FEATURE (1)..(1): X is I, M, V, or A
XCXSXXNXXXXXXXXXXXNXXLCY

SEQ ID NO: 134 - protein, 24 aa, Human immunodeficiency virus
VCNSSFNITTELRDKKQKAYALCY

SEQ ID NO: 135 - protein, 24 aa, Human immunodeficiency virus
VCHSSFNITTDVKDRKQKVNATCY

SEQ ID NO: 136 - protein, 42 aa, Human immunodeficiency virus
MCMPCFTTDHQMARKCDDCCGGKGRGKCACVGAGSCCTCLCR

SEQ ID NO: 137 - protein, 53 aa, Human immunodeficiency virus
DKCKKVYENYPVSKCQLANQCNYDCKLDKHARSGECFCVGAGSCQCICDYCEY

SEQ ID NO: 138 - protein, 56 aa, Human immunodeficiency virus
AHMDCTEFNPLCRCNKMLGDLICACVGAGSCQTHRNMCALCCEHPGGFEYSNGPCE

SEQ ID NO: 139 - protein, 58 aa, Human immunodeficiency virus
MTTFKLAACVGAGSCQTTTTEAVDAATAEKVFKQYANDNGIDGEWTYDDATKTFTVTE

SEQ ID NO: 140 - protein, 64 aa, Human immunodeficiency virus
GPWATALYDYDAAEDNELTFKEGDKIINIEFVDDDWWLGELECVGAGSCGSKGLFPSNYVSLGN

SEQ ID NO: 141 - protein, 62 aa, Human immunodeficiency virus
TGKELVLVLYDYQEKSPRELTVKKGDILTLLNSTNKDWWKVECVGAGSCQGFIPAAYLKKLD

SEQ ID NO: 142 - protein, 67 aa, Human immunodeficiency virus
LECHNQQSSQTPTTTGCSGGENNCYKKEWRLCVGAGSCNYRTERGCGCPSVKKGIGINCCTTDRCNN

SEQ ID NO: 143 - protein, 69 aa, Human immunodeficiency virus
KSIWCSPQEIMAADGMPGSVAGVHYRANVQGWTKRKFCVGAGSCTVEYDVMSMPTKEREQVIAHLGLST

SEQ ID NO: 144 - protein, 81 aa, Human immunodeficiency virus
MQIFVKTLTGKTITLEVEPSDTIENVKAKIQDKEGIPPDQQRLIACVGAGSCQQLEDGRTLSDYNIQKESTLHLVLRLRGG

SEQ ID NO: 145 - protein, 90 aa, Human immunodeficiency virus
GAMGSSTSGLKTTKIKFYLCVGAGSCNIFALMLKGDTTYKELRSKIAPRIDTDNFKLQTKLFDGSGEEIKTDSQVSNIIQAKLKISVHPI

SEQ ID NO: 146 - protein, 92 aa, Human immunodeficiency virus
TGCSVTATRAEEWSDRFNVTYSVSGSSAWTVNLALNGSQTIQASWNANVTTDCVGAGSCTRTVTPNGSGNTFGVTVMKNGSSTTPAATCAGS

SEQ ID NO: 147 - protein, 143 aa, Human immunodeficiency virus
LDARQFLIYNEDHKRCVDAVGSCVGAGSCYFFVQTATCNPEAESQKFRWVSDSQIMSVAFKLCLGVPSKTDWASVTLYACDSKSEYQKWECKNDTLFGIKGTELYFNYGNRQEKNIKLYKGSGLWSRWKVYGTTDDLCSRGYE

SEQ ID NO: 148 - protein, 179 aa, Human immunodeficiency virus
EVVLLDFAAAGGELGWLTHPYGKGWDLMQVLVCVGAGSCLFVYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKC

SEQ ID NO: 149 - protein, 548 aa, Human immunodeficiency virus
GTTGMPQYSTFHSENRDWTFNHLTVHRRTGAVYVGAINRVYKLTGNLTIQVAHKTGPEEDNKACYPPLIVQPCSEVLTLTNNVNKLLIIDYSENRLLACGSLYQGVCKLLRLDDLFILVEPSHKKEHYLSSVNKTGTMYGVIVRSEGEDGKLFIGTAVDGKQDYFPTLSSRKLPRDPESSAMLDYELHSDFVSSLIKIPSDTLALVSHFDIFYIYGFASGGFVYFLTVQPETPDGMAINSAGDLFYTSRIVRLCKDDPKFHSYVSLPFGCWYEDCVGAGSCDRVFYRLLQAAYLAKPGEALAQAFNISSDEDVLFAIFSKGQKQYHHPPDDSALCAFPIRAINLQIKERLQSCYHGEGNLELNWLLGKDVQCTKAPVPIDDNFCGLDINQPLGGSTPVEGLTLYTTSRDRLTSVASYVYNGYSVVFVGTKSGKLKKIRADGPPHGGVQYEMVSVFKDGSPILRDMAFSINQLYLYVMSERQVTRVPVESCEQYTTCGECLSSGDPHCGWCALHNMCSRRDKCQRAWEANRFAASISQCMSSRENLYFQ

SEQ ID NO: 150 - protein, 558 aa, Human immunodeficiency virus
LPTLGPGWQRQNPDPPVSRTRSLLLDAASGQLRLEDGFHPDAVAWANLTNAIRETGWAYLDLSTNGRYNDSLQAYAAGVVEASVSEELIYMHWMNTVVNYCGPFEYEVGYCEKLKNFLEANLEWMQREMELNPDSPYWHQVRLTLLQLKGLEDSYEGRLTFPTGRFTIKPLGFLLLQISGDLEDLEPALNKTNTKPSLGSGSSALIKLYAWCVGAGSCLGMLLVAHNTWNSYQNMLRIIKKYRLQFREGPQEEYPLVAGNNLVFSSYPGTIFSGDDFYILGSGLVTLETTIGNKNPALWKYVQPQGCVLEWIRNVVANRLALDGATWADVFKRFNSGTYNNQWMIVDYKAFLPGGPSPGSRVLTILEQIPGMVVVADKTAELYKTTYWASYNIPYFETVFNASGLQALVAQYGDWFSYTKNPRAKIFQRDQSLVEDMDAMVRLMRYNDFLHDPLSLCEACNPKPNAENAISARSDLNPANGSYPFQALHQRAHGGIDVKVTSFTLAKYMSMLAASGPTWDQCPPFQWSKSPFHSMLHMGQPDLWMFSPIRVPWDGRGS

SEQ ID NO: 151 - protein, 584 aa, Human immunodeficiency virus
AKLGSVYTEGGFVEGVNKDGTCVGAGSCLVPVDIFKGIPFAAAPKALEKPERHPGWQGTLKAKSFKKRCLQATLTQDSTYGNEDCLYLNIWVPQGRKEVSHDLPVMIWIYGGAFLMGASQGANFLSNYLYDGEEIATRGNVIVVTFNYRVGPLGFLSTGDSNLPGNYGLWDQHMAIAWVKRNIEAFGGDPDQITLFGESAGGASVSLQTLSPYNKGLIKRAISQSGVGLCPWAIQQDPLFWAKRIAEKVGCPVDDTSKMAGCLKITDPRALTLAYKLPLGSTEYPKLHYLSFVPVIDGDFIPDDPVNLYANAADVDYIAGTNDMDGHLFVGMDVPAINSNKQDVTEEDFYKLVSGLTVTKGLRGAQATYEVYTEPWAQDSSQETRKKTMVDLETDILFLIPTKIAVAQHKSHAKSANTYTYLFSQPSRMPIYPKWMGADHADDLQYVFGKPFATPLGYRAQDRTVSKAMIAYWTNFARTGDPNTGHSTVPANWDPYTLEDDNYLEINKQMDSNSMKLHLRTNYLQFWTQTYQALPTVTSAGASLLPPEDNSQASPVPPADNSGAPTEPSAGDSEVAQMPVVIGF

SEQ ID NO: 152 - protein, 4 aa, Human immunodeficiency virus
GGSG

SEQ ID NO: 153 - protein, 8 aa, Human immunodeficiency virus
GGSGGSGG

SEQ ID NO: 154 - protein, 857 aa, Human immunodeficiency virus
MRVMGIERNYPCWWTWGIMILGMIIICNTAENLWVTVYYGVPIWKDANTTLFCASDAKAYDTEVHNVWATHACVPTDPSPQELKMENVTEEFNMWKNNMVEQMHTDIISLWDQSLKPCVQLTPLCVTLDCSYNITNNITNSITNSSVNMREEIKNCSFNMTTELRDKNRKVYSLFYKLDVVQINNGNNSSNLYRLINCNTSALTQARPKVTFEPIPIHYCAPAGYAILKCNDKEFNGTGLCKNVSTVQCTHGIRPVVSTQLLLNGSLAEGKVMIRSENITNNVKNIIVQLNESVTINCTRPNNNTRRSVRIGPGQTFYATGDIIGDIRQAHCNVSGSQWNKTLHQVVEQLRKYWNNNTIIFNSSSGGDLEITTHSFNCAGEFFYCNTSGLFNSTWVNGTTSSTSNGTITLPCRIKQIINMWQRVGQAMYAPPIQGVIKCESNITGLILTRDGGVNSSDSETFRPGGGDMRDNWRSELYKYKVVKIEPLGVAPTKARRRVVEREKRAVTLGAVFIGFLGTAGSTMGAASITLTVQARKLLSGIVQQQSNLLRAIEAQQHLLKLTVWGIKQLQARVLAVERYLRDQQLLGIWGCSGKLICPTNVPWNSSWSNKSLDEIWENMTWLQWDKEISNYTIKIYELIEESQIQQERNEKDLLELDKWASLWNWFDISKWLWYIKIFIMIVGGLIGLRIVFAVLSVINRVRQGYSPLSFQTHTPNPRGLDRPGRIEEEGGEQDRGRSIRLVSGFLALAWDDLRNLCLFSYHRLRDFILIAARTVELPGHSSLKGLRLGWEGLKYLGNLLLYWGRELKISAINLLDTIAIAVAGWTDRVIETVQRLGRAILNIPRRIRQGFERALL

SEQ ID NO: 155 - protein, 846 aa, Human immunodeficiency virus
MRVRGIQTSWQNLWRWGTMILGMLMIYSAAENLWVTVYYGVPVWKDAETTLFCASDAKAYDTEVHNVWATHACVPTDPNPQEIHLENVTEDFNMWKNNMVEQMHTDIISLWDQSLKPCVKLTPLCVTLDCNATASNVTNEMRNCSFNITTELKDKKQQVYSLFYKLDVVQINEKNETDKYRLINCNTSAITQACPKVSFEPIPIHYCAPAGFAILKCKDTEFNGTGPCKNVSTVQCTHGIRPVISTQLLLNGSLAEEGIQIRSENITNNAKTIIVQLDKAVKINCTRPNNNTRKGVRIGPGQAFYATGGIIGDIRQAHCNVSRAKWNDTLRGVAKKLREHFKNKTIIFEKSSGGDIEITTHSFNCGGEFFYCNTSGLFNSTWESNSTESNNTTSNDTITLTCRIKQIINMWQKVGQAMYAPPIQGVIRCESNITGLLLTRDGGNNSTNEIFRPGGGNMRDNWRSELYKYKVVKIEPLGVAPSRAKRRVVEREKRAVGIGAVFLGFLGAAGSTMGAASITLTAQARQLLSGIVQQQSNLLRAIEAQQHMLKLTVWGIKQLQARVLAVERYLKDQQLLGIWGCSGKLICTTNVPWNSSWSNKSMNEIWDNMTWLQWDKEISNYTQIIYNLIEESQNQQEKNEQDLLALDKWASLWNWFDISRWLWYIKIFIMIVGGLIGLRIVFAVLSVINRVRQGYSPLSFQIRTPNPKEPDRLGRIDGEGGEQDRDRSIRLVSGFLALAWDDLRSLCLFSYHRLRDFISIAARTVELLGHSSLKGLRLGWEGLKYLWNLLLYWGRELKTSAVNLVDTIAIAVAGWTDRVIEVGQRIFRAILNIPRRIRQGLERGLL

SEQ ID NO: 156 - protein, 848 aa, Human immunodeficiency virus
MRVKGIRKNYQHLWKGGILLLGTLIICSAVEKLWVTVYYGVPVWKETTTTLFCASDAKAYDTEVHNVWATHACVPTDPNPQEVVLENVTEDFNMWKNNMVEQMQEDVINLWDQSLKPCVKLTPLCVTLNCKDVNATNTTSSSEGMMERGEIKNCSFNITKSIRNKVQKEYALFYKLDVVPIDNKNNTKYRLISCNTSVITQACPKVSFEPIPIHYCAPAGFAILKCNNKTFNGKGQCKNVSTVQCTHGIRPVVSTQLLLNGSLAEEKVVIRSDNFTDNAKTIIVQLNESVKINCTRPNNNTRKSIHIGPRRAFYTTGEIIGDIRQAHCNISRAQWNNTLKQIVEKLREQFNNKTIVFTHSSGGDPEIVMHSFNCGGEFFYCNSTQLFNSTWNDTEKSSGTEGNDTIILPCRIKQIINMWQEVGKAMYAPPIKGQIRCSSNITGLLLTRDGGKNESEIEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHMLQLTVWGIKQLQARVLAVERYLKDQQLMGIWGCSGKLICTTAVPWNTSWSNKSLDSIWNNMTWMEWEKEIENYTNTIYTLIEESQIQQEKNEQELLELDKWASLWNWFDITKWLWYIKIFIMIVGGLIGLRIVFSVLSIVNRVRQGYSPLSFQTLLPATRGPDRPEGIEEEGGERDRDRSGQLVNGFLALIWVDLRSLFLFSYHRLRDLLLTVTRIVELLGRRGWEILKYWWNLLQYWSQELKNSAVSLLNATAIAVAEGTDRIIEVVQRVYRAILHIPTRIRQGLERALL

SEQ ID NO: 157 - protein, 855 aa, Human immunodeficiency virus
MKVKGIRRNYQHLWRWGIMLLGILMICSATEKLWVTVYYGVPVWKEATTTLFCASDAKAYDQEIHNIWATHACVPTDPNPQEVELKNVTENFNMWKSNMVEQMHEDIISLWDQSLKPCVKLTPLCVTLKCTDLNVTNSNSTDHSTNSSLETKGEIKNCSFNITTTPRDKIQKEYAIFYKQDVVPIKNDNISYRLISCNTSVITQACPKVTFEPIPIHYCAPAGFAILKCNDKGFNGTGPCTNVSTVQCTHGIRPVISTQLLLNGSLAEDKVVIRSENFTDNAKIIIVHLNETVKINCTRPNNNTRKSIHIAPGRAFYATGEIIGDIRKAYCTINESEWNNTLQKIVVTLREQFRNKTIVFNQSSGGDPEVTMHTFNCGGEFFYCNTAQLFNSSWDTNTNGNDTQGPSENNTIILPCRIKQIINMWQRVGKAIYAPPISGQIRCLSNITGLILTRDGGNSSLSSPEIFRPGGGDMRDNWRSELYKYKVVQIEPLGIAPTRAKRRAVQREKRAVGIGALFLGFLGAAGSTMGAASVTLTVQARQLLSGIVQQQSNLLRAIEAQQHLLQLTVWGIKQLQARVLAMESYLKDQQLLGIWGCSGKLICTTTVPWNTSWSNKSLDQIWNNMTWREWEKEIDNYTDLIYTLIEKSQNQQEKNEQELLELDKWASLWNWFDITNWLWYIKIFIMVVGGLVGLRIVFAVLSIINRVRQGYSPLSFQTHLPAPRGPDRPEGIGEEGGERDSDRSGRSVDGFLPLIWVDLRSLFLFSYHRLTDLLLIVTRIVELLGRRGWGILKYWWSLLQYWSQELKNSAVSLLNATAIAVAERTDRIIEIVQRVFRALLHIPRRIRQGFERALL

SEQ ID NO: 158 - protein, 841 aa, Human immunodeficiency virus
MRVRGIKRNYPHLWIWGTMLLGMLLMSYSAANNLWVTVYYGVPVWKEATTTLFCASDAKAYKAEAHNIWATHACVPTDPNPQEIELKNVTENFNMWRNNMVEQMHEDIISLWDQSLKPCVKLTPLCVTLNCTNVTSSNNGTVGNTEDMKNCSFNITTIVRDKKKQEYALFYRLDIVEINPNDTSYRLINCNTSAITQACPKMSFEPIPIHYCAPAGFAILKCNDKKFKGTGPCSNVSTVQCTHGIRPVVSTQLLLNGSLAEEEIMIRSEDFTNNVKNIIVQFNKSVEIVCIRPGNNTKRSIHFGPGQALYAYSIIGDIRNANCTINKTSWHDTLQKVEKELEKIYNKKINFEPSSGGDLEITTHSFNCGGEFFYCNTSKLFNSTWANSTWDNSNITNITIPCRIKQIINMWQGVGRAMYAPPIAGEIRCTSNITGLLLTRDGGSNNTNETETFRPGGGNMKDNWRSELYKYKVVRIEPLGVAPTRAKRRVVGREKRAIGLGALFLGFLGAAGSTMGAASVTLTVQARELLSGIVQQQNNLLRAIEAQQHLLQLTVWGIKQLQARVLAVERYLKDQQLLGIWGCSGKHICTTTVPWNSSWSNKSLDDIWQNMTWMQWEKEIENYTGVIYNLIEDSQIQQEKNEKELLELDKWASLWNWFDITNWLWYIKIFIMIVGGLIGLRIVFAVLSMVNRVRQGYSPLSFQTLFPVPRGPDRPEGIEEEGGEQDRGRSIRLVNGFSALIWDDLRNLCLFSYHRLRDLILIAARIVDLLGRRGWEALKYLWNLLKYWSQELENSAISLYNATAIAVAEGTDRVIELVQRAFRAVLNIPRRIRQGLERALL

SEQ ID NO: 159 - protein, 850 aa, Human immunodeficiency virus
MRVRGIERNYQHLWRWGTMLLGILMICSAAGQLWVTVYYGVPVWKEATTTLFCASDAKAYKAEAHNIWATHACVPTDPNPQEIVLGNVTENFNMWKNDMVEQMHEDIISLWEQSLKPCVKLTPLCVTLNCTNAKIEQNVTVAGMRNCSFNMTTELKDKKKQEYALFYKLDVVQIDNSSTNTDYRLINCNTSAITQACPKITFEPIPIHYCAPAGYAILKCNNKTFNGMGPCKNVSTVQCTHGIRPVVSTQLLLNGSLAEEEIVIRSENLTNNAKIIIVQLNKSVEINCTRPSNNTRKGVHIGPGQAIYSTGQIIGDIRKAHCNISRKEWNSTLQQVTKKLGSLFNTTKIIFNASSGGDPEITTHSFNCNGEFFYCNTAGLFNSTWNRTNSEWINSKWTNKTEDVNITLQCRIKQIINTWQGVGKAMYAPPVSGIIRCSSNITGLLLTRDGGGADNNRQNETFRPGGGDMRDNWRSELYKYKVVRIEPLGIAPTKARRRVVEREKRAIGLGALFLGFLGTAGSPMGAVSMTLTVQARQVLSGIVQQQNNLLRAIEAQQHLLQLTVWGIKQLQARILAVESYLKDQQLLGIWGCSGKHICTTNVPWNSSWSNKSLDYIWKNMTWMEWEKEIDNYTELIYSLIEVSQIQQEKNEQELLKLDSWASLWNWFSITKWLWYIKIFIMIVGGLIGLRIVFAVLSLVNRVRQGYSPLSFQTLLPAPRGPDRPEGTEGEGGEQGRDRSIRLLNGFSAIIWDDLRNLCLFSYHRLTDLILIATRIVTLLGRRGWEAIKYLWNLLQYWIQELKNSAISLFDATAIAVAEGTDRAIEIIQRVGRAILNIPTRIRQGLERALL

SEQ ID NO: 160 - protein, 859 aa, Human immunodeficiency virus
  misc_feature (284)..(284): Xaa can be any naturally occurring amino acid
MRVKETQMNWPNLWKLGTLILGLVIICSASBNLWVTVYYGVPVWRDADTTLFCASDAKAHETEMHNVWATHACVPTDPNPQEIHLENVTENFNMWKNNMVEQMQEDVISLWDQSLKPCVKLTPLCVTLKCTNANLANVNNRTNDSNIIGNITDEIRNCSFNMTTEIRDRKQKVHALFYKLDIVQIEDDKNSSEEYRLINCNTSVIKQACPKISFDPIPIHYCTPAGYAILKCNDKNFNGTGPCKNVSSVQCTHGIKPVVSTQLLLNGSLAEEEIIIRSENLTNXAKTIIVHLNKSVEINCTRPSNNTRTSITIGPGQVFYRTGDIIGDIRKAYCEINGTKXNEALKQVAEKLKEHFNNKTIIFQPPSGGDLEITTHHFNCRGEFFYCNTTQLFNSTCIGNETMEGCNGTIILPXKIKQIINMWQGVGQAMYAPPISGRINCVSNITGILLTRDGGANTTNNETFRPGGGNIKDNWRSELYKYKVVQIEPLGIAPTRAKRRVVEREKRAVGIGAMIFGFLGAAGSTMGAASITLTVQARQLLSGIVQQQSNLLRAIEAQQHLLQLTVWGIKQLQARVLAVERYLKDQKFLGLWGCSGKIICTTAVPWNSTWSNRSFEEIWNNMTWIEWEREISNYTNQIYEILTESQNQQDRNEKDLLELDKWASLWNWFDITNWLWYIKIFIMIVGGLIGLRIIFAVLSIVNRVRQGYSPLSFQTPSHHQRELDRPERIEEGGXEQGRDRSVRLVSGFLALAWDDLRSLCLFSYHHLRDFILIAARTVELLGRSSLKGLRRGWEGLKYLGNLLLYWGQELKISAISLLNVTAIAVAGWTDRVIEVAQRAWRALLHIPRRIRQGFERALL

SEQ ID NO: 161 - protein, 47 aa, Human immunodeficiency virus
TATYFCVREAGGPDYRNGYNYYDFYDGYYNYHYMDVWGKGTTVTVSS

SEQ ID NO: 162 - protein, 47 aa, Human immunodeficiency virus
TAMFFCAREAGGPIWHDDVKYYDFNDGYYNYHYMDVWGKGTTVTVSS

SEQ ID NO: 163 - protein, 43 aa, Human immunodeficiency virus
TAFYYCARGTDYTIDDAGIHYQGSGTFWYFDLWGRGTLVSVSS

SEQ ID NO: 164 - protein, 43 aa, Human immunodeficiency virus
TALYYCARGTDYTIDDQGRFYQGSGTFWYFDLWGRGTLVSVSS

SEQ ID NO: 165 - protein, 43 aa, Human immunodeficiency virus
TALYYCARGTDYTIDDQGIFYKGSGTFWYFDLWGRGTLVSVSS

SEQ ID NO: 166 - protein, 43 aa, Human immunodeficiency virus
TAIYYCARGTDYTIDDQGIRYDGSGTFWYFDLWGRGTLVSVSS

SEQ ID NO: 167 - protein, 51 aa, Human immunodeficiency virus
TAIYYCTRGSKHRLRDYVLYDDYGLINYQEWNDYLEFLDVWGHGTAVTVSS

SEQ ID NO: 168 - protein, 51 aa, Human immunodeficiency virus; identical to SEQ ID NO: 167

SEQ ID NO: 169 - protein, 51 aa, Human immunodeficiency virus; identical to SEQ ID NO: 167

SEQ ID NO: 170 - protein, 51 aa, Human immunodeficiency virus
TAIYYCTGGSKHRLRDYVLYDDYGLINQQEWNDYLEFLDVWGHGTAVTVSS

SEQ ID NO: 171 - protein, 50 aa, Human immunodeficiency virus
TAIYYCLTGSKHRLRDYVLYNEYGPNYEEWGDYLATLDVWGHGTAVTVSS

SEQ ID NO: 172 - protein, 40 aa, Human immunodeficiency virus
TAFYYCAKDKGDSDYDYNLGYSYFYYMDGWGKGTTVTVSS

SEQ ID NO: 173 - protein, 745 aa, Human immunodeficiency virus
TPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSGSIGASDFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKIKWALCVGAGSCQFFMCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFLAA

SEQ ID NO: 174 - protein, 31 aa, Human immunodeficiency virus
VKNCSFNITTELRDKKQKAYALFYRPDVVPL

SEQ ID NO: 175 - protein, 31 aa, Human immunodeficiency virus
MKNCSFNATTEIRDKKKEMYALFYKLDIVSL

SEQ ID NO: 176 - protein, 31 aa, Human immunodeficiency virus
MTNCSFNATTELRNKEKKEYALFYRLDVVKL

SEQ ID NO: 177 - protein, 30 aa, Human immunodeficiency virus
LRNCSFNITTSIQDKVQDYAIFYKLDIVPI

SEQ ID NO: 178 - protein, 31 aa, Human immunodeficiency virus
IKNCSFNITTSIRDEVQKEYALFYKLDVVPI

SEQ ID NO: 179 - protein, 31 aa, Human immunodeficiency virus
IKNCSFNMTTELKDKTKKMYALFNRYDVVQI

SEQ ID NO: 180 - protein, 29 aa, Human immunodeficiency virus
MKNCSFNVTTELRDKEKEQYALFYTVDVV

SEQ ID NO: 181 - protein, 30 aa, Human immunodeficiency virus
MKNCSFNITTSTSTKMTGYAVFYNLDVVPI

SEQ ID NO: 182 - protein, 31 aa, Human immunodeficiency virus
MRNCSFNTTTFISDKHKKEHALFYRLDIVPL

SEQ ID NO: 183 - protein, 31 aa, Human immunodeficiency virus
IKNCSFNITTTIRDKVQKEEALFYRLDLVPI

SEQ ID NO: 184 - protein, 31 aa, Human immunodeficiency virus
INNCSYNITTELRDREQKVYSLFYRSDIVQM

SEQ ID NO: 185 - protein, 31 aa, Human immunodeficiency virus
MRNCSFNMTTELRDKKKNVSALFYKLDVVPI

SEQ ID NO: 186 - protein, 31 aa, Human immunodeficiency virus
RMNCSFNATTVVNDKQKKVHALFYRLDIEPI

SEQ ID NO: 187 - protein, 31 aa, Human immunodeficiency virus
MKNCSFNLTTEIRDRKKQVHALFYKLDVVPI

SEQ ID NO: 188 - protein, 31 aa, Human immunodeficiency virus
IANCTFNMTTELIDKTKQVYALFYKLDIVQI

SEQ ID NO: 189 - protein, 31 aa, Human immunodeficiency virus
IKNCSFNVTTELTDKKKNMRALFYRADIEPL

SEQ ID NO: 190 - protein, 31 aa, Human immunodeficiency virus
RKNCSFNITTELRDKSKQVYSLFYRLDIVPI

SEQ ID NO: 191 - protein, 30 aa, Human immunodeficiency virus
IKNCSFNITTGIRGRVQEYSLFYKLDVIPI

SEQ ID NO: 192 - protein, 31 aa, Human immunodeficiency virus
MKNCTFNITTEIRDKKKEEYALFYKLDIEQI

SEQ ID NO: 193 - protein, 31 aa, Human immunodeficiency virus
MRNCSFNMTTEVRDRQKQVYSLFYRLDIVQI

SEQ ID NO: 194 - protein, 31 aa, Human immunodeficiency virus
MKNCSFNVTSGIRDKVQKEYALLYKLDIVQI

SEQ ID NO: 195 - protein, 31 aa, Human immunodeficiency virus
MKNCSFNITTELKDKKKNVYALFYKLDIVSL

SEQ ID NO: 196 - protein, 31 aa, Human immunodeficiency virus
MKNCSFNITTSIGDKMQKEYALLYKLDIVSI

(19) United States
(12) Patent Application Publication
(10) Pub. No.: US 2014/0348865 A1
(43) Pub. Date: Nov. 27, 2014

(54) IMMUNOGENS BASED ON AN HIV-1 V1V2 SITE-OF-VULNERABILITY

(71) Applicants: The United States of America, as represented by the Secretary, Department of Health and Human Ser., Bethesda, MD (US); University of Maryland, Baltimore, Baltimore, MD (US); University of Washington, Seattle, WA (US)

(72) Inventors: Peter Kwong, Washington, DC (US); Jason McLellan, Hanover, NH (US); Marie Pancera, McLean, VA (US); Jason Gorman, Washington, DC (US); Mallika Sastry, Rockville, MD (US); Kaifan Dai, La Jolla, CA (US); Tongqing Zhou, Boyds, MD (US); John Mascola, Rockville, MD (US); Gary Nabel, Cambridge, MA (US); Masaru Kanekiyo, Chevy Chase, MD (US); Yongping Yang, Potomac, MD (US); Jiang Zhu, Ashburn, VA (US); Lai-Xi Wang, Ellicott City, MD (US); William Schief, Encinitas, CA (US); Chris Carrico, San Francisco, CA (US); Mohammed Amin, Baltimore, MD (US)

(21) Appl. No.: 14/344,589

(22) PCT Filed: Sep. 7, 2012
(86) PCT No.: PCT/US2012/054295

§ 371 Date: Mar. 12, 2014

Related U.S. Application Data
(60) Provisional application No. 61/533,721, filed on Sep. 12, 2011.

(51) Int. Cl. C07K 14/005; C12N 7/00

(52) U.S. Cl. CPC C07K 14/005; C12N 7/00; C12N 2740/16134
USPC 424/188.1; 530/405; 536/23.72; 435/320.1

(57) ABSTRACT

Disclosed are HIV immunogens. Also disclosed are nucleic acids encoding these immunogens and methods of producing these antigens. Methods for generating an immune response in a subject are also disclosed. In some embodiments, the method is a method for treating or preventing a human immunodeficiency type 1 (HIV-1) infection in a subject.

### RELATED APPLICATIONS

This application claims the benefit of U.S. Provisional Application No. 61/533,721, filed Sep. 12, 2011, which is incorporated by reference in its entirety.

### STATEMENT OF JOINT RESEARCH

The work described here was performed under a Cooperative Research and Development Agreement (CRADA) between the U.S. Government (NIAID CRADA AI-0156 (2006-0370)) and International AIDS Vaccine Initiative (IAVI) entitled “Phenotypic characterization, monoclonal isolation, and structural definition of sera and antibodies that neutralize HIV-1.”

### FIELD

The present disclosure relates to immunogenic polypeptides, and specifically to polypeptides that can provoke an immune response to human immunodeficiency virus (HIV).

### BACKGROUND

Over 30 million people are infected with HIV worldwide, and 2.5 to 3 million new infections have been estimated to occur yearly. Although effective antiretroviral therapies are available, millions succumb to AIDS every year, especially in sub-Saharan Africa, underscoring the need to develop measures to prevent the spread of this disease.

An enveloped virus, HIV-1 hides from humoral recognition behind a protective lipid bilayer. The major envelope protein of HIV-1 is a glycoprotein of approximately 160 kD (gp160). During infection proteases of the host cell cleave gp160 into gp120 and gp41. The gp41 is an integral membrane protein, while gp120 protrudes from the mature virus. The mature gp120 glycoprotein is approximately 470-490 amino acids long depending on the HIV strain of origin. N-linked glycosylation at approximately 20-25 sites makes up nearly half of the mass of the molecule. Sequence analysis shows that the polypeptide is composed of five conserved regions (C1-C5) and five regions of high variability (V1-V5). Together gp120 and gp41 make up the HIV envelope spike, which is a target for neutralizing antibodies.

It is believed that immunization with effectively immunogenic HIV gp120 envelope glycoprotein can elicit a neutralizing response directed against gp120, and thus HIV. Despite extensive effort, a need remains for immunogens that are capable of eliciting such an immunogenic response. In order to be effective, the antibodies raised to the immunogen must be capable of neutralizing a broad range of HIV strains and subtypes.

### SUMMARY

Disclosed herein are immunogenic polypeptides including a PG9 epitope (“PG9 epitope antigens”) nucleic acid molecules encoding such polypeptides, and protein nanoparticles including such polypeptides, which are useful to induce an immune response to HIV (for example HIV-1) in a subject. The immunogens have utility, for example, as both potential vaccines for HIV and as diagnostic molecules (for example, to detect and quantify target antibodies in a polyclonal serum response).

Elucidation of these immunogenic polypeptides was accomplished by achieving, for the first time, the crystallization and three-dimensional structure determination of a complex of the V1/V2 domain of HIV-1 gp120 bound to the broadly neutralizing antibody PG9. The crystal structure of the PG9 bound to the V1/V2 domain from two different HIV strains shows that, when bound to PG9, the V1/V2 domain adopts a four-stranded anti-parallel beta-sheet, with PG9 forming contacts with a first N-linked glycan at gp120 position 160 and a second N-linked glycan at gp120 position 156 or position 173. Due to the conformation of the underlying beta-sheet, the N-linked glycan at position 156 of HIV-1 occupies substantially the same three-dimensional space as the N-linked glycan at position 173, when bound to PG9. These structures illustrate that the minimal PG9 epitope on gp120 includes a two stranded anti-parallel beta-sheet including gp120 positions 154-177, with a first N-linked glycan at gp120 position 160 and a second N-linked glycan at gp120 position 156 or position 173, but not both.

Several embodiments include an isolated antigen comprising a polypeptide comprising a PG9 epitope stabilized in a PG9-bound conformation by at least one pair of crosslinked cysteines. The PG9 epitope comprises gp120 positions 154-177 according to the HXB2 numbering system and corresponding to the amino acid positions in the amino acid sequence set forth as SEQ ID NO: 1. The PG9 epitope further comprises a pair of crosslinked cysteines at positions 155 and 176 and no cysteine residues at positions 154, 156-175 and 177. The PG9 epitope further comprises a first N-linked glycosylation site comprising an asparagine residue at position 160 and a second N-linked glycosylation site comprising an asparagine residue at position 156 or position 173, wherein the first and second glycosylation sites are glycosylated, and at most four additional amino acid substitutions compared to a wild-type HIV-1 gp120. In several such embodiments monoclonal antibody PG9 specifically binds to the antigen.

Additional embodiments include an isolated antigen comprising an epitope-scaffold protein, wherein the epitope scaffold protein comprises a heterologous scaffold protein covalently linked to the antigen described above, or to a polypeptide comprising a PG9 epitope comprising gp120 positions 154-177 according to the HXB2 numbering system and corresponding to the amino acid positions in the amino acid sequence set forth as SEQ ID NO: 1, a first N-linked glycosylation site comprising an asparagine residue at position 160 and a second N-linked glycosylation site comprising an asparagine residue at position 156 or position 173, wherein the first and second glycosylation sites are glycosylated, and at most four additional amino acid substitutions compared to a wild-type HIV-1 gp120, wherein monoclonal antibody PG9 specifically binds to the antigen.

In several embodiments, the isolated antigen includes a multimer the polypeptide comprising the PG9 epitope stabilized in a PG9-bound conformation. Some embodiments include an isolated antigen, comprising a multimer comprising a first polypeptide and a second polypeptide, each polypeptide comprising a PG9 epitope stabilized in a PG9-bound conformation by two pairs of crosslinked cysteines, and further comprising gp120 positions 126-196 according to the HXB2 numbering system and corresponding to the amino acid positions in the amino acid sequence set forth as SEQ ID NO: 1. The first pair of cross-linked cysteines is at positions 126 and 196, and the second pair of cross-linked cysteines is at positions 131 and 157. In several embodiments, the PG9 epitope does not include any cysteine residues at positions 127-130, 132-156 and 158-195. The PG9 epitope include a first N-linked glycosylation site comprising an asparagine residue at position 160 and a second N-linked glycosylation site comprising an asparagine residue at position 156 or position 173, wherein the first and second glycosylation sites are glycosylated. In several such embodiments, the PG9 epitope includes at most 12 additional amino acid substitutions compared to a wild-type HIV-1 gp120. In several such embodiments, monoclonal antibody PG9 specifically binds to the antigen.

In several embodiments, the antigen is glycosylated at gp120 position 160 and gp120 position 156 or the antigen is glycosylated at gp120 position 160 and gp120 position 173. In some such embodiments, the asparagine at position 160 is linked to an oligomannose glycan and the asparagine at position 156 is linked to a complex glycan, or the asparagine at position 160 is linked to an oligomannose glycan and the asparagine at position 173 is linked to a complex glycan.

In additional embodiments, the antigen is included on a protein nanoparticle. Some embodiments include a protein nanoparticle comprising an antigen comprising a polypeptide comprising a PG9 epitope. In some such embodiments, the PG9 epitope comprises gp120 positions 154-177 according to the HXB2 numbering system and corresponding to the amino acid positions in the amino acid sequence set forth as SEQ ID NO: 1, a first N-linked glycosylation site comprising an asparagine residue at position 160 and a second N-linked glycosylation site comprising an asparagine residue at position 156 or position 173, wherein the first and second glycosylation sites are glycosylated; and at most four additional amino acid substitutions compared to a wild-type HIV-1 gp120. In several such embodiments, monoclonal antibody PG9 specifically binds to the protein nanoparticle.

Methods of generating an immune response in a subject are disclosed, as are methods of treating, inhibiting or preventing a HIV-1 infection in a subject. In such methods a subject, such as a human subject, is administered and effective amount of a disclosed antigen.

Methods for detecting or isolating an HIV-1 binding antibody in a subject infected with HIV-1 are disclosed. In such methods, a disclosed immunogen is contacted with an amount of bodily fluid from a subject and the binding of the HIV-1 binding antibody to the immunogen is detected, thereby detecting or isolating the HIV-1 binding antibody in a subject.

The foregoing and other objects, features, and advantages of the embodiments will become more apparent from the following detailed description, which proceeds with reference to the accompanying figures.

### BRIEF DESCRIPTION OF THE FIGURES

FIGS. 1A-1F illustrate PG9-V1V2 interactions. Glycan, electrostatic, and sequence-independent interactions of antibody PG9 facilitate recognition of V1V2 from the ZM109 strain of HIV-1 gp120. A, PG9 is shown as a grey molecular surface, and strands B and C of V1V2 are shown as green ribbons. Mannose and N-acetylglucosamine residues are shown in stick representation, as are the side chains of Asn160 and 173. Electron density (2Fₒ-F_{c}) is contoured at 16 and shown as a blue mesh. B, Ribbon representations of strands B and C of ZM109 V1V2 (dark grey), PG9 heavy chain (medium grey) and PG9 light chain (dark grey). V1V2 glycans and PG9 residues that hydrogen bond are shown as sticks. Nitrogen atoms are colored dark grey, oxygen atoms are colored light grey, and dotted lines represent hydrogen bonds. C, Schematic of the Man₅GlcNac₂ moiety attached to Asn160. GlcNacs are shown as dark grey squares, and mannoses as lighter grey circles. Hydrogen bonds to PG9 are listed to the right of the symbols, as is the total surface area buried at the interface between PG9 and each sugar. D, Schematic of the PG9-main-chain interaction with V1V2. Disulfide bonds in V1V2 are shown as light grey sticks. E,F, Ribbon representation of V1V2 (dark grey) and PG9 CDR H3 (light grey). Hydrogen bonds are represented by dotted lines. Main-chain interactions are shown in E, and side chain interactions in F (with the two images related by a 90° rotation about a vertical axis). Details of PG9 interaction with V1V2 from the CAP45 strain of HIV-1 are shown in FIG. 14.

FIGS. 2A-2I illustrate the structure of the V1V2 domain of HIV-1 gp120. The four anti-parallel strands that define V1V2 fold as a single domain, in a topology known as “Greek key”, which is observed in many proteins. A, Schematic of V1V2 topology. V1V2 resides between strands P2 and P3 of core gp120, and its structure completes the crystallographic determination of all portions of HIV-1 gp120. Strands are depicted as arrows and disulfide bonds as light grey lines. B, C, Ribbon diagram of V1V2 residues 126-196 from HIV-1 strains CAP45 (dark grey) and ZM109 (light grey). Conserved disulfide bonds are represented as ball and stick, and the beginning and terminating residues of each strand are labeled. D, Superposition of the structures shown in B, C, and E, Amino acid conservation of V1V2. The backbone is shown as a tube of variable thickness, colored as a rainbow from cold (dark grey) to hot (light grey), corresponding to conserved (thin) and to variable (thick), respectively, based on an alignment of 166 HIV-1 sequences. Aliphatic and aromatic side chains are shown as sticks with semi-transparent molecular surface, shaded by conservation as in I, F, Electrostatic surface potentials of CAP45 V1V2 colored dark to light grey, corresponding to positive and negative surface potentials, respectively. G, Molecular surfaces corresponding to main-chain atoms including C_{β} are colored grey, with other surfaces colored white. H, Superposition of ZM109 and CAP45 models containing V1 and V2 loops and associated glycans. For each glycosylated asparagine, only the first N-acetylglucosamine attached to the asparagine is shown and represented as sticks with a transparent molecular surface. Modeled amino acids and glycans that are disordered in the crystal structures are shown in gray. I, Sequence alignment of positions 126-196 of nine HIV-1 strains that are potently neutralized by PG9 (positions 126-196 of SEQ ID NOs: 2, 3, and 154-160, respectively). Glycosylated asparagine residues are boxed and in bold. Identical residues have a dark green background with white characters, while conserved residues have white backgrounds with dark green characters. Above the alignment, β-strands are shown as arrows, colored magenta and green for CAP45 and ZM109, respectively. Residues and attached glycans that make hydrogen bonds to PG9 are denoted with symbols above the alignment (side-chain hydrogen bonds ¤, main-chain hydrogen bonds •, or both).

FIG. 3 illustrates the overall structure of V1V2 domain of HIV-1 gp120 in complex with PG9. V1V2 from the CAP45 strain of HIV-1 is indicated and shown in dark grey ribbons, in complex with the antigen-binding fragment (Fab) of antibody PG9. The PG9 heavy and light chains are indicated and shown as light and dark grey ribbons, respectively, with complementarity determining regions (CDRs) in different shades. Although the rest of HIV-1 gp120 has been replaced by the 1FD6 scaffold (shown in light grey ribbons), the positions of V1V2, PG9, and scaffold are consistent with the proposal that the viral spike, and hence the viral membrane, is positioned towards the top of the page. The extended CDR H3 of PG9 is able to penetrate the glycan shield that covers the V1V2 cap on the spike and to reach conserved elements of polypeptide, while residues in heavy and light chain combining regions recognize N-linked glycans. The disordered region of the V2 loop is represented by a dashed line. Perpendicular views of V1V2 are shown in FIGS. 2 and 6, and the structure of PG9 in complex with V1V2 from HIV-1 strain ZM109 is shown in FIG. 13.

FIGS. 4A-4C illustrate PG9 and PG16 recognition of the HIV-1 viral spike, monomeric gp120, and scaffolded-V1V2. Quaternary-structure-preferring antibodies display different affinities for oligomeric, monomeric, and scaffolded V1V2. Both structural and arginine-scanning mapping, however, suggest that the epitopes of PG9 and PG16 are mostly present in scaffolded V1V2. A, Affinities of PG9 (filled symbols) and PG16 (open symbols) are shown for the functional viral spike (gp120/gp41)₃ (circles), monomeric gp120 (triangles), and scaffold-V1V2 (squares), based upon neutralization (black), ELISA (dark grey) and surface plasmon resonance (light grey). B, Negative stained images are shown for ternary complexes of wild-type gp120 (HIV-1 strain 16055) in complex with antibody PG9 and the CD4-binding-site antibody T13. Six different classifications were observed, and are superimposed in the upper left panel and labeled, PG9-1 through PG9-6. Individual fitting for classes PG9-1, PG9-3 and PG9-5 are shown after rigid-body alignment of Fab PG9-scaffold-V1V2, Fab T13 and core gp120 (in the conformation bound by the CD4-binding site antibody F105). C, Comparison of crystallographically-defined PG9 paratope with neutralization-defined PG16 paratope. Scaffold-V1V2 interactive surface of PG9 in ZM109 (left) and CAP45 (middle) contexts is shown along with the PG16 paratope (right) as defined by “arginine-scanning” mutagenesis (orange-highlighted residue is Trp64 in the CDR H2). Perpendicular views of the paratope, rotated by 90° about a horizontal axis, are shown in top and bottom rows.

FIGS. 5A-5B illustrate CDR H3 features of V1/V2-directed broadly neutralizing antibodies. A protruding anionic CDR H3 is preserved in members of this broadly neutralizing class of antibodies. A, CDR H3 sequence alignment (showing kabat positions 87-117 of SEQ ID NOs 158-169, respectively). Cohort, donor information, and sequences in the CDR H3 (Kabat definition and numbering) are shown for V1V2-directed antibodies. Positively and negatively charged residues are boxed. Residues that make hydrogen bonds to CAP45 residues (dark grey) or glycans (light grey) are denoted with symbols above the alignment (side-chain hydrogen bonds ¤, main-chain hydrogen bonds •, or both). Similar contacts are shown for ZM109 residues (dark grey) or glycans (light gray). Sulfated tyrosines are circled or squared if the post-translational modification has been confirmed crystallographically or by mass spectrometry, respectively. The sequence for the V1V2-directed strain-specific antibody, 2909, is also included. B, Protruding CDR H3, displayed as ribbon diagrams with sulfated tyrosines shown in spheres and paired with electrostatic surface potentials shaded to indicate positive and negative surface potentials. All CDR H3s are aligned so that the light chain would be on the left and heavy chain on the right (as in FIG. 13). Average surface electrostatic potentials are shown.

FIGS. 6A-6B illustrate two glycans and a strand comprise a V1V2 site-of-vulnerability. Glycan, electrostatic, and sequence-independent interactions allow PG9 to recognize a glycopeptide site on V1V2. A, Site characteristics in CAP45 strain of HIV-1. Glycans 160 and 156 (173 with ZM109) are highlighted in light grey, and strands B and C are highlighted in dark grey, with the rest of V1V2 in semi-transparent white. The interactive surface of V1V2 with PG9 is shown, colored according the local electrostatic potential as in FIG. 5B. The contribution of each structural element to that surface is provided as a percentage of the total. Although the scaffolded V1V2s used here do not allow a comprehensive analysis of the overall antibody response to this region of gp120, in addition to assisting with structural definition of effective V1V2-directed neutralization, the V1V2 scaffolds may have utility in attempts to direct the V1V2-elicited response away from the hypervariable loops to the conserved strands—especially the site-of-vulnerability highlighted here. B, Saturation transfer difference (STD) NMR for Man₅GlcNAc₂-Asn binding to PG9. the graph shows STD spectrum of 1.5 mM Man₅GlcNAc₂-Asn in the presence of 15 μM Fab PG9 (lower spectrum) is paired with the corresponding reference spectrum (upper spectrum). C, Langmuir binding curve used to obtain the K_{D} a function of glycan concentration (A signals correspond to N-acetyl protons, which are shown in the boxed area of the upper panel). D, Stacked STD NMR spectra as a function of Man₅GlcNAc₂-Asn concentration.

FIGS. 7A-7F illustrate β-hairpins in core structures of HIV-1 and SIV. Bridging sheet conformations of previously determined HIV-1 gp120 structures. Inner domain is shown in light grey, outer domain in dark grey and bridging sheet region in medium grey. Residues corresponding to the V1V2 stem are highlighted: 119-205 (HXbc2 numbering) and 103-215 (SIV). A, Schematic of the bridging sheet and variable region V1V2. B, 48d- and CD4-bound gp120. C, b12-bound. D, b13-bound. E, F105-bound. F, unliganded SIV core.

FIG. 8 illustrates scaffold proteins used to host V1V2 regions. Structures of the scaffold proteins before transplantation of the V1V2 region are shown as grey ribbon diagrams, with their PDB ID codes listed above. The dark grey segment in each scaffold was removed for insertion of the V1V2 region.

FIGS. 9A-9B illustrate HIV-1 gp120 V1V2 Scaffolds interact with the gut homing receptor α₄β₇. YU2 V1V2 scaffold proteins interaction with α₄β₇ was studied by an indirect and direct binding assay. A, Indirect binding assay: % inhibition of AN1 gp120 binding to α₄β₇ on CD4+ T cells by three YU2 V1V2 scaffold proteins (1JO8, 1E6G, 1FD6). In the competition assay, purified CD4+ T cells were preincubated with an anti-CD4 antibody (Leu3A) and YU2 V1V2 scaffold proteins in divalent cation containing buffer (1 mM MnCl₂ and 100 um CaCl₂) followed by the addition of biotin labeled ancestral gp120 (AN1 gp120). Mean fluorescence intensity (MFI) was measured to determine the extent of inhibition of AN1 gp120 binding to α₄β₇ by the YU2 V1V2 scaffold proteins. This experiment was performed with 5-fold molar excess scaffold proteins over AN1 gp120. This initial competition assay indicated that two of the scaffolds, 1FD6A and 1JO8, provided the most pronounced inhibition of all scaffolds tested, therefore, a direct binding assay was performed with YU2 V1V2 1JO8. B, Direct binding assay: % reactivity of YU2 V1V2 1JO8 scaffold protein to α₄β₇ on CD4+ T cells. The scaffold protein was biotinylated and used to bind directly to CD4+ T cells in the presence of Leu3A and divalent cations (1 mM MnCl₂ and 100 μM CaCl₂). Binding of AN1 gp120 and YU2 V1V2 1JO8 to CD4+ T cells is reduced to background levels in the presence of HP2/1, an anti α₄ antibody. All experiments were performed in duplicate and SEM error bars are shown (except for 1JO8 binding to α₄β₇ in EDTA containing buffer and its inhibition by HP2/1). Note that PG9 does not inhibit gp120 binding to α₄β₇ in these assays. The gp120s were derived from subtype A/E and bound PG9.

FIG. 10 is a set of graphs illustrating binding of HIV-1 ZM109 gp120 and V1V2 scaffolds to antibody PG9. Surface-plasmon resonance sensorgrams with their respective fitted curves (black) are shown, with the highest concentration of each 2-fold dilution series labeled. The association and dissociation rates as well as the affinity values are shown to the right of the sensorgrams. In curves fitted with a heterogenous model, separate kinetics data are listed, along with contributing percentages for each component. Data were processed as described in Example 1.

FIGS. 11A-11D illustrate PG9 tyrosine sulfate (TYS) characterization. A, PG9 Fab has two sulfated tyrosines although there is some heterogeneity. B, Sulfation is controlled by tyrosyl protein sulfotransferase (TPST) and co-expression of TPST-1 promotes hypersulfation of PG9 (up to quintuple). Hypersulfated PG9 Fab was produced by co-expression of human tyrosyl protein sulfotransferase (TPST-1) in HEK 293T. Hyposulfated PG9 Fab was produced in Sf9 cells using a recombinant baculovirus, pFastBac Dual, expressing both the heavy and light chains under the control of the polyhedron and p10 promoters, respectively. Fabs were purified by anti-lambda affinity (CaptureSelect, BAC) and cation exchange using Mono S (GE HealthCare). Fractionation of PG9 sulfoforms was achieved by a shallow KCl gradient and individual fractions were characterized by electrospray time-of-flight mass spectrometry (ESI-TOF). C, Sulfation enhances PG9 association with gp120. Hypersulfated PG9 Fab (co-expressed with TPST-1) shows higher affinity for monomer than not hypersulfated PG9 Fab, however PG9 binary complex does not completely survive SEC. D, Effect of neutralization of hyper-sulfated PG9. Tyrosine to phenylalanine CDR H3 mutants (H100A, H100E, H100G, H100H, and H100K) were generated by the polymerase incomplete primer extension method (PIPE), expressed, purified, and fractionated as for wild-type.

FIGS. 12A-12B illustrate on-column complex formation and purification. A, Schematic of the on-column complex formation between PG9 and scaffolded V1V2s, as described in Example 1. B, Gel filtration result and the elution shown for 1JO8 ZM109. A coomassie blue-stained SDS-PAGE gel is shown for fractions 18-25. MW=molecular weight standards. L=purified 1JO8 ZM109 before passage over the PG9-bound resin. FT=flow through of purified 1JO8 ZM109 after passage over the PG9-bound resin.

FIG. 13 illustrates structure of PG9 in complex with the V1V2 region from HIV-1 strain ZM109. The PG9 heavy and light chains are shown as light and dark grey ribbons, respectively, with CDRs colored different shades. V1V2 residues 126-196 from HIV-1 strain ZM109 are indicated and shown as medium grey ribbons, and attached glycans are shown as sticks with a transparent molecular surface. Residues that are different from the CAP45 strain are shown as opaque molecular surfaces, shaded according to chemical properties as shown in the legend. The 1FD6 scaffold is shown as white ribbons, with side chains shown as sticks and shaded for those residues that were altered during the scaffolding process, including a Glu to Ala mutation that ablated IgG binding.

FIGS. 14A-14F illustrate glycan recognition of CAP45 V1V2 by PG9. PG9 recognizes the Man₅GlcNAc₂ glycan attached to Asn160 of CAP45 V1V2 through interactions analogous to those observed for ZM109. Additionally, the CAP45 V1V2 structure also reveals several interactions between PG9 and the Asn156-glycan. A, PG9 is represented as a light grey molecular surface, and CAP45 V1V2 is shown as a ribbon diagram (dark grey). Mannose and GlcNac residues are shown as sticks, as are the side-chains of Asn160 and Ans156. 2Fₒ-F_{c} electron density contoured at 16 is shown as a blue mesh. B, Ribbon representations of CAP45 V1V2 (medium grey), PG9 heavy chain (light grey) and PG9 light chain (dark grey). Glycans and PG9 residues hydrogen-bonding to the glycans are shown as sticks. Nitrogen atoms are colored dark grey, oxygen atoms are colored light grey, and black dotted lines represent hydrogen bonds. C, Schematic of the Man₅GlcNac₂ moeity attached to Asn160. GlcNac is shown as squares, and mannose is shown as circles. Hydrogen bonds to PG9 are listed to the right of the symbols, as is the total surface area buried at the interface between PG9 and each sugar. D, E, F, An orientation of the structure highlighting the interactions between PG9 and the Asn156-glycan of CAP45 V1V2 is presented with representations corresponding to panels A, B, C, respectively.

FIGS. 15A-15B illustrate HIV-1 strains with V1V2 regions missing a glycan at position 156. Electrostatic surface potentials of V1V2, with modeled V1 and V2 loops. A, CAP45. B, ZM109 along with models of five additional strains lacking glycan 156. Sanding corresponds to positive and negative surface potentials. Potential glycosylation sites are shown for glycans 160 (medium grey), 156/173 (light grey) and other glycosylation sites within strands A-D. Glycans for the modeled V1 and V2 loops are not shown.

FIG. 16 illustrates negative stained reference free 2D class averages of the 128 classes calculated from the untilted micrographs collected for the RCT (Random Conical Tilt). Class averages with white numbers in the top left were used to generate the RCT volumes. The white numbers represent the RCT volumes shown in FIG. 4b. Numbers in the lower left represent the total number of particles in each average. Reference free hierarchical class averaging within each class average produced indistinguishable results to the parent class average An RCT volume was calculated from the appropriately combined class averages shown in this figure. RCTs were only calculated from class averages where the hole in the center of the T13 and PG9 Fabs were clearly visible. This hole in the center of the Fabs was used as a biophysical restraint to support the authenticity of the class averages.

FIG. 17 illustrates negative stained reference free 2D class averages compared to raw particles. First column entries represent the RCT volume designation shown in FIG. 4b. Second column entries are reference free class averages determined from the untilted micrographs collected at a 150,000× magnification. Classes 7 and 8 are the binary complex of T13 in complex with gp120, and the PG9 Fab, respectively. Third column entries are the reference free class averages determined from the untilted micrographs collected at 62,000× for the RCT image reconstruction. The scale bar in each column is 100 Å long. Columns 4-25 are representative raw particles for each class average at the 62,000× magnification. The particles are extracted from CTF corrected images. The final column depicts the total number of particles in each class. A total of 11,997 particles were extracted from the untilted micrographs collected at a 62,000× magnification.

FIG. 18 illustrates 6 Å crystal structure of JR-FL gp120 core bound to T13 Fab. Ribbon representation of JR-FL gp120 core (medium grey) in complex with T13 Fab (light grey) at 6 Å with 2Fₒ-F_{c} electron density shown in mesh. JR-FL gp120 core was expressed in HEK 293S GnTI^{-/-} cells using a codon-optimized synthetic gene incorporating an Ig kappa signal peptide inserted into the vector phCMV (Genlantis). Cells were transfected with PEIMAX™ (PolySciences) and allowed to secrete Env for 72 hours. Cell supernatant was concentrated and filtered and loaded on to Galanthus nivalis lectin agarose beads (Vector labs) and eluted with 1.0 M methyl-α-d-mannopyranoside. The eluted gp120 was further purified by SEC using SUPERDEX™ 200 16/60 (GE Healthcare). T13 Fab was expressed by periplasmic secretion of both the light and heavy chains using pET-Duet. Cells were induced with IPTG and allowed to express Fab overnight at 16° C. Cells were then harvested by centrifugation, protease inhibitor cocktail set V (CalBiochem) was added, and passaged three times through a cell disruptor. Clarified cell lysate was loaded on a 5 mL HiTrap Protein G column and Fab was eluted using 1 M glycine pH 2.8. Affinity-purified Fab was then purified further by Mono S cation exchange. A complex of JR-FL gp120 core and T13 Fab was concentrated to 16 mg/ml and crystallized by sitting drop vapor diffusion in 20% PEG 3350, 0.2 M lithium chloride, 12.5 mM Tris, pH 8.0. Crystals were cryoprotected by addition of 30% glycerol to the mother liquor, and a data set to 6.0 Å was collected. Molecular replacement was carried out with PHASER. A shell script was used to cycle through 176 different Fab models using an in-house database of structurally aligned Fab coordinates derived from the PDB. A solution using F105-bound gp120, truncated V1/V2 stem and β20-21 loop, and the 176 Fab database placed gp120 and two different Fabs, which yielded the same solution. Env residues 91-116, 210-297, 330-395, 412-491 were used in the structure solution, and Fabs 1HZH and 1DFB. 1 HZH yielded the best overall Phaser solution. Rigid body refinement was undertaken with PHENIX, and the structure was refined to an R_{cryst} of 0.31 (R_{free} of 0.46). No coordinate refinement was performed.

FIGS. 19A-19D illustrate negative stain of gp120-T13 and gp120-T13-PG9 complex. A, Crystal structure of gp120-T13 complex at 6 Å. B, 2D class average of the same complex by EM. This view corresponds to view 7 in FIG. 17. C, 2D class average of ternary complex of gp120-T13-PG9. D, Same as B but colored by component. This view corresponds to view 1 in FIG. 17. Thus, the binary crystal and EM structures unambiguously define the location of T13 on one side of the strong rod-shaped gp120 density. These fits all orient the V1/V2/V3 loops into the additional plume of density adjacent to the other strong density for an Fab, which then is PG9. Additional evidence for this arrangement is provided by an EM titration experiment required to get higher populations of the ternary complex. Briefly, it was necessary to add excess PG9 to the stoichiometric, purified gp120-T13-PG9 complex after diluting the sample in preparation for deposition on the EM grid. Failure to do so resulted in a proportionally higher population of view 7 (FIG. 17), which represents the gp120-T13 complex as discussed above.

FIG. 20 illustrates functional definition of PG16 paratope by “arginine-scanning” mutagenesis. Twenty-two individual arginine mutants were assessed for neutralization on nine different strains of HIV-1. Residues mutated to arginine are displayed as spheres on a ribbon diagram of the unbound PG16 structure (Pancera et al., J. Virol., 2010), and shaded according to the fold-increase in IC₅₀ for the mutant relative to wild-type.

FIG. 21 illustrates effects of gp120 V3 loop binding to antibodies PG9 and PG16. Full-length gp120 monomers (left column) or V3-deleted gp120 monomers (right column) were tested for binding to PG9 (top four panels) and PG16 (bottom four panels). Surface-plasmon resonance sensorgrams with their respective fitted curves (black) are shown, with the highest concentration of each 2-fold dilution series labeled. The equilibrium dissociation constant (K_{D}) is shown above the sensorgrams. In curves fitted with a heterogenous model, separate K_{D}s are listed, along with contributing percentages for each component. Data were processed as described in Example 1.

FIGS. 22A-22B illustrate comparison of PG9 CDR H3 electron density for unbound and V1V2-bound structures. To determine the degree that unbound structures resembled complexed ones, the structure of unbound PG9. PG9 crystals diffracted to 3.3 Å with 4 molecules in the asymmetric unit was determined. In three of the four molecules that comprise the asymmetric unit, the CDR H3 appeared to be completely disordered, with weak density observed for only one molecule, consistent with the unbound PG9 CDR H3 being a highly mobile subdomain; in contrast, other regions of the unbound PG9-variable domains closely resembled the bound structures. It was determined the unbound structure of PG16, which also displayed a flexible or more mobile CDR H3. Superposition of the unbound PG16 structure with that of PG9 in the PG9-V1V2 complex indicated that somatic differences focused primarily at the region N-terminal to the V1V2-interactive strand of the CDR H3 and to residues involved in glycan recognition. Overall, unbound PG9 and PG16 structures were compatible with an induced fit mechanism of recognition, where CDR H3 mobility enhances the ability of PG9 and PG16 to penetrate the flexible glycan shield that covers V1V2. A, Ribbon representation of the unbound PG9 Fab, zoomed in on the CDR H3. Heavy chain is yellow, and light chain is blue. 2Fₒ-F_{c} electron density within 6 Å of the CDR H3 and contoured at 0.7σ is shown as a light blue mesh B, Ribbon representation of the 1FD6-ZM109-bound PG9 Fab, zoomed in on the CDR H3. 2Fₒ-F_{c} electron density within 1.5 Å of the CDR H3 and contoured at 1.0σ is shown as a light blue mesh.

FIGS. 23A-23D illustrate unbound CH04 Fab and chimeric CH04H/CH02L Fab structures. Antibodies CH01-CH04 form a clonal lineage, identified from a Glade A-infected donor (CHAVI-0219), with heavy chain-derived from the VH3 family, the same as PG9/PG16 (Bonsignori et al., J. Virol., 2011). Neutralization characteristics of CH01-04 closely resemble those of PG9 and PG16, with a highly similar, alanine-mutagenesis-defined, target epitope. Fabs of CH01-CH03 formed small needles, which were not suitable for structural analysis (Supplementary Table 20 shown in FIG. 46). CH04 formed orthorhombic crystals that diffracted to 1.9 Å, with two molecules in the asymmetric unit, and structure determination and refinement led to an R_{cryst} of 19.6% (R_{free}=23.8%) (Supplementary Table 19 shown in FIG. 45). Chimeric Fabs of CH04H/CH02L formed orthorhombic and tetragonal crystals that diffracted to 2.9 Å. A. Unbound structure of Fab CH04. Ribbon diagram displays heavy and light (blue) chains, with CDRs shaded as indicated. B. Unbound structure of orthorhombic Fab CH04H/CH02L. Ribbon diagram displays heavy (medium grey) and light (light grey) chains C Unbound structure of tetragonal Fab CH04H/CH02L. Ribbon diagram displays heavy (medium grey) and light (light grey) chains. D. Superposition of the CDR H3s with shading from A, B and C. E. CDR H3 lattice contacts.

FIGS. 24A-24B illustrate unbound PGT145 Fab structure. Antibodies PGT141-145 form a clonal lineage, identified from a Glade A- or D-infected donor (IAVI protocol G-84), with heavy chain-derived from the VH1 family (Walker et al., Nature, 2011). Neutralization characteristics of PGT141-145 closely resemble those of PG9 and PG16, although PGT145, the most effective member of this lineage, appeared to have greater tolerance for the type of glycan. Crystals of PGT145 diffracted to 2.3 Å, with 1 molecule in the asymmetric unit, and structure determination and refinement lead to an R_{cryst} of 19.1% (R_{free}=22.6%) (Supplementary Table 19 shown in FIG. 45). A. Ribbon diagram displays heavy (medium grey) and light (light grey) chains, with CDRs shaded as indicated. B. PGT145 CDR H3 details with 2Fₒ-F_{c} electron contoured at 1σ shown in brown.

FIG. 25 illustrates binding of GlcNAc2 to PG9 by NMR. STD (lower trace) and reference (upper trace) NMR spectra of 1.5 mM GlcNAc2 in the presence of 15 μM Fab PG9. (*) Buffer impurity exhibiting nonspecific binding to PG9.

FIG. 26 illustrates binding of mannopentaose to PG9 by NMR. STD (lower trace) and reference (upper trace) NMR spectra of 1.5 mM mannopentaose (structure shown above) in the presence of 15 μM Fab PG9. Protons that exhibit STD enhancements are labeled.

FIG. 27 shows Supplementary Table 1. With reference to the table, Mammalian codon-optimized genes encoding full length, 44-492 (HXBc2 numbering), or V3 loop-deleted gp120s from various strains were synthesized with a human CD5 leader (ΔV3: V3 residues have been replaced as follows: 297-GAG-330, ΔV3 new: V3 residues have been replaced as follows: 302-GGSGSGG-325). The genes were cloned into the XbaI/BamHI sites of the mammalian expression vector pVRC8400, and transiently transfected into HEK293S GnTI^{-/-} cells. gp120 proteins were purified from the media using a 17b affinity column, eluted with IgG elution buffer (Pierce) and immediately neutralized by adding 1M Tris-HCl pH 8.5. The proteins were flash frozen in liquid nitrogen and stored at −80° C. until further use. Complexes or unbound gp120 (with and without N-linked glycans) were used for crystallization screening. All proteins were passed over a 16/60 S 200 size exclusion column. Monodisperse fractions were pooled, and after concentration, proteins were screened against 576 crystallization conditions using a Cartesian Honeybee crystallization robot. Initial crystals were grown by the vapor diffusion method in sitting drops at 20° C. by mixing 0.2 μl of protein complex with 0.2 μl of reservoir solution.

FIG. 28 shows Supplementary Table 2.

FIGS. 29A-C show Supplementary Table 3. With reference to the Table, (i) indicates the number of residues before deletion of native segment and insertion of V1V2 stub; (ii) indicates the residue range listed was removed from the native structure for the V1V2 insertion procedure; and (iii) indicates that CVGAGSC is a placeholder sequence for the V1V2 stub used in for modeling software, derived from PDB ID 1RZJ. Any V1V2 sequence can likely be inserted in place of the stub.

FIG. 30 is Supplementary Table 4. With reference to the table, monoclonal antibodies against the variable region V1V2 were obtained from ProSci. These antibodies were generated by immunizing mice with YU2 gp120, and the sera were tested against YU2 gp120 ΔV1V2 to select positive wells. Six monoclonal antibodies (SBS01-06, subtype IgG1, IgG2a) were obtained that were YU2 V1V2 specific. Peptide mapping was performed by ELISA. Serial dilutions of the six V1V2-directed antibodies were added to YU2 V1V2 peptide-coated wells and binding was probed with horseradish peroxidase-conjugated anti-mouse IgG antibody. YU2 gp120 and gp120 ΔV1V2 were used as positive and negative controls, respectively. Anti-HIV antibody F105 and anti influenza hemagglutinin antibody 9E8 were also used as control antibodies.

FIG. 31 shows Supplementary Table 5. (*) indicates that 1FD6 scaffold protein is a variant of the B1 domain of streptococcal protein G, which binds the Fc region of antibodies and could contribute to binding in the ELISA assay, however this scaffold also binds α₄β₇ in the competition assay; and (#) indicates that these scaffold proteins were tested with surface plasmon resonance and biolayer interferometry. Antigenic analysis of the YU2 V1V2 scaffolds was initially performed by sandwich ELISA. YU2 V1V2 scaffolds were expressed as GFP fusion proteins. The expressed V1V2 scaffold proteins in culture supernatants were added in duplicate to wells coated with a goat polyclonal anti-GFP antibody (Santa Cruz) to allow capture of the desired protein. SBS01-06 proteins were used as detection antibodies and binding was probed with horseradish peroxidase-conjugated anti-mouse IgG antibody. Full length YU2 gp120, ΔV1V2, secreted GFP, anti-HIV antibody F105 and anti influenza hemagglutinin antibody 9E8 were used as control proteins and antibodies. A subset of purified V1V2 scaffold proteins was antigenically characterized by surface plasmon resonance and biolayer interferometry.

FIG. 32 shows Supplementary Table 6. Purified recombinant gp120 (200 ng) was adsorbed onto Reacti-Bind 96-well plates (Pierce), followed by blocking and incubation of serially diluted antibodies. Bound antibody was detected using a horseradish peroxidase-conjugated goat anti-human IgG Fc antibody (Jackson ImmunoResearch Laboratories). Plates were developed using SureBlue 3,3′,5,5′-tetramethylbenzidine (Kirkegaard & Perry Laboratories). gp120 proteins were purchased from Immune Technology Corp. or were expressed and purified as described in Supplementary Table 1 (shown in FIG. 27). Binding was categorized based on the OD₄₅₀ value at the highest concentration tested (5 mg/ml for mAbs, 50 mg/ml for HIV-IG) and EC₅₀ values as follows: ‘++++’=OD₄₅₀≧3.0 and EC₅₀≦0.10; ‘+++’=OD₄₅₀≧3.0 and EC₅₀>0.10; ‘++’=1.0<OD₄₅₀<3.0; ‘+’=0.2<OD₄₅₀<1.0; ‘−’=OD₄₅₀<0.2. OD values were rounded to the nearest tenth and EC₅₀ values to the nearest hundredth before categorization. mAb VRC01 and HIV-IG were included as control antibodies and SIV gp140 proteins and avian influenza hemagglutinin HA1 (H5 HA1) were included as control proteins.

FIGS. 33-36 show Supplementary Tables 7-10.

FIG. 37 shows Supplementary Table 11. For the 1FD6 CAP45 scaffold, a combination of multiple glycosylation mutants was also tested. 156D/N160Q did not bind PG9 nor PG16. N143D/N147D/N192D bound PG9 with an EC₅₀of 0.1 μg/ml and PG16 with an EC₅₀ of 15.1 μg/ml. In regard to ELISA assay with purified protein: WT and site mutated 1JO8 ZM109 V1V2 proteins produced in 293F cell (10 mg/swainsonine) in PBS (pH 7.4) at 2 μg/ml were used to coat plates for two hours at room temperature (RT). The plates were washed five times with 0.05% Tween 20 in PBS (PBS-T), blocked with 300 μl per well of blocking buffer (5% skim milk and 2% bovine albumin in PBS-T) for 1 hour at RT. 100 μl of each monoclonal antibodies 5-fold serially diluted in blocking buffer were added and incubated for 1 hour at RT. Horseradish peroxidase (HRP)-conjugated goat anti-human IgG (H+L) antibody (Jackson ImmunoResearch Laboratories Inc., West Grove, Pa.) at 1:5,000 was added for 1 hour at RT. The plates were washed five times with PBS-T and then developed using 3,3′,5,5′-tetramethylbenzidine (TMB) (Kirkegaard & Perry Laboratories) at RT for 10 min. The reaction was stopped by the addition of 100 μl 1 N H2S04 to each well. The readout was measured at a wavelength of 450 nm. All samples were performed in duplicate. In regard to ELISA assay with supernatant: Culture supernatants from 293F cell (10 mg/L, swainsonine) transfected with WT and site mutated 1FD6 CAP45 V1V2 were used to coat His grab plates (150 μL/well) for overnight at 4° C. 100 μL of each monoclonal antibodies 5-fold serially diluted in blocking buffer were added and incubated for 1 hour at RT. Horseradish peroxidase (HRP)-conjugated goat anti-human IgG (H+L) antibody (Jackson ImmunoResearch Laboratories Inc., West Grove, Pa.) at 1:5,000 was added for 1 hour at RT. The plates were washed five times with PBS-T and then developed using 3,3′,5,5′-tetramethylbenzidine (TMB) (Kirkegaard & Perry Laboratories) at RT for 10 min. The reaction was stopped by the addition of 100 μL 1 N H2SO4 to each well. The readout was measured at a wavelength of 450 nm. All samples were performed in duplicate.

FIGS. 38-40 show Supplementary Tables 12-14.

FIG. 41 shows Supplementary Table 15. Neutralization was measured using single-round-of-infection HIV-1 Env-pseudoviruses and TZM-bl target cells, as described previously (Wu et al., Science, 2010; Li et al., J. Virol., 2005; Seaman et al., J. Virol., 2010). Neutralization curves were fit by nonlinear regression using a 5-parameter hill slope equation as previously described (Li et al., J. Virol., 2005). The 50% and 80% inhibitory concentrations (IC₅₀ and IC₈₀) were reported as the antibody concentrations required to inhibit infection by 50% and 80%, respectively.

FIGS. 42-47 show Supplementary Tables 18-21

FIG. 48 is an illustration showing the minimal PG9 epitope including gp120 residues 154-177, N-linked glycans at positions 156 and 160 and an introduced cross-linked pair of cysteines at positions 155 and 176, which stabilize the glycopeptide in a PG9 bound conformation. The minimal PG9 epitope can be synthesized in vitro.

FIG. 49 shows a series of illustrations showing the indicated PG9 epitope glycopeptides based on the ZM109 HIV-1 strain, which includes asparagine residues at gp120 positions 160 and 173. The affinity of the indicated glycopeptides for monoclonal antibodies PG9 and PG16 is shown.

FIG. 50 shows a series of illustrations showing the indicated PG9 epitope glycopeptides based on the CAP45 HIV-1 strain, which includes asparagine residues at gp120 positions 156 and 160. The affinity of the indicated glycopeptides for monoclonal antibodies PG9 and PG16 is shown.

FIG. 51 illustrates the transplantation of PG9 epitopes on to a scaffold protein to generate PG9-epitope scaffolds.

FIGS. 52A-52D illustrate the design of PG9 Epitope-Scaffold proteins for use as immunogens.

FIG. 53 is a graph illustrating binding of monoclonal antibody PG9 to Epitope-Scaffold proteins containing the minimal PG9 epitope (gp120 positions 154-177).

FIG. 54 is a set of graph illustrating binding of the monoclonal antibodies PG9, PG16, PGT141, PGT142, PTG143, PGT144, PGT145, CH01, CH02, CH03, and CH04 to the indicated Epitope-Scaffold proteins containing the minimal PG9 epitope (gp120 positions 154-177).

FIG. 55 is a table illustrating binding of the monoclonal antibodies PG9, PGT142, PGT145, and CH01, to the indicated PG9 Epitope-Scaffold proteins.

FIG. 56 is a set of three graphs and an image illustrating binding of the monoclonal antibodies PG9, PG16, PGT141, PGT142, PTG143, PGT144, PGT145, CH01, CH02, CH03, and CH04 to the indicated Epitope-Scaffold proteins containing the minimal PG9 epitope (gp120 positions 154-177). 1VH8-ZM109 corresponds to 1VH8_C in Table 2. 1VH8-A244 is the same scaffold presented with 1VH8_C in Table 2 but with the a different HIV strain (A244) inserted into the scaffold.

FIG. 57 is a set of two graphs illustrating binding of the monoclonal antibodies PG9, PG16, PGT141, PGT142, PTG143, PGT144, PGT145, CH01, CH02, CH03, and CH04, which are specific for the V1/V2 domain of gp120, to the indicated Epitope-Scaffold proteins containing the minimal PG9 epitope (gp120 positions 154-177).

FIG. 58 is a set of images and a graph illustrating that the 2ZJR [[which one-2ZJR_A or 2ZJR_B?]] forms a stable complex with the Fab fragment of PG9 through gel filtration.

FIG. 59 is a series of digital images illustrating Ferritin-, encapsulin- and sulfur oxygenase reductase (SOR)-based protein nanoparticles

FIG. 60 shows an image of a coomassie-stained polyacrylamide gels illustrating that the indicated chimeric nanoparticles are immunoprecipitated by monoclonal antibody PG9 (specific for the gp120 V1/V2 domain), but not by monoclonal antibody VRC01 (specific for the gp120 CD4 binding site).

FIG. 61 shows images of set of coomassie-stained polyacrylamide gels illustrating that the chimeric nanoparticles are immunoprecipitated by monoclonal antibody PG9, PG16 or VRC01. The sequence of the minimal PG9 epitope (gp120 positions 154-177) of HIV-1 strain ZM109 (SEQ ID NO: 2) is shown without substitutions (top sequence), with a C157S substitution (middle sequence) and with K155C, C157S and F176C substitutions (lower sequence).

FIG. 62 shows a digital image illustrating a linked dimer of the gp120 V1/V2 domain binding to monoclonal antibody PG9.

FIG. 63 shows a series of digital images and graphs illustrating binding of a linked dimer of the gp120 V1/V2 domain binding to monoclonal antibody PG9.

FIG. 64 shows a graph and a digital image illustrating that a linked dimer of the gp120 V1/V2 domain binds to monoclonal antibody PG9 through gel filtration.

FIG. 65 shows a schematic diagram and set of three graphs illustrating the affinity of a linked dimer of gp120 V1/V2 domains for monoclonal antibody PG9, and also the affinity of a liked dimer of gp120 V1/V2 domain including truncated V1 and V2 variable loops for monoclonal antibody PG9. The sequence of the V1/V2 domain of HIV-1 strain A244 (SEQ ID NO: 5) is shown, with the A, B, C and D beta-strands, the V1 variable loop, the V2 variable loop, and variable loop substitutions indicated.

FIG. 66 is a table showing neutralization IC50 values for a panel of PG9 resistant HIV-1 Env-pseudoviruses and their corresponding gain of function mutations.

FIG. 67 is a dendrogram illustrating PG9 neutralization sensitivity/resistance. Neighbor-joining dendrogram constructed from full gp160 sequences of 172 virus strains representing the major HIV-1 genetic subtypes (labeled branches). Neutralization sensitivity of each Env-pseudovirus is indicated: PG9-resistant strains not containing a PNGS at residue 160 (black), PG9-sensitive strains (*), and all other PG9-resistant strains (grey).

FIGS. 68A and 68B are a chart and a sequence alignment showing design of gain-of-sensitivity mutants among PG9-resistant strains. (A) V1/V2 amino acid frequency analysis. Symbols correspond to the respective amino acids, with A representing sequence gaps at the given position. For each residue position in the 154-184 range (HXB2-relative numbering), the resistance score for a given amino acid (or a gap) was defined as the ratio of its number of occurrences in resistant sequences vs. its overall number of occurrences for the given residue position. A higher score indicates that the amino acid was preferentially found among resistant sequences, with a score of 1 indicating that the amino was only found among resistant sequences. Residues selected for gain-of-sensitivity studies (and the residue to which they were mutated include F164E, N166R, E168K, (H169K, E169K, T169K, E171K, E173Y and were mutated to the amino acid types shown in green for the specified residue positions. (B) PG9-resistant strains selected for gain-of-function experiments, with residues selected for point-mutations (small boxes) and/or swaps (long boxes). The PG9-sensitive CAP45 sequence, used to determine the atomic structure of V1/V2, is shown as a reference, the long box was used for the swaps. Strands B and C of V1/V2 shown at the top of the figure are based on the CAP45 structure. Residue positions with no variation are shown in white font on black background, while conserved residue positions are shown in bold and boxed in black.

FIG. 69 is a diagram showing the structure-based explanation of gain-of-sensitivity results for V1/V2-directed broadly neutralizing antibodies. The structure of scaffolded-V1/V2 from the CAP45 strain of HIV-1 (dark ribbon with labeled strands and molecular surfaces of glycans 156 and 160) is shown in complex with PG9 (light grey—heavy chain; dark grey—light chain). The side-chains of V1/V2 residues selected for gain-of-sensitivity mutation are shown as sticks and labeled by residue number; side-chains of proximal interacting residues in PG9 CDR H3 are shown as sticks and labeled.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file in the form of the file named Sequence.txt (˜80 kb), which was created on Aug. 27, 2012, and is incorporated by reference herein. In the accompanying Sequence Listing:

SEQ ID NO: 1 is the amino acid sequence of gp120 from HIV-1 strain HXB2 (GENBANK® Accession No. K03455, incorporated by reference herein as present in the database on Jul. 27, 2012).

SEQ ID NO: 2 is the amino acid sequence of gp120 from HIV-1 strain ZM109 (GENBANK® Accession No. AAR09542.2, incorporated by reference herein as present in the database on Jul. 27, 2012).

SEQ ID NO: 3 is the amino acid sequence of gp120 from HIV-1 strain CAP45 (GENBANK® Accession No. ABE02700.1, incorporated by reference herein as present in the database on Jul. 27, 2012).

SEQ ID NO: 4 is the amino acid sequence of gp120 from HIV-1 strain ZM53 (Clade C; GENBANK® Accession No. AAR09394.2, incorporated by reference herein as present in the database on Jul. 27, 2012).

SEQ ID NO: 5 is the amino acid sequence of gp120 from HIV-1 strain A244 (Clade AE; GENBANK® Accession No. AAW57760.1, incorporated by reference herein as present in the database on Jul. 27, 2012).

SEQ ID NO: 6 is the amino acid sequence of gp120 from HIV-1 strain 16055 (Clade C; GENBANK® Accession No. ABL67444.1, incorporated by reference herein as present in the database on Jul. 27, 2012).

SEQ ID NO: 7 is the amino acid sequence of gp120 from HIV-1 strain TRJO (Clade B; GENBANK® Accession No. AAW64265.1, incorporated by reference herein as present in the database on Jul. 27, 2012).

SEQ ID NO: 8 is the amino acid sequence of gp120 from HIV-1 strain ZM233 (Clade C; GENBANK® Accession No. ABD49684.1, incorporated by reference herein as present in the database on Jul. 27, 2012).

SEQ ID NOs: 9-77 are the amino acid sequences of minimal PG9 Epitope-Scaffold proteins.

SEQ ID NOs: 78-112 are the amino acid sequences of native Scaffold proteins.

SEQ ID NO: 113 is the amino acid sequence of a linked dimer of the V1/V2 domain from the CAP45 strain of HIV-1.

SEQ ID NO: 114 is the amino acid sequence of a linked dimer of the V1/V2 domain from the CAP210 strain of HIV-1.

SEQ ID NO: 115 is the amino acid sequence of a linked dimer of the V1/V2 domain from the CA244 strain of HIV-1.

SEQ ID NO: 116 is the amino acid sequence of a linked dimer of the V1/V2 domain from the ZM233 strain of HIV-1.

SEQ ID NO: 117 is the amino acid sequence of a linked dimer of the V1/V2 domain (with truncated variable loops) from the A244 strain of HIV-1.

SEQ ID NO: 118 is the amino acid sequence of a linked dimer of the V1/V2 domain (with truncated variable loops) from the ZM233 strain of HIV-1.

SEQ ID NO: 119 is the amino acid sequence of a Helicobacter pylori ferritin protein (GENBANK® Accession No. EJB64322.1, incorporated by reference herein as present in the database on Jul. 27, 2012).

SEQ ID NO: 120 is the amino acid sequence of a minimal PG9 epitope based on HIV-1 strain ZM109 linked to ferritin.

SEQ ID NO: 121 is the amino acid sequence of a minimal PG9 epitope based on HIV-1 strain CAP45 linked to ferritin.

SEQ ID NO: 122 is the amino acid sequence of a minimal PG9 epitope based on HIV-1 strain A244 linked to ferritin.

SEQ ID NO: 123 is the amino acid sequence of a linked dimer of the V1/V2 domain from the CAP45 strain of HIV-1 linked to ferritin.

SEQ ID NO: 124 is the amino acid sequence of a linked dimer of the V1/V2 domain from the ZM109 strain of HIV-1 linked to ferritin.

SEQ ID NO: 125 is the amino acid sequence of a linked dimer of the V1/V2 domain from the A244 strain of HIV-1 linked to ferritin.

SEQ ID NO: 126 is the amino acid sequence of a linked dimer of the V1/V2 domain (with truncated variable loops) from the A244 strain of HIV-1 linked to ferritin.

SEQ ID NO: 127 is the amino acid sequence of a V1/V2 domain the CAP45 strain of HIV-1 linked to the V1/V2 domain from the A244 strain of HIV-1 linked to ferritin.

SEQ ID NO: 128 is the amino acid sequence of an encapsulin protein (GENBANK® Accession No. YP_{—}001738186.1, incorporated by reference herein as present in the database on Jul. 27, 2012).

SEQ ID NO: 129 is the amino acid sequence of a minimal PG9 epitope based on HIV-1 strain ZM109 linked to encapsulin.

SEQ ID NO: 130 is the amino acid sequence of a minimal PG9 epitope based on HIV-1 strain CAP45 linked to encapsulin.

SEQ ID NO: 131 is the amino acid sequence of a minimal PG9 epitope based on HIV-1 strain A244 linked to encapsulin.

SEQ ID NO: 132 is a consensus amino acid sequence for a minimal PG9 epitope of HIV-1 gp120 including asparagine residues at gp120 positions 156 and 160, and cysteine residues at gp120 positions 155 and 176.

SEQ ID NO: 133 is a consensus amino acid sequence for the minimal PG9 epitope of HIV-1 gp120 including asparagine residues at gp120 positions 160 and 173, and cysteine residues at gp120 positions 155 and 176.

SEQ ID NO: 134 is the amino acid sequence of a minimal PG9 epitope of HIV-1 gp120 including asparagine residues at gp120 positions 156 and 160, and cysteine residues at gp120 positions 155 and 176.

SEQ ID NO: 135 is the amino acid sequence of a minimal PG9 epitope of HIV-1 gp120 including asparagine residues at gp120 positions 160 and 173, and cysteine residues at gp120 positions 155 and 176.

SEQ ID NOs: 136-151 are the amino acid sequences of V1/V2 domain epitope-scaffolds.

SEQ ID NO: 152 is the amino acid sequence of a peptide linker

SEQ ID NO: 153 is the amino acid sequence of a peptide linker

SEQ ID NO: 154 is the amino acid sequence of the Envelope protein including gp120 from the HIV-1 strain 92UG037 (Clade A; GENBANK® Acc. No. AAC97548.1, incorporated by reference herein in its entirety as present in the database on Aug. 27, 2012).

SEQ ID NO: 155 is the amino acid sequence of the Envelope protein including gp120 from the HIV-1 strain 92RW020 (Clade A; GENBANK® Acc. No. AAT67478.1, incorporated by reference herein in its entirety as present in the database on Aug. 27, 2012).

SEQ ID NO: 156 is the amino acid sequence of the Envelope protein including gp120 from the HIV-1 strain JRCSF (Clade B; GENBANK® Acc. No. AAR05850.1, incorporated by reference herein in its entirety as present in the database on Aug. 27, 2012).

SEQ ID NO: 157 is the amino acid sequence of the Envelope protein including gp120 from the HIV-1 strain REJO (Clade B; GENBANK® Acc. No. AET76122.1, incorporated by reference herein in its entirety as present in the database on Aug. 27, 2012).

SEQ ID NO: 158 is the amino acid sequence of the Envelope protein including gp120 from the HIV-1 strain 247-23 (Clade D; GENBANK® Acc. No. ACD63071.1, incorporated by reference herein in its entirety as present in the database on Aug. 27, 2012).

SEQ ID NO: 159 is the amino acid sequence of the Envelope protein including gp120 from the HIV-1 strain 98UG57128 (Clade D; GENBANK® Acc. No. AAN73661.1, incorporated by reference herein in its entirety as present in the database on Aug. 27, 2012).

SEQ ID NO: 160 is the amino acid sequence of the Envelope protein including gp120 from the HIV-1 strain 92TH021 (Clade AE; GENBANK® Acc. No. AAT67547.1, incorporated by reference herein in its entirety as present in the database on Aug. 27, 2012).

SEQ ID NOs: 161-172 are the amino acid sequences of kabat positions 87-115 of the heavy chain variable regions of the PG9, PG16, CH01, CH02, CH03, CH04, PGT141, PGT142, PGT143, PGT144, PGT145 and 2909, respectively.

SEQ ID NO: 173 is the amino acid sequences of a V1/V2 domain epitope-scaffold.

SEQ ID NOs: 174-196 are the amino acid sequences of positions 154-184 (HXB2 numbering) of HIV-1 gp120 strains.

### DETAILED DESCRIPTION

### I. Terms

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.”

As used herein, the term “comprises” means “includes.” Thus, “comprising an antigen” means “including an antigen” without excluding other elements.

It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting.

To facilitate review of the various embodiments, the following explanations of terms are provided:

Adjuvant: A vehicle used to enhance antigenicity. Adjuvants include a suspension of minerals (alum, aluminum hydroxide, or phosphate) on which antigen is adsorbed; or water-in-oil emulsion in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). Immunostimulatory oligonucleotides (such as those including a CpG motif) can also be used as adjuvants (for example see U.S. Pat. No. 6,194,388; U.S. Pat. No. 6,207,646; U.S. Pat. No. 6,214,806; U.S. Pat. No. 6,218,371; U.S. Pat. No. 6,239,116; U.S. Pat. No. 6,339,068; U.S. Pat. No. 6,406,705; and U.S. Pat. No. 6,429,199). Adjuvants include biological molecules (a “biological adjuvant”), such as costimulatory molecules. Exemplary adjuvants include IL-2, RANTES, GM-CSF, TNF-α, IFN-γ, G-CSF, LFA-3, CD72, B7-1, B7-2, OX-40L and 41 BBL. Adjuvants can be used in combination with the disclosed antigens containing a PG9 epitope.

Administration: The introduction of a composition into a subject by a chosen route. Administration can be local or systemic. For example, if the chosen route is intravenous, the composition (such as a composition including a disclosed immunogen) is administered by introducing the composition into a vein of the subject.

Agent: Any substance or any combination of substances that is useful for achieving an end or result; for example, a substance or combination of substances useful for inhibiting HIV infection in a subject. Agents include proteins, nucleic acid molecules, compounds, small molecules, organic compounds, inorganic compounds, or other molecules of interest, such as viruses, such as recombinant viruses. An agent can include a therapeutic agent (such as an anti-retroviral agent), a diagnostic agent or a pharmaceutical agent. In some embodiments, the agent is a polypeptide agent (such as a HIV-neutralizing polypeptide), or an anti-viral agent. The skilled artisan will understand that particular agents may be useful to achieve more than one result.

Amino acid substitutions: The replacement of one amino acid in an antigen with a different amino acid. In some examples, an amino acid in an antigen is substituted with an amino acid from a homologous antigen.

Animal: A living multicellular vertebrate organism, a category that includes, for example, mammals and birds. A “mammal” includes both human and non-human mammals, such as mice. The term “subject” includes both human and animal subjects, such as non-human primates.

Antibody: A polypeptide substantially encoded by an immunoglobulin gene or immunoglobulin genes, or fragments thereof, which specifically binds and recognizes an analyte (such as an antigen or immunogen) such as a gp120 polypeptide or antigenic fragment thereof, such as a PG9 epitope on a resurfaced gp120 polypeptide or antigenic fragment thereof. Immunoglobulin genes include the kappa, lambda, alpha, gamma, delta, epsilon and mu constant region genes, as well as the myriad immunoglobulin variable region genes.

Antibodies exist, for example as intact immunoglobulins and as a number of well characterized fragments produced by digestion with various peptidases. For instance, Fabs, Fvs, and single-chain Fvs (SCFvs) that bind to gp120, would be gp120-specific binding agents. This includes intact immunoglobulins and the variants and portions of them well known in the art, such as Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (such as humanized murine antibodies), heteroconjugate antibodies (such as bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

Antibody fragments are defined as follows: (1) Fab, the fragment which contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)2, the fragment of the antibody obtained by treating whole antibody with the enzyme pepsin without subsequent reduction; (4) F(ab′)2, a dimer of two Fab′ fragments held together by two disulfide bonds; (5) Fv, a genetically engineered fragment containing the variable region of the light chain and the variable region of the heavy chain expressed as two chains; and (6) single chain antibody (“SCA”), a genetically engineered molecule containing the variable region of the light chain, the variable region of the heavy chain, linked by a suitable polypeptide linker as a genetically fused single chain molecule. The term “antibody,” as used herein, also includes antibody fragments either produced by the modification of whole antibodies or those synthesized de novo using recombinant DNA methodologies.

Typically, a naturally occurring immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs.” The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. Light chain CDRs are sometimes referred to as CDR L1, CDR L2, and CDR L3. Heavy chain CDRs are sometimes referred to as CDR H1, CDR H2, and CDR H3.

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. These fused cells and their progeny are termed “hybridomas.” Monoclonal antibodies include humanized monoclonal antibodies.

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous antigens, such as the disclosed PG9 epitope antigens. “Epitope” or “antigenic determinant” refers to the region of an antigen to which B and/or T cells respond. In one embodiment, T cells respond to the epitope, when the epitope is presented in conjunction with an MHC molecule. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and nuclear magnetic resonance.

Examples of antigens include, but are not limited to, polypeptides, peptides, lipids, polysaccharides, combinations thereof (such as glycopeptides) and nucleic acids containing antigenic determinants, such as those recognized by an immune cell. In some examples, antigens include peptides derived from a pathogen of interest, such as HIV. Exemplary pathogens include bacteria, fungi, viruses and parasites. In specific examples, an antigen is derived from HIV, such as an antigen including a PG9 epitope.

A “target epitope” is a specific epitope on an antigen that specifically binds an antibody of interest, such as a monoclonal antibody. In some examples, a target epitope includes the amino acid residues that contact the antibody of interest, such that the target epitope can be selected by the amino acid residues determined to be in contact with the antibody of interest. A PG9 epitope antigen is an antigen that includes a PG9 epitope.

Anti-retroviral agent: An agent that specifically inhibits a retrovirus from replicating or infecting cells. Non-limiting examples of antiretroviral drugs include entry inhibitors (e.g., enfuvirtide), CCR5 receptor antagonists (e.g., aplaviroc, vicriviroc, maraviroc), reverse transcriptase inhibitors (e.g., lamivudine, zidovudine, abacavir, tenofovir, emtricitabine, efavirenz), protease inhibitors (e.g., lopivar, ritonavir, raltegravir, darunavir, atazanavir), maturation inhibitors (e.g., alpha interferon, bevirimat and vivecon).

Atomic Coordinates or Structure coordinates: Mathematical coordinates derived from mathematical equations related to the patterns obtained on diffraction of a monochromatic beam of X-rays by the atoms (scattering centers) such as an antigen, or an antigen in complex with an antibody. In some examples that antigen can be gp120, a gp120:antibody complex, or combinations thereof in a crystal. The diffraction data are used to calculate an electron density map of the repeating unit of the crystal. The electron density maps are used to establish the positions of the individual atoms within the unit cell of the crystal. In one example, the term “structure coordinates” refers to Cartesian coordinates derived from mathematical equations related to the patterns obtained on diffraction of a monochromatic beam of X-rays, such as by the atoms of a gp120 in crystal form.

Those of ordinary skill in the art understand that a set of structure coordinates determined by X-ray crystallography is not without standard error. For the purpose of this disclosure, any set of structure coordinates that have a root mean square deviation of protein backbone atoms (N, Ca, C and O) of less than about 1.0 Angstroms when superimposed, such as about 0.75, or about 0.5, or about 0.25 Angstroms, using backbone atoms, shall (in the absence of an explicit statement to the contrary) be considered identical.

Contacting: Placement in direct physical association; includes both in solid and liquid form. Contacting includes contact between one molecule and another molecule, for example the amino acid on the surface of one polypeptide, such as an antigen, that contact another polypeptide, such as an antibody. Contacting also includes administration, such as administration of a disclosed antigen to a subject by a chosen route.

Control: A reference standard. In some embodiments, the control is a negative control sample obtained from a healthy patient. In other embodiments, the control is a positive control sample obtained from a patient diagnosed with HIV infection. In still other embodiments, the control is a historical control or standard reference value or range of values (such as a previously tested control sample, such as a group of HIV patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Degenerate variant and conservative variant: A polynucleotide encoding a polypeptide or an antibody that includes a sequence that is degenerate as a result of the genetic code. For example, a polynucleotide encoding a disclosed antigen or an antibody that specifically binds a disclosed antigen includes a sequence that is degenerate as a result of the genetic code. There are 20 natural amino acids, most of which are specified by more than one codon. Therefore, all degenerate nucleotide sequences are included as long as the amino acid sequence of the antigen or antibody that binds the antigen encoded by the nucleotide sequence is unchanged. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified within a protein encoding sequence, the codon can be altered to any of the corresponding codons described without altering the encoded protein. Such nucleic acid variations are “silent variations,” which are one species of conservative variations. Each nucleic acid sequence herein that encodes a polypeptide also describes every possible silent variation. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

One of ordinary skill will recognize that individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

Conservative amino acid substitutions providing functionally similar amino acids are well known in the art. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

Not all residue positions within a protein will tolerate an otherwise “conservative” substitution. For instance, if an amino acid residue is essential for a function of the protein, even an otherwise conservative substitution may disrupt that activity, for example the specific binding of an antibody to a target epitope may be disrupted by a conservative mutation in the target epitope.

Epitope: An antigenic determinant. These are particular chemical groups or peptide sequences on a molecule that are antigenic, such that they elicit a specific immune response, for example, an epitope is the region of an antigen to which B and/or T cells respond. An antibody binds a particular antigenic epitope, such as an epitope of a gp120 polypeptide, for example a PG9 epitope.

Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and nuclear magnetic resonance. Epitopes can also include post-translation modification of amino acids, such as N-linked glycosylation.

Epitope-Scaffold Protein: A chimeric protein that includes an epitope sequence fused to a heterologous “acceptor” scaffold protein. Design of the epitope-scaffold is performed, for example, computationally in a manner that preserves the native structure and conformation of the epitope when it is fused onto the heterologous scaffold protein. In several embodiments, mutations (such as amino acid substitutions, insertions and/or deletions) within the epitope sequence or the heterologous scaffold are made in order to accommodate the epitope fusion. Several embodiments include an epitope scaffold protein with a PG9 epitope included on a heterologous scaffold protein. Methods for the design and construction of epitope—scaffold proteins are described herein and also familiar to the person of ordinary skill in the art (see, for example, U.S. Patent Application Publication No. 2010/0068217, incorporated by reference herein in its entirety).

Effective amount: An amount of agent, such as nucleic acid vaccine or other agent that is sufficient to generate a desired response, such as reduce or eliminate a sign or symptom of a condition or disease, such as AIDS. For instance, this can be the amount necessary to inhibit viral replication or to measurably alter outward symptoms of the viral infection, such as increase of T cell counts in the case of an HIV-1 infection. In general, this amount will be sufficient to measurably inhibit virus (for example, HIV) replication or infectivity. When administered to a subject, a dosage will generally be used that will achieve target tissue concentrations (for example, in lymphocytes) that has been shown to achieve in vitro inhibition of viral replication. In some examples, an “effective amount” is one that treats (including prophylaxis) one or more symptoms and/or underlying causes of any of a disorder or disease, for example to treat HIV. In one example, an effective amount is a therapeutically effective amount. In one example, an effective amount is an amount that prevents one or more signs or symptoms of a particular disease or condition from developing, such as one or more signs or symptoms associated with AIDS.

Expression: Translation of a nucleic acid into a protein. Proteins may be expressed and remain intracellular, become a component of the cell surface membrane, or be secreted into the extracellular matrix or medium.

Expression Control Sequences: Nucleic acid sequences that regulate the expression of a heterologous nucleic acid sequence to which it is operatively linked Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

A polynucleotide can be inserted into an expression vector that contains a promoter sequence, which facilitates the efficient transcription of the inserted genetic sequence of the host. The expression vector typically contains an origin of replication, a promoter, as well as specific nucleic acid sequences that allow phenotypic selection of the transformed cells.

Foldon domain: An amino acid sequence that naturally forms a trimeric structure. In some examples, a foldon domain can be included in the amino acid sequence of a disclosed PG9 epitope antigen so that the antigen will form a trimer. In one example, a foldon domain is the T4 foldon domain.

Glycoprotein (gp): A protein that contains oligosaccharide chains (glycans) covalently attached to polypeptide side-chains. The carbohydrate is attached to the protein in a cotranslational or posttranslational modification. This process is known as glycosylation. In proteins that have segments extending extracellularly, the extracellular segments are often glycosylated. Glycoproteins are often important integral membrane proteins, where they play a role in cell-cell interactions. In some examples a glycoprotein is an HIV glycoprotein, such as a HIV gp120, gp140 or an immunogenic fragment thereof.

Glycosylation site: An amino acid sequence on the surface of a polypeptide, such as a protein, which accommodates the attachment of a glycan. An N-linked glycosylation site is triplet sequence of NXS/T in which N is asparagine, X is any residues except proline, S/T means serine or threonine. A glycan is a polysaccharide or oligosaccharide. Glycan may also be used to refer to the carbohydrate portion of a glycoconjugate, such as a glycoprotein, glycolipid, or a proteoglycan.

gp120: The envelope protein from Human Immunodeficiency Virus (HIV). The envelope protein is initially synthesized as a longer precursor protein of 845-870 amino acids in size, designated gp160. Gp160 forms a homotrimer and undergoes glycosylation within the Golgi apparatus. It is then cleaved by a cellular protease into gp120 and gp41. Gp41 contains a transmembrane domain and remains in a trimeric configuration; it interacts with gp120 in a non-covalent manner. Gp120 contains most of the external, surface-exposed, domains of the envelope glycoprotein complex, and it is gp120 which binds both to the cellular CD4 receptor and to the cellular chemokine receptors (such as CCR5).

The mature gp120 wildtype polypeptides have about 500 amino acids in the primary sequence. Gp120 is heavily N-glycosylated giving rise to an apparent molecular weight of 120 kD. The polypeptide is comprised of five conserved regions (C1-05) and five regions of high variability (V1-V5). Exemplary sequence of wt gp160 polypeptides are shown on GENBANK, for example accession numbers AAB05604 and AAD12142

Variable region 1 and Variable Region 2 (V1/V2 domain) of gp120 are comprised of ˜50-90 residues which contain two of the most variable portions of HIV-1 (the V1 loop and the V2 loop), and one in ten residues of the V1/V2 domain are N-glycosylated. Despite the diversity and glycosylation of the V1/V2 domain, a number of broadly neutralizing human antibodies have been identified that target this region, including the somatically related antibodies PG9 and PG16 (Walker et al., Science, 326:285-289, 2009). In certain examples the V1/V2 domain includes gp120 position 126-196.

gp140: An oligomeric form of HIV envelope protein, which contains all of gp120 and the entire gp41 ectodomain.

gp41: A HIV protein that contains a transmembrane domain and remains in a trimeric configuration; it interacts with gp120 in a non-covalent manner. The envelope protein of HIV-1 is initially synthesized as a longer precursor protein of 845-870 amino acids in size, designated gp160. gp160 forms a homotrimer and undergoes glycosylation within the Golgi apparatus. In vivo, it is then cleaved by a cellular protease into gp120 and gp41. The amino acid sequence of an exemplary gp41 is set forth in GENBANK® Accession No. CAD20975 (as available on Aug. 27, 2009) which is incorporated by reference herein. gp41 contains a transmembrane domain and typically remains in a trimeric configuration; it interacts with gp120 in a non-covalent manner.

Highly active anti-retroviral therapy (HAART): A therapeutic treatment for HIV infection involving administration of multiple anti-retroviral agents (e.g., two, three or four anti-retroviral agents) to an HIV infected individual during a course of treatment. Non-limiting examples of antiretroviral agents include entry inhibitors (e.g., enfuvirtide), CCR5 receptor antagonists (e.g., aplaviroc, vicriviroc, maraviroc), reverse transcriptase inhibitors (e.g., lamivudine, zidovudine, abacavir, tenofovir, emtricitabine, efavirenz), protease inhibitors (e.g., lopivar, ritonavir, raltegravir, darunavir, atazanavir), maturation inhibitors (e.g., alpha interferon, bevirimat and vivecon). One example of a HAART regimen includes treatment with a combination of tenofovir, emtricitabine and efavirenz.

HIV Envelope protein (Env): The HIV envelope protein is initially synthesized as a longer precursor protein of 845-870 amino acids in size, designated gp160. gp160 forms a homotrimer and undergoes glycosylation within the Golgi apparatus. In vivo, it is then cleaved by a cellular protease into gp120 and gp41. gp120 contains most of the external, surface-exposed, domains of the HIV envelope glycoprotein complex, and it is gp120 which binds both to cellular CD4 receptors and to cellular chemokine receptors (such as CCR5). gp41 contains a transmembrane domain and remains in a trimeric configuration; it interacts with gp120 in a non-covalent manner.

Homologous proteins: Proteins from two or more species that have a similar structure and function in the two or more species. For example a gp120 antigen from one species of lentivirus such as HIV-1 is a homologous antigen to a gp120 antigen from a related species such as HIV-2 or SIV. Homologous proteins share the same protein fold and can be considered structural homologs.

Homologous proteins typically share a high degree of sequence conservation, such as at least 50%, at least 60%, at least 70%, at least 80% or at least 90% sequence conservation. Homologous proteins can share a high degree of sequence identity, such as at least 30% at least 40% at least 50%, at least 60%, at least 70%, at least 80% or at least 90% sequence identity.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Human Immunodeficiency Virus (HIV): A retrovirus that causes immunosuppression in humans (HIV disease), and leads to a disease complex known as the acquired immunodeficiency syndrome (AIDS). “HIV disease” refers to a well-recognized constellation of signs and symptoms (including the development of opportunistic infections) in persons who are infected by an HIV virus, as determined by antibody or western blot studies. Laboratory findings associated with this disease include a progressive decline in T cells. HIV includes HIV type 1 (HIV-1) and HIV type 2 (HIV-2). Related viruses that are used as animal models include simian immunodeficiency virus (SIV), and feline immunodeficiency virus (FIV). Treatment of HIV-1 with HAART has been effective in reducing the viral burden and ameliorating the effects of HIV-1 infection in infected individuals.

HXB2 numbering system: A reference numbering system for HIV protein and nucleic acid sequences, using HIV-1 HXB2 strain sequences as a reference for all other HIV strain sequences. The person of ordinary skill in the art is familiar with the HXB2 numbering system, and this system is set forth in “Numbering Positions in HIV Relative to HXB2CG,” Bette Korber et al., Human Retroviruses and AIDS 1998: A Compilation and Analysis of Nucleic Acid and Amino Acid Sequences. Korber B, Kuiken C L, Foley B, Hahn B, McCutchan F, Mellors J W, and Sodroski J, Eds. Theoretical Biology and Biophysics Group, Los Alamos National Laboratory, Los Alamos, N. Mex., which is incorporated by reference herein in its entirety. For reference, the amino acid sequence of HXB2CG is provided as SEQ ID NO: 1. HXB2 is also known as: HXBc2, for HXB clone 2; HXB2R, in the Los Alamos HIV database, with the R for revised, as it was slightly revised relative to the original HXB2 sequence; and HXB2CG in GENBANK™, for HXB2 complete genome. The numbering used in gp120 polypeptides disclosed herein is relative to the HXB2 numbering scheme.

Immunogen: A protein or a portion thereof that is capable of inducing an immune response in a mammal, such as a mammal infected or at risk of infection with a pathogen. Administration of an immunogen can lead to protective immunity and/or proactive immunity against a pathogen of interest. In some examples, an immunogen is an PG9 epitope antigen, such as a PG9 epitope antigen including a PG9 epitope stabilized in a PG9 bound conformation.

Immunogenic surface: A surface of a molecule, for example a protein such as gp120, capable of eliciting an immune response. An immunogenic surface includes the defining features of that surface, for example the three-dimensional shape and the surface charge. In some examples, an immunogenic surface is defined by the amino acids on the surface of a protein or peptide that are in contact with an antibody, such as a neutralizing antibody, when the protein and the antibody are bound together. A target epitope includes an immunogenic surface. Immunogenic surface is synonymous with antigenic surface.

Immune response: A response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies.

Immunogenic composition: A composition comprising an immunogenic polypeptide that induces a measurable CTL response against virus expressing the immunogenic polypeptide, or induces a measurable B cell response (such as production of antibodies) against the immunogenic polypeptide. In one example, an “immunogenic composition” is composition includes a disclosed PG9 epitope antigen derived from a gp120, that induces a measurable CTL response against virus expressing gp120 polypeptide, or induces a measurable B cell response (such as production of antibodies) against a gp120 polypeptide. It further refers to isolated nucleic acids encoding an antigen, such as a nucleic acid that can be used to express the antigen (and thus be used to elicit an immune response against this polypeptide).

For in vitro use, an immunogenic composition may consist of the isolated protein, peptide epitope, or nucleic acid encoding the protein, or peptide epitope. For in vivo use, the immunogenic composition will typically include the protein, immunogenic peptide or nucleic acid in pharmaceutically acceptable carriers, and/or other agents. Any particular peptide, such as a disclosed PG9 epitope antigen or a nucleic acid encoding the antigen, can be readily tested for its ability to induce a CTL or B cell response by art-recognized assays. Immunogenic compositions can include adjuvants, which are well known to one of skill in the art.

Immunological Probe: A molecule that can be used for selection of antibodies from sera which are directed against a specific epitope, including from human patient sera. The epitope scaffolds, along with related point mutants, can be used as immunological probes in both positive and negative selection of antibodies against the epitope graft. In some examples immunological probes are engineered variants of gp120.

Inhibiting or treating a disease: Inhibiting the full development of a disease or condition, for example, in a subject who is at risk for a disease such as acquired immune deficiency syndrome (AIDS), AIDS related conditions, HIV-1 infection, or combinations thereof. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of metastases, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

Isolated: An “isolated” biological component (such as a protein, for example a disclosed PG9 epitope antigen or nucleic acid encoding such an antigen) has been substantially separated or purified away from other biological components in which the component naturally occurs, such as other chromosomal and extrachromosomal DNA, RNA, and proteins. Proteins, peptides and nucleic acids that have been “isolated” include proteins purified by standard purification methods. The term also embraces proteins or peptides prepared by recombinant expression in a host cell as well as chemically synthesized proteins, peptides and nucleic acid molecules. Isolated does not require absolute purity, and can include protein, peptide, or nucleic acid molecules that are at least 50% isolated, such as at least 75%, 80%, 90%, 95%, 98%, 99%, or even 99.9% isolated.

Label: A detectable compound or composition that is conjugated directly or indirectly to another molecule to facilitate detection of that molecule. Specific, non-limiting examples of labels include fluorescent tags, enzymatic linkages, and radioactive isotopes. In some examples, a disclosed PG9 epitope antigen is labeled with a detectable label. In some examples, label is attached to a disclosed antigen or nucleic acid encoding such an antigen.

Native antigen or native sequence: An antigen or sequence that has not been modified by selective mutation, for example, selective mutation to focus the antigenicity of the antigen to a target epitope. Native antigen or native sequence are also referred to as wild-type antigen or wild-type sequence.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. In some examples, a nucleic acid encodes a disclosed PG9 epitope antigen.

“Recombinant nucleic acid” refers to a nucleic acid having nucleotide sequences that are not naturally joined together. This includes nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce, such as a “recombinant polypeptide.” A recombinant nucleic acid may serve a non-coding function (such as a promoter, origin of replication, ribosome-binding site, etc.) as well.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Peptide: Any compound composed of amino acids, amino acid analogs, chemically bound together. Peptide as used herein includes oligomers of amino acids, amino acid analog, or small and large peptides, including polypeptides or proteins. Peptides include any chain of amino acids, regardless of length or post-translational modification (such as glycosylation or phosphorylation). “Peptide” applies to amino acid polymers to naturally occurring amino acid polymers and non-naturally occurring amino acid polymer as well as in which one or more amino acid residue is a non-natural amino acid, for example an artificial chemical mimetic of a corresponding naturally occurring amino acid. A “residue” refers to an amino acid or amino acid mimetic incorporated in a polypeptide by an amide bond or amide bond mimetic. A peptide has an amino terminal (N-terminal) end and a carboxy terminal (C-terminal) end.

A “protein” or “polypeptide” is a peptide that folds into a specific three-dimensional structure. A protein can include surface-exposed amino acid resides and non-surface-exposed amino acid resides. “Surface-exposed amino acid residues” are those amino acids that have some degree of exposure on the surface of the protein, for example such that they can contact the solvent when the protein is in solution. In contrast, non-surface-exposed amino acids are those amino acid residues that are not exposed on the surface of the protein, such that they do not contact solution when the protein is in solution. In some examples, the non-surface-exposed amino acid residues are part of the protein core.

A “protein core” is the interior of a folded protein, which is substantially free of solvent exposure, such as solvent in the form of water molecules in solution. Typically, the protein core is predominately composed of hydrophobic or apolar amino acids. In some examples, a protein core may contain charged amino acids, for example aspartic acid, glutamic acid, arginine, and/or lysine. The inclusion of uncompensated charged amino acids (a compensated charged amino can be in the form of a salt bridge) in the protein core can lead to a destabilized protein. That is, a protein with a lower Tₘ then a similar protein without an uncompensated charged amino acid in the protein core. In other examples, a protein core may have a cavity within the protein core. Cavities are essentially voids within a folded protein where amino acids or amino acid side chains are not present. Such cavities can also destabilize a protein relative to a similar protein without a cavity. Thus, when creating a stabilized form of a protein, it may be advantageous to substitute amino acid residues within the core in order to fill cavities present in the wild-type protein.

Amino acids in a peptide, polypeptide or protein generally are chemically bound together via amide linkages (CONH). Additionally, amino acids may be bound together by other chemical bonds. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO— (These and others can be found in Spatola, in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci pp. 463-468, 1980; Hudson, et al., Int J Pept Prot Res 14:177-185, 1979; Spatola et al. Life Sci 38:1243-1249, 1986; Harm J. Chem. Soc Perkin Trans. 1307-314, 1982; Almquist et al. J. Med. Chem. 23:1392-1398, 1980; Jennings-White et al. Tetrahedron Lett 23:2533, 1982; Holladay et al. Tetrahedron. Lett 24:4401-4404, 1983; and Hruby Life Sci 31:189-199, 1982.

Peptide modifications: Peptides, such as the HIV immunogens disclosed herein can be modified by a variety of chemical techniques to produce derivatives having essentially the same activity as the unmodified peptides, and optionally having other desirable properties. For example, carboxylic acid groups of the protein, whether carboxyl-terminal or side chain, may be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring. Amino groups of the peptide, whether amino-terminal or side chain, may be in the form of a pharmaceutically-acceptable acid addition salt, such as the HCl, HBr, acetic, benzoic, toluene sulfonic, maleic, tartaric and other organic salts, or may be modified to C₁-C₁₆ alkyl or dialkyl amino or further converted to an amide.

Hydroxyl groups of the peptide side chains can be converted to C₁-C₁₆ alkoxy or to a C₁-C₁₆ ester using well-recognized techniques. Phenyl and phenolic rings of the peptide side chains can be substituted with one or more halogen atoms, such as F, Cl, Br or I, or with C₁-C₁₆ alkyl, C₁-C₁₆ alkoxy, carboxylic acids and esters thereof, or amides of such carboxylic acids. Methylene groups of the peptide side chains can be extended to homologous C₂-C₄ alkylenes. Thiols can be protected with any one of a number of well-recognized protecting groups, such as acetamide groups. Those skilled in the art will also recognize methods for introducing cyclic structures into the peptides of this disclosure to select and provide conformational constraints to the structure that result in enhanced stability. For example, a C- or N-terminal cysteine can be added to the peptide, so that when oxidized the peptide will contain a disulfide bond, generating a cyclic peptide. Other peptide cyclizing methods include the formation of thioethers and carboxyl- and amino-terminal amides and esters.

PG9: A broadly neutralizing monoclonal antibody that specifically binds to the V1/V2 domain of HIV-1 gp120 and prevents HIV-1 infection of target cells (see, for example, PCT Publication No. WO/2010/107939, and Walker et al., Nature, 477:466-470, 2011, each of which is incorporated by reference herein). PG9 protein and nucleic acid sequences are known, for example, the heavy and light chain amino acid sequences of the PG9 antibody are set forth as SEQ ID NO: 28 and SEQ ID NO: 30, respectively, of PCT Publication No. WO/2010/107939. Exemplary nucleic acid sequences encoding the heavy and light chains of the PG9 antibody are set forth as SEQ ID NO: 27 and SEQ ID NO: 29, respectively, of PCT Publication No. WO/2010/107939. The person of ordinary skill in the art is familiar with monoclonal antibody PG9 and with methods of producing this antibody.

PG9-bound conformation: The three-dimensional structure of the PG9 epitope of gp120 when bound by PG9, as described herein. In several embodiments, isolated antigens are disclosed herein that include a PG9 epitope from a HIV-1 gp120 polypeptide (referred to herein as “PG9-epitope antigens”). Several such embodiments include an antigen including a PG9 epitope in a PG9 bound conformation. The three-dimensional structure of the PG9 Fab fragment in complex with the V1/V2 domain of gp120 from two different HIV-1 strains (CAP 45 and ZM109) is disclosed herein (see Example 1). The coordinates for these three-dimensional structures are deposited in the Protein Data Bank (PDB) and are set forth as PDB Accession Nos. 3U4E (showing V1/V2 from HIV-1 CAP45 in complex with PG9 Fab) and 3U2S (showing V1/V2 from HIV-1 ZM109 in complex with PG9 Fab), each of which is incorporated by reference herein in their entirety as present in the database on Aug. 27, 2012. These two structures illustrate PG9 epitopes in a PG9-bound conformation, wherein the gp120 V1/V2 domain adopts a four-stranded anti-parallel beta-sheet, with PG9 forming hydrogen bonds with a first N-linked glycan at gp120 position 160 and a second N-linked glycan at gp120 position 156 of CAP45, or position 173 of ZM109. Due to the conformation of the underlying beta-sheet, the N-linked glycan at position 156 of HIV-1 CAP45 occupies substantially the same three-dimensional space as the N-linked glycan at position 173 of HIV-1 ZM109, when bound to PG9. These structures also illustrate that the minimal PG9 epitope includes a two stranded anti-parallel beta-sheet including gp120 positions 154-177, with a first N-linked glycan at gp120 position 160 and a second N-linked glycan at gp120 position 156 or position 173, but not both. Methods of determining if a disclosed antigen includes a PG9 epitope in a PG9-bound conformation are known to the person of ordinary skill in the art and further disclosed herein (see, for example, McLellan et al., Nature, 480:336-343, 2011; and U.S. Patent Application Publication No. 2010/0068217, incorporated by reference herein in its entirety).

Pharmaceutical agent or drug: A chemical compound or composition capable of inducing a desired therapeutic or prophylactic effect when properly administered to a subject.

Pharmaceutically acceptable carriers: The pharmaceutically acceptable carriers useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of the proteins and other compositions herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions, powder, pill, tablet, or capsule forms, conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Purified: The term purified does not require absolute purity; rather, it is intended as a relative term. Thus, for example, a purified protein is one in which the protein is more enriched than the protein is in its natural environment within a cell. Preferably, a preparation is purified such that the protein represents at least 50% of the protein content of the preparation.

The immunogens disclosed herein, or antibodies that specifically bind the disclosed resurfaced immunogens, can be purified by any of the means known in the art. See for example Guide to Protein Purification, ed. Deutscher, Meth. Enzymol. 185, Academic Press, San Diego, 1990; and Scopes, Protein Purification: Principles and Practice, Springer Verlag, New York, 1982. Substantial purification denotes purification from other proteins or cellular components. A substantially purified protein is at least 60%, 70%, 80%, 90%, 95% or 98% pure. Thus, in one specific, non-limiting example, a substantially purified protein is 90% free of other proteins or cellular components.

Protein nanoparticle: A multi-subunit, protein-based polyhedron shaped structure. The subunits are each composed of proteins or polypeptides (for example a glycosylated polypeptide), and, optionally of single or multiple features of the following: nucleic acids, prosthetic groups, organic and inorganic compounds. Non-limiting examples of protein nanoparticles include ferritin nanoparticles (see, e.g., Zhang, Y. Int. J. Mol. Sci., 12:5406-5421, 2011, encapsulin nanoparticles (see, e.g., Sutter et al., Nature Struct. and Mol. Biol., 15:939-947, 2008 and Sulfur Oxygenase Reductase (SOR) nanoparticles (see, e.g., Urich et al., Science, 311:996-1000, 2006). Ferritin, encapsulin and SOR are monomeric proteins that self-assemble into a globular protein complexes that in some cases consists of 24, 60 and 24 protein subunits, respectively. In some examples, ferritin, encapsulin and SOR monomers are linked to a disclosed antigen (for example, an antigen including a PG9 epitope) and self-assembled into a protein nanoparticle presenting the disclosed antigens on its surface, which can be administered to a subject to stimulate an immune response to the antigen.

Resurfaced antigen or resurfaced immunogen: A polypeptide immunogen derived from a wild-type antigen in which amino acid residues outside or exterior to a target epitope are mutated in a systematic way to focus the immunogenicity of the antigen to the selected target epitope. In some examples a resurfaced antigen is referred to as an antigenically-cloaked immunogen or antigenically-cloaked antigen.

Root mean square deviation (RMSD): The square root of the arithmetic mean of the squares of the deviations from the mean. In several embodiments, RMSD is used as a way of expressing deviation or variation from the structural coordinates of a reference three dimensional structure. This number is typically calculated after optimal superposition of two structures, as the square root of the mean square distances between equivalent C_{α} atoms. In some embodiments, the reference three-dimensional structure includes the structural coordinates of the V1/V2 domain of HIV-1 gp120 bound to monoclonal antibody PG9, set forth as Protein Data Bank Accession Nos 3U4E (CAP45 gp120) and 3U2S (ZM109 gp120), each of which is incorporated by reference herein in their entirety as present in the database on Aug. 27, 2012.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a peptide sequence that has 1166 matches when aligned with a test sequence having 1554 nucleotides is 75.0 percent identical to the test sequence (1166+1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer.

For sequence comparison of nucleic acid sequences and amino acids sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, for example, by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see for example, Current Protocols in Molecular Biology (Ausubel et al., eds 1995 supplement)). The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (World Wide Web address ncbi.nlm.nih.gov). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring Matrix (see Henikoff & Henikoff, Proc. Natl. Acad. Sci. USA 89:10915, 1989).

Another indicia of sequence similarity between two nucleic acids is the ability to hybridize. The more similar are the sequences of the two nucleic acids, the more stringent the conditions at which they will hybridize. The stringency of hybridization conditions are sequence-dependent and are different under different environmental parameters. Thus, hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found, for example, in Sambrook et al., Molecular Cloning: A Laboratory Manual, Cold Spring Harbor Laboratory Press, Cold Spring Harbor, N.Y., 2001; Tijssen, Hybridization With Nucleic Acid Probes, Part I: Theory and Nucleic Acid Preparation, Laboratory Techniques in Biochemistry and Molecular Biology, Elsevier Science Ltd., NY, N.Y., 1993. and Ausubel et al. Short Protocols in Molecular Biology, 4^{th} ed., John Wiley & Sons, Inc., 1999.

“Stringent conditions” encompass conditions under which hybridization will only occur if there is less than 25% mismatch between the hybridization molecule and the target sequence. “Stringent conditions” may be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize. In contrast nucleic acids that hybridize under “low stringency conditions include those with much less sequence identity, or with sequence identity over only short subsequences of the nucleic acid.

Specifically bind: When referring to the formation of an antibody:antigen protein complex, refers to a binding reaction which determines the presence of a target protein, peptide, or polysaccharide (for example a glycoprotein), in the presence of a heterogeneous population of proteins and other biologics. Thus, under designated conditions, an antibody binds preferentially to a particular target protein, peptide or polysaccharide (such as an antigen present on the surface of a pathogen, for example gp120) and does not bind in a significant amount to other proteins or polysaccharides present in the sample or subject. Specific binding can be determined by methods known in the art. With reference to an antibody:antigen complex, specific binding of the antigen and antibody has a K_{d} of less than about 10⁻⁶ Molar, such as less than about 10⁻⁷ Molar, 10⁻⁸ Molar, 10⁻⁹, or even less than about 10⁻¹⁰ Molar.

T Cell: A white blood cell critical to the immune response. T cells include, but are not limited to, CD4⁺ T cells and CD8⁺ T cells. A CD4⁺ T lymphocyte is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. In one embodiment, a CD8 T cells is a cytotoxic T lymphocytes. In another embodiment, a CD8 cell is a suppressor T cell.

Therapeutic agent: A chemical compound, small molecule, or other composition, such as nucleic acid molecule, capable of inducing a desired therapeutic or prophylactic effect when properly administered to a subject.

Therapeutically effective amount or Effective amount: The amount of agent, such as a disclosed antigen, that is sufficient to prevent, treat (including prophylaxis), reduce and/or ameliorate the symptoms and/or underlying causes of any of a disorder or disease, for example to prevent, inhibit, and/or treat HIV. In some embodiments, an “effective amount” is sufficient to reduce or eliminate a symptom of a disease, such as AIDS. For instance, this can be the amount necessary to inhibit viral replication or to measurably alter outward symptoms of the viral infection, such as increase of T cell counts in the case of an HIV-1 infection. In general, this amount will be sufficient to measurably inhibit virus (for example, HIV) replication or infectivity. When administered to a subject, a dosage will generally be used that will achieve target tissue concentrations (for example, in lymphocytes) that has been shown to achieve in vitro inhibition of viral replication. An “anti-viral agent” or “anti-viral drug” is an agent that specifically inhibits a virus from replicating or infecting cells. Similarly, an “anti-retroviral agent” is an agent that specifically inhibits a retrovirus from replicating or infecting cells.

Transformed: A transformed cell is a cell into which has been introduced a nucleic acid molecule by molecular biology techniques. As used herein, the term transformation encompasses all techniques by which a nucleic acid molecule might be introduced into such a cell, including transfection with viral vectors, transformation with plasmid vectors, and introduction of DNA by electroporation, lipofection, and particle gun acceleration.

Vaccine: A pharmaceutical composition that elicits a prophylactic or therapeutic immune response in a subject. In some cases, the immune response is a protective immune response. Typically, a vaccine elicits an antigen-specific immune response to an antigen of a pathogen, for example a viral pathogen, or to a cellular constituent correlated with a pathological condition. A vaccine may include a polynucleotide (such as a nucleic acid encoding a disclosed antigen), a peptide or polypeptide (such as a disclosed antigen), a virus, a cell or one or more cellular constituents.

Vector: A nucleic acid molecule as introduced into a host cell, thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant DNA vectors having at least some nucleic acid sequences derived from one or more viruses.

Virus: A virus consists essentially of a core of nucleic acid surrounded by a protein coat, and has the ability to replicate only inside a living cell. “Viral replication” is the production of additional virus by the occurrence of at least one viral life cycle. A virus may subvert the host cells' normal functions, causing the cell to behave in a manner determined by the virus. For example, a viral infection may result in a cell producing a cytokine, or responding to a cytokine, when the uninfected cell does not normally do so. In some examples, a virus is a pathogen.

“Retroviruses” are RNA viruses wherein the viral genome is RNA. When a host cell is infected with a retrovirus, the genomic RNA is reverse transcribed into a DNA intermediate which is integrated very efficiently into the chromosomal DNA of infected cells. The integrated DNA intermediate is referred to as a provirus. The term “lentivirus” is used in its conventional sense to describe a genus of viruses containing reverse transcriptase. The lentiviruses include the “immunodeficiency viruses” which include human immunodeficiency virus (HIV) type 1 and type 2 (HIV-1 and HIV-2), simian immunodeficiency virus (SIV), and feline immunodeficiency virus (FIV).

HIV-1 is a retrovirus that causes immunosuppression in humans (HIV disease), and leads to a disease complex known as the acquired immunodeficiency syndrome (AIDS). “HIV disease” refers to a well-recognized constellation of signs and symptoms (including the development of opportunistic infections) in persons who are infected by an HIV virus, as determined by antibody or western blot studies. Laboratory findings associated with this disease are a progressive decline in T cells.

Virus-like particle (VLP): A non-replicating, viral shell, derived from any of several viruses. VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. Methods for producing particular VLPs are known in the art. The presence of VLPs following recombinant expression of viral proteins can be detected using conventional techniques known in the art, such as by electron microscopy, biophysical characterization, and the like. See, for example, Baker et al. (1991) Biophys. J. 60:1445-1456; and Hagensee et al. (1994) J. Virol. 68:4503-4505. For example, VLPs can be isolated by density gradient centrifugation and/or identified by characteristic density banding. Alternatively, cryoelectron microscopy can be performed on vitrified aqueous samples of the VLP preparation in question, and images recorded under appropriate exposure conditions.

### II. Description of Several Embodiments

As the sole viral target of neutralizing antibodies, the HIV-1 viral spike has evolved to evade antibody-mediated neutralization. Variable region 1 and Variable Region 2 (V1/V2) of the gp120 component of the viral spike are critical to this evasion. Localized by electron microscopy to a membrane-distal “cap,” which holds the spike in a neutralization-resistant conformation, V1/V2 is not essential for entry. However, its removal renders the virus profoundly sensitive to antibody-mediated neutralization.

The ˜50-90 residues that comprise V1/V2 contain two of the most variable portions of the virus, and one in ten residues of V1/V2 are N-glycosylated. Despite the diversity and glycosylation of V1/V2, a number of broadly neutralizing human antibodies have been identified that target this region, including the somatically related antibodies PG9 and PG16, which neutralize 70-80% of circulating HIV-1 isolates (Walker et al., Science, 326:285-289, 2009), antibodies CH01-CH04, which neutralize 40-50% (Bonsignori et al., J Virol, 85:9998-10009, 2011), and antibodies PGT141-145, which neutralize 40-80% (Walker et al., Nature, 477:466-470, 2011). These antibodies all share specificity for an N-linked glycan at residue 160 in V1V2 (HXB2 numbering) and show a preferential binding to the assembled viral spike over monomeric gp120 as well as a sensitivity to changes in V1V2 and some V3 residues. Sera with these characteristics have been identified in a number of HIV-1 donor cohorts, and these quaternary-structure-preferring V1V2-directed antibodies are among the most common broadly neutralizing responses in infected donors (Walker et al., PLoS Pathog, 6:e1001028, 2010 and Moore et al., J Virol, 85:3128-3141, 2011).

Despite extensive effort, immunogens based on V1V2 have proven ineffective and V1V2 had resisted atomic-level characterization that would allow definition of effective V1/V2 immunogens. The current disclosure provides crystal structures of the V1/V2 domain of HIV-1 gp120 in complexes with the antigen-binding fragment (Fab) of PG9 and immunogens based on this structure, for example, protein nanoparticles including these immunogens. Such molecules have utility as both potential vaccines for HIV and as diagnostic molecules (for example, to detect and quantify target antibodies in a polyclonal serum response).

### A. Antigens Including PG9 Epitopes

Isolated antigens are disclosed herein that include a PG9 epitope from a HIV-1 gp120 polypeptide (referred to herein as “PG9-epitope antigens”). In several embodiments, the antigens include the minimal PG9 epitope of gp120 as disclosed herein, including gp120 positions 154-177 (HXB2 numbering). In additional embodiments the antigens include the V1/V2 domain of gp120 (for example, gp120 positions 126-196). In several embodiments, the disclosed PG9-epitope antigens have been modified from their native form to increase immunogenicity, for example, in several embodiments, the disclosed antigens have been modified from the native HIV-1 sequence to be stabilized in a PG9-bound conformation. The person of ordinary skill in the art will appreciate that the disclosed antigens are useful to induce immunogenic responses in vertebrate animals (such as mammals, for example primates, such as humans) to HIV (for example HIV-1). Thus, in several embodiments, the disclosed antigens are immunogens.

The isolated antigens include gp120 positions 154-177 (HXBC numbering), and include asparagine residues at positions 160 and 156 or at positions 160 and 173. In several such embodiments, the antigens are stabilized in a PG9-bound conformation by at least one pair of cross-linked cysteines.

HIV-I can be classified into four groups: the “major” group M, the “outlier” group O, group N, and group P. Within group M, there are several genetically distinct clades (or subtypes) of HIV-I. The disclosed PG9 epitope antigens can be derived from any subtype of HIV, such as groups M, N, O, or P or Glade A, B, C, D, F, G, H, J or K and the like. HIV gp120 proteins from the different HIV clades, as well as nucleic acid sequences encoding such proteins and methods for the manipulation and insertion of such nucleic acid sequences into vectors, are known (see, e.g., HIV Sequence Compendium, Division of AIDS, National Institute of Allergy and Infectious Diseases (2003); HIV Sequence Database (hiv-web.lanl.gov/content/hiv-db/mainpage.html); Sambrook et al., Molecular Cloning, a Laboratory Manual, 2d edition, Cold Spring Harbor Press, Cold Spring Harbor, N.Y. (1989); Ausubel et al., Current Protocols in Molecular Biology, Greene Publishing Associates and John Wiley & Sons, New York, N.Y. (1994)).

In some examples, the disclosed PG9 epitope antigen is a PG9 binding fragment from a HIV-1 Clade A virus, for example, for example a Clade A virus listed in Table 1. In some examples, the disclosed PG9 epitope antigen is a PG9 binding fragment from a HIV-1 Clade B virus, for example, a Clade B virus listed in Table 1. In some examples, the disclosed PG9 epitope antigen is a PG9 binding fragment from a HIV-1 Clade C virus, for example, a Clade C virus listed in Table 1. In some examples, the disclosed PG9 epitope antigen is a PG9 binding fragment from a HIV-1 Clade D virus, for example, a Clade D virus listed in Table 1. In some examples, the disclosed PG9 epitope antigen is a PG9 binding fragment from a HIV-1 Clade AE virus, for example, a Clade AE virus listed in Table 1. The person of ordinary skill in the art will appreciate that the disclosed PG9 epitope antigens can include modifications of the native HIV-1 gp120 sequences, such as amino acid substitutions, deletions or insertions, glycosylation and/or covalent linkage to unrelated proteins, as long as the antigen includes a PG9 epitope, that is, as long as the antigen specifically binds to PG9.

TABLE 1
Exemplary HIV-1 virus strains, Clades and gp120 sequence

| Clade | Virus Strain | gp120 Sequence |
|---|---|---|
| A | 92UG037 | SEQ ID NO: 154 |
| A | 92RW020 | SEQ ID NO: 155 |
| B | TRJO | SEQ ID NO: 7 |
| B | JRCSF | SEQ ID NO: 156 |
| B | REJO | SEQ ID NO: 157 |
| C | CAP45 | SEQ ID NO: 3 |
| C | ZM109 | SEQ ID NO: 2 |
| C | ZM53 | SEQ ID NO: 4 |
| C | 16055 | SEQ ID NO: 6 |
| C | ZM233 | SEQ ID NO: 8 |
| D | 247-23 | SEQ ID NO: 158 |
| D | 92RW020 | SEQ ID NO: 159 |
| AE | A244 | SEQ ID NO: 5 |
| AE | 92TH021 | SEQ ID NO: 160 |

In some examples, the disclosed PG9 epitope antigen is a PG9 binding fragment from a HIV-1 Clade A virus, for example, for example a Clade A virus listed in Table 1.

In several embodiments, the PG9 epitope antigen includes or consists of at least 23 consecutive amino acids (such as at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or at least 100 consecutive amino acids) from a native HIV-1 gp120 polypeptide sequence, such as any one of SEQ ID NOs: 1-8 and 154-160, including any polypeptide sequences having at least 75% (for example at least 85%, 90%, 95%, 96%, 97%, 98% or 99%) sequence identity to a native HIV-1 gp120 polypeptide sequence, such as any one of SEQ ID NOs: 1-8 and 154-160, wherein the PG9 epitope antigen maintains PG9 specific binding activity and/or includes a PG9-bound conformation in the absence of PG9. For example, in some embodiments, the PG9 epitope antigen includes or consists of 23-100 consecutive amino acids (such as 23-24, 23-25, 23-26, 23-27, 23-28, 23-29, 23-30, 23-40, 23-50, 30-40, 40-50, 50-60, 60-70, 70-80, 80-90, 90-100, 60-80, 65-75, 66-74, 67-73, 68-72, 69-71, 70-75, 71-72, 71-73, 71-74, 71-75, 71-80, 71-85, 71-90, 71-95 or 71-100 consecutive amino acids) from a native HIV-1 gp120 polypeptide sequence, such as any one of SEQ ID NOs: 1-8 and 154-160, or any polypeptide sequences having at least 75% (for example at least 85%, 90%, 95%, 96%, 97%, 98% or 99%) sequence identity to a native HIV-1 gp120 polypeptide sequence, such as any one of SEQ ID NOs: 1-8 and 154-160, wherein the PG9 epitope antigen maintains PG9 specific binding activity and/or includes a PG9-bound conformation in the absence of PG9.

In some embodiments, the PG9 epitope antigen is also of a maximum length, for example no more than 23, 24, 25, 30, 35, 40, 45, 50, 55, 60, 65, 70, 71, 75, 80, 85, 90, 95 or 100, amino acids in length. The antigen may include, consist or consist essentially of the disclosed sequences. The disclosed contiguous sequences may also be joined at either end to other unrelated sequences (for examiner, non-gp120, non-HIV-1, non-viral envelope, or non-viral protein sequences).

It is understood in the art that some variations can be made in the amino acid sequence of a protein without affecting the activity of the protein. Such variations include insertion of amino acid residues, deletions of amino acid residues, and substitutions of amino acid residues. These variations in sequence can be naturally occurring variations or they can be engineered through the use of genetic engineering technique known to those skilled in the art. Examples of such techniques are found in Sambrook J, Fritsch E F, Maniatis T et al., in Molecular Cloning—A Laboratory Manual, 2nd Edition, Cold Spring Harbor Laboratory Press, 1989, pp. 9.31-9.57), or in Current Protocols in Molecular Biology, John Wiley & Sons, N.Y. (1989), 6.3.1-6.3.6, both of which are incorporated herein by reference in their entirety. Thus, in additional embodiments, the PG9 epitope antigen includes one or more amino acid substitutions compared to the native gp120 sequence. For example, in some embodiments, the PG9 epitope antigen includes up to 20 amino acid substitutions compared to the native gp120 polypeptide sequence, such as any one of SEQ ID NOs: 1-8 or 154-160, wherein the PG9 epitope antigen maintains PG9 specific binding activity and/or includes a PG9-bound conformation in the absence of PG9. Alternatively, the polypeptide can have none, or up to 1, 2, 3, 4, 5, 6, 7, 8, 9, 10, 11, 12, 13, 14, 15, 16, 17, 18 or 19 amino acid substitutions compared to the native gp120 polypeptide sequence, wherein the PG9 epitope antigen maintains PG9 specific binding activity and/or includes a PG9-bound conformation in the absence of PG9. Manipulation of the nucleotide sequence encoding the PG9 epitope antigen using standard procedures, including in one specific, non-limiting, embodiment, site-directed mutagenesis or in another specific, non-limiting, embodiment, PCR, can be used to produce such variants. Alternatively, the PG9 epitope antigen can be synthesized using standard methods. The simplest modifications involve the substitution of one or more amino acids for amino acids having similar biochemical properties. These so-called conservative substitutions are likely to have minimal impact on the activity of the resultant protein.

In several embodiments, any of the disclosed PG9 epitope antigens is stabilized in a PG9-bound conformation by at least one pair of cross-linked cysteine residues. For example, in some embodiments, any of the disclosed PG9 epitope antigens is stabilized in a PG9-bound conformation by any one of 1, 2, 3, 4, 5, 6, 7, 8, 9, or 10 pairs of cross-linked cysteine residues. In one specific non-limiting example, any of the disclosed PG9 epitope antigens is stabilized in a PG9-bound conformation by a single pair of cross-linked cysteine residues. In another non-limiting example, any of the disclosed PG9 epitope antigens is stabilized in a PG9-bound conformation by two pairs of crosslinked cysteine residues.

In some embodiments, the disclosed HIV-1 gp120 polypeptide, or PG9 binding fragment thereof, has been substantially resurfaced from the native gp120 sequence, such that the surface of the HIV-1 gp120 polypeptide or PG9 binding fragment thereof has been altered to focus the immune response to the PG9 epitope on the HIV-1 gp120 polypeptide or PG9 binding fragment thereof. For example, the method can remove non-target epitopes that might interfere with specific binding of an antibody to the PG9 epitope. In some embodiments, the amino acid substitutions alter antigenicity in vivo as compared to the wild-type antigen (unsubstituted antigen), but do not introduce additional glycosylation sites as compared to the wild-type antigen. In other embodiments, that PG9 epitope antigen is glycosylated. Examples of antigen resurfacing methods are given in PCT Publication Nos. WO 09/100,376 and WO/2012/006180, which are specifically incorporated by reference in its entirety.

For example, in several embodiments, any of the disclosed PG9 epitope antigens include or consist of HIV-1 gp120 positions 154-177, wherein the amino acids at positions 155 and 176 are cysteine residues. In additional embodiments, any of the disclosed PG9 epitope antigens include or consist of HIV-1 gp120 positions 154-177, wherein the amino acids at positions 155 and 176 are cysteine residues and wherein the PG9 epitope antigen does not include any cysteine residues at gp120 positions 154, 156-175 or 177. For example, the amino acids at positions 155 and 176 can be substituted for cysteine residues, and the amino acids at positions 154, 156-175 or 177 can be substituted for a residue other than cysteine (such as a serine residue or a conservative amino acid substitution), if the native gp120 sequence does not include cysteine residues, or does include cysteine residues, respectively, at these positions.

In several embodiments, any of the disclosed PG9 epitope antigens include or consist of HIV-1 gp120 positions 154-177, wherein the amino acids at positions 155 and 176 are cysteine residues, and wherein the PG9 epitope antigen includes a first pair of cross-linked cysteines at gp120 positions 155 and 176. In additional embodiments, any of the disclosed PG9 epitope antigens include or consist of HIV-1 gp120 positions 154-177, wherein the amino acids at positions 155 and 176 are cysteine residues, wherein the PG9 epitope antigen does not include any cysteine residues at gp120 positions 154, 156-175 or 177, and wherein the PG9 epitope antigen includes a first pair of cross-linked cysteines at gp120 positions 155 and 176.

In additional embodiments, the PG9 epitope antigen includes or consists of a V1/V2 domain of HIV-1 gp120 as disclosed herein, for example, the PG9 epitope antigen can include or consist of HIV-1 gp120 positions 126-196. In some such embodiments, any of the disclosed PG9 epitope antigens including or consisting of HIV-1 gp120 positions 126-196, include cysteine residues at positions 126, 196, 131 and 157. In additional embodiments, any of the disclosed PG9 epitope antigens including or consisting of HIV-1 gp120 positions 126-196, include cysteine residues at positions 126, 196, 131 and 157, and include residues other than cysteine at gp120 positions 127-130, 132-156 and 158-195. For example, the amino acids at positions 126, 196, 131 and 157 can be substituted for cysteine residues, the amino acids at positions 127-130, 132-156 or 158-195 can be substituted for a residue other than cysteine (such as a serine residue or a conservative amino acid substitution), if the native gp120 sequence does not include cysteine residues, or does include cysteine residues, respectively, at these positions.

In additional embodiments, any of the disclosed PG9 epitope antigens including or consisting of a gp120 V1/V2 domain (such as HIV-1 gp120 positions 126-196) include at least two pairs of cross-linked cysteine residues including a first pair of cross-linked cysteine residues at gp120 positions 126 and 196 and a second pair of crosslinked cysteines at gp120 positions 131 and 157. In some embodiments, any of the disclosed PG9 epitope antigens including or consisting of a gp120 V1/V2 domain (such as HIV-1 gp120 positions 126-196) includes two pairs of cross-linked cysteines residues including a first pair of cross-linked cysteine residues at gp120 positions 126 and 196, a second pair of crosslinked cysteines at gp120 positions 131 and 157, and does not includes any cysteine residues at gp120 positions 127-130, 132-156 or 158-195.

In several embodiments, any of the disclosed PG9 epitope antigens include a first asparagine residue at gp120 position 160 and a second asparagine residue at gp120 position 156 or 173, but not both positions 156 and 173. In some embodiments, the PG9 epitope antigen includes a first N-linked glycosylation site including an asparagine residue at gp120 position 160 and a serine or threonine residue at gp120 position 162, and a second N-linked glycosylation site including an asparagine residue at gp120 position 156 and a serine or threonine residue at gp120 position 158. In additional embodiments, the PG9 epitope antigen includes a first N-linked glycosylation site including an asparagine residue at gp120 position 160 and a serine or threonine residue at gp120 position 162, and a second N-linked glycosylation site including an asparagine residue at gp120 position 173 and a serine or threonine residue at gp120 position 175.

In some embodiments, the PG9 epitope antigen includes or consists of gp120 positions 154-177, wherein the PG9 epitope antigen includes an amino acid sequence set forth as: X₁CNSX₂X₃NX₄X₅X₆X₇X₈X₉X₁₀X₁₁X₁₂X₁₃X₁₄X₁₅X₁₆X₁₇LCY, wherein X₁ is I, M, V, or A; X₂ is S or T; X₃ is F or Y; X₄ is I, M, V, or A; X₅ is S or T; X₆ is S or T; X₇ is any amino acid; X₈ is any amino acid; X₉ is R or K; X₁₀ is D or E; X₁₁ is K or R; X₁₂ is any amino acid; X₁₃ is K, R, or Q; X₁₄ is K, R, or Q; X₁₅ E, D, or V; X₁₆ is Y, F, or H; and X₁₇ is S or A (SEQ ID NO: 132). In one example, the PG9 epitope antigen includes or consists of an amino acid sequence set forth as VCNSSFNITTELRDKKQKAYALCY (SEQ ID NO: 134).

In additional embodiments, the PG9 epitope antigen the PG9 epitope antigen includes or consists of gp120 positions 154-177, wherein the PG9 epitope antigen includes or consists of an amino acid sequence set forth as: X₁CX₂SX₃X₄NX₅X₆X₇X₈X₉X₁₀X₁₁X₁₂X₁₃X₁₄X₁₅NX₁₆X₁₇LCY, wherein X₁ is I, M, V, or A; X₂ is any amino acid; X₃ is S or T; X₄ is F or Y; X₅ is I, M, V, or A; X₆ is S or T; X₇ is S or T; X₈ is any amino acid; X₉ is any amino acid; X₁₀ is R or K; X₁₁ is D or E; X₁₂ is K or R; X₁₃ is any amino acid; X₁₄ is K, R, or Q; X₁₅ is K, R, or Q; X₁₆ is S or A; and X₁₇ is S or T (SEQ ID NO: 133). In one example, the PG9 epitope antigen includes or consists of an amino acid sequence set forth as VCHSSFNITTDVKDRKQKVNATCY (SEQ ID NO: 135).

In some examples, the disclosed PG9 epitope antigen includes or consists of an amino acid sequence including gp120 positions 154-177, wherein position 156 is an asparagine, position 160 is an asparagine, position 155 is a cysteine, position 176 is a cysteine, positions 154, 157-159, 161-175 and 177 do not include any cysteine residues, and positions 154, 157-159, 161-175 and 177 correspond to the amino acid sequence of a native gp120 (for example, a native HIV-1 gp120 as set forth in “HIV Sequence Compendium 2010,” Kuiken et al., Eds. Published by Theoretical Biology and Biophysics Group, Los Alamos National Laboratory, NM, LA-UR 10-03684, which is incorporated by reference herein in its entirety; or, for example, a native HIV-1 gp120 as set forth in the HIV Sequence Database, as present on Aug. 27, 2012 and available on the world wide web at “hiv.lanl.gov/”), and wherein the PG9 epitope antigen specifically binds to monoclonal antibody PG9, induces an immune response to HIV-1 when administered to a subject.

In some examples, the disclosed PG9 epitope antigen includes or consists of an amino acid sequence including gp120 positions 154-177, wherein position 160 is an asparagine, position 173 is an asparagine, position 155 is a cysteine, position 176 is a cysteine, positions 154, 157-175 and 177 do not include any cysteine residues, and positions 154, 157-159, 161-175 and 177 correspond to the amino acid sequence of a native gp120 (for example, a native HIV-1 gp120 as set forth in “HIV Sequence Compendium 2010,” Kuiken et al., Eds. Published by Theoretical Biology and Biophysics Group, Los Alamos National Laboratory, NM, LA-UR 10-03684, which is incorporated by reference herein in its entirety; or, for example, a native HIV-1 gp120 as set forth in the HIV Sequence Database, as present on Aug. 27, 2012 and available on the world wide web at “hiv.lanl.gov/”), and wherein the PG9 epitope antigen specifically binds to monoclonal antibody PG9, induces an immune response to HIV-1 when administered to a subject.

In some examples, the disclosed PG9 epitope antigen includes or consists of an amino acid sequence including gp120 positions 154-177, wherein position 156 is an asparagine, position 160 is an asparagine, position 155 is a cysteine, position 176 is a cysteine, positions 154-155, 157-159 and 161-177 do not include any asparagine residues, positions 154, 157-159, 161-175 and 177 do not include any cysteine residues, and positions 154, 157-159, 161-175 and 177 correspond to the amino acid sequence of a native gp120 (for example, a native HIV-1 gp120 as set forth in “HIV Sequence Compendium 2010,” Kuiken et al., Eds. Published by Theoretical Biology and Biophysics Group, Los Alamos National Laboratory, NM, LA-UR 10-03684, which is incorporated by reference herein in its entirety; or, for example, a native HIV-1 gp120 as set forth in the HIV Sequence Database, as present on Aug. 27, 2012 and available on the world wide web at “hiv.lanl.gov/”), and wherein the PG9 epitope antigen specifically binds to monoclonal antibody PG9, induces an immune response to HIV-1 when administered to a subject.

In some examples, the disclosed PG9 epitope antigen includes or consists of an amino acid sequence including gp120 positions 154-177, wherein position 160 is an asparagine, position 173 is an asparagine, position 155 is a cysteine, position 176 is a cysteine, positions 154-159, 161-172 and 174-177 do not include any asparagine residues, positions 154, 157-175 and 177 do not include any cysteine residues, and positions 154, 157-159, 161-175 and 177 correspond to the amino acid sequence of a native gp120 (for example, a native HIV-1 gp120 as set forth in “HIV Sequence Compendium 2010,” Kuiken et al., Eds. Published by Theoretical Biology and Biophysics Group, Los Alamos National Laboratory, NM, LA-UR 10-03684, which is incorporated by reference herein in its entirety; or, for example, a native HIV-1 gp120 as set forth in the HIV Sequence Database, as present on Aug. 27, 2012 and available on the world wide web at “hiv.lanl.gov/”), and wherein the PG9 epitope antigen specifically binds to monoclonal antibody PG9, induces an immune response to HIV-1 when administered to a subject.

In further embodiments, any of the disclosed PG9 epitope antigen including or consisting of a gp120 V1/V2 domain (such as HIV-1 gp120 positions 126-196), further include truncation of the V1 variable loop, the V2 variable loop, or both. For example, in some such embodiments, the V1 variable loop is replaced with the amino acid sequence GGSG (SEQ ID NO: 152) and/or the V2 variable loop is replaced with the amino acid sequence GGSGGSGG (SEQ ID NO: 153). In one example the PG9 epitope antigen includes or consists of a gp120 V1/V2 domain (such as HIV-1 gp120 positions 126-196), wherein the amino acids at positions 135-152 are substituted with the amino acid sequence GGSG (SEQ ID NO: 152), and the amino acids at positions 181-188 are substituted with the amino acid sequence GGSGGSGG (SEQ ID NO: 153).

Several embodiments include a multimer of any of the disclosed PG9 epitope antigens including a V1/V2 domain of gp120 (such as gp120 positions 126-196), for example, a multimer including 2, 3, 4, 5, 6, 7, 8, 9, or 10 or more of the disclosed PG9 epitope antigens. In several examples, any of the disclosed PG9 epitope antigens can be linked to another of the disclosed PG9 epitope antigens to form the multimer. In specific non-limiting examples, the multimer includes a first V1/V2 domain linked to a second V1/V2 domain, for example the multimer includes the amino acid sequence set forth as SEQ ID NO: 113 (linked dimer of the V1/V2 domain from the CAP45 strain of HIV-1), SEQ ID NO: 114 (linked dimer of the V1/V2 domain from the CAP210 strain of HIV-1), SEQ ID NO: 115 (linked dimer of the V1/V2 domain from the A244 strain of HIV-1), or SEQ ID NO: 116 (linked dimer of the V1/V2 domain from the ZM233 strain of HIV-1). In additional embodiments, the multimer includes a first a first V1/V2 domain with truncated V1 and V2 variable loops linked to a second V1/V2 domain with truncated V1 and V2 variable loops, for example a multimer includes the amino acid sequence set forth as SEQ ID NO: 117 (linked dimer of the V1/V2 domain from the A244 strain of HIV-1 with truncated V1 and V2 variable loops) and SEQ ID NO: 118 (linked dimer of the V1/V2 domain from the ZM233 strain of HIV-1 with truncated V1 and V2 variable loops).

In several embodiments, any of the disclosed PG9 epitope antigens are glycosylated. For example, PG9 epitope antigens including asparagine residues at gp120 positions 160 and 173 or at positions 156 and 160 can be glycosylated at these positions. In several embodiments, the PG9 epitope antigen includes a first N-linked glycan moiety at position 160, and a second N-linked glycan moiety at position 156 or positions 173, but not both. In additional embodiments, the PG9 epitope antigen includes a first N-linked glycan moiety at position 160, a second N-linked glycan moiety at position 156 or position 173, but not both, and does not include any other glycan moieties.

N-linked glycans are based on the common core pentasaccharide, Man₃GlcNAc₂, which includes the chitobiose (GlcNAc₂) core (see Structure I). Further processing in the Golgi results in three main classes of N-linked glycan classes: oligomannose, hybrid and complex glycans. Oligomannose glycans contain unsubstituted terminal mannose sugars (see, for example, Structures II-V). These glycans typically contain between five and nine mannose residues attached to chitobiose. In several embodiments, the glycan moiety at position 160 is an oligomannose glycan moiety, for example a Man₄GlcNac₂, Man₅GlcNac₂, Man₆GlcNac₂, Man₇GlcNac₂Man₄ glycan moiety. In some examples, the glycan moiety at position 160 has a formula according to any one of Structure I-V. In one example, the glycan moiety at position 160 has a formula according to Structure II.

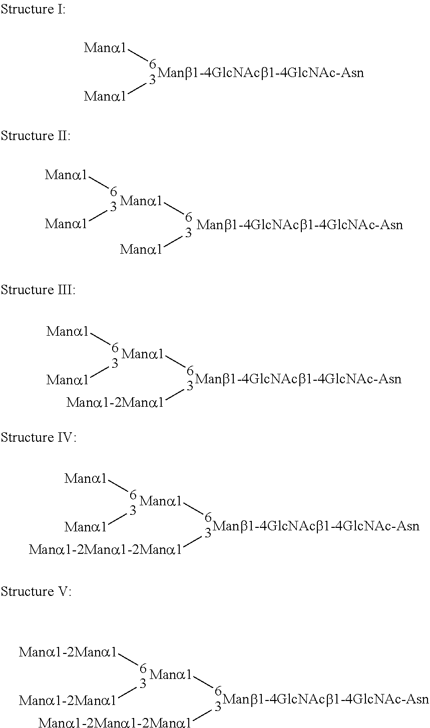

Hybrid glycans include both unsubstituted terminal mannose residues (as present in oligomannose glycans) and substituted mannose residues with an N-acetylglucosamine (GlcNAc) linkage (as present in complex glycans) (see, for example, Structures VI-VII). Structures VI and VII show a glycan with two or three GlcNAc branches linked to the chitobiose core, respectively. In several embodiments, the glycan moiety at position 156 or position 173 is a hybrid glycan, for example, a hybrid glycan having a formula according to Structure VI or Structure VII.

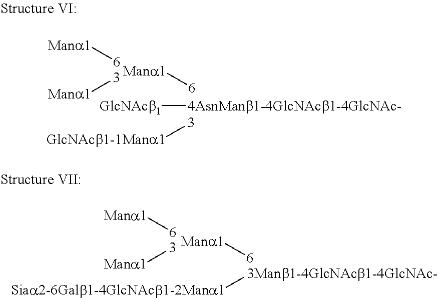

Complex N-linked glycans differ from the oligomannose and hybrid glycans by having added N-acetylglucosamine residues at both the α-3 and α-6 mannose sites (see, for example, Structures VIII-XIII). Unlike oligomannose glycans, complex glycans do not include mannose residues except for the core pentasaccharide (Man₃GlcNAc₂) structure. Additional monosaccharides may occur in repeating lactosamine GlcNAc-β(1-4)Gal) units. Complex glycans comprise the majority of cell surface and secreted N-glycans and can include multiple branches off of the core pentasaccharide unit. In several embodiments, the complex glycan terminates with sialic acid residues (Sia). Additional modifications such as the addition of a bisecting GlcNAc at the mannosyl core and/or a fucosyl residue on the innermost GlcNAc (as indicated in Structure XIII) are also possible. In several embodiments, the glycan moiety at position 156 or position 173 is a complex glycan, for example, a complex glycan having a formula according to any one of Structures VIII-XIII. In one embodiment, the glycan moiety at position 156 or position 173 is a complex glycan having a formula according to Structure VIII.

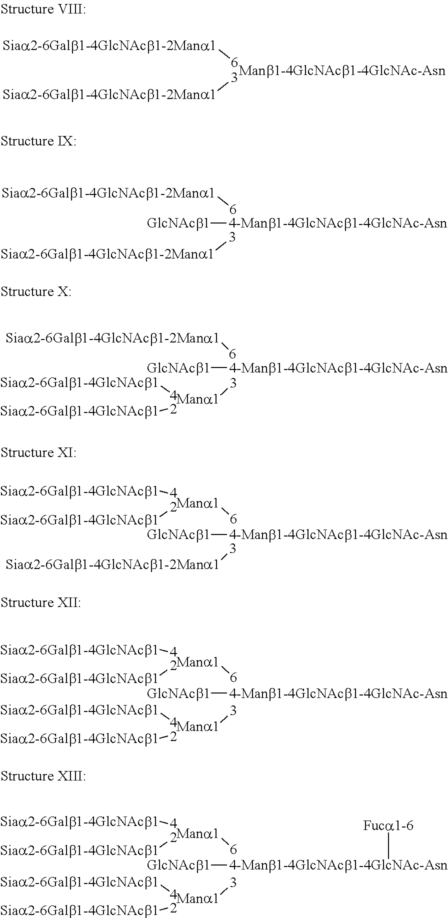

The person of ordinary skill in the art will understand that additional glycan structures can be included on the antigen, and that the bond numbering shown above is representative, and that other glycan bonds are available. For example Siaα2-3Gal bonds can be present in the glycan. In several embodiments, the hybrid or complex glycan includes at least one Siaα2-6Galβ1-4GlcNAcβ1-2Manα1-3 moiety on an arm of the glycan.

In some embodiments, the PG9 epitope antigen includes a first N-linked glycan moiety at position 160, wherein the first N-linked glycan is a oligomannose glycan (such as a oligomannose glycan having a structure set forth as any one of Structures I-V), and the PG9 epitope-antigen further includes a second N-linked glycan at position 156 or position 173 (but not both), wherein the second N-linked glycan is a hybrid glycan (such as a hybrid glycan set forth as any one of Structures VI-VII). In several embodiments, the PG9 epitope antigen includes a first N-linked glycan moiety at position 160, wherein the first N-linked glycan is a oligomannose glycan (such as a oligomannose glycan having a structure set forth as any one of Structures I-V), and the PG9 epitope-antigen further includes a second N-linked glycan at position 156 or position 173 (but not both), wherein the second N-linked glycan is a hybrid glycan (such as a hybrid glycan set forth as any one of Structures VI-VII), and does not include any other glycan moieties.

In several embodiments, the PG9 epitope antigen includes a first N-linked glycan moiety at position 160, wherein the first N-linked glycan is a oligomannose glycan (such as a oligomannose glycan having a structure set forth as any one of Structures I-V), and the PG9 epitope-antigen further includes a second N-linked glycan at position 156 or position 173 (but not both), wherein the second N-linked glycan is a complex glycan (such as a complex glycan set forth as any one of Structures VIII-XIII). In several embodiments, the PG9 epitope antigen includes a first N-linked glycan moiety at position 160, wherein the first N-linked glycan is a oligomannose glycan (such as a oligomannose glycan having a structure set forth as any one of Structures I-V), and the PG9 epitope-antigen further includes a second N-linked glycan at position 156 or position 173 (but not both), wherein the second N-linked glycan is a complex glycan (such as a complex glycan set forth as any one of Structures VIII-XIII), and does not include any other glycan moieties.

In some embodiments, the PG9 epitope antigen includes a first N-linked glycan moiety at position 160, wherein the first N-linked glycan is a oligomannose glycan (such as a oligomannose glycan having a structure set forth as Structure II), and the PG9 epitope-antigen further includes a second N-linked glycan at position 156 or position 173 (but not both), wherein the second N-linked glycan is a complex glycan (such as a complex glycan set forth as Structure VIII). In several embodiments, the PG9 epitope antigen includes a first N-linked glycan moiety at position 160, wherein the first N-linked glycan is a oligomannose glycan (such as a oligomannose glycan having a structure set forth as Structure II), and the PG9 epitope-antigen further includes a second N-linked glycan at position 156 or position 173 (but not both), wherein the second N-linked glycan is a complex glycan (such as a complex glycan set forth as Structure VIII), and does not include any other glycan moieties.

Methods of making glycosylated polypeptides are disclosed herein and are familiar to the person of ordinary skill in the art. For example, such methods are disclosed herein and described in U.S. Patent Application Pub. No. 2007/0224211, U.S. Pat. Nos. 7,029,872; 7,834,159, 7,807,405, Wang and Lomino, ACS Chem. Biol., 7:110-122, 2011, and Nettleship et al., Methods Mol. Biol, 498:245-263, 2009, each of which is incorporated by reference herein. In some embodiments, glycosylated PG9 epitope antigens are produced by expression the PG9 epitope antigen in mammalian cells, such as HEK293 cells or derivatives thereof, such as GnTI^{-/-} cells (ATCC® No. CRL-3022). In some embodiments, the PG9 epitope antigens are produced by expression the PG9 epitope antigen in mammalian cells, such as HEK293 cells or derivatives thereof, with swainsonine added to the media in order to inhibit certain aspects of the glycosylation machinery, for example to promote production of hybrid glycans.

In several embodiments, the disclosed PG9 epitope antigens specifically bind to PG9. In several examples, the dissociation constant for PG9 binding to the HIV-1 gp120 polypeptide, or PG9 binding fragment thereof, is less than about 10⁻⁶ Molar, such as less than about 10⁻⁶ Molar, 10⁻⁷ Molar, 10⁻⁸ Molar, or less than 10⁻⁹ Molar. Specific binding can be determined by methods known in the art. The determination that a particular agent binds substantially only to a specific polypeptide may readily be made by using or adapting routine procedures. One suitable in vitro assay makes use of the Western blotting procedure (described in many standard texts, including Harlow and Lane, Using Antibodies: A Laboratory Manual, CSHL, New York, 1999).

In several embodiments, any of the PG9 epitope antigens disclosed includes a PG9 epitope in a PG9-bound conformation. In another embodiment, any of the PG9 epitope antigens disclosed includes a PG9 epitope in a PG16-bound conformation. Methods of determining if a disclosed PG9 epitope antigen includes a PG9 epitope in a PG9-bound or PG16-bound conformation are known to the person of ordinary skill in the art and further disclosed herein (see, for example, McLellan et al., Nature, 480:336-343, 2011; and U.S. Patent Application Publication No. 2010/0068217, each of which is incorporated by reference herein in its entirety). For example, the three-dimensional structures of the PG9 Fab fragment in complex with the V1/V2 domain of gp120 from two different HIV-1 strains (CAP 45 and ZM109) are disclosed herein. The coordinates for these three-dimensional structures are deposited in the Protein Data Bank (PDB) and are set forth as PDB Accession Nos. 3U4E (showing V1/V2 from HIV-1 CAP45 in complex with PG9 Fab) and 3U2S (showing V1/V2 from HIV-1 ZM109 in complex with PG9 Fab), each of which is incorporated by reference herein in their entirety as present in the database on Aug. 27, 2012. The three-dimensional structure of the disclosed PG9 epitope antigen can be determined and compared with the structure disclosed in PDB Accession No. 3U4E or 3U2S.

The disclosed three-dimensional structure of the PG9 Fab fragment in complex with the V1/V2 domain of gp120 can be compared with three-dimensional structure of any of the disclosed PG9 epitope antigens. The person of ordinary skill in the art will appreciate that a disclosed antigen can include an epitope in a PG9-bound conformation even though the structural coordinates of antigen are not identical to those of the PG9 epitope bound to PG disclosed herein. For example, In several embodiments, any of the disclosed PG9 epitope antigens include a PG9 epitope that in the absence of monoclonal antibody PG9 can be structurally superimposed onto the PG9 epitope in complex with monoclonal antibody PG9 with a root mean square deviation (RMSD) of their coordinates of less than 0.5, 0.45, 0.4, 0.35, 0.3 or 0.25 Å/residue, wherein the RMSD is measured over the polypeptide backbone atoms N, CA, C, O, for at least three consecutive amino acids.

These two disclosed structures of PG9 in complex with the V1/V2 domain illustrate gp120 PG9 epitope antigens in a PG9-bound conformation, wherein the gp120 V1/V2 domain adopts a four-stranded anti-parallel beta-sheet, with PG9 forming hydrogen bonds with a first N-linked glycan at gp120 position 160 and a second N-linked glycan at gp120 position 156 of CAP45, or position 173 of ZM109. Due to the conformation of the underlying beta-sheet, the N-linked glycan at position 156 of HIV-1 CAP45 occupies substantially the same three-dimensional space as the N-linked glycan at position 173 of HIV-1 ZM109, when bound to PG9.

In several embodiments, any of the disclosed PG9 epitope antigens can be used to induce an immune response to HIV-1 in a subject. In several such embodiments, induction of the immune response include production of broadly neutralizing antibodies to HIV-1. Methods to assay for neutralization activity are known to the person of ordinary skill in the art and further described herein, and include, but are not limited to, a single-cycle infection assay as described in Martin et al. (2003) Nature Biotechnology 21:71-76. In this assay, the level of viral activity is measured via a selectable marker whose activity is reflective of the amount of viable virus in the sample, and the IC₅₀ is determined. In other assays, acute infection can be monitored in the PM1 cell line or in primary cells (normal PBMC). In this assay, the level of viral activity can be monitored by determining the p24 concentrations using ELISA. See, for example, Martin et al. (2003) Nature Biotechnology 21:71-76. Additional neutralization assays are described in the disclosed examples.

### Epitope-Scaffold Proteins

In several embodiments, any of the disclosed PG9 epitope antigens is included on a scaffold protein to generate an epitope-scaffold protein. The PG9 epitope antigen can be placed anywhere in the scaffold protein (for example, on the N-terminus, C-terminus, or an internal loop), as long as the epitope scaffold protein retains the characteristics of the native epitope (such as specific binding to PG9 and/or a PG9-bound conformation).

Methods for identifying and selecting scaffolds are disclosed herein and known to the person of ordinary skill in the art. For example, methods for superposition, grafting and de novo design of epitope-scaffolds are disclosed in U.S. Patent Application Publication No. 2010/0068217, incorporated by reference herein in its entirety.

“Superposition” epitope-scaffolds are based on scaffold proteins having an exposed segment with similar conformation as the target epitope—the backbone atoms in this “superposition-region” can be structurally superposed onto the target epitope with minimal root mean square deviation (RMSD) of their coordinates. Suitable scaffolds are identified by computationally searching through a library of protein crystal structures; epitope-scaffolds are designed by putting the epitope residues in the superposition region and making additional mutations on the surrounding surface of the scaffold to prevent clash or other interactions with the antibody.

“Grafting” epitope-scaffolds utilize scaffold proteins that can accommodate replacement of an exposed segment with the crystallized conformation of the target epitope. For each suitable scaffold identified by computationally searching through all protein crystal structures, an exposed segment is replaced by the target epitope and the surrounding sidechains are redesigned (mutated) to accommodate and stabilize the inserted epitope. Finally, as with superposition epitope-scaffolds, mutations are made on the surface of the scaffold and outside the epitope, to prevent clash or other interactions with the antibody. Grafting scaffolds require that the replaced segment and inserted epitope have similar translation and rotation transformations between their N- and C-termini, and that the surrounding peptide backbone does not clash with the inserted epitope. One difference between grafting and superposition is that grafting attempts to mimic the epitope conformation exactly, whereas superposition allows for small structural deviations.

“De novo” epitope-scaffolds are computationally designed from scratch to optimally present the crystallized conformation of the epitope. This method is based on computational design of a novel fold (Kuhlman, B. et al. 2003 Science 302:1364-1368). The de novo allows design of immunogens that are both minimal in size, so they do not present unwanted epitopes, and also highly stable against thermal or chemical denaturation.

In several embodiments, the native scaffold protein (without epitope insertion) is not a viral envelope protein. In additional embodiments, the scaffold protein is not an HIV protein. In still further embodiments, the scaffold protein is not a viral protein. In some embodiments, the native scaffold protein includes an amino acid sequence set forth as any one of SEQ ID NOs: 78-112.

In additional embodiments, the epitope-scaffold protein is any one of 1VH8_C (SEQ ID NO: 65), 1YN3_A (SEQ ID NO: 28), 1X3E_C (SEQ ID NO: 67), 2VXS_A (SEQ ID NO: 37), 1VH8_B (SEQ ID NO: 64), 2ZJR_A (SEQ ID NO: 17), 2ZJR_B (SEQ ID NO: 18), 1VH8_A (SEQ ID NO: 63), 1X3E_A (SEQ ID NO: 66), 3PYR_A (SEQ ID NO: 76), 1T0A_A (SEQ ID NO: 77), 2F7S_B (SEQ ID NO: 52), and 2F7S_C (SEQ ID NO: 53), or a polypeptide with at least 80% sequence identity (such as at least 85%, at least 90%, at least 95%, at least 96%, at least 97%, at least 98% or at least 99% sequence identity) to any one of 1VH8_C (SEQ ID NO: 65), 1YN3_A (SEQ ID NO: 28), 1X3E_C (SEQ ID NO: 67), 2VXS_A (SEQ ID NO: 37), 1VH8_B (SEQ ID NO: 64), 2ZJR_A (SEQ ID NO: 17), 2ZJR_B (SEQ ID NO: 18), 1VH8_A (SEQ ID NO: 63), 1X3E_A (SEQ ID NO: 66), 3PYR_A (SEQ ID NO: 76), 1T0A_A (SEQ ID NO: 77), 2F7S_B (SEQ ID NO: 52), and 2F7S_C (SEQ ID NO: 53) and wherein the epitope-scaffold protein specifically binds to PG9 and/or the PG9 epitope on the Epitope Scaffold includes a PG9-bound conformation in the absence of PG9. In additional embodiments, the PG9-epitope scaffold protein is any one of 1VH8_C (SEQ ID NO: 65), 1YN3_A (SEQ ID NO: 28), 1X3E_C (SEQ ID NO: 67), 2VXS_A (SEQ ID NO: 37), 1VH8_B (SEQ ID NO: 64), 2ZJR_A (SEQ ID NO: 17), 2ZJR_B (SEQ ID NO: 18), 1VH8_A (SEQ ID NO: 63), 1X3E_A (SEQ ID NO: 66), 3PYR_A (SEQ ID NO: 76), 1T0A_A (SEQ ID NO: 77), 2F7S_B (SEQ ID NO: 52), and 2F7S_C (SEQ ID NO: 53), wherein the amino acid sequence of the PG9 epitope-scaffold protein has up to 20 amino acid substitutions, and wherein the epitope-scaffold protein specifically binds to PG9 and/or the PG9 epitope in the Epitope-Scaffold protein includes a PG9-bound conformation in the absence of PG9. Alternatively, the polypeptide can have none, or up to 1, 2, 3, 4, 5, 6, 7, 8, 9, 10, 11, 12, 13, 14, 15, 16, 17, 18 or 19 amino acid substitutions.

The PG9 epitope antigen can be placed anywhere in the scaffold, as long as the resulting epitope-scaffold protein specifically binds to PG9 and/or the PG9 epitope on the Epitope-Scaffold protein includes a PG9-bound conformation in the absence of PG9. Methods for determining if a particular epitope-scaffold protein specifically binds to PG9 are disclosed herein and known to the person of ordinary skill in the art (see, for example, International Application Pub. Nos. WO 2006/091455 and WO 2005/111621). In addition, the formation of an antibody-antigen complex can be assayed using a number of well-defined diagnostic assays including conventional immunoassay formats to detect and/or quantitate antigen-specific antibodies. Such assays include, for example, enzyme immunoassays, e.g., ELISA, cell-based assays, flow cytometry, radioimmunoassays, and immunohistochemical staining. Numerous competitive and non-competitive protein binding assays are known in the art and many are commercially available. Methods for determining if a particular epitope-scaffold protein includes a PG9 epitope having a PG9-bound conformation in the absence of PG9 are also described herein and further known to the person of ordinary skill in the art.

### Particles

Several embodiments include a protein nanoparticle including one or more of any of the disclosed PG9 epitope antigens. Non-limiting example of nanoparticles include ferritin nanoparticles, an encapsulin nanoparticles and Sulfur Oxygenase Reductase (SOR) nanoparticles, which are comprised of an assembly of monomeric subunits including ferritin proteins, encapsulin proteins and SOR proteins, respectively. To construct protein nanoparticles including the disclosed PG9 epitope antigens, the antigen is linked to a subunit of a protein nanoparticle (such as a ferritin protein, an encapsulin protein or a SOR protein), the fusion protein is expressed, and will self-assemble into a nanoparticle under appropriate conditions.

In some embodiments, any of the disclosed PG9 epitope antigens are linked to a ferritin polypeptide or hybrid of different ferritin polypeptides, to construct a ferritin nanoparticle. Ferritin is a globular protein that is found in all animals, bacteria, and plants, and which acts primarily to control the rate and location of polynuclear Fe(III)₂O₃ formation through the transportation of hydrated iron ions and protons to and from a mineralized core. The globular form of ferritin is made up of monomeric subunits, which are polypeptides having a molecule weight of approximately 17-20 kDa. An example of the sequence of one such monomeric subunit is represented by SEQ ID NO: 119. Each monomeric subunit has the topology of a helix bundle which includes a four antiparallel helix motif, with a fifth shorter helix (the c-terminal helix) lying roughly perpendicular to the long axis of the 4 helix bundle. According to convention, the helices are labeled ‘A, B, C, D & E’ from the N-terminus respectively. The N-terminal sequence lies adjacent to the capsid three-fold axis and extends to the surface, while the E helices pack together at the four-fold axis with the C-terminus extending into the capsid core. The consequence of this packing creates two pores on the capsid surface. It is expected that one or both of these pores represent the point by which the hydrated iron diffuses into and out of the capsid. Following production, these monomeric subunit proteins self-assemble into the globular ferritin protein. Thus, the globular form of ferritin comprises 24 monomeric, subunit proteins, and has a capsid-like structure having 432 symmetry. Methods of constructing ferritin nanoparticles are known to the person of ordinary skill in the art and are further described herein (see, e.g., Zhang, Y. Int. J. Mol. Sci., 12:5406-5421, 2011, which is incorporated herein by reference in its entirety

In specific examples, the ferritin polypeptide is E. coli ferritin, Helicobacter pylori ferritin, human light chain ferritin, bullfrog ferritin or a hybrid thereof, such as E. coli-human hybrid ferritin, E. coli-bullfrog hybrid ferritin, or human-bullfrog hybrid ferritin. Exemplary amino acid sequences of ferritin polypeptides and nucleic acid sequences encoding ferritin polypeptides for use in the disclosed PG9 epitope antigens can be found in GENBANK®, for example at accession numbers ZP_{—}03085328, ZP_{—}06990637, EJB64322.1, AAA35832, NP_{—}000137 AAA49532, AAA49525, AAA49524 and AAA49523, which are specifically incorporated by reference herein in their entirety as available Aug. 27, 2012. In one embodiment, any of the disclosed PG9 epitope antigens is linked to a ferritin protein including an amino acid sequence at least 80% (such as at least 85%, at least 90%, at least 95%, or at least 97%) identical to amino acid sequence set forth as SEQ ID NO: 119.

Specific examples of the disclosed PG9 epitope antigens including a minimal PG9 binding epitope (gp120 positions 154-177) linked to a ferritin protein include the amino acid sequence set forth as SEQ ID NO: 120 (minimal PG9 epitope based on HIV-1 strain ZM109 linked to ferritin), SEQ ID NO: 121 (minimal PG9 epitope based on HIV-1 strain CAP45 linked to ferritin) and SEQ ID NO: 122 (minimal PG9 epitope based on HIV-1 strain A244 linked to ferritin). Additional substitutions to the minimal epitope present on a ferritin protein can be made, for example substitutions of cysteine residues for the amino acids at gp120 positions 155 and 176 of the minimal PG9 epitope on the PG9 epitope-ferritin fusion protein. Specific examples of the disclosed PG9 epitope antigens including a dimer of the V1/V2 domain (a dimer of gp120 positions 126-196) linked to a ferritin protein include the amino acid sequence set forth as SEQ ID NO: 123 (linked dimer of the V1/V2 domain from the CAP45 strain of HIV-1 linked to ferritin) and SEQ ID NO: 124 (linked dimer of the V1/V2 domain from the ZM109 strain of HIV-1 linked to ferritin). Specific examples of the disclosed PG9 epitope antigens including a dimer of the V1/V2 domain with truncated V1 and V2 variable loops (a dimer of gp120 positions 126-196, having truncated V1 and V2 variable loops) linked to a ferritin protein include the amino acid sequence set forth as SEQ ID NO: 126 (linked dimer of the V1/V2 domain from the CAP45 strain of HIV-1 with truncated V1 and V2 variable loops linked to ferritin) and SEQ ID NO: 127 (linked dimer of the V1/V2 domain from the ZM109 strain of HIV-1 with truncated V1 and V2 variable loops linked to ferritin).

In additional embodiments, any of the disclosed PG9 epitope antigens are linked to an encapsulin polypeptide to construct an encapsulin nanoparticle. Encapsulin proteins are a conserved family of bacterial proteins also known as linocin-like proteins that form large protein assemblies that function as a minimal compartment to package enzymes. The encapsulin assembly is made up of monomeric subunits, which are polypeptides having a molecule weight of approximately 30 kDa. An example of the sequence of one such monomeric subunit is provided as SEQ ID NO: 128. Following production, the monomeric subunits self-assemble into the globular encapsulin assembly including 60 monomeric subunits. Methods of constructing encapsulin nanoparticles are known to the person of ordinary skill in the art, and further described herein (see, for example, Sutter et al., Nature Struct. and Mol. Biol., 15:939-947, 2008, which is incorporated by reference herein in its entirety).

In specific examples, the encapsulin polypeptide is bacterial encapsulin, such as E. coli or Thermotoga maritime encapsulin. An exemplary encapsulin sequence for use with the disclosed PG9 epitope antigens is set forth as SEQ ID NO: 128. Specific examples of the disclosed PG9 epitope antigens including a minimal PG9 binding epitope (gp120 positions 154-177) linked to encapsulin proteins include the amino acid sequence set forth as SEQ ID NO: 129 (minimal PG9 epitope based on HIV-1 strain ZM109 linked to encapsulin), SEQ ID NO: 130 (minimal PG9 epitope based on HIV-1 strain CAP45 linked to encapsulin) and SEQ ID NO: 131 (minimal PG9 epitope based on HIV-1 strain A244 linked to encapsulin). Additional substitutions to the minimal epitope present on a encapsulin protein can be made, for example substitutions of cysteine residues for the amino acids at gp120 positions 155 and 176 of the minimal PG9 epitope on the PG9 epitope-encapsulin fusion protein.

In additional embodiments, any of the disclosed PG9 epitope antigens are linked to a Sulfer Oxygenase Reductase (SOR) polypeptide to construct a SOR nanoparticle. SOR proteins are microbial proteins (for example from the thermoacidophilic archaeon Acidianus ambivalens that form 24 subunit protein assemblies. Methods of constructing SOR nanoparticles are known to the person of ordinary skill in the art (see, e.g., Urich et al., Science, 311:996-1000, 2006, which is incorporated by reference herein in its entirety).

In some examples, any of the disclosed PG9 epitope antigens is genetically fused to the N- or C-terminus of a ferritin protein, an encapsulin protein or a SOR protein, for example with a Ser-Gly linker. When the constructs have been made in HEK 293 Freestyle cells, the fusion proteins are secreted from the cells and self-assembled into particles. The particles can be purified using known techniques, for example by a few different chromatography procedures, e.g. Mono Q (anion exchange) followed by size exclusion (SUPEROSE® 6) chromatography.

Several embodiments include a monomeric subunit of a ferritin protein, an encapsulin protein or a SOR protein, or any portion thereof which is capable of directing self-assembly of monomeric subunits into the globular form of the protein. Amino acid sequences from monomeric subunits of any known ferritin protein, an encapsulin protein or a SOR protein can be used to produce fusion proteins with the disclosed PG9 epitope antigens, so long as the monomeric subunit is capable of self-assembling into a nanoparticle displaying the gp120 polypeptide on its surface.

The fusion proteins need not comprise the full-length sequence of a monomeric subunit polypeptide of a ferritin protein, an encapsulin protein or a SOR protein. Portions, or regions, of the monomeric subunit polypeptide can be utilized so long as the portion comprises amino acid sequences that direct self-assembly of monomeric subunits into the globular form of the protein.

In some embodiments, it may be useful to engineer mutations into the amino acid sequence of the monomeric ferritin, encapsulin or SOR subunits. For example, it may be useful to alter sites such as enzyme recognition sites or glycosylation sites in order to give the fusion protein beneficial properties (e.g., half-life).

It will be understood by those skilled in the art that fusion of any of the disclosed PG9 epitope antigens to the ferritin protein, an encapsulin protein or a SOR protein should be done such that the disclosed PG9 epitope antigen portion of the fusion protein does not interfere with self-assembly of the monomeric ferritin, encapsulin or SOR subunits into the globular protein, and the ferritin protein, an encapsulin protein or a SOR protein portion of the fusion protein does not interfere with the ability of the disclosed PG9 epitope antigen to elicit an immune response to HIV-1. In some embodiments, the ferritin protein, an encapsulin protein or a SOR protein and disclosed PG9 epitope antigen can be joined together directly without affecting the activity of either portion. In other embodiments, the ferritin protein, an encapsulin protein or a SOR protein and the disclosed PG9 epitope antigen are joined using a linker (also referred to as a spacer) sequence. The linker sequence is designed to position the ferritin, encapsulin or SOR portion of the fusion protein and the disclosed PG9 epitope antigen portion of the fusion protein, with regard to one another, such that the fusion protein maintains the ability to assemble into nanoparticles, and also elicit an immune response to HIV-1. In several embodiments, the linker sequences comprise amino acids. Preferable amino acids to use are those having small side chains and/or those which are not charged. Such amino acids are less likely to interfere with proper folding and activity of the fusion protein. Accordingly, preferred amino acids to use in linker sequences, either alone or in combination are serine, glycine and alanine. One example of such a linker sequence is SGG. Amino acids can be added or subtracted as needed. Those skilled in the art are capable of determining appropriate linker sequences for construction of protein nanoparticles.

In certain embodiments, the protein nanoparticles have a molecular weight of from 100 to 4000 kDa, such as 500 to 2100 kDa. In some embodiments, a Ferritin nanoparticle has an approximate molecular weight of 650 kDa, an Encapsulin nanoparticle has an approximate molecular weight of 2100 kDa and a has SOR nanoparticle has an approximate molecular weight of 1000 kDa, when the protein nanoparticle include a PG9 epitope antigen including amino acids 154-177 of gp120 and id glycosylated a position 160 and 156 or 173.

The disclosed PG9 epitope antigens linked to ferritin, encapsulin or SOR proteins can self-assemble into multi-subunit protein nanoparticles, termed ferritin nanoparticles, encapsulin nanoparticles and SOR nanoparticles, respectively. The nanoparticles includes the disclosed PG9 epitope antigens have the same structural characteristics as the native ferritin, encapsulin or SOR nanoparticles that do not include the disclosed PG9 epitope antigens. That is, they contain 24, 60, or 24 subunits (respectively) and have similar corresponding symmetry. In the case of nanoparticles constructed of monomer subunits including a disclosed PG9 epitope antigen, such nanoparticles display at least a portion of the disclosed PG9 epitope antigen on their surface in a PG9-bound conformation. In such a construction, the PG9-bound conformation of the disclosed PG9 epitope antigen is accessible to the immune system and thus can elicit an immune response to HIV-1.

### B. Polynucleotides Encoding Antigens

Polynucleotides encoding the antigens disclosed herein are also provided. These polynucleotides include DNA, cDNA and RNA sequences which encode the antigen.

Methods for the manipulation and insertion of the nucleic acids of this disclosure into vectors are well known in the art (see for example, Sambrook et al., Molecular Cloning, a Laboratory Manual, 2d edition, Cold Spring Harbor Press, Cold Spring Harbor, N.Y., 1989, and Ausubel et al., Current Protocols in Molecular Biology, Greene Publishing Associates and John Wiley & Sons, New York, N.Y., 1994).

A nucleic acid encoding an antigen can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). For example, a polynucleotide encoding the protein can be isolated by polymerase chain reaction of cDNA using primers based on the DNA sequence of the molecule. A wide variety of cloning and in vitro amplification methodologies are well known to persons skilled in the art. PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, NY, 1989). Polynucleotides also can be isolated by screening genomic or cDNA libraries with probes selected from the sequences of the desired polynucleotide under stringent hybridization conditions.

The polynucleotides encoding an antigen include a recombinant DNA which is incorporated into a vector into an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single and double forms of DNA.

DNA sequences encoding the antigen can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Polynucleotide sequences encoding antigens can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

Hosts can include microbial, yeast, insect and mammalian organisms. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, Jakoby and Pastan (eds), 1979, Cell Culture. Methods in Enzymology, volume 58, Academic Press, Inc., Harcourt Brace Jovanovich, N.Y.). Examples of commonly used mammalian host cell lines are VERO and HeLa cells, CHO cells, and WI38, BHK, and COS cell lines, although cell lines may be used, such as cells designed to provide higher expression, desirable glycosylation patterns, or other features. In some embodiments, the host cells include HEK293 cells or derivatives thereof, such as GnTI_{-/-} cells (ATCC® No. CRL-3022).

Transformation of a host cell with recombinant DNA can be carried out by conventional techniques as are well known to those skilled in the art. Where the host is prokaryotic, such as, but not limited to, E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or viral vectors can be used. Eukaryotic cells can also be co-transformed with polynucleotide sequences encoding a disclosed antigen, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982).

A number of viral vectors have been constructed, that can be used to express the disclosed antigens, including polyoma, i.e., SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Natl. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell. Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell. Biol., 5:431-437; Sorge et al., 1984, Mol. Cell. Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

### C. Compositions

The disclosed antigens (for example, a polypeptide including a PG9 epitope or a protein nanoparticle including a PG9 epitope), or nucleic acid molecule a disclosed antigen, can be included in a pharmaceutical composition (including therapeutic and prophylactic formulations), often combined together with one or more pharmaceutically acceptable vehicles and, optionally, other therapeutic ingredients (for example, antibiotics or antiviral drugs). The disclosed antigens are immunogens; therefore, pharmaceutical compositions including one or more of the disclosed antigens are immunogenic compositions.

Such pharmaceutical compositions can be administered to subjects by a variety of administration modes known to the person of ordinary skill in the art, for example, intramuscular, subcutaneous, intravenous, intra-arterial, intra-articular, intraperitoneal, or parenteral routes.

To formulate the pharmaceutical compositions, the disclosed antigens (for example, a polypeptide including a PG9 epitope or a protein nanoparticle including a PG9 epitope), or nucleic acid molecules encoding a disclosed antigen can be combined with various pharmaceutically acceptable additives, as well as a base or vehicle for dispersion of the conjugate. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, TWEEN® 80), solubility enhancing agents (for example, cyclodextrins and derivatives thereof), stabilizers (for example, serum albumin), and reducing agents (for example, glutathione) can be included. Adjuvants, such as aluminum hydroxide (ALHYDROGEL®, available from Brenntag Biosector, Copenhagen, Denmark and AMPHOGEL®, Wyeth Laboratories, Madison, N.J.), Freund's adjuvant, MPL™ (3-O-deacylated monophosphoryl lipid A; Corixa, Hamilton, Ind.) and IL-12 (Genetics Institute, Cambridge, Mass.), among many other suitable adjuvants well known in the art, can be included in the compositions.

When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7.

The disclosed antigens (for example, a polypeptide including a PG9 epitope or a protein nanoparticle including a PG9 epitope), or nucleic acid molecule a disclosed antigen can be dispersed in a base or vehicle, which can include a hydrophilic compound having a capacity to disperse the antigens, and any desired additives. The base can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters and the like can be employed as vehicles. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including fluid or viscous solutions, gels, pastes, powders, microspheres and films, for examples for direct application to a mucosal surface.

The disclosed antigens (for example, a polypeptide including a PG9 epitope or a protein nanoparticle including a PG9 epitope), or nucleic acid molecule a disclosed antigen can be combined with the base or vehicle according to a variety of methods, and release of the antigens can be by diffusion, disintegration of the vehicle, or associated formation of water channels. In some circumstances, the disclosed antigens (for example, a polypeptide including a PG9 epitope or a protein nanoparticle including a PG9 epitope), or nucleic acid molecule a disclosed antigen is dispersed in microcapsules (microspheres) or nanocapsules (nanospheres) prepared from a suitable polymer, for example, isobutyl 2-cyanoacrylate (see, for example, Michael et al., J. Pharmacy Pharmacol. 43:1-5, 1991), and dispersed in a biocompatible dispersing medium, which yields sustained delivery and biological activity over a protracted time.

The pharmaceutical compositions of the disclosure can alternatively contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like.

Pharmaceutical compositions for administering the immunogenic compositions can also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of the disclosed antigens can be brought about by including in the composition an agent which delays absorption, for example, monostearate salts and gelatin.

In certain embodiments, the disclosed antigens (for example, a polypeptide including a PG9 epitope or a protein nanoparticle including a PG9 epitope), or nucleic acid molecule a disclosed antigen can be administered in a time-release formulation, for example in a composition that includes a slow release polymer. These compositions can be prepared with vehicles that will protect against rapid release, for example a controlled release vehicle such as a polymer, microencapsulated delivery system or bioadhesive gel. Prolonged delivery in various compositions of the disclosure can be brought about by including in the composition agents that delay absorption, for example, aluminum monostearate hydrogels and gelatin. When controlled release formulations are desired, controlled release binders suitable for use in accordance with the disclosure include any biocompatible controlled release material which is inert to the active agent and which is capable of incorporating the disclosed antigen and/or other biologically active agent. Numerous such materials are known in the art. Useful controlled-release binders are materials that are metabolized slowly under physiological conditions following their delivery (for example, at a mucosal surface, or in the presence of bodily fluids). Appropriate binders include, but are not limited to, biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body. Numerous systems for controlled delivery of therapeutic proteins are known (e.g., U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; and U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342; and U.S. Pat. No. 5,534,496).

Exemplary polymeric materials for use in the present disclosure include, but are not limited to, polymeric matrices derived from copolymeric and homopolymeric polyesters having hydrolyzable ester linkages. A number of these are known in the art to be biodegradable and to lead to degradation products having no or low toxicity. Exemplary polymers include polyglycolic acids and polylactic acids, poly(DL-lactic acid-co-glycolic acid), poly(D-lactic acid-co-glycolic acid), and poly(L-lactic acid-co-glycolic acid). Other useful biodegradable or bioerodable polymers include, but are not limited to, such polymers as poly(epsilon-caprolactone), poly(epsilon-aprolactone-CO-lactic acid), poly(epsilon.-aprolactone-CO-glycolic acid), poly(beta-hydroxy butyric acid), poly(alkyl-2-cyanoacrilate), hydrogels, such as poly(hydroxyethyl methacrylate), polyamides, poly(amino acids) (for example, L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides, and copolymers thereof. Many methods for preparing such formulations are well known to those skilled in the art (see, for example, Sustained and Controlled Release Drug Delivery Systems, J. R. Robinson, ed., Marcel Dekker, Inc., New York, 1978). Other useful formulations include controlled-release microcapsules (U.S. Pat. Nos. 4,652,441 and 4,917,893), lactic acid-glycolic acid copolymers useful in making microcapsules and other formulations (U.S. Pat. Nos. 4,677,191 and 4,728,721) and sustained-release compositions for water-soluble peptides (U.S. Pat. No. 4,675,189).

The pharmaceutical compositions of the disclosure typically are sterile and stable under conditions of manufacture, storage and use. Sterile solutions can be prepared by incorporating the conjugate in the required amount in an appropriate solvent with one or a combination of ingredients enumerated herein, as required, followed by filtered sterilization. Generally, dispersions are prepared by incorporating the disclosed antigen and/or other biologically active agent into a sterile vehicle that contains a basic dispersion medium and the required other ingredients from those enumerated herein. In the case of sterile powders, methods of preparation include vacuum drying and freeze-drying which yields a powder of the disclosed antigen plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

In one specific, non-limiting example, a pharmaceutical composition for intravenous administration would include about 0.1 μg to 10 mg of a disclosed antigens (for example, a polypeptide including a PG9 epitope or a protein nanoparticle including a PG9 epitope) per subject per day. Dosages from 0.1 up to about 100 mg per subject per day can be used, particularly if the agent is administered to a secluded site and not into the circulatory or lymph system, such as into a body cavity or into a lumen of an organ. Actual methods for preparing administrable compositions will be known or apparent to those skilled in the art and are described in more detail in such publications as Remingtons Pharmaceutical Sciences, 19^{th} Ed., Mack Publishing Company, Easton, Pa., 1995.

### D. Methods of Treatment

The disclosed antigens (for example, a polypeptide including a PG9 epitope or a protein nanoparticle including a PG9 epitope) are immunogens. Thus, in several embodiments, a therapeutically effective amount of an immunogenic composition including one or more of the disclosed antigens (for example, a polypeptide including a PG9 epitope or a protein nanoparticle including a PG9 epitope), can be administered to a subject in order to generate an immune response to a pathogen, for example HIV-1.

In accordance with the disclosure herein, a prophylactically or therapeutically effective amount of a disclosed immunogenic composition (for example, a composition including a disclosed antigen, such as a polypeptide including a PG9 epitope or a protein nanoparticle including a PG9 epitope as disclosed herein) is administered to a subject in need of such treatment for a time and under conditions sufficient to prevent, inhibit, and/or ameliorate a selected disease or condition or one or more symptom(s) thereof. The immunogenic composition is administered in an amount sufficient to raise an immune response against an HIV polypeptide (such as gp120) in the subject. In some embodiments, administration of a disclosed immunogenic composition to a subject elicits an immune response against an HIV in the subject, for example an immune response against a HIV-1 protein, such as gp120.

In some embodiments, a subject is selected for treatment that has, or is at risk for developing, an HIV infection, for example because of exposure or the possibility of exposure to HIV. Following administration of a therapeutically effective amount of the disclosed therapeutic compositions, the subject can be monitored for HIV-1 infection, symptoms associated with HIV-1 infection, or both.

Typical subjects intended for treatment with the compositions and methods of the present disclosure include humans, as well as non-human primates and other animals. To identify subjects for prophylaxis or treatment according to the methods of the disclosure, accepted screening methods are employed to determine risk factors associated with a targeted or suspected disease or condition, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods, which are available and well known in the art to detect and/or characterize HIV infection. These and other routine methods allow the clinician to select patients in need of therapy using the methods and pharmaceutical compositions of the disclosure. In accordance with these methods and principles, an immunogenic composition can be administered according to the teachings herein, or other conventional methods known to the person of ordinary skill in the art, as an independent prophylaxis or treatment program, or as a follow-up, adjunct or coordinate treatment regimen to other treatments.

The immunogenic composition can be used in coordinate vaccination protocols or combinatorial formulations. In certain embodiments, novel combinatorial immunogenic compositions and coordinate immunization protocols employ separate immunogens or formulations, each directed toward eliciting an anti-HIV immune response, such as an immune response to HIV-1 gp120 protein. Separate immunogenic compositions that elicit the anti-HIV immune response can be combined in a polyvalent immunogenic composition administered to a subject in a single immunization step, or they can be administered separately (in monovalent immunogenic compositions) in a coordinate immunization protocol.

The administration of the immunogenic compositions of the disclosure can be for either prophylactic or therapeutic purpose. When provided prophylactically, the immunogenic composition is provided in advance of any symptom, for example in advance of infection. The prophylactic administration of the immunogenic compositions serves to prevent or ameliorate any subsequent infection. When provided therapeutically, the immunogenic compositions is provided at or after the onset of a symptom of disease or infection, for example after development of a symptom of HIV-1 infection, or after diagnosis of HIV-1 infection. The immunogenic composition can thus be provided prior to the anticipated exposure to HIV virus so as to attenuate the anticipated severity, duration or extent of an infection and/or associated disease symptoms, after exposure or suspected exposure to the virus, or after the actual initiation of an infection.

Administration induces a sufficient immune response to treat the pathogenic infection, for example, to inhibit the infection and/or reduce the signs and/or symptoms of the infection. Amounts effective for this use will depend upon the severity of the disease, the general state of the subject's health, and the robustness of the subject's immune system. A therapeutically effective amount of the disclosed immunogenic compositions is that which provides either subjective relief of a symptom(s) or an objectively identifiable improvement as noted by the clinician or other qualified observer.

For prophylactic and therapeutic purposes, the immunogenic composition can be administered to the subject in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the immunogenic composition can be provided as repeated doses within a prolonged prophylaxis or treatment regimen that will yield clinically significant results to alleviate one or more symptoms or detectable conditions associated with a targeted disease or condition as set forth herein. Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, rat, porcine, feline, ferret, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer a therapeutically effective amount of the immunogenic composition (for example, amounts that are effective to elicit a desired immune response or alleviate one or more symptoms of a targeted disease). In alternative embodiments, an effective amount or effective dose of the immunogenic composition may simply inhibit or enhance one or more selected biological activities correlated with a disease or condition, as set forth herein, for either therapeutic or diagnostic purposes.

In one embodiment, a suitable immunization regimen includes at least three separate inoculations with one or more immunogenic compositions, with a second inoculation being administered more than about two, about three to eight, or about four, weeks following the first inoculation. Generally, the third inoculation is administered several months after the second inoculation, and in specific embodiments, more than about five months after the first inoculation, more than about six months to about two years after the first inoculation, or about eight months to about one year after the first inoculation. Periodic inoculations beyond the third are also desirable to enhance the subject's “immune memory.” The adequacy of the vaccination parameters chosen, e.g., formulation, dose, regimen and the like, can be determined by taking aliquots of serum from the subject and assaying antibody titers during the course of the immunization program. Alternatively, the T cell populations can be monitored by conventional methods. In addition, the clinical condition of the subject can be monitored for the desired effect, e.g., prevention of HIV-1 infection or progression to AIDS, improvement in disease state (e.g., reduction in viral load), or reduction in transmission frequency to an uninfected partner. If such monitoring indicates that vaccination is sub-optimal, the subject can be boosted with an additional dose of immunogenic composition, and the vaccination parameters can be modified in a fashion expected to potentiate the immune response. Thus, for example, the dose of the chimeric non-HIV polypeptide or polynucleotide and/or adjuvant can be increased or the route of administration can be changed.

It is contemplated that there can be several boosts, and that each boost can be a different PG9 antigen or immunogenic fragment thereof. It is also contemplated that in some examples that the boost may be the same disclosed PG9 epitope antigen as another boost, or the prime.

The prime can be administered as a single dose or multiple doses, for example two doses, three doses, four doses, five doses, six doses or more can be administered to a subject over days, weeks or months. The boost can be administered as a single dose or multiple doses, for example two to six doses, or more can be administered to a subject over a day, a week or months. Multiple boosts can also be given, such one to five, or more. Different dosages can be used in a series of sequential inoculations. For example a relatively large dose in a primary inoculation and then a boost with relatively smaller doses. The immune response against the selected antigenic surface can be generated by one or more inoculations of a subject with an immunogenic composition disclosed herein.

The actual dosage of the immunogenic composition will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the immunogenic composition for eliciting the desired activity or biological response in the subject. Dosage regimens can be adjusted to provide an optimum prophylactic or therapeutic response. As described above in the forgoing listing of terms, an effective amount is also one in which any toxic or detrimental side effects of the disclosed antigen and/or other biologically active agent is outweighed in clinical terms by therapeutically beneficial effects. A non-limiting range for a therapeutically effective amount of the disclosed antigens (for example, a polypeptide including a PG9 epitope or a protein nanoparticle including a PG9 epitope) within the methods and immunogenic compositions of the disclosure is about 0.01 mg/kg body weight to about 10 mg/kg body weight, such as about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.5 mg/kg, about 2 mg/kg, about 2.5 mg/kg, about 3 mg/kg, about 4 mg/kg, about 5 mg/kg, or about 10 mg/kg, for example 0.01 mg/kg to about 1 mg/kg body weight, about 0.05 mg/kg to about 5 mg/kg body weight, about 0.2 mg/kg to about 2 mg/kg body weight, or about 1.0 mg/kg to about 10 mg/kg body weight.

In one specific, non-limiting example, an immunogenic composition for intravenous administration would include about 0.1 μg to 10 mg of a disclosed antigen per subject per day. In another example, the dosage can range from 0.1 up to about 100 mg per subject per day, particularly if the agent is administered to a secluded site and not into the circulatory or lymph system, such as into a body cavity or into a lumen of an organ. Actual methods for preparing administrable compositions will be known or apparent to those skilled in the art and are described in more detail in such publications as Remingtons Pharmaceutical Sciences, 19^{th} Ed., Mack Publishing Company, Easton, Pa., 1995.

Dosage can be varied by the attending clinician to maintain a desired concentration at a target site (for example, systemic circulation). Higher or lower concentrations can be selected based on the mode of delivery, for example, trans-epidermal, rectal, oral, pulmonary, or intranasal delivery versus intravenous or subcutaneous delivery. Dosage can also be adjusted based on the release rate of the administered formulation, for example, of an intrapulmonary spray versus powder, sustained release oral versus injected particulate or transdermal delivery formulations, and so forth. To achieve the same serum concentration level, for example, slow-release particles with a release rate of 5 nanomolar (under standard conditions) would be administered at about twice the dosage of particles with a release rate of 10 nanomolar.

Upon administration of an immunogenic composition of this disclosure, the immune system of the subject typically responds to the immunogenic composition by producing antibodies specific for HIV-1 gp120 protein. Such a response signifies that an immunologically effective dose of the immunogenic composition was delivered.

An immunologically effective dosage can be achieved by single or multiple administrations (including, for example, multiple administrations per day), daily, or weekly administrations. For each particular subject, specific dosage regimens can be evaluated and adjusted over time according to the individual need and professional judgment of the person administering or supervising the administration of the immunogenic composition. In some embodiments, the antibody response of a subject administered the compositions of the disclosure will be determined in the context of evaluating effective dosages/immunization protocols. In most instances it will be sufficient to assess the antibody titer in serum or plasma obtained from the subject. Decisions as to whether to administer booster inoculations and/or to change the amount of the composition administered to the individual can be at least partially based on the antibody titer level. The antibody titer level can be based on, for example, an immunobinding assay which measures the concentration of antibodies in the serum which bind to an antigen including the PG9 epitope, for example, HIV-1 gp120 protein. The methods of using immunogenic composition, and the related compositions and methods of the disclosure are useful in increasing resistance to, preventing, ameliorating, and/or treating infection and disease caused by HIV (such as HIV-1) in animal hosts, and other, in vitro applications.

In several embodiments, it may be advantageous to administer the immunogenic compositions disclosed herein with other agents such as proteins, peptides, antibodies, and other antiviral agents, such as anti-HIV agents. Examples of such anti-HIV therapeutic agents include nucleoside reverse transcriptase inhibitors, such as abacavir, AZT, didanosine, emtricitabine, lamivudine, stavudine, tenofovir, zalcitabine, zidovudine, and the like, non-nucleoside reverse transcriptase inhibitors, such as delavirdine, efavirenz, nevirapine, protease inhibitors such as amprenavir, atazanavir, indinavir, lopinavir, nelfinavir, osamprenavir, ritonavir, saquinavir, tipranavir, and the like, and fusion protein inhibitors such as enfuvirtide and the like. In certain embodiments, immunogenic compositions are administered concurrently with other anti-HIV therapeutic agents. In some examples, the disclosed PG9 epitope antigens are administered with T-helper cells, such as exogenous T-helper cells. Exemplary methods for the producing and administering T-helper cells can be found in International Patent Publication WO 03/020904, which is incorporated herein by reference.

In certain embodiments, the immunogenic compositions are administered sequentially with other anti-HIV therapeutic agents, such as before or after the other agent. One of ordinary skill in the art would know that sequential administration can mean immediately following or after an appropriate period of time, such as hours, days, weeks, months, or even years later.

In additional embodiments, a therapeutically effective amount of a pharmaceutical composition including a nucleic acid encoding a disclosed antigen is administered to a subject in order to generate an immune response. In one specific, non-limiting example, a therapeutically effective amount of a nucleic acid encoding a disclosed antigen is administered to a subject to treat or prevent or inhibit HIV infection.

One approach to administration of nucleic acids is direct immunization with plasmid DNA, such as with a mammalian expression plasmid. As described above, the nucleotide sequence encoding a disclosed antigen can be placed under the control of a promoter to increase expression of the molecule.

Immunization by nucleic acid constructs is well known in the art and taught, for example, in U.S. Pat. No. 5,643,578 (which describes methods of immunizing vertebrates by introducing DNA encoding a desired antigen to elicit a cell-mediated or a humoral response), and U.S. Pat. No. 5,593,972 and U.S. Pat. No. 5,817,637 (which describe operably linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids (or of the synthetic peptides themselves), and immune-stimulating constructs, or ISCOMS™, negatively charged cage-like structures of 30-40 nm in size formed spontaneously on mixing cholesterol and Quil A™ (saponin). Protective immunity has been generated in a variety of experimental models of infection, including toxoplasmosis and Epstein-Barr virus-induced tumors, using ISCOMS™ as the delivery vehicle for antigens (Mowat and Donachie, Immunol. Today 12:383, 1991). Doses of antigen as low as 1 μg encapsulated in ISCOMS™ have been found to produce Class I mediated CTL responses (Takahashi et al., Nature 344:873, 1990).

In another approach to using nucleic acids for immunization, a disclosed antigen can also be expressed by attenuated viral hosts or vectors or bacterial vectors. Recombinant vaccinia virus, adeno-associated virus (AAV), herpes virus, retrovirus, cytogmeglo virus or other viral vectors can be used to express the peptide or protein, thereby eliciting a CTL response. For example, vaccinia vectors and methods useful in immunization protocols are described in U.S. Pat. No. 4,722,848. BCG (Bacillus Calmette Guerin) provides another vector for expression of the peptides (see Stover, Nature 351:456-460, 1991).

In one embodiment, a nucleic acid encoding a disclosed antigen is introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites, including tissues in proximity to metastases. Dosages for injection are usually around 0.5 μg/kg to about 50 mg/kg, and typically are about 0.005 mg/kg to about 5 mg/kg (see, e.g., U.S. Pat. No. 5,589,466).

### D. Immunodiagnostic Reagents and Kits

In addition to the therapeutic methods provided above, any of the disclosed antigens (for example, a polypeptide including a PG9 epitope or a protein nanoparticle including a PG9 epitope) can be utilized to produce antigen specific immunodiagnostic reagents, for example, for serosurveillance. Immunodiagnostic reagents can be designed from any of the antigenic polypeptide described herein. For example, in the case of the disclosed antigens, the presence of serum antibodies to HIV is monitored using the isolated antigens disclosed herein, such as to detect an HIV infection and/or the presence of antibodies that specifically bind to the PG9 epitope of gp120.

Generally, the method includes contacting a sample from a subject, such as, but not limited to a blood, serum, plasma, urine or sputum sample from the subject with one or more of the disclosed PG9 epitope antigens disclosed herein (including a polymeric form thereof) and detecting binding of antibodies in the sample to the disclosed immunogens. The binding can be detected by any means known to one of skill in the art, including the use of labeled secondary antibodies that specifically bind the antibodies from the sample. Labels include radiolabels, enzymatic labels, and fluorescent labels.

Any such immunodiagnostic reagents can be provided as components of a kit. Optionally, such a kit includes additional components including packaging, instructions and various other reagents, such as buffers, substrates, antibodies or ligands, such as control antibodies or ligands, and detection reagents.

Methods are further provided for a diagnostic assay to monitor HIV-1 induced disease in a subject and/or to monitor the response of the subject to immunization with one or more of the disclosed antigens. By “HIV-1 induced disease” is intended any disease caused, directly or indirectly, by HIV. An example of an HIV-1 induced disease is acquired immunodeficiency syndrome (AIDS). The method includes contacting a disclosed antigen with a sample of bodily fluid from the subject, and detecting binding of antibodies in the sample to the disclosed immunogens. In addition, the detection of the HIV-1 binding antibody also allows the response of the subject to immunization with the disclosed antigen to be monitored. In still other embodiments, the titer of the HIV-1 binding antibodies is determined. The binding can be detected by any means known to one of skill in the art, including the use of labeled secondary antibodies that specifically bind the antibodies from the sample. Labels include radiolabels, enzymatic labels, and fluorescent labels. In other embodiments, a disclosed immunogen is used to isolate antibodies present in a subject or biological sample obtained from a subject.

### III. Examples

The following examples are provided to illustrate particular features of certain embodiments, but the scope of the claims should not be limited to those features exemplified.

### Example 1

### Structure of HIV-1 Gp120 V1V2 Domain with Broadly Neutralizing Antibody PG9

This example illustrates the structure of the V1V2 domain in complex with monoclonal antibody PG9. V1V2 forms a 4-stranded β-sheet domain, in which sequence diversity and glycosylation are largely segregated to strand-connecting loops. PG9 recognition involves electrostatic, sequence-independent, and glycan interactions: the latter account for over half the interactive surface but are of sufficiently weak affinity to avoid auto-reactivity. The results structurally define V1V2 and identify PG9 antibody recognition for the V1V2 domain of HIV-1.

### Introduction

As the sole viral target of neutralizing antibodies, the HIV-1 viral spike has evolved to evade antibody-mediated neutralization. Variable regions 1 and 2 (V1V2) of the gp120 component of the viral spike are critical to this evasion. Localized by electron microscopy to a membrane-distal “cap,” which holds the spike in a neutralization-resistant conformation, V1V2 is not essential for entry: its removal, however, renders the virus profoundly sensitive to antibody-mediated neutralization.

The ˜50-90 residues that comprise V1V2 contain two of the most variable portions of the virus, and 1 in 10 residues of V1V2 are N-glycosylated. Despite the diversity and glycosylation of V1V2, a number of broadly neutralizing human antibodies have been identified that target this region, including the somatically related antibodies PG9 and PG16, which neutralize 70-80% of circulating HIV-1 isolates (Walker et al., Science, 326:285-289, 2009), antibodies CH01-CH04, which neutralize 40-50% (Bonsignori et al., J Virol, 85:9998-10009, 2011), and antibodies PGT141-145, which neutralize 40-80% (Walker et al., Nature, 477:466-470, 2011). These antibodies all share specificity for an N-linked glycan at residue 160 in V1V2 (HXB2 numbering) and show a preferential binding to the assembled viral spike over monomeric gp120 as well as a sensitivity to changes in V1V2 and some V3 residues. Sera with these characteristics have been identified in a number of HIV-1 donor cohorts, and these quaternary-structure-preferring V1V2-directed antibodies are among the most common broadly neutralizing responses in infected donors (Walker et al., PLoS Pathog, 6:e1001028, 2010 and Moore et al., J Virol, 85:3128-3141, 2011).

Despite extensive effort, V1V2 had resisted atomic-level characterization. This example provides crystal structures of the V1V2 domain of HIV-1 gp120 from strains CAP45 and ZM109 in complexes with the antigen-binding fragment (Fab) of PG9 at 2.19 and 1.80 Å resolution, respectively.

### Structure Determination

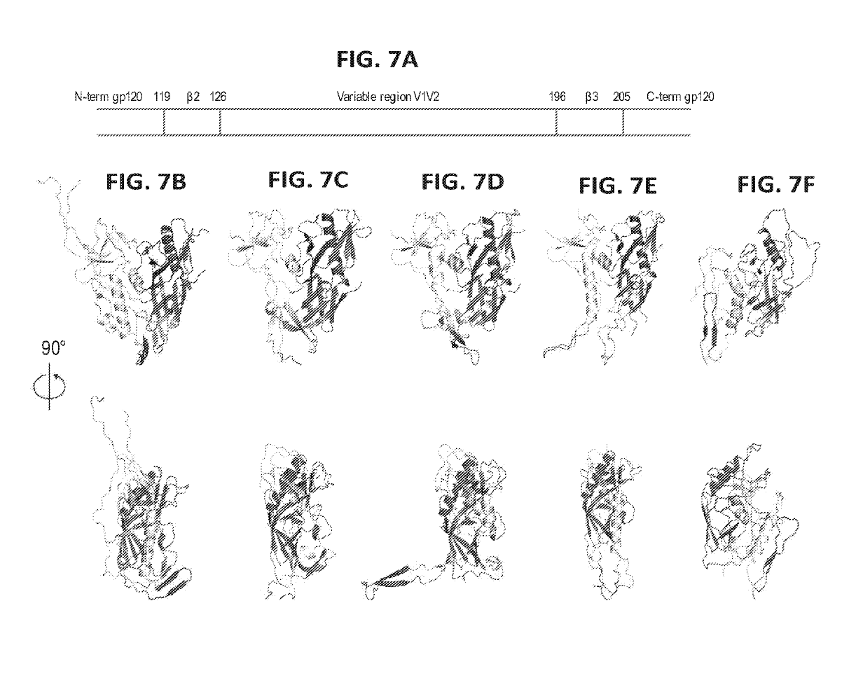

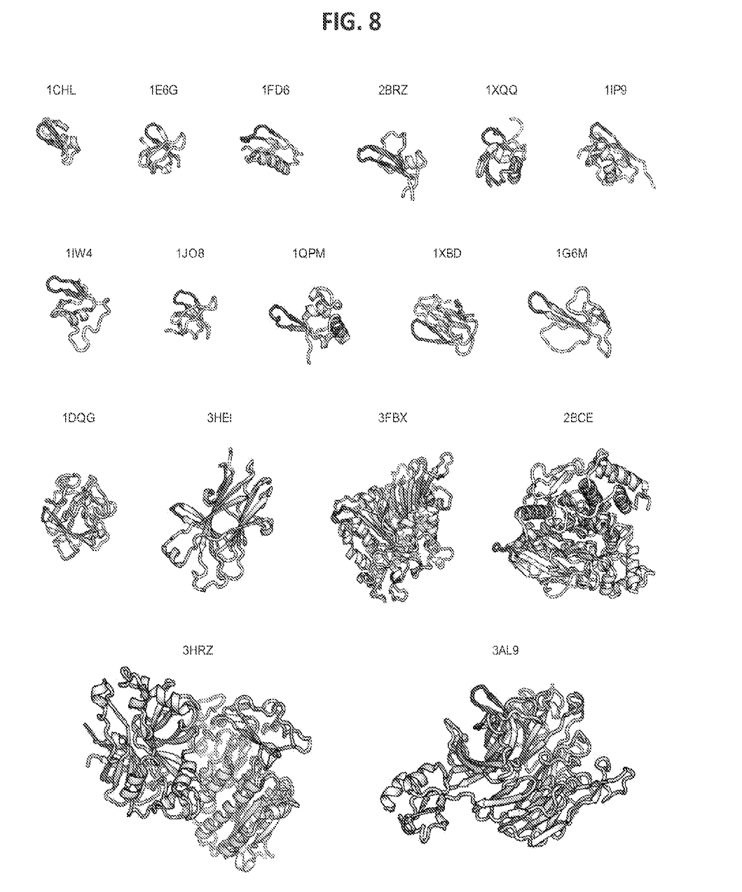

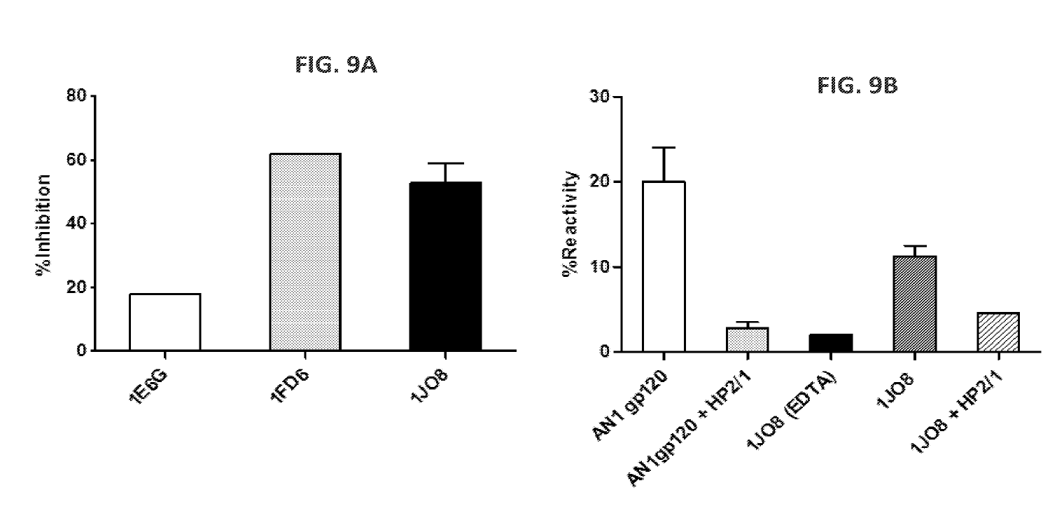

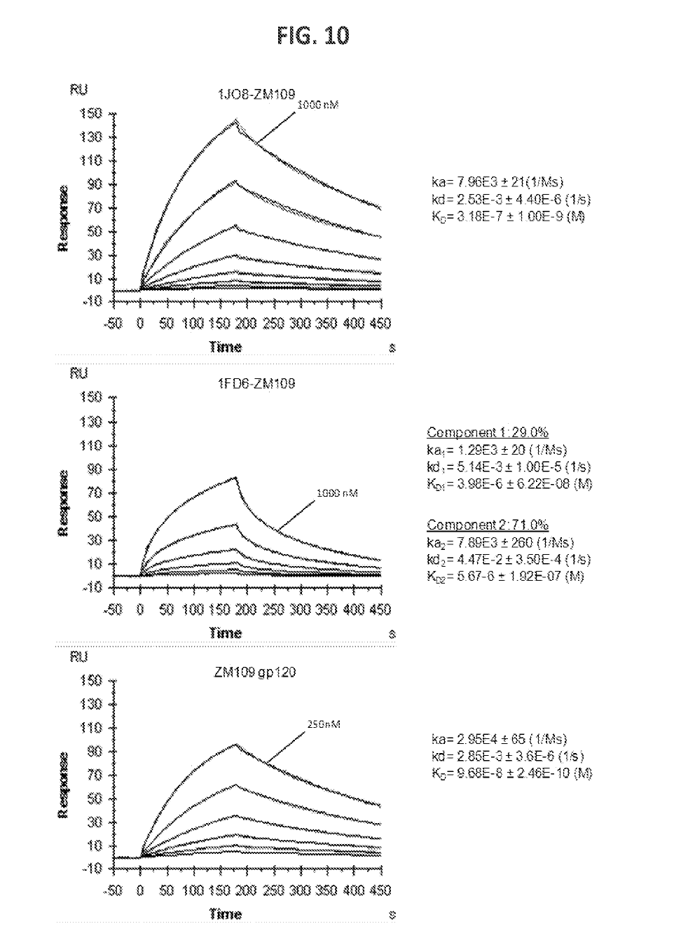

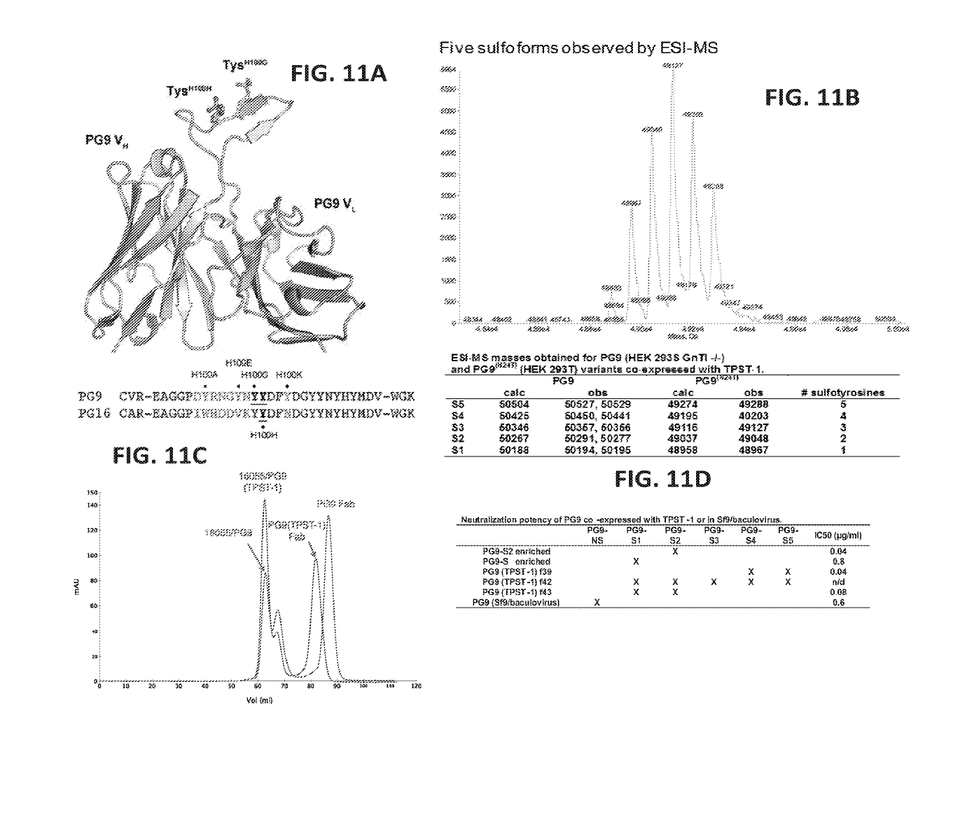

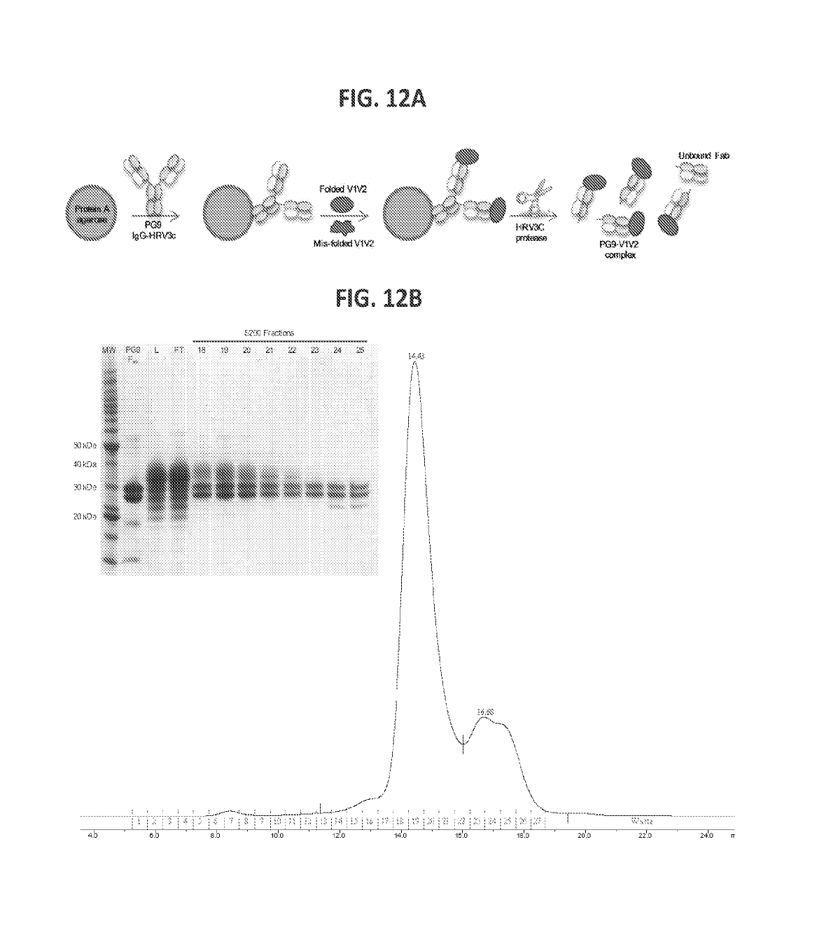

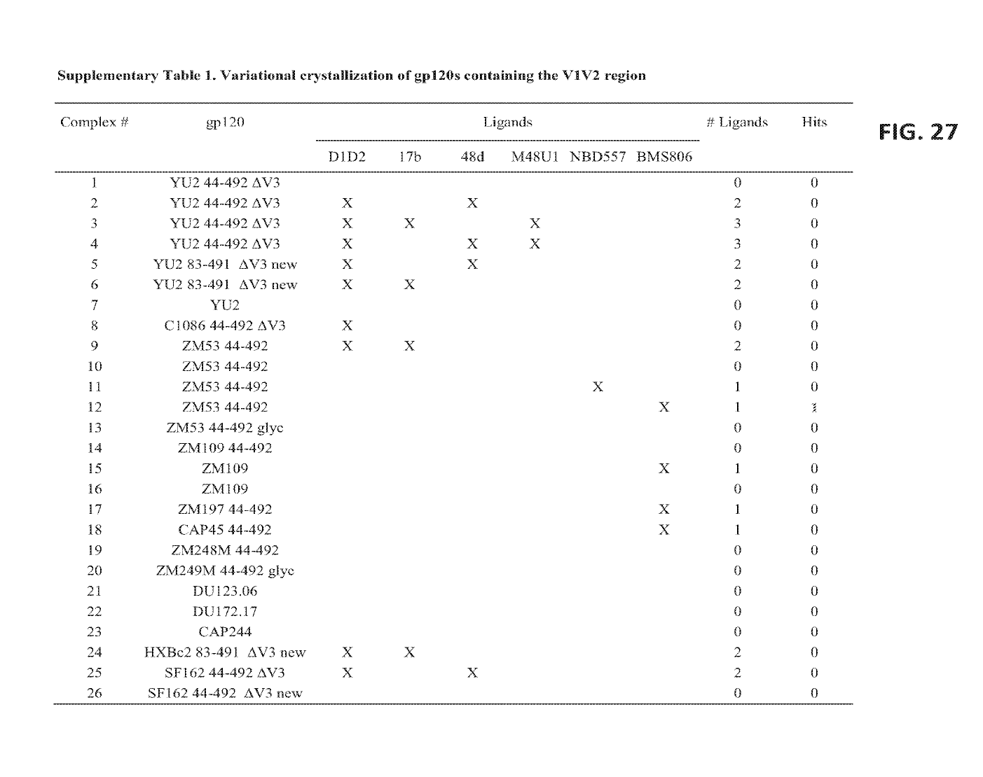

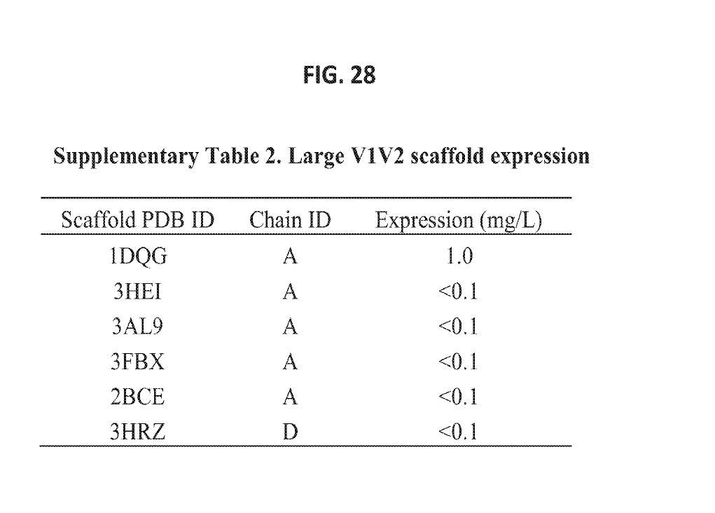

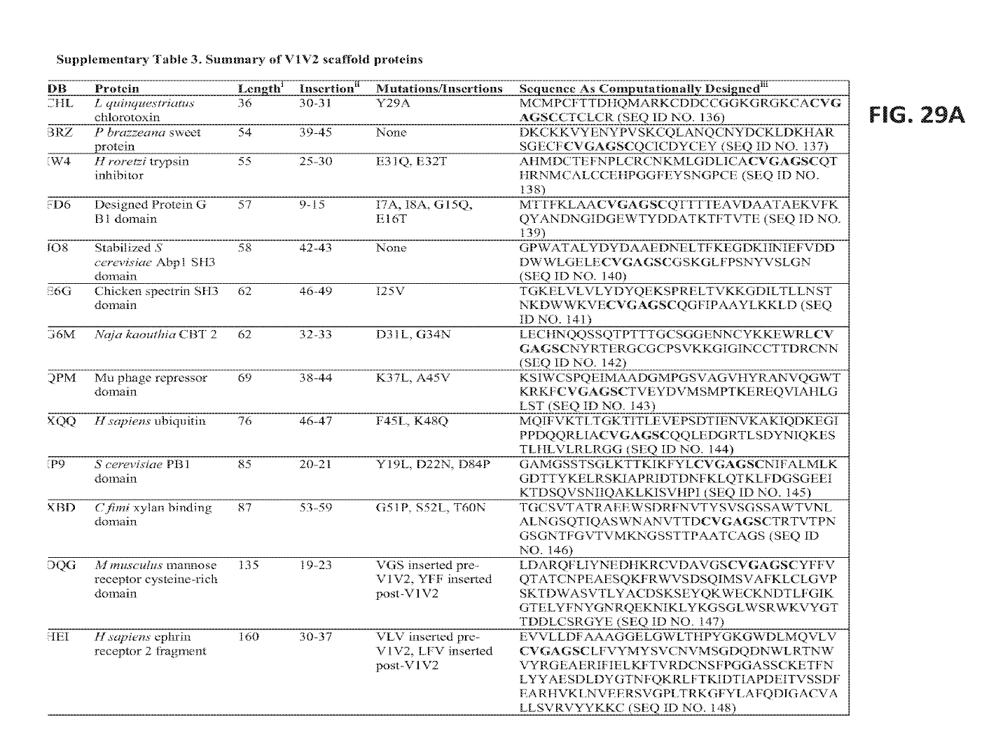

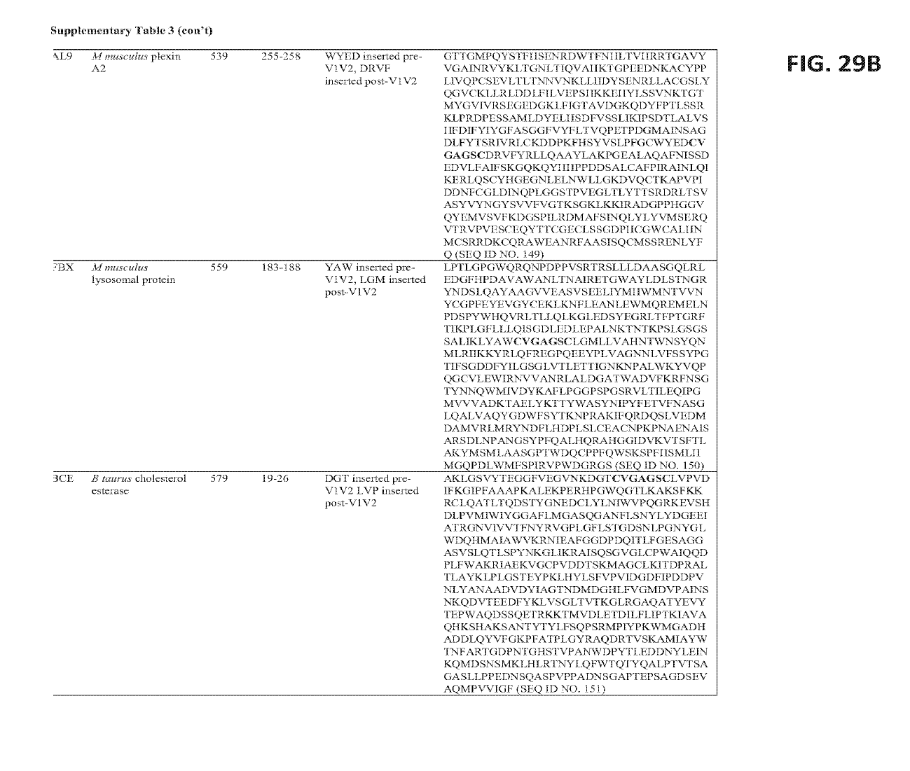

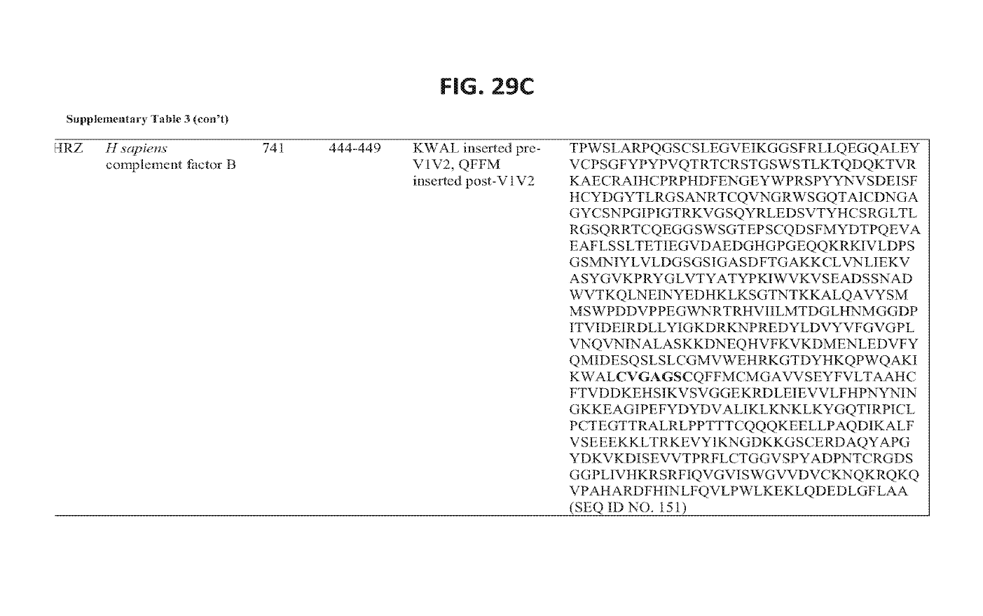

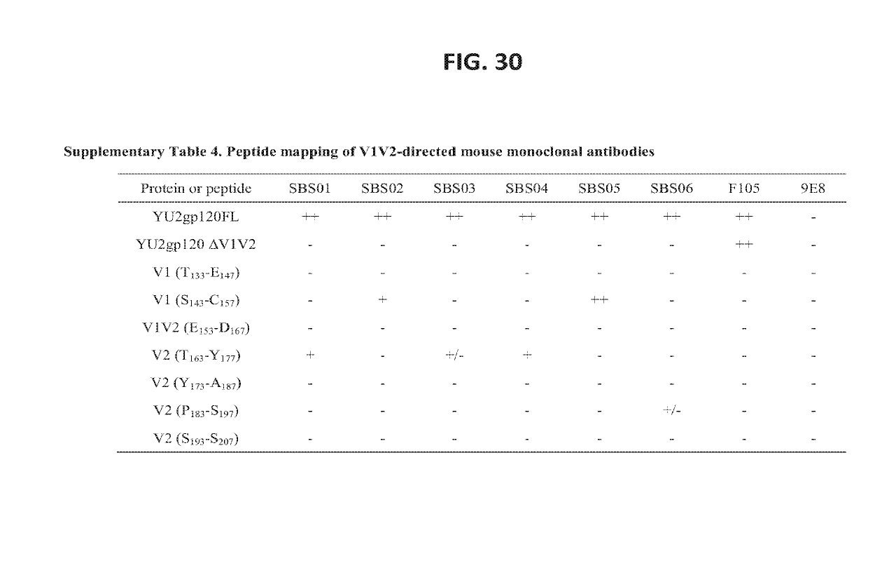

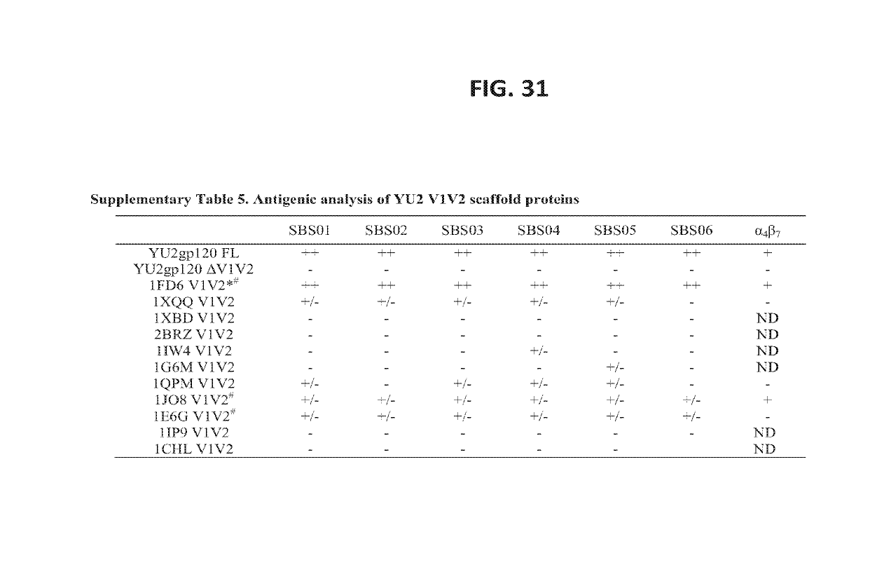

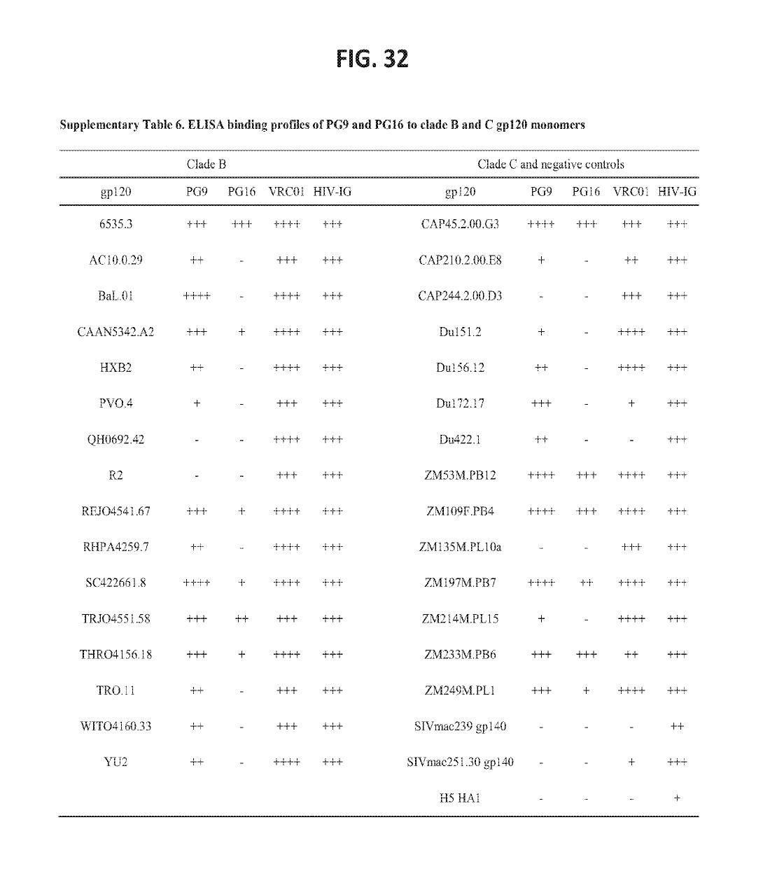

Variational crystallization of HIV-1 gp120 with V1V2 was attempted following strategies that were successful with structural determination for other portions of HIV-1 gp120; this failed to produce V1V2-containing crystals suitable for structural analysis (Supplementary Table 1 shown in FIG. 27). Because V1V2 emanates from similar hairpins in core structures of HIV-1 and SIV (FIG. 7), protein scaffolds that provided an appropriate hairpin might suitably incorporate and express an ectopic V1V2 region. Six proteins with potentially suitable acceptor β-hairpins that ranged in size from 135 to 741 amino acids were tested. Only the smallest of these expressed in transfected 293F cells when scaffolded with V1V2 (Supplementary Table 2 shown in FIG. 28), but it behaved poorly in solution. Eleven smaller proteins of 36-87 amino acids in size were identified and chimeric proteins encoding V1V2 from the YU2 strain of HIV-1 were constructed (FIG. 8 and Supplementary Table 3 shown in FIGS. 29A-29C). The expressed chimeric glycoproteins from these smaller scaffolds were mostly soluble, permitting us to characterize them antigenically against a panel of six YU2-specific V1V2 antibodies (Supplementary Tables 4 and 5 shown in FIG. 30 and FIG. 31, respectively). Three of the smaller scaffolded-YU2 V1V2 chimeras showed reactivity with all six YU2-specific antibodies, and two (1FD6 (Ross et al., Protein Sci, 10:450-454, 2011) and 1JO8 (Fazi et al., J Biol Chem, 277:5290-5298, 2002) were also recognized by the α₄β₇ integrin (Arthos et al., Nat Immunol, 9:301-309, 2008), suggesting that they retained biological integrity (FIG. 9 and Supplementary Table 5 shown in FIG. 31). Next, strains of gp120 that retained PG9 recognition in the gp120 monomer context were identified, including Clade B strain TRJO and Clade C strains 16055, CAP45, ZM53 and ZM109 (Supplementary Table 6 shown in FIG. 32). V1V2 sequences (residues 126-196) from these strains were placed into the 1FD6 and 1JO8 scaffolds, and assessed PG9 binding. Notably, affinities of PG9 for 1FD6-ZM109 and 1JO8-ZM109 were only 50-fold and 3-fold lower than wild-type ZM109 gp120, respectively (FIG. 10). Scaffold-V1V2 heterogeneity was apparent after expression in GnTI^{-/-} cells (Reeves et al., Proc Natl Acad Sci USA, 99:13419-13424, 2002) as was sulfation heterogeneity on antibody PG9 (Pejchal et al., Proc Natl Acad Sci USA, 107:11483-11488, 2010) (FIG. 11). An on-column selection procedure coupled to on-column protease cleavage of Fab was used to obtain homogeneous complexes of scaffold-V1V2 with PG9 (FIG. 12).

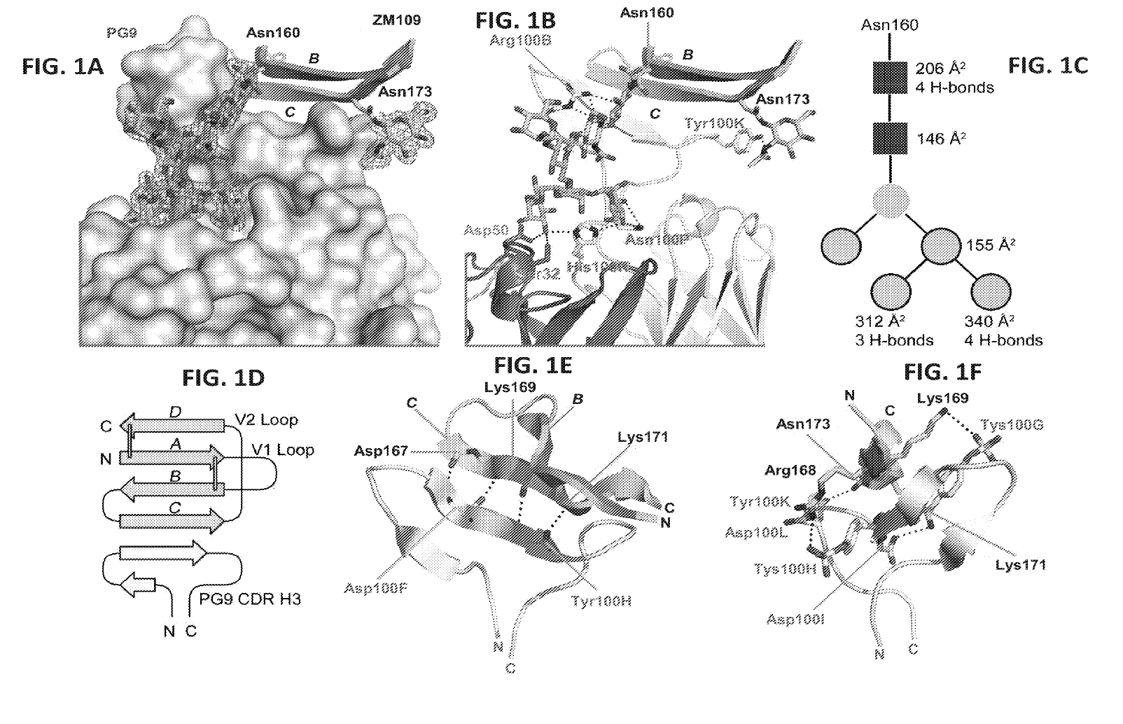

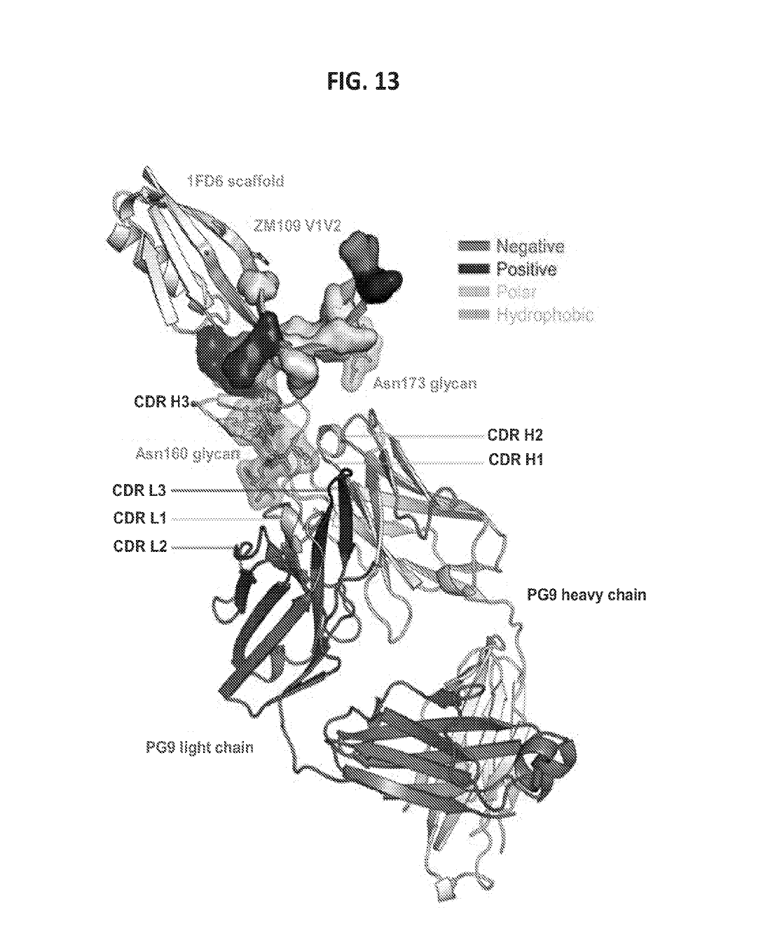

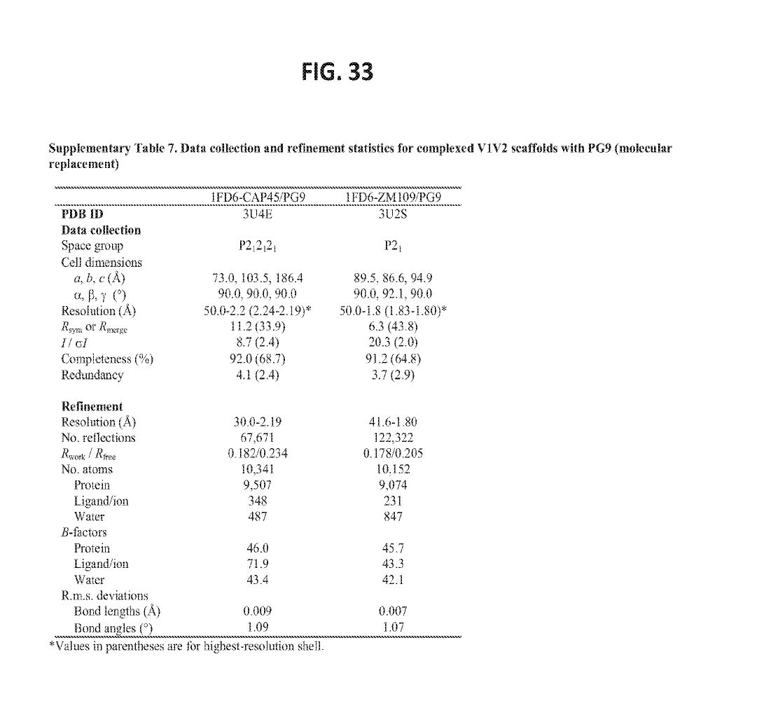

Two 1FD6-V1V2 scaffolds were crystallized in complex with PG9. One scaffold contained the V1V2 region from the CAP45 strain of HIV-1 gp120 with five sites of potential N-linked glycosylation. Crystals of this CAP45 construct with the Fab of PG9 diffracted to 2.19 Å, and the structure was refined to an R_{cryst} of 18.2% (R_{free}=23.4%) (FIG. 1, Supplementary Table 7 shown in FIG. 33). A second scaffold included the V1V2 region from the ZM109 strain of HIV-1 gp120 with N-linked glycans at positions 160 and 173, and asparagine to alanine mutations at four other potential N-linked sites. Crystals of this ZM109 construct with the Fab of PG9 diffracted to 1.80 Å, and the structure was refined to an of 17.8% (R_{free}=20.5%) (FIG. 13 and Supplementary Table 7 shown in FIG. 33).

### Structure of V1V2

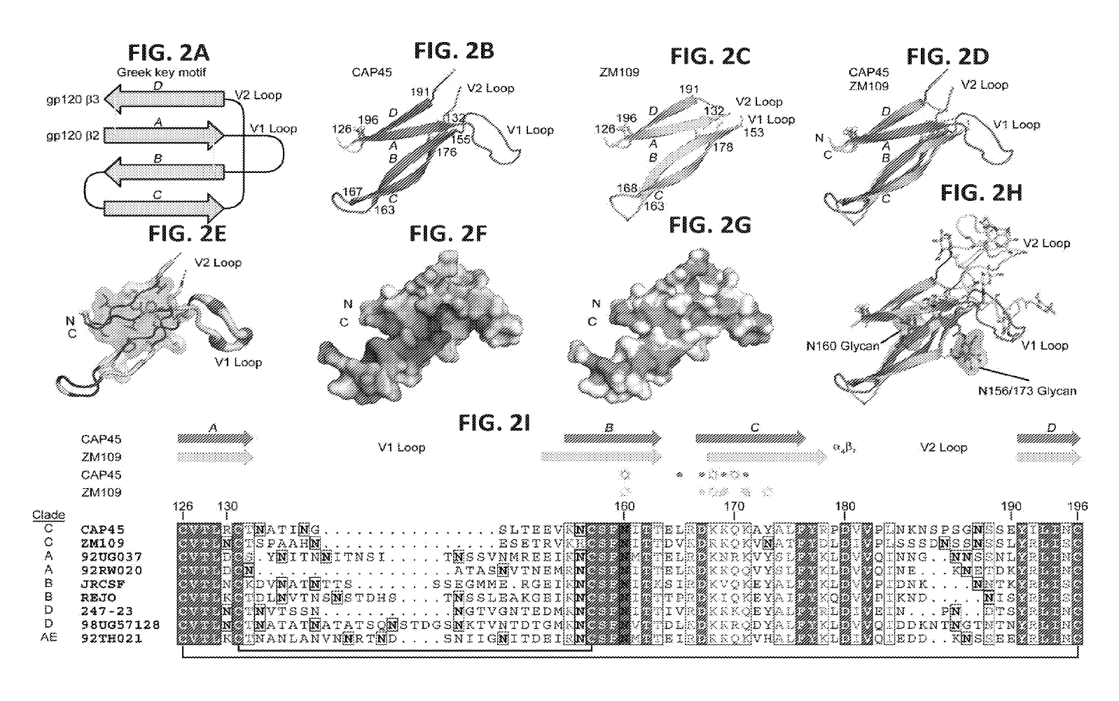

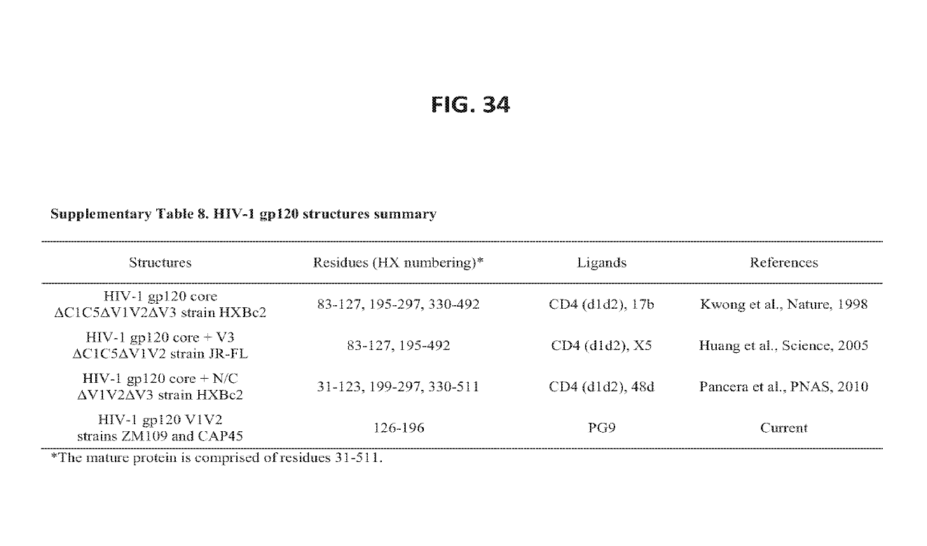

The V1V2 structure, in the context of scaffold and PG9, folds as four anti-parallel β-strands (labeled A, B, C, D) arranged in (−1, −1, +3) topology (Richardson, Adv Protein Chem, 34:167-339, 1981) (FIGS. 2A-D and Supplementary Table 8 shown in FIG. 34). Important structural elements such as a hydrophobic core, connecting loops, and disulfides bonds cross between each of the four strands, indicating that, biologically, the V1V2 domain should be considered a single topological entity.

Overall, the 4-stranded V1V2 sheet presents an elegant solution for maintaining a common fold while accommodating V1V2 diversity and glycosylation. Strands contain mostly conserved residues and are welded in place by inter-strand disulfide bonds (between strand A and neighboring strands B and D) and extensive hydrogen bonding (between strands A and D and between strands B and C). The two faces of the sheet—concave and convex—have very different character. The concave face of the sheet is glycan-free and hydrophobic (FIG. 2e), with a cluster of aliphatic and aromatic side chains surrounding the disulfide bond that links strands A and B. This conserved hydrophobic cluster continues onto strand D at the sheet edge, to form a half-exposed hydrophobic core for this domain. The convex face of the sheet is cationic (FIG. 2f) with the main-chain atoms of the conserved strands of the sheet forming stripes on the V1V2 surface (FIG. 2g), and the N-linked glycan 160 situated at its center (FIG. 2h). In contrast, two strand-connecting loops—emanating from the same end of the sheet—are highly glycosylated and variable in sequence (FIG. 2i). Thus, the “V1 loop” can be refined as the residues connecting strands A and B and the “V2 loop” as those residues between strands C and D (FIG. 2h,i). Of these, the V1 loop is most variable, ranging in length from ˜10-30 residues. The V2 loop is less variable and contains at its start the tripeptide motif recognized by integrin α4β7, the gut homing receptor for HIV-1 (Arthos et al., Nat Immunol, 9:301-309, 2008).

### PG9-V1V2 Interactions

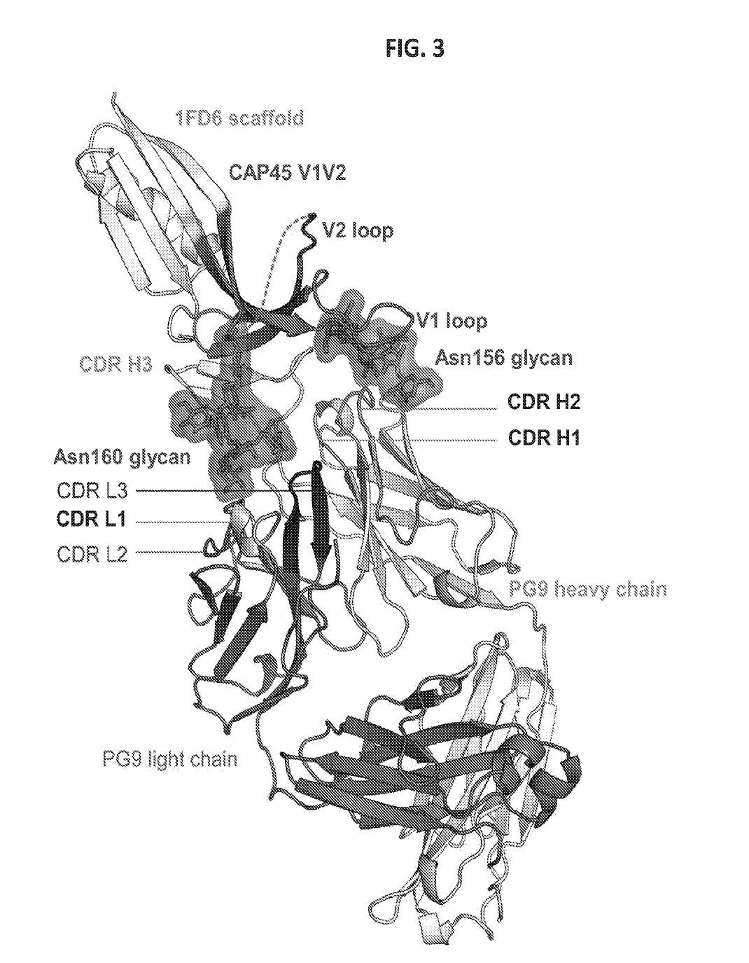

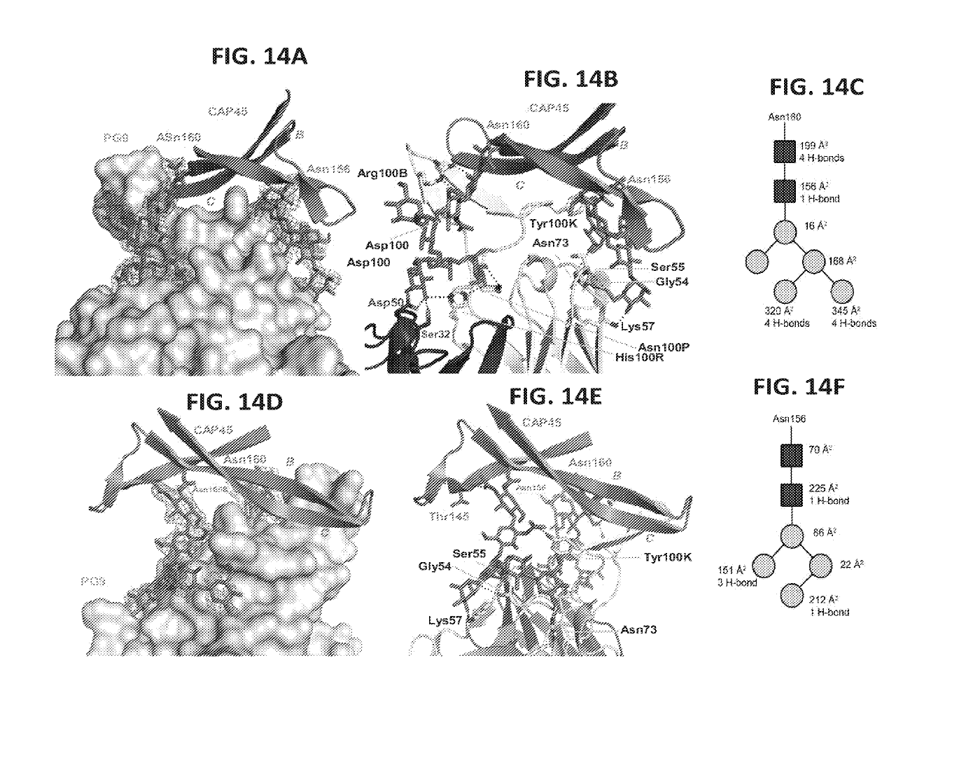

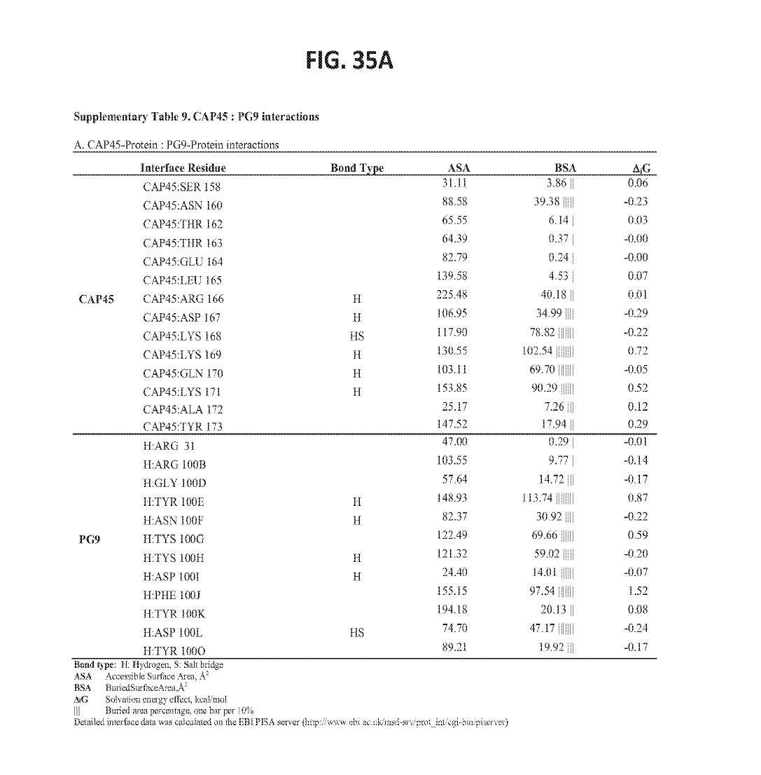

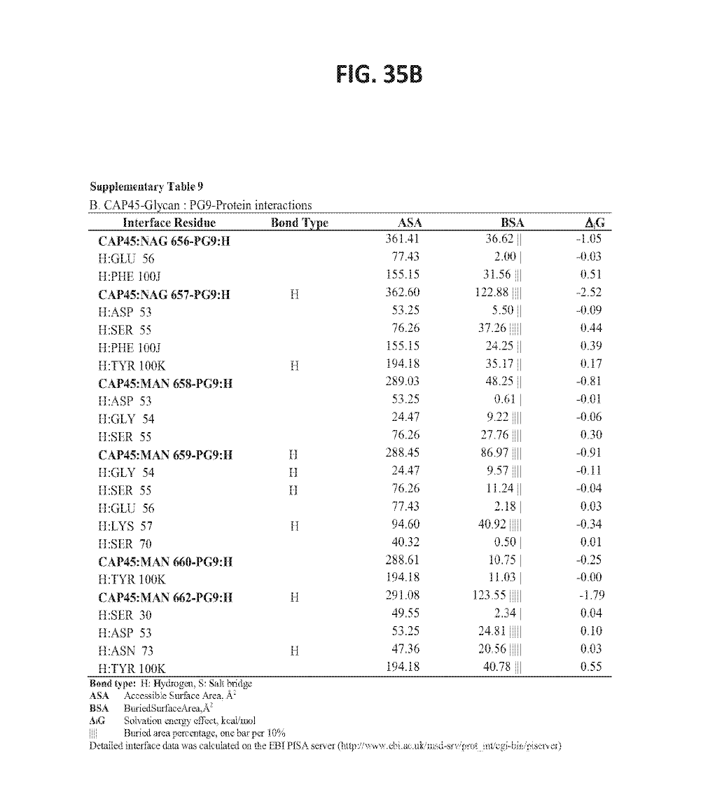

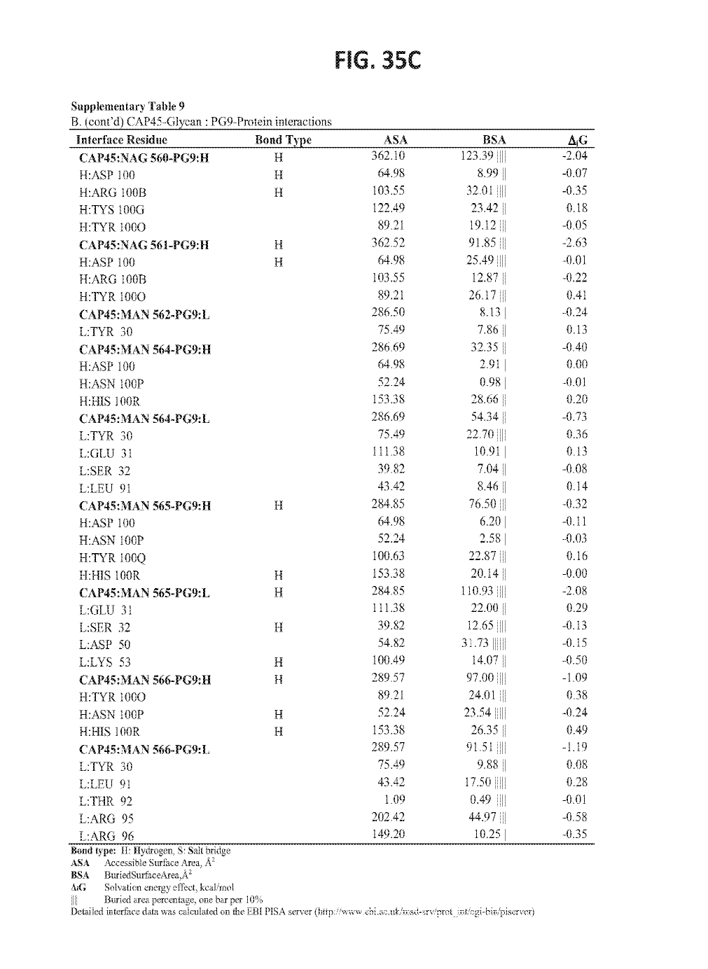

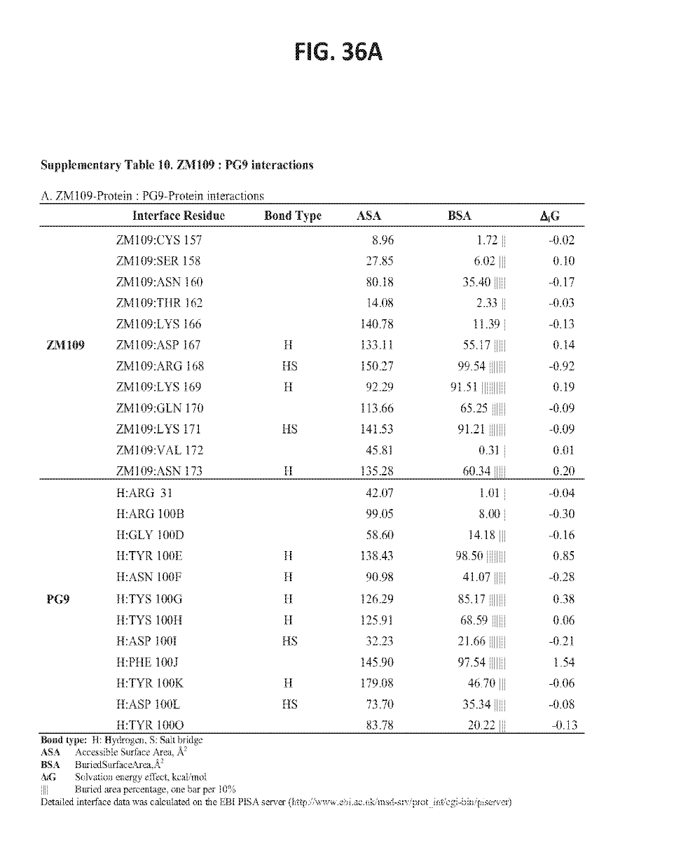

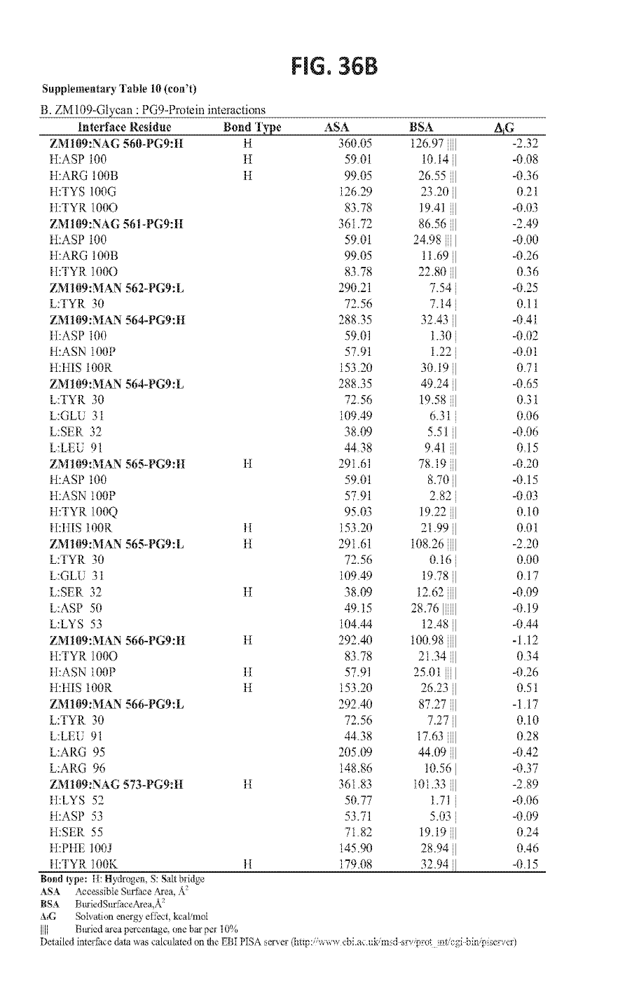

The most prominent interaction between antibody PG9 and V1V2 occurs with N-linked glycan (FIG. 3, FIG. 14, Supplementary Tables 9 and 10 shown in FIG. 35A-36B). PG9 grasps the entire 160 glycan (FIG. 3a). Its protruding third complementarity determining region of the heavy chain (CDR H3) reaches through the glycan shield to contact the protein-proximal N-acetyl glucosamine, burying 200 Å² of total surface area, with Asp100 and Arg100B of PG9 making four hydrogen bonds (FIG. 3b,c) (Kabat numbering is used in description of antibody sequences). Additional hydrogen bonds are made by the base of the CDR H3 (by Asn100P and by the double mannose-interacting His100R) to terminal mannose residues, with Ser32 and Asp50 of the light chain contributing three additional hydrogen bonds (FIG. 3b). In sum, a total of 11 hydrogen bonds and over 1150 Å² of surface area are buried in the PG9-glycan 160 interface (489 Å² on PG9 and 670 Å² on glycan 160), with PG9 contacting 5 of the 7 saccharide moieties of the Man₅GlcNAc₂ glycan (FIG. 3c). Similar extensive interactions are observed with residue 160 of CAP45 (FIG. 14a-c). The preference of PG9 for a Man₅GlcNAc₂ glycan at residue 160 is now clear: a larger glycan would clash with the antibody light chain and a shorter glycan would not stretch between tip and base of the PG9 CDR H3.

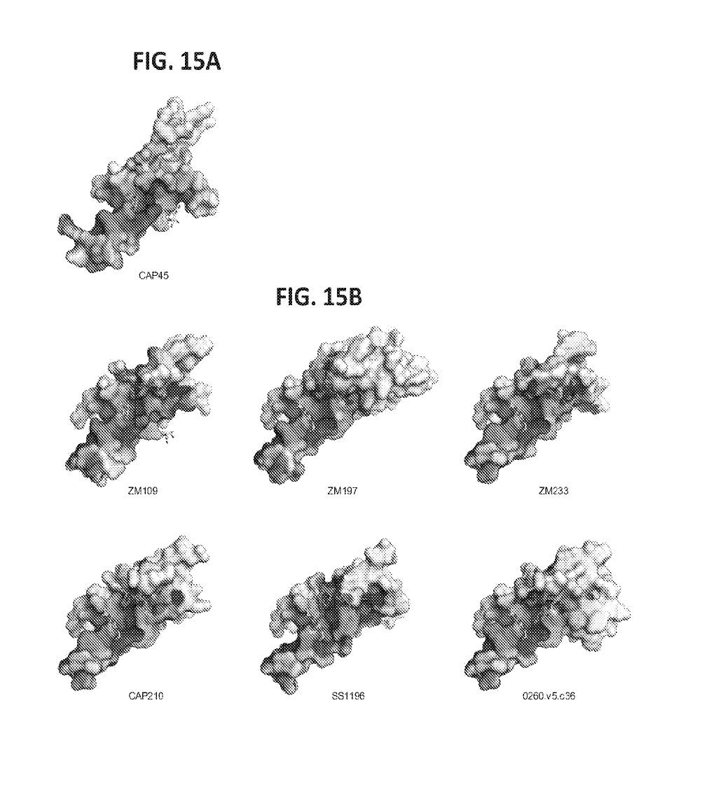

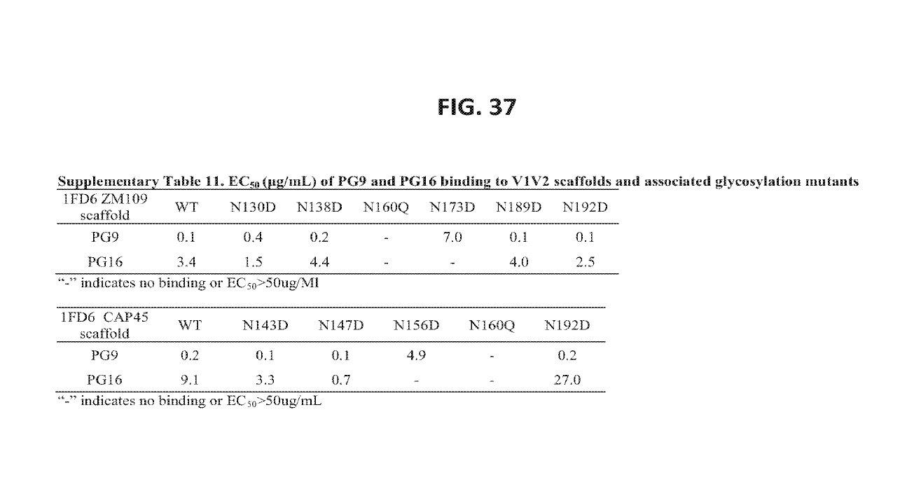

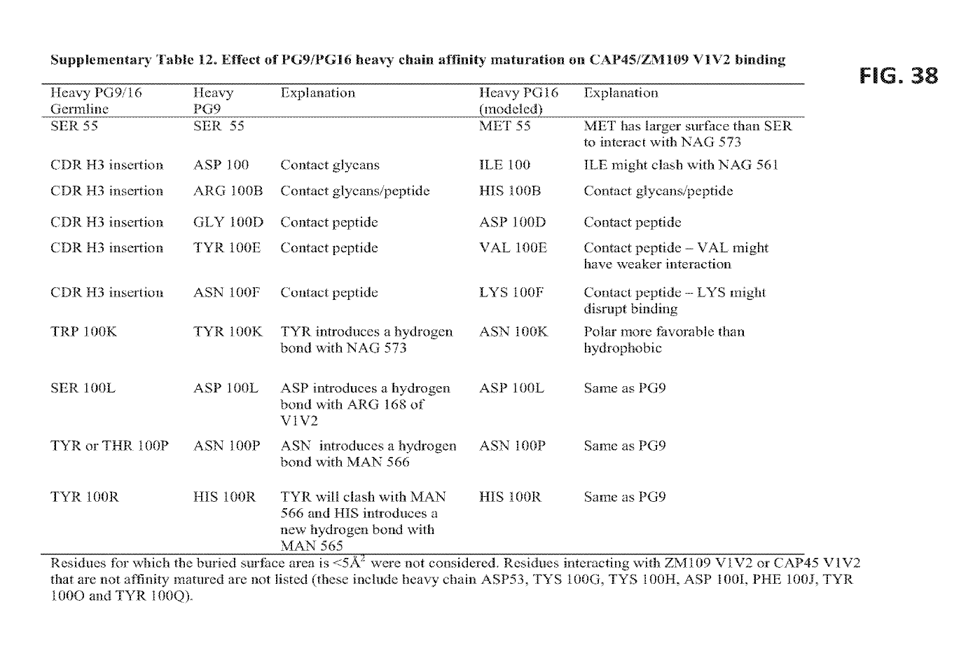

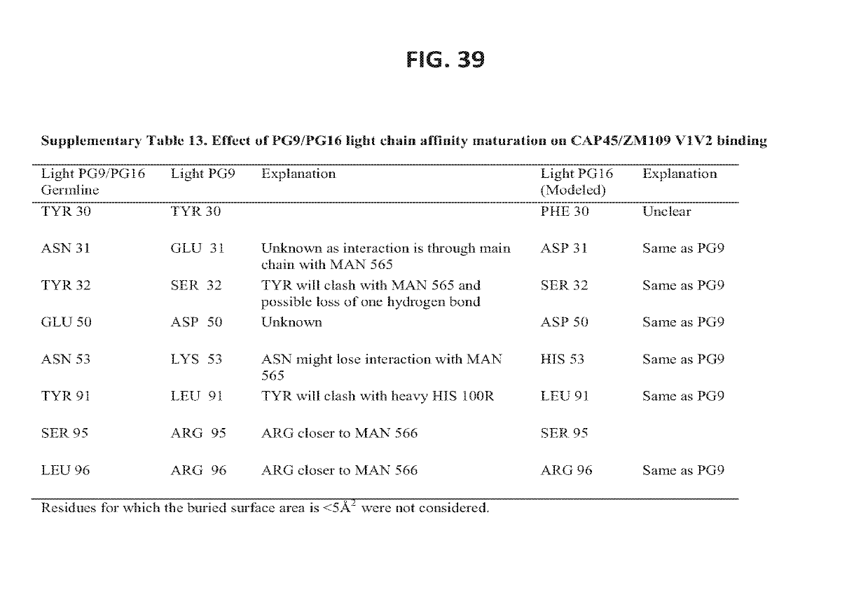

Interactions also occur between PG9 and the N-linked glycan at residue 156 (CAP45) or residue 173 (ZM109). With CAP45, much of the 156 glycan is ordered, stabilizing six of the seven sugars, including four of the five mannose residues (FIG. 14). Hydrogen bonds are observed between the 156 glycan and the side chains of Asn73 and Tyr100K of the PG9 heavy chain, and 766 Å² of total buried surface area (337 Å² on PG9 and 429 Å² of glycan). Glycan 156 is not preserved in the ZM109 sequence, where residue 156 is a histidine (FIG. 2i); an additional site of N-linked glycosylation, however, occurs in ZM109 at residue 173, in the middle of strand C. In the ZM109 structure, glycan 173 is in virtually the same spatial location as glycan 156 in the CAP45 structure (FIG. 2h). PG9 binds to the protein-proximal N-acetylglucosamine, with Tyr100K making a hydrogen bond and a total of 189 Å² surface area buried (FIG. 3b). Notably, mutational alteration of V1V2 glycans indicate that glycan at 160 is critical for PG9 recognition (Supplementary Table 11 shown in FIG. 37), and 156/173 is important (although PG9 recognizes strains of HIV-1 lacking a 156/173 glycan; FIG. 15). Many of the changes in the heavy and light chains that allow for glycan recognition occur during affinity maturation (Supplementary Tables 12 and 13 shown in FIG. 38 and FIG. 39, respectively), providing a possible explanation for the observed increase in PG9 (and PG16) breadth and affinity during affinity maturation (Pancera et al., J. Virol., 84:8098-8110, 2010).

In addition to glycan recognition, a strand in the CDR H3 of PG9 forms intermolecular parallel β-sheet-like hydrogen bonds to strand C of V1V2 (FIG. 3d, e). Strand C is the most variable of the V1V2 strands, and this sequence-independent means of recognition likely allows for increased recognition breadth. Specific electrostatic interactions are also made between cationic residues of strand C and acidic residues on PG9. Notably, several of these occur with sulfated tyrosines on CDR H3. Because parallel β-strand-hydrogen bonding would tend to align main-chain atoms of CDR H3 and strand C, the charged tips of Lys and Arg residues would protrude beyond the standard acidic Asp and Glu side chains, whereas tyrosine sulfates provide a closer match to the side-chain length of basic Lys/Arg residues.

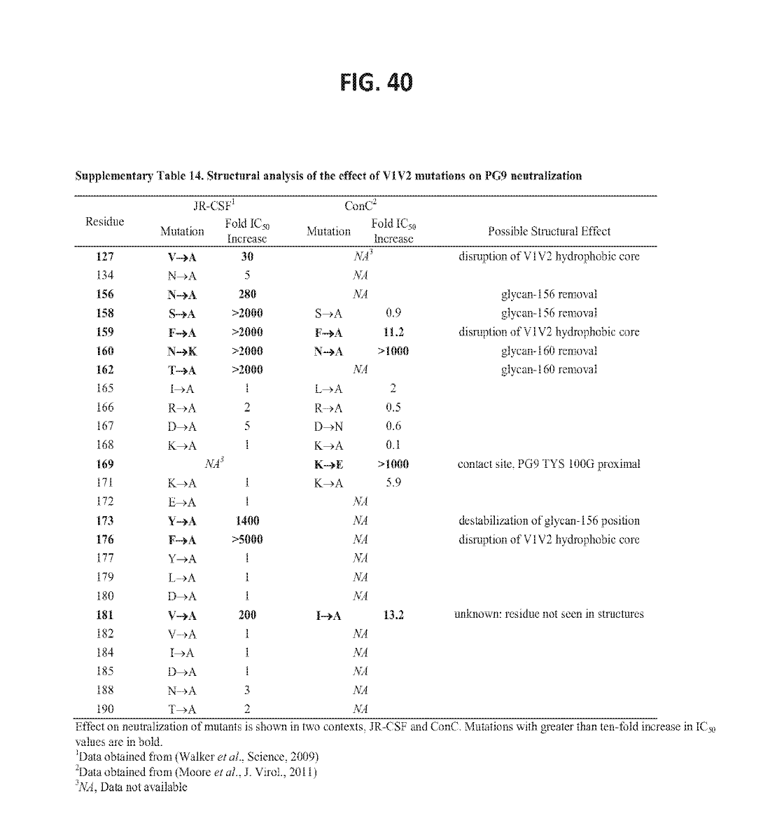

Overall, the structure of PG9 is consistent with published mutational data (Walker et al., Science, 326:285-289, 2009 and Moore et al., J Virol, 85: 3128-3141, 2011) (Supplementary Table 14, shown in FIG. 40). Some residues such as Phe176 are critical because they form part of the hydrophobic core on the concave face of the V1V2 sheet. Others form direct contacts: for example, the tyrosine sulfate at residue 100H of PG9 interacts with residue 168 when it is an Arg (strain ZM109) or Lys (strain CAP45), but would be repelled by a Glu (as in strain JR-FL); JR-FL is resistant to neutralization by PG9, but becomes sensitive if Glu168 is changed to Lys (Walker et al., Science, 326:285-289, 2009).

### Quaternary Preferences of PG9 and PG16

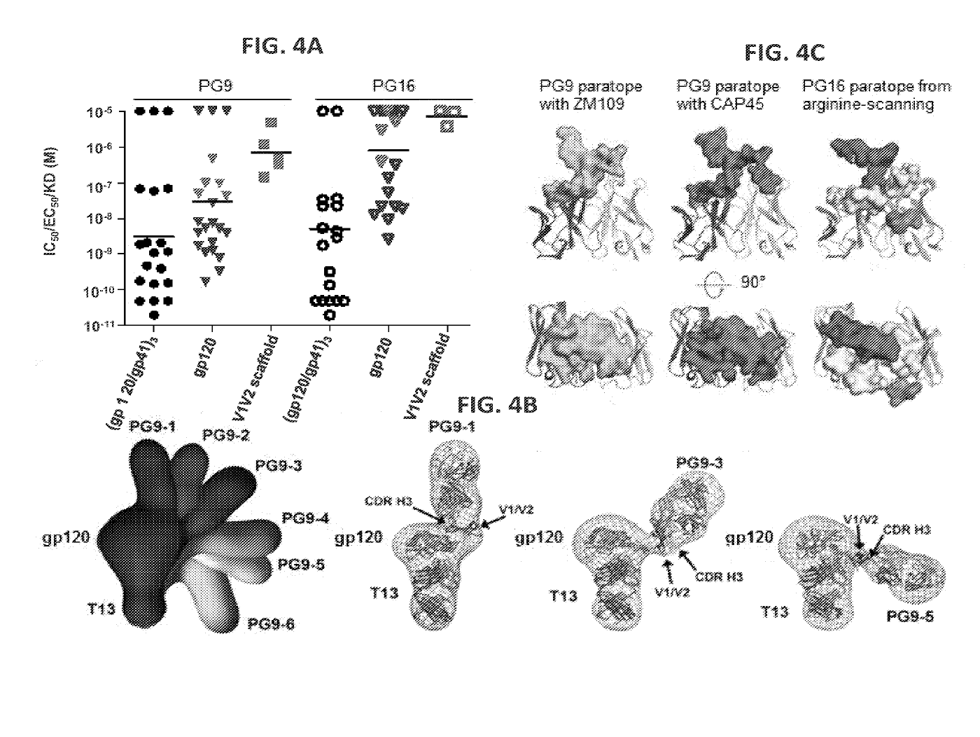

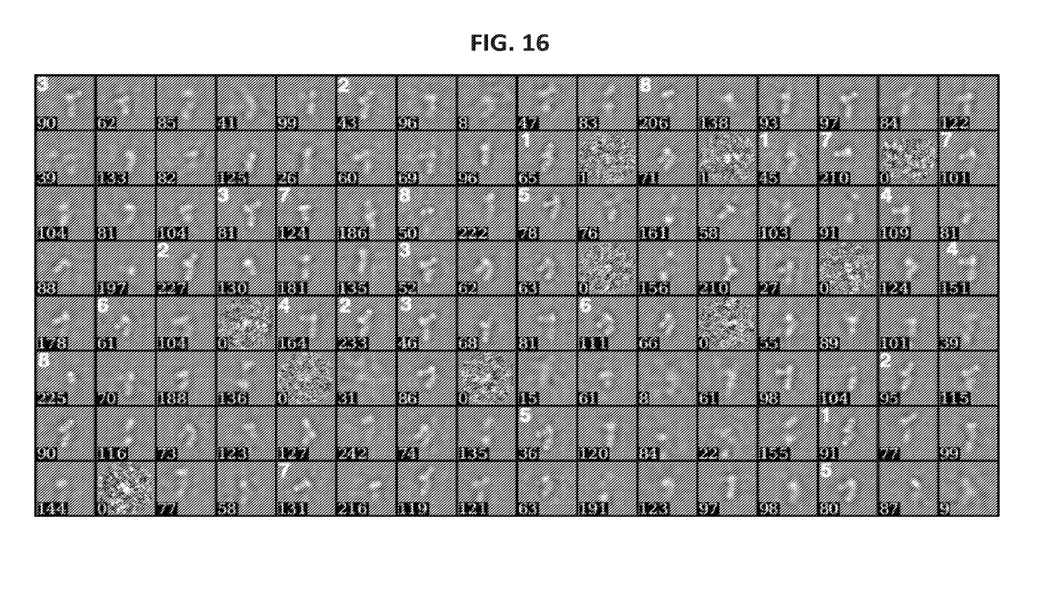

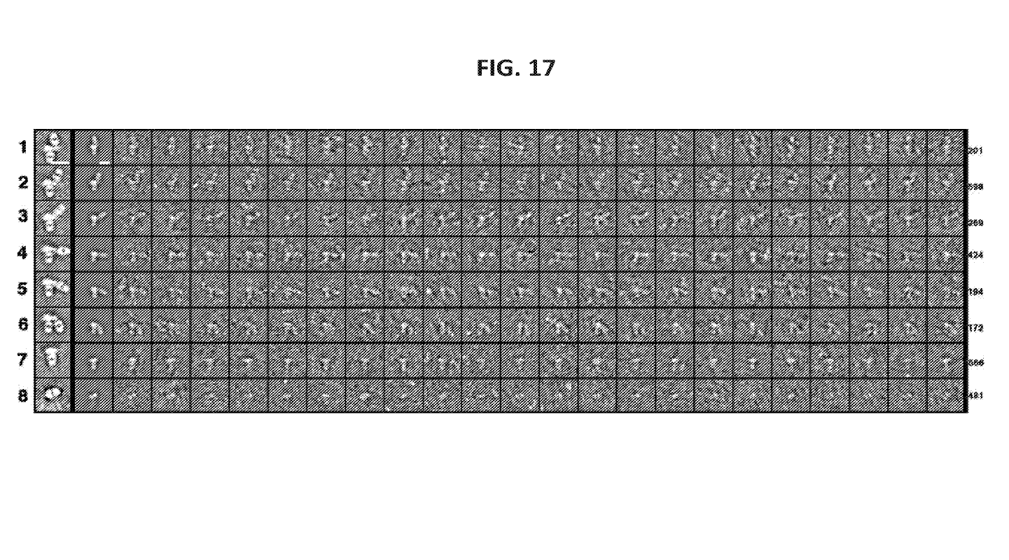

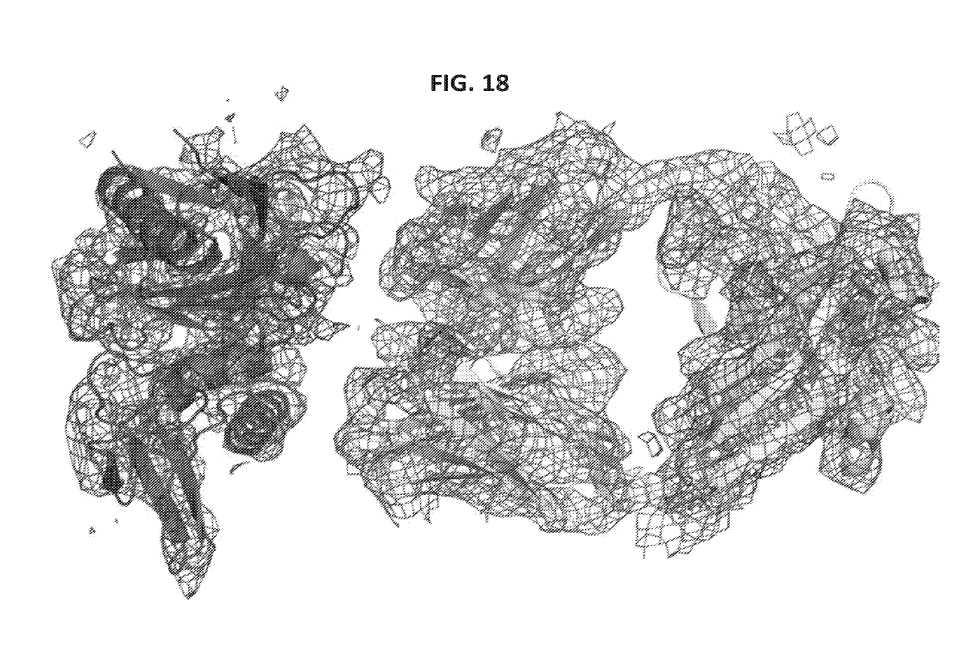

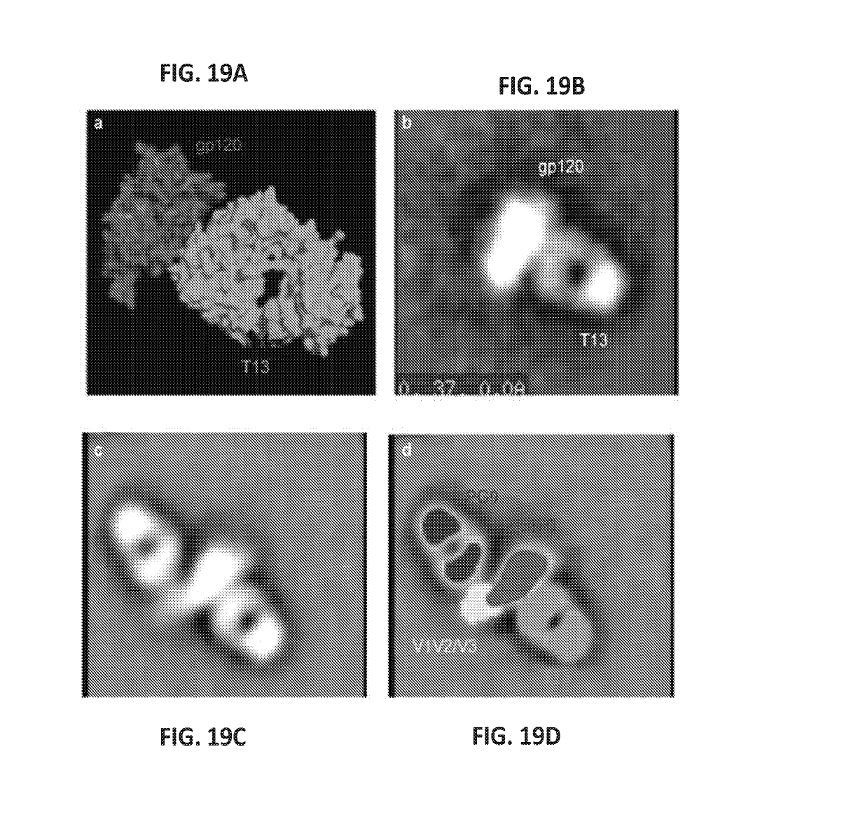

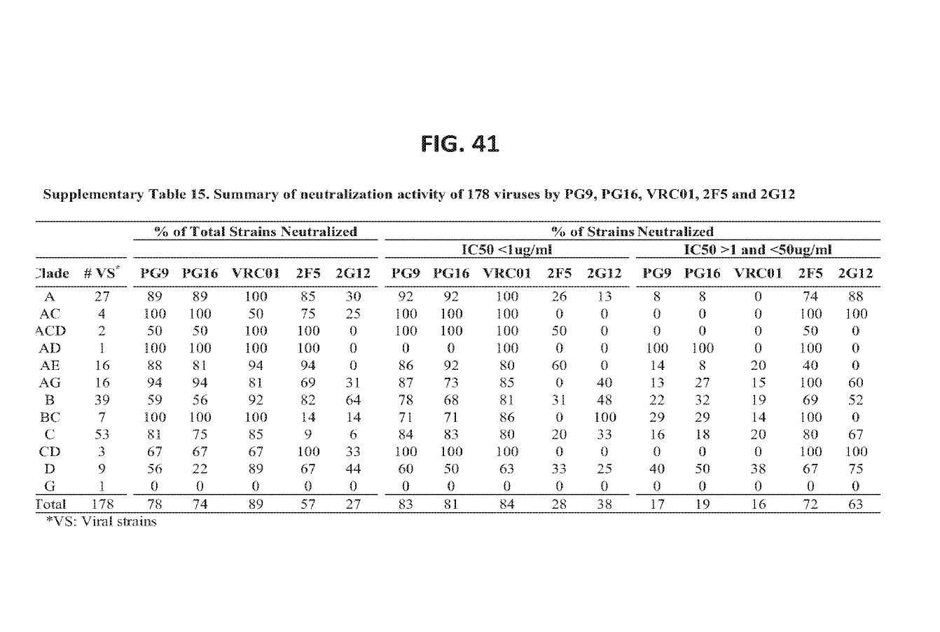

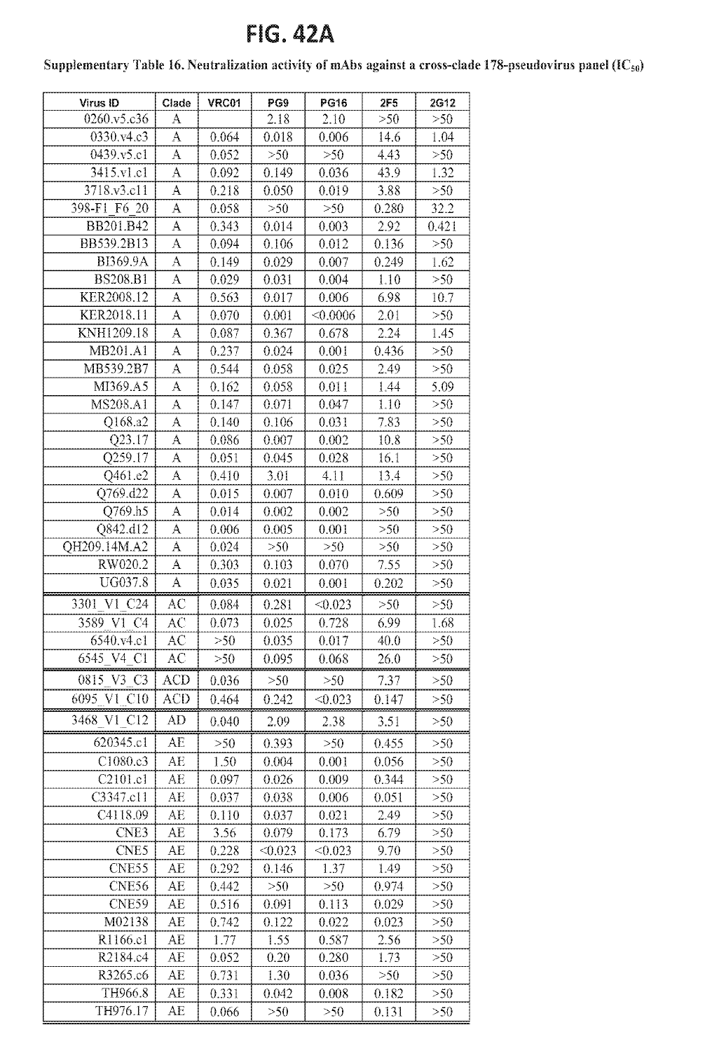

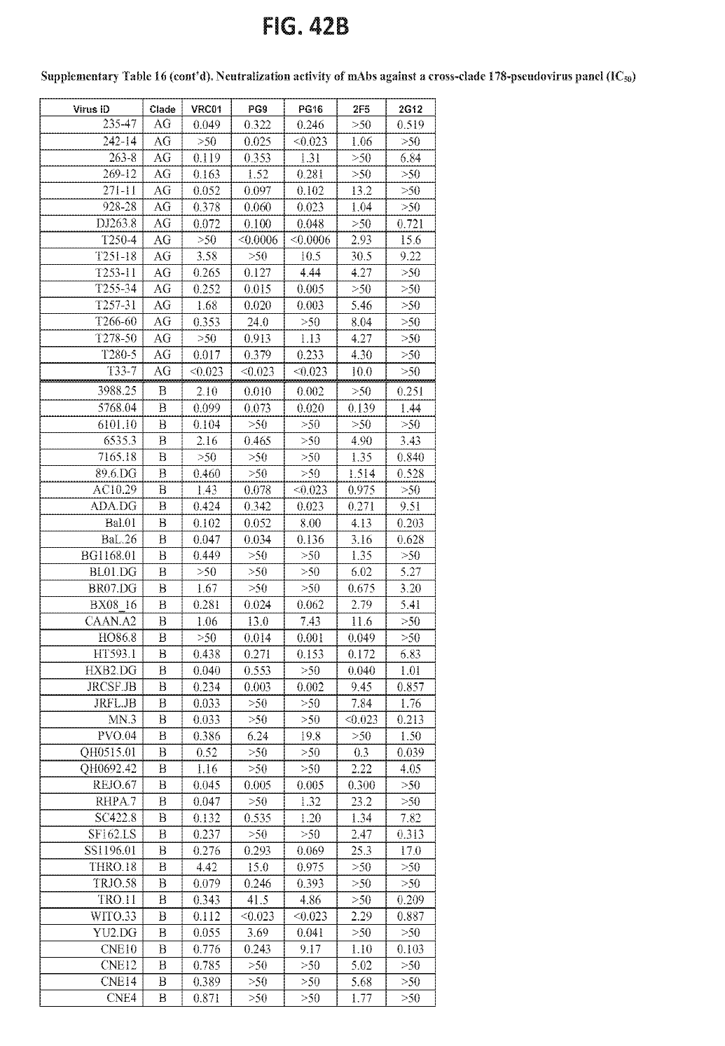

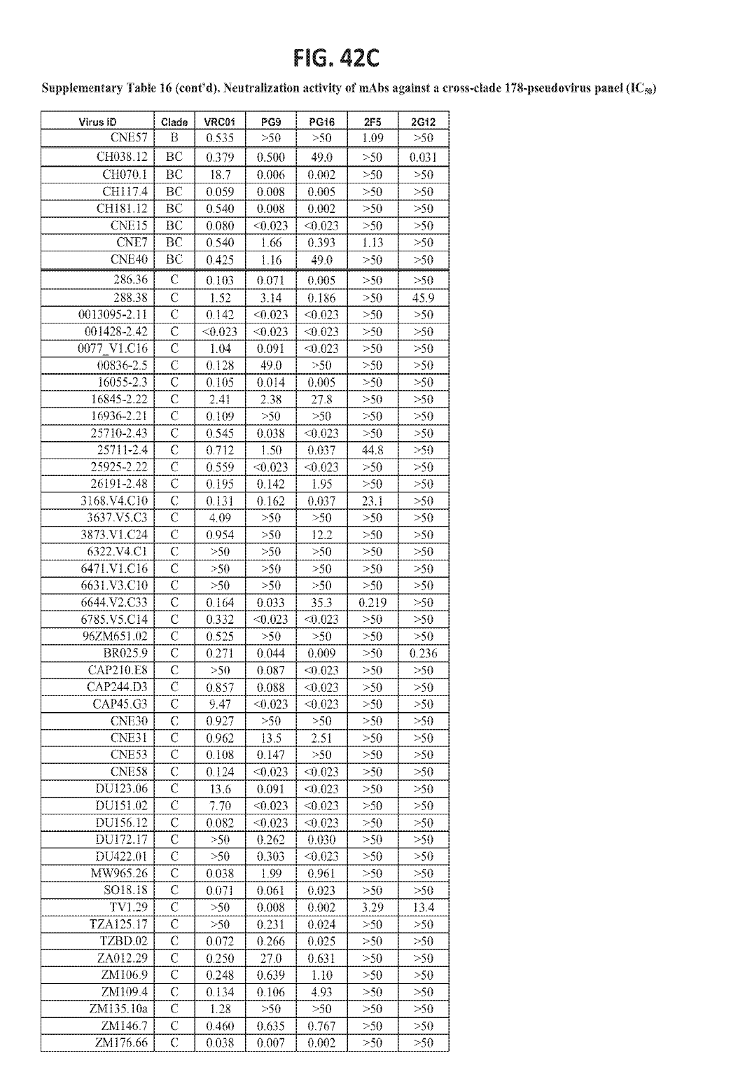

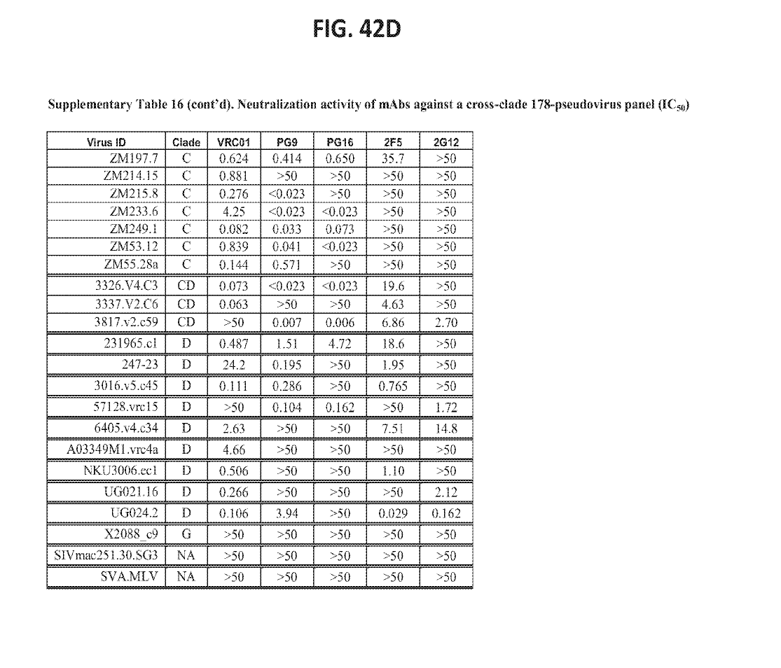

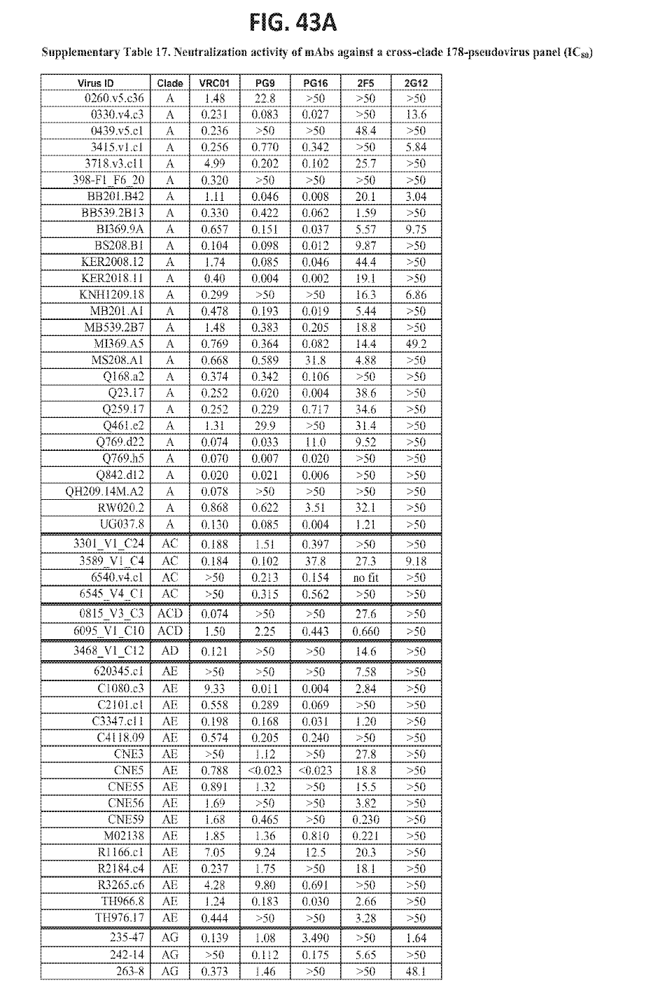

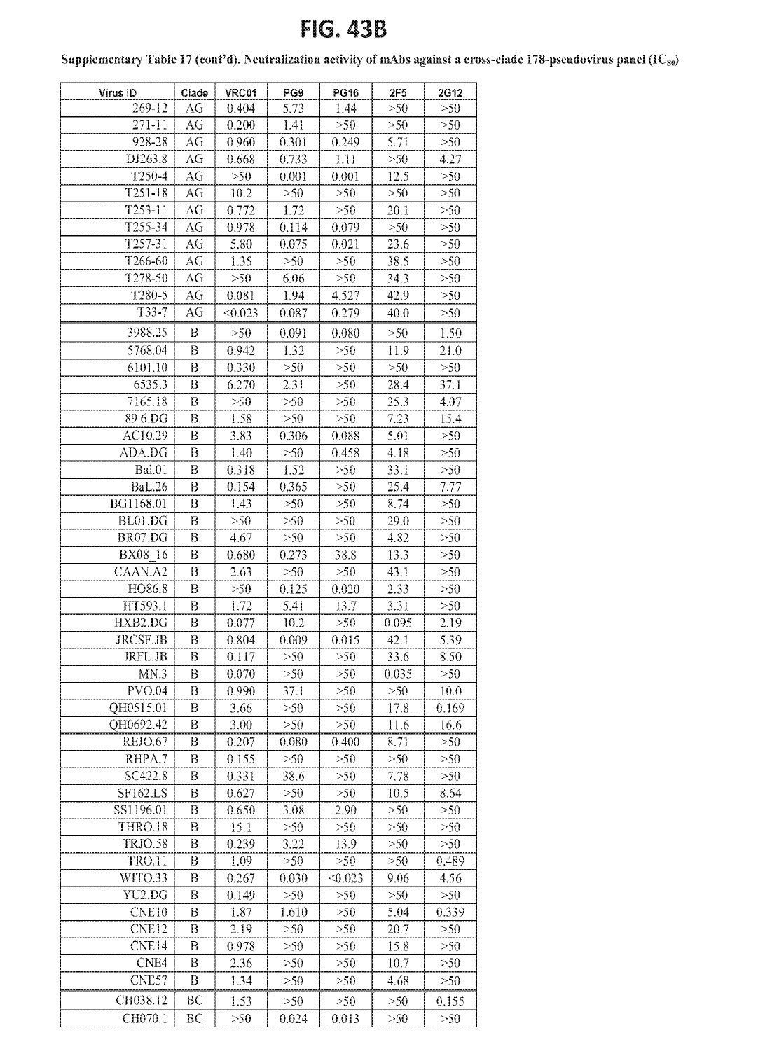

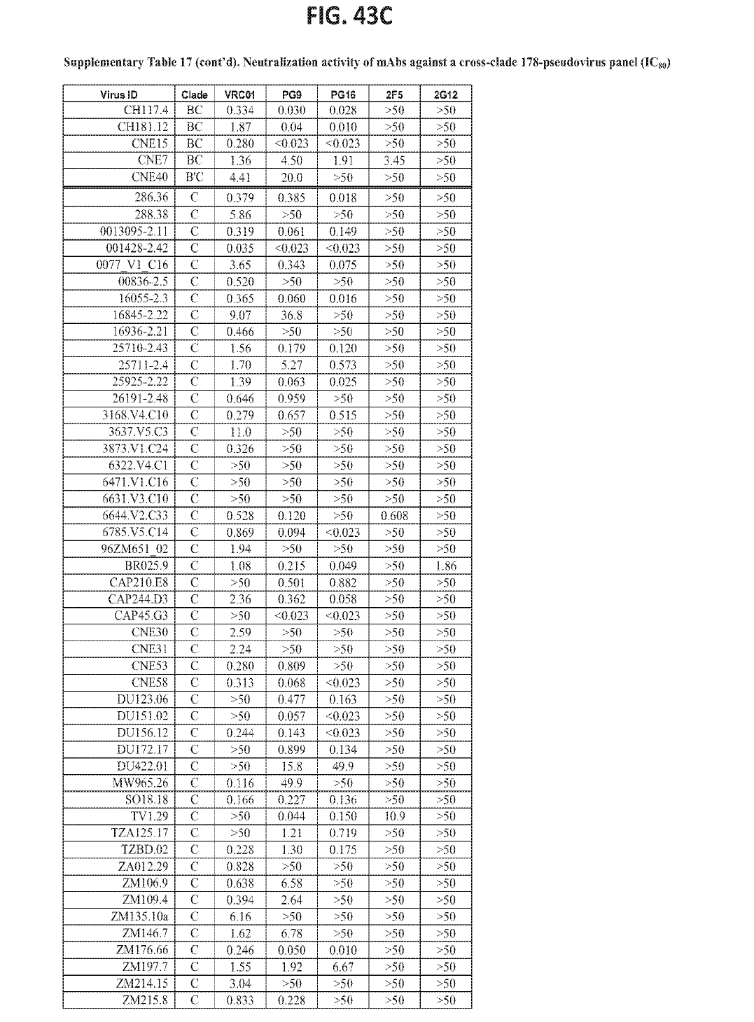

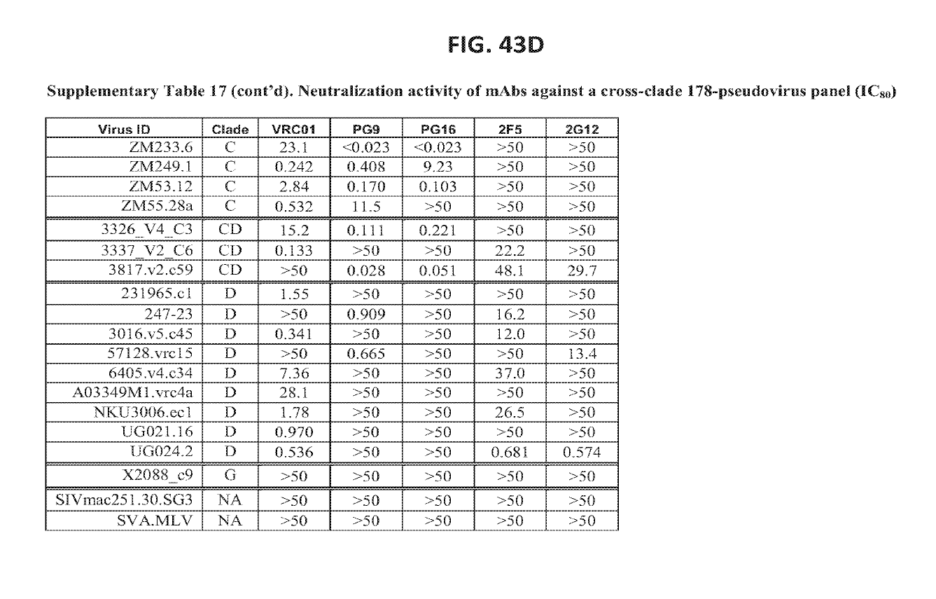

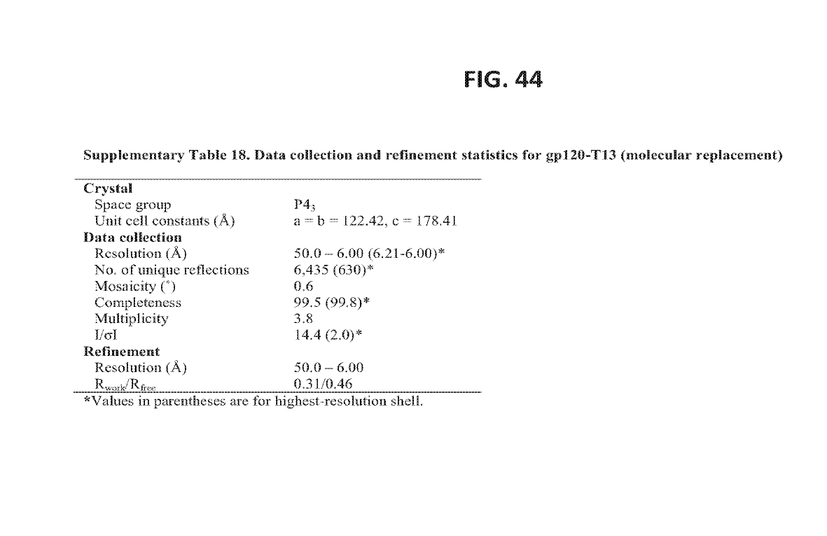

PG9 and the somatically related PG16 recognize the assembled viral spike with higher affinity than monomeric gp120 (Walker et al., Science, 326:285-289, 2009). For PG9, the average monomeric gp120 affinity, as assessed by ELISA or surface plasmon resonance, was at least 10-fold weaker than viral spike affinity, as assessed by neutralization; with PG16, the difference was at least 100-fold (FIG. 4a, Supplementary Tables 6 and 15-17, shown in FIGS. 32 and 41A-43D). Such differences are likely greater as the concentration required for neutralization (IC₅₀) is often higher than the affinity (EC₅₀ or K_{D}). To investigate differences between monomeric and oligomeric contexts, negatively stained-electron microscopy images of PG9 in complex with monomeric gp120 were acquired (FIG. 4b, FIGS. 16 and 17). To define the orientation of monomeric gp120, the CD4-binding-site directed antibody T13 was used, for which the crystal structure of gp120-bound T13 Fab was defined at 6 Å resolution (FIGS. 18 and 19, Supplementary Table 18 shown in FIG. 44). This structure along with the V1V2-PG9 structure allowed for the definition of 6 classes of relative gp120-PG9 orientations, indicating that the position of V1V2 varies in the monomeric gp120 context. In contrast, prior EM results indicate the position of V1V2 in the unliganded Env trimer spike is fixed (Liu et al., Nature, 455:109-113, 2008; Wu et al., Proc Natl Acad Sci USA, 107:18844-18849, 2010; White et al., PLoS Pathog, 6:e1001249, 2010; Hu et al., J Virol, 85:2741-2750, 2011).

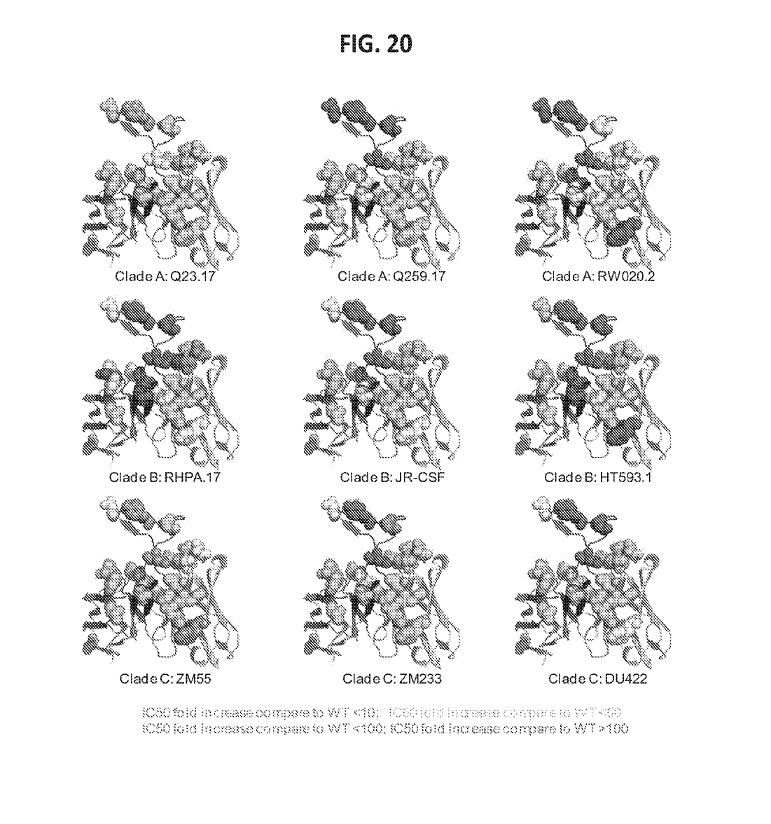

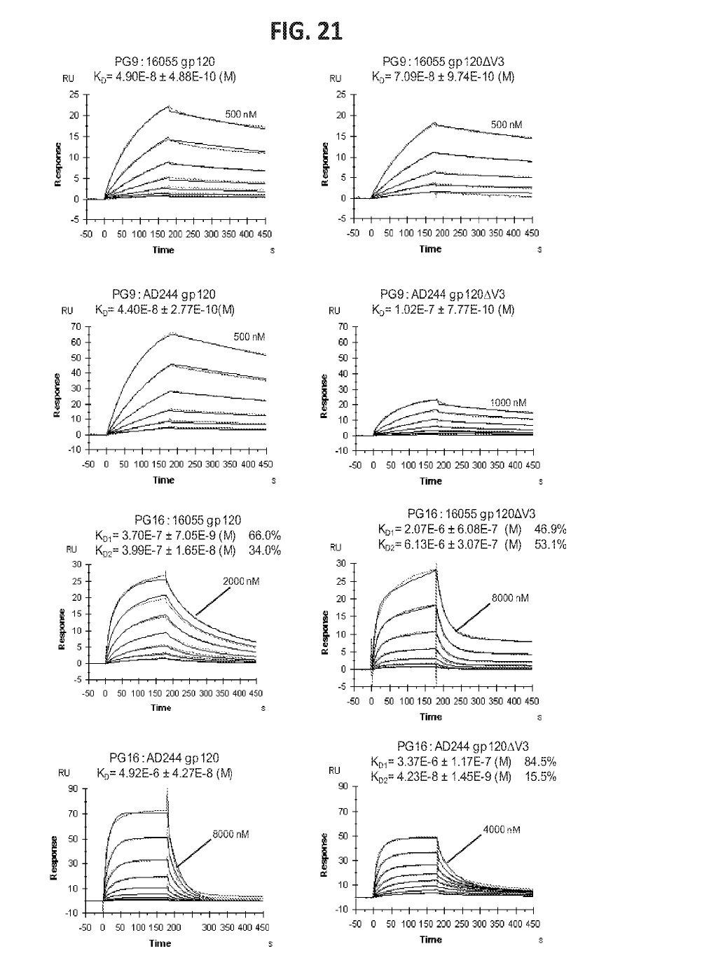

Additionally, the antibody paratope was mapped by assessing neutralization with arginine mutants. The PG16 paratope was selected for characterization, as its recognition appeared to be both more quaternary-structure-preferring (FIG. 4a) and more V3-dependent (Walker et al., Science, 326:285-289, 2009) than that of PG9. The combining site was parsed into 21 surface segments plus 1 in the framework as a control. Each of these was altered by the introduction of a single arginine mutation, expressed as an immunoglobulin, and assessed for neutralization on a panel of diverse HIV-1 isolates (FIG. 20). The resultant “arginine-scanning”-mutagenesis revealed a close match to the observed V1V2 interface for PG9 (FIG. 4c). The binding of PG9 and PG16 to monomeric gp120 in wild-type and V3-deleted contexts was measured, and similar affinities observed, indicating that—in the context of monomeric gp120-V3 does not play a substantial role in PG9 or PG16 recognition (FIG. 21). Lastly, accumulating data suggest that V1V2 in the viral spike both shields and interacts with V3 (Cao et al., J Virol, 71:9808-9812, 1997; Stamatatos et al., J Virol, 72:7840-7845, 1998; Pinter et al., J Virol, 78:5205-5215, 2004; Rusert et al., J Exp Med, 208:1419-1433, 2011).

Collectively, these results suggest that the V1V2-PG9 interaction observed in the scaffolded-V1V2-PG9 crystal structures encompasses much of the PG9/PG16 epitope, and that the structural integrity of this epitope is sensitive to appropriate assembly of the viral spike. The ability of the PG9/PG16-recognized epitope to be preferentially present in the assembled viral spike provides a useful strategy to hide this potential site of vulnerability. That is, the site may be preferentially present on the assembled viral spike, but not on shed or other monomeric forms of gp120, which are thought to be the predominant form of Env in infected individuals; in this regard that many V1V2-directed antibodies are substantially more quaternary-structure-preferring than PG9. The quaternary-specific nature of the epitope may thus reflect a functional adaptation of HIV-1.

### Conserved Structural Motif for V1V2-Directed Broadly Neutralizing Antibodies

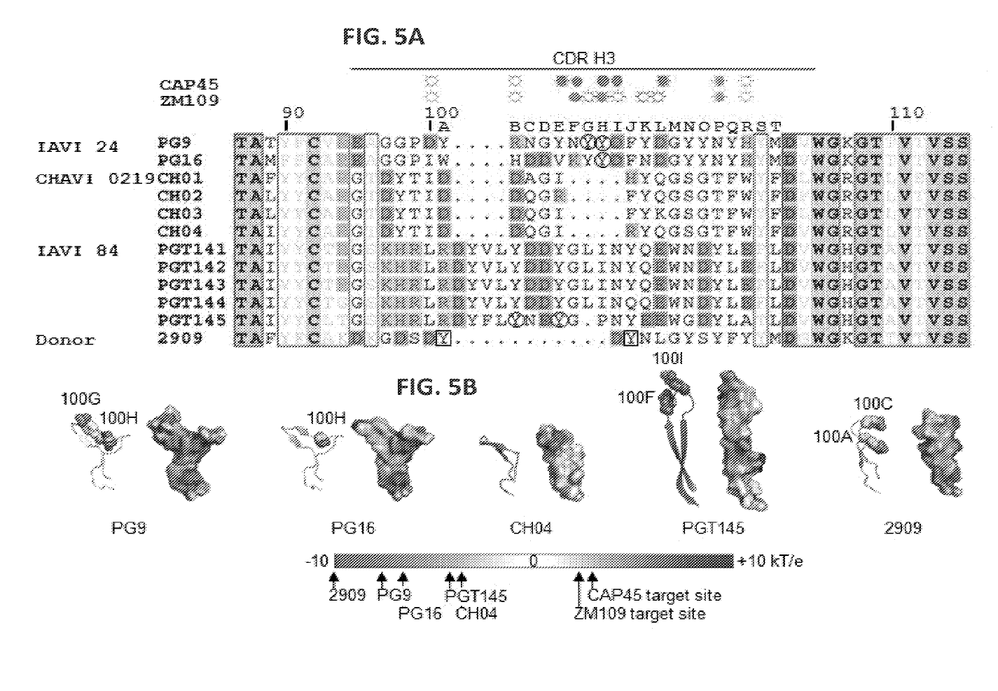

Sequences of other V1V2-directed broadly neutralizing antibodies indicate the presence of long CDR H3s, but little other sequence conservation (FIG. 5a). The structures of other class members in complex with V1V2 have not yet been determined, but nonetheless sought to provide insight into their conserved features of recognition by analyzing unbound Fab structures.

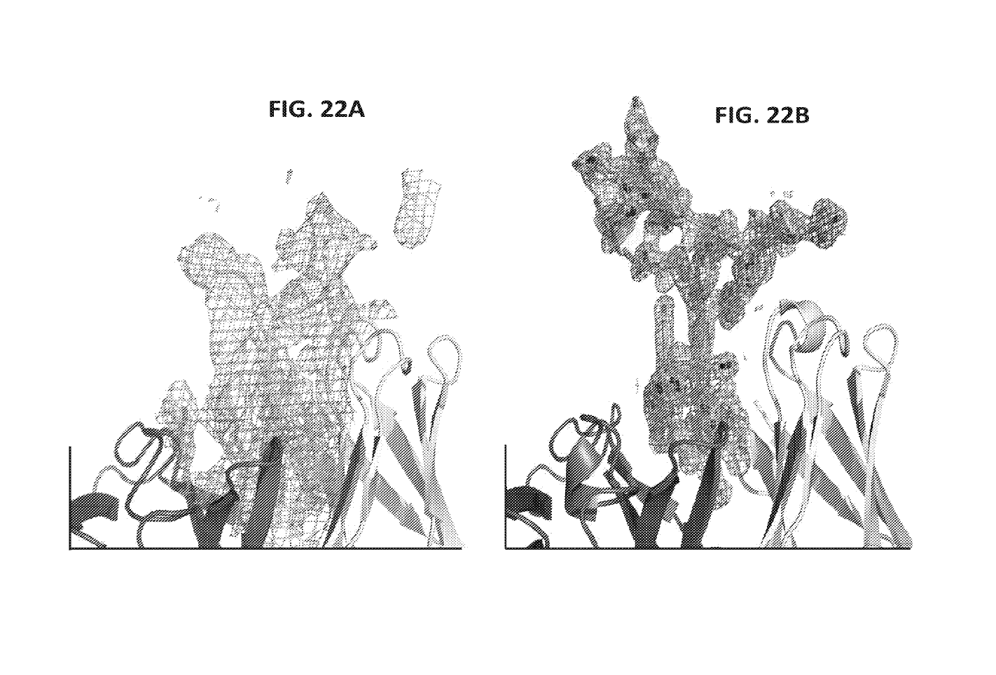

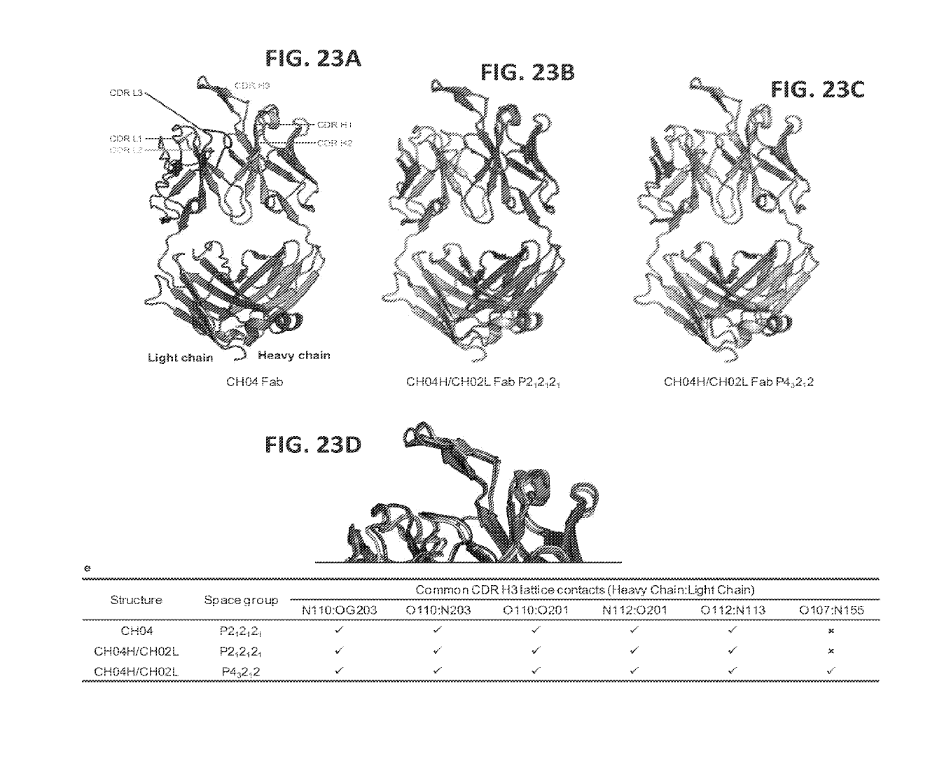

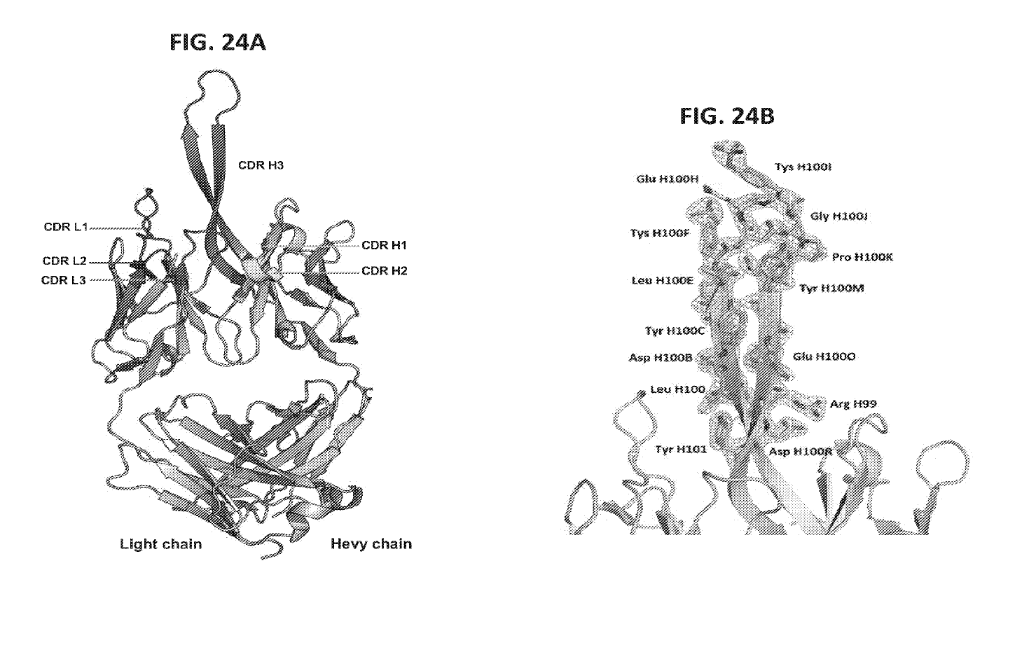

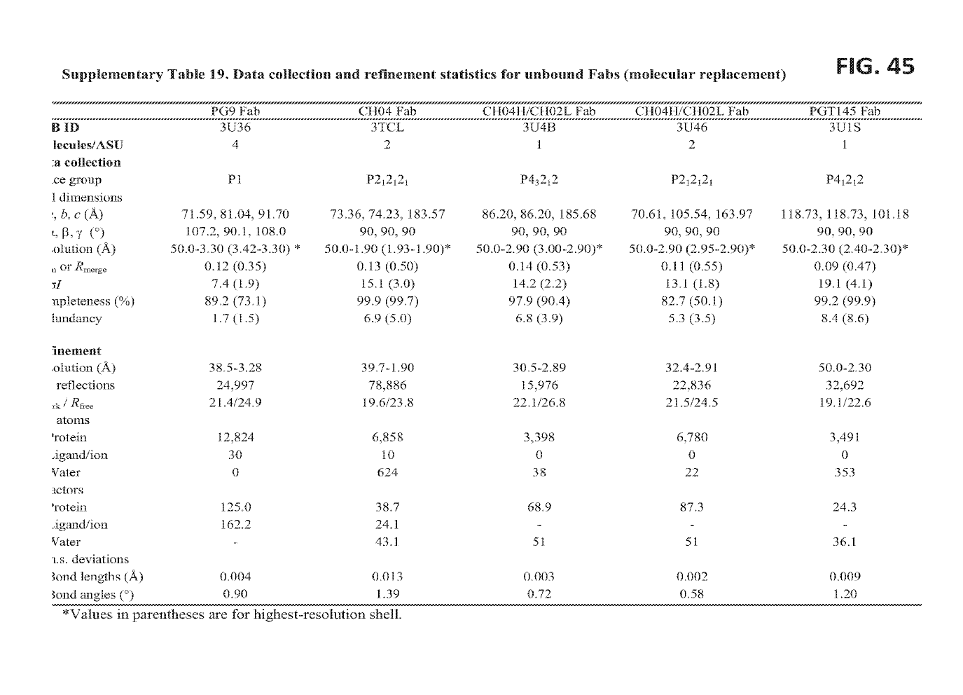

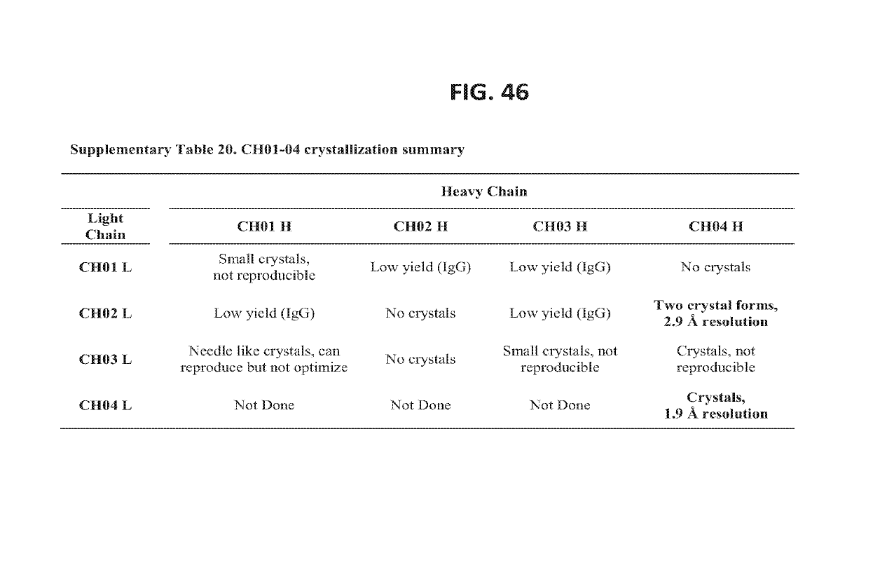

The structure of unbound PG9 Fab (3.3 Å resolution, 4 molecules/asymmetric unit, FIG. 22 and Supplementary Table 19 shown in FIG. 45) revealed significant CDR H3 flexibility, similar to that observed previously with PG16 (Pancera et al., J. Virol., 84:8098-8110, 2010). For CH01-CH04 antibodies (Bonsignori et al., J Virol, 85:9998-10009, 2011), crystallization was attempted for Fabs and for six heavy/light-chain somatic chimeras (Supplementary Table 20 shown in FIG. 46). Structures were determined for CH04 and also for the CH04H/CH02L, the latter in two different crystal forms (FIG. 23 and Supplementary Table 19 shown in FIG. 45). These structures revealed an anionic CDR H3 for CH04, which extended above the rest of the combining site in a manner similar to the CDR H3s of PG9 and PG16 (FIG. 5b). With CH04, however, the extended hairpin was twisted ˜90°, to an orientation that bisected heavy and light chains. The spacing between the protruding CDR H3 and the rest of the combining region was reduced by 8 Å relative to that of PG9, and no CDR H3 tyrosine sulfation was observed.

With PGT141-145 antibodies (Walker et al., Nature, 477:466-470, 2011), crystals of unbound PGT145 diffracted to 2.3 Å and revealed an extended, tyrosine sulfated, CDR H3 loop, which like those of PG9, PG16 and CH04 reached substantially beyond the rest of the CDR loops. In contrast, the β-hairpin of CDR H3 extended vertically (parallel to the long axis of the Fab) (FIG. 5b, FIG. 24 and Supplementary Table 19 shown in FIG. 45) and was rigidified by extensive tyrosine stacking (along with the standard strand-strand hydrogen bonding). Its negatively charged tip (including two sulfated tyrosines) was followed by a Gly-containing potential “hinge” and resembled an extended version of the CDR H3 of antibody 2909 (Changela et al., J. Virol, 85:2524-2535, 2011 and Spurrier et al., Structure, 19:691-699, 2011), a highly quaternary-structure-sensitive antibody (Gorny et al., J Virol, 79:5232-5237, 2005 and Honnen et al., J Virol, 81:1424-1432, 2007), which recognizes an immunotype variant of the V1V2 target site in which a Lys is substituted for the N-linked glycan at position 160 (Wu et al., J Virol, 85:4578-7585, 2011).

Thus, despite having been derived from three different individuals, antibodies of this class of V1V2-directed broadly neutralizing antibodies all displayed anionic protruding CDR H3s (FIG. 5b), most of which were tyrosine sulfated. All also displayed β-hairpins, and although these varied substantially in orientation relative to the rest of the combining site, all appeared capable of penetrating an N-linked glycan shield to reach a cationic protein surface.

### A V1V2 Site of HIV-1 Vulnerability

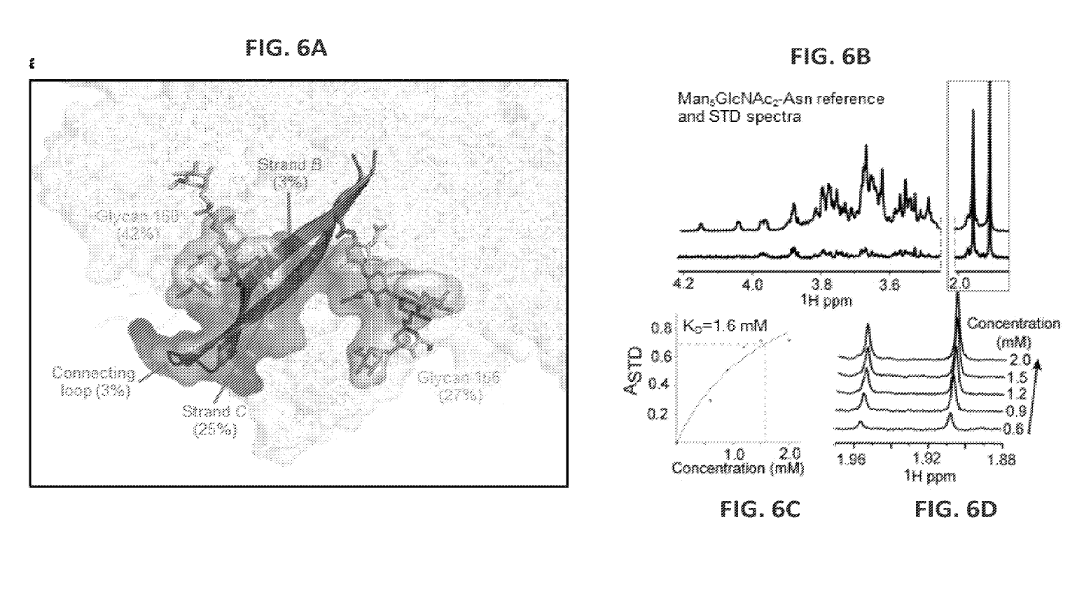

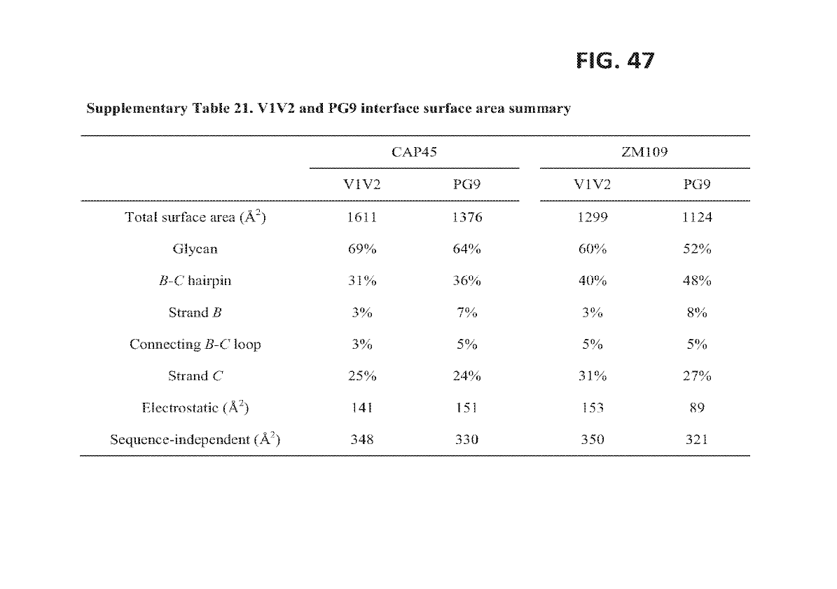

With both CAP45 and ZM109 strains of gp120, the V1V2 site recognized by PG9 consists primarily of two glycans and a strand (FIG. 6a). Minor interaction with strand B and the B-C connecting loop (3% and 3-5% of the total interactive surface, respectively) complete the epitope, with the entire PG9-recognized surface of V1V2 contained within the B-C hairpin (Supplementary Table 21 shown in FIG. 47). The minimal nature of this epitope suggests that it might be easier to engineer and to present to the immune system than other, more complex, epitopes. The epitopes for antibodies b12 and VRC01, for example, comprise seven- and six-independent protein segments, respectively. The presence of N-linked glycosylation in the PG9 epitope, which is added by host cell machinery, does provides a potentially complicating factor to humoral recognition.

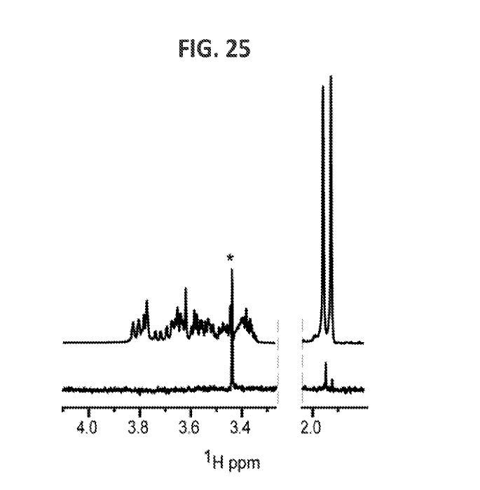

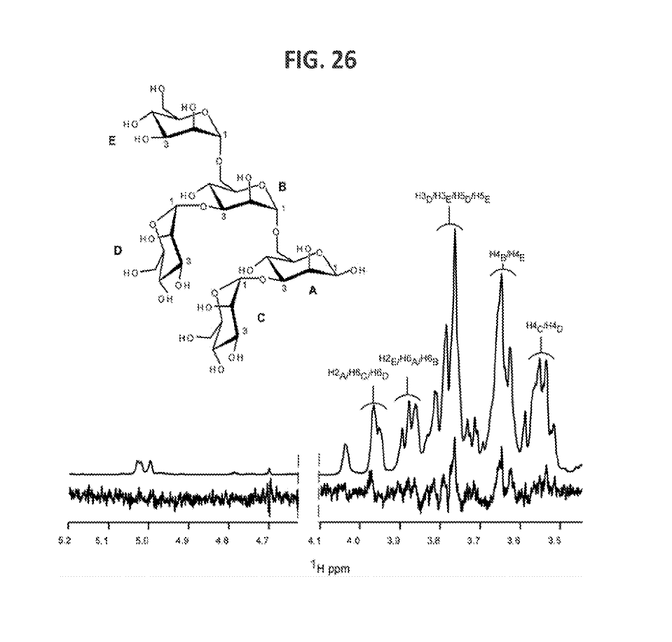

To assess glycan affinities, saturation transfer difference NMR was used. Recognition by PG9 occurs with protein-proximal N-acetylglucosamines and terminal mannose saccharides. With 1.5 mM (N-acetylglucosamine)₂, interaction with PG9 was not observed (FIG. 25), whereas with 1.5 mM oligomannose-5, weak interactions were observed (FIG. 26). A titration series with Asn-(N-actylglucosamine)₂(mannose)₅ was conducted and determined its affinity for PG9 to be 1.6±0.9 mM (FIG. 6b). The weak affinity for glycan (surprising in the face of such large contact surface and hydrogen bonds) provides a potential explanation for the reported lack of PG9 auto-reactivity despite its N-glycan-dependence (Walker et al., Science, 326:285-289, 2009) (specificity for oligomannose-5 likely also reduces PG9 auto-reactivity, as this glycan is infrequently displayed on the surface of mammalian cells).

Strand C is the most cationic of the V1V2 strands. This conserved cationic character—present in the target cell-facing V1V2 cap of the viral spike—may relate to the observed anionic interactions of the viral spike, both with dextran sulfate (Mitsuya et al., Science, 240:646-649, 1988 and Schols et al., Virology, 175:556-561, 1990) and other polyanions (Moulard et al., J Virol, 74:1948-1960, 2000 and Fletcher et al., Retrovirology, 3:46, 2006) or with heparan sulfate on the cell surface (Mondor et al., J Virol, 72:3623-3634, 1998). In terms of the ionic interactions of PG9 itself, sulfation to increase affinity and neutralization potency by ˜10-fold was observed (Walker et al., Nature, 477:466-470, 2011 and Pejchal et al., Proc Natl Acad Sci USA, 107:11483-11488, 2010) (FIG. 11). Ionic PG9 interactions may thus mimic functional polyanion-V1V2 interactions that HIV-1 uses for cell surface attachment during the initial stages of virus-cell entry.

Strand C is also the most variable of the V1V2 strands. Its location, at the edge of the sheet, however, provides an opportunity for sequence-independent recognition, through its exposed main-chain atoms. While the four hydrogen bonds made by the main chain of PG9 likely contribute only a small portion of the overall binding energy, the main chain-interactive surface of V1V2 totals 348 and 350 Å² in CAP45 and ZM109 complexes, respectively, potentially providing substantial contribution to the overall binding energy (Supplementary Table 21 shown in FIG. 47). This type of β-sheet interaction, for example, is the primary interaction between the CDR H3 of antibody 447-52D with the V3 of gp120 in a 3-and-almost-4 stranded (3-sheet (Stanfield et al., Structure, 12:193-204, 2004).

Without being bound by theory, the different types of PG9 interaction, involving glycan, electrostatics, and sequence-independent interactions, is each implicated for PG9 function. Such multicomponent recognition may also provide a mechanism that enables the immune system to overcome evasion associated with individual components of the interaction. Thus, for example, glycan-only affinity might lead to auto-reactivity, and surface areas of electrostatic and sequence-independent interactions might be individually too small to generate sufficient affinity for tight interactions. Together, however, the glycan, electrostatic and sequence-independent interactions achieve the substantial level of affinity required for potent neutralization.

In longitudinal studies, antibody recognition requiring glycan, either at residue 160, as described here, or at residue 332, are the most commonly elicited initial broadly neutralizing responses (Gray et al., J Virol, 85:4828-4840, 2011), an observation also seen with elite neutralizers (Walker et al., PLoS Pathog, 6:e1001028, 2010). In longitudinal studies, transmitted viruses in some cases do not have canonical glycosylation (e.g. at positions 160 or 332), but acquired these under immune selection (Moore et al., AIDS Res Hum Retroviruses, 27:A-29, 2011). Thus it appears that N-linked glycosylation at particular residues is selected as a means of immune evasion, but that these same glycans—now part of a homogeneous glycan array—can be recognized by very broadly neutralizing antibodies. Recent structural results indicate a number of 332-glycan dependent antibodies also use protruding CDR H3s, and, in at least one case, the antibody (PGT128) recognizes an epitope composed of two glycans and a strand. Collectively these results suggest that a penetrating CDR H3 recognizing conserved glycan and neighboring polypeptide is a paradigm for humoral recognition of heavily glycosylated antigens.

### Coordinate Deposition Information.

Coordinates and structure factors for PG9 Fab in complexes with V1V2 from CAP45 and ZM109 strains of HIV-1 have been deposited with the Protein Data Bank under accession codes 3U4E and 3U2S, respectively. Coordinates and structure factors unbound Fab structures of PG9, CH04, CH04H/CH02L (in two lattices), and PGT145 have been deposited with the Protein Data Bank under accession codes, 3U36, 3TCL, 3U46, 3U4B, and 3US1, respectively.

### Methods

Design of Large V1V2 Scaffolds.

Large V1V2 scaffolds were identified by a search of a culled database of high resolution crystal structures from the PDB, using the Multigraft Match algorithm implemented in Rosetta Multigraft (Azoitei et al., Science, 334: 373-376, 2011). Briefly, the stub of the V1V2 region from gp120 (PDB code 1RZJ) was treated as an epitope, and an exhaustive search was conducted for scaffolds that could accommodate backbone grafting of the V1V2 stub while maintaining backbone continuity and avoiding steric clash. Multiple combinations of endpoints on the V1V2 stub were tested, including the following pairs of endpoints in 1RZJ: (124,196), (125,196), (126,196), (124,197), (125, 197), (126,197), (124,198), (125, 198), (126,198). Matches were initially accepted with a loop closure RMSD of <2.0 Å and a steric clash between the V1V2 stub and the scaffold of less than 1.0 Rosetta units with all atoms present and having allowed for side-chain repacking. Only three scaffolds with >500 residues were identified with very low RMSD loop closure (<0.5 Å) for the V1V2 stub. To obtain additional scaffolds, a list of high resolution structures of large chains was constructed (346 chains included) and the V1V2 stub was grafted at manually selected sites on all unique proteins in that list, using explicit flexible backbone loop closure in RosettaRemodel (Huang et al., PLoS ONE, 6: e24109, 2011). If RosettaRemodel could produce a grafted V1V2 stub with a fully closed chain while maintaining hydrogen bonding in the remodeled region and without creating significant pockets in the structure, the output model was accepted as a scaffold candidate. The final scaffold sequences included the full length YU2 V1V2 sequence in place of the stub.

Design of Small V1V2 Scaffolds.

A database of small protein structures was created, with ligands removed and non-standard amino acids replaced by appropriate analogues. Candidate scaffolds were identified using the Multigraft Match algorithm as described above (Azoitei et al., Science, 334: 373-376, 2011). From the thousands of matches that passed these filters, the lowest RMSD match for each PDB code was examined manually to identify scaffolds with good packing, adequate tertiary structure supporting the V1V2 stub, a minimum of buried unsatisfied polar residues, and adequate space to accommodate the large, glycosylated V1V2 loops. In some cases scaffolds were re-designed to improve these features using human-guided computational (fixed backbone) design. Once the scaffold design and grafting of the V1V2 stub was completed, it was considered possible to insert any desired full-length V1V2 sequence. This study initially employed the YU2 V1V2 sequence. A total of 11 scaffolds were designed in this manner, based on the following PDB entries: 1CHLA, 1FD6A, 1G6MA, I1P9A, I1W4A, 1JLZA, 1QPMA, 1XBDA, 1XQQA, 1YWJA, 1BRZ. Two additional scaffolds were selected manually from crystal structures of small, stable proteins but were designed similarly using Multigraft Match; these scaffolds were based on PDB entries: 1E6G and 1JO8.

Expression and Purification of V1V2 Scaffolds.

Mammalian codon-optimized genes encoding V1V2 scaffolds were synthesized with an artificial N-terminal secretion signal and a C-terminal HRV3C recognition site followed by an 8×-His tag and a StreptagII. V1V2 sequences were from HIV-1 strains TRJO, CAP45, ZM53, ZM109 or 16055. The genes were cloned into the XbaI/BamHI sites of the mammalian expression vector pVRC8400, and transiently transfected into HEK293S GnTI^{-/-} cells (Reeves et al., Proc Natl Acad Sci USA, 99: 13419-13424, 2000), which were used due to a requirement for a Man₅GlcNac₂ at position 160 by PG9 and other broadly neutralizing V1V2-directed antibodies. Scaffolds were purified from the media using Ni²⁺-NTA resin (Qiagen), and the eluted proteins were digested with HRV3C (Novagen) before passage over a 16/60 S200 size exclusion column. Monodisperse fractions were pooled and passed over Ni²⁺-NTA resin to remove any uncleaved scaffold or residual HRV3C protease. The scaffolds were flash frozen in liquid nitrogen and stored at −80° C. Glycosylation mutants were expressed and purified in a similar manner.

Expression and Purification of PG9 N23Q HRV3C.

A mammalian codon-optimized gene encoding the PG9 heavy chain with an HRV3C recognition site (GLEVLFQGP) inserted after Lys235 was synthesized and cloned into pVRC8400. Similarly, the PG9 light chain was synthesized and cloned into the pVRC8400 vector, and an N23Q mutation was introduced to remove the sole glycosylation site on PG9. The modified PG9 heavy and light chain plasmids were transiently co-transfected into HEK293F cells, and IgG was purified from the supernatant after five days using Protein A agarose (Pierce).

Formation and Purification of PG9/V1V2 Scaffold Complexes.

Approximately 3 mg of purified PG9 N23Q HRV3C IgG was bound to 750 μl Protein A Plus agarose (Pierce) in a disposable 10 ml column. To this resin was added 6 mg of purified V1V2 scaffold (−20-fold molar excess over PG9 IgG). After washing away unbound scaffold with PBS, the column was capped and 40 μl of HRV3C protease at 2 U/μl was added to the resin along with 1 ml of PBS. After one hour at room temperature, the resin was drained, the eluate collected and passed over a 16/60 S200 column. Fractions corresponding to the PG9/V1V2 complex were pooled and concentrated to ˜5 mg/ml.

PG9/V1V2 Complex Crystallization and Data Collection.

A complex of PG9 complexed with 1FD6-ZM109 with four N-linked asparagines mutated to alanine (except Asn160 and Asn173) was screened against 576 crystallization conditions using a Cartesian Honeybee crystallization robot. Initial crystals were grown by the vapor diffusion method in sitting drops at 20° C. by mixing 0.2 μl of protein complex with 0.2 μl of reservoir solution (17% (w/v) PEG 3350, 10% (v/v) 2-methyl-2,4-pentanediol, 0.2 M lithium sulfate, 0.1 M imidazole pH 6.5). Crystals suitable for diffraction were manually reproduced in hanging drops by mixing equal volumes of protein complex with reservoir solution (8% (w/v) PEG 3350, 5% (v/v) 2-methyl-2,4-pentanediol, 90 mM lithium sulfate, 45 mM imidazole pH 6.5). Single crystals were flash frozen in liquid nitrogen in 12% (w/v) PEG 3350, 0.2 M lithium sulfate, 0.1 M imidazole pH 6.5, and 15% (v/v) 2R,3R-butanediol. Data to 1.80 Å were collected at a wavelength of 1.00 Å at the SER-CAT beamline ID-22 (Advanced Photon Source, Argonne National Laboratory).

A complex of PG9 and 1FD6-CAP45 at 2.2 mg/ml was also screened against 576 crystallization conditions. Initial crystals were grown in the same reservoir solution as for PG9/1FD6-ZM109. Crystals were manually reproduced in hanging drops by mixing equal volumes of protein complex with reservoir solution (13% (w/v) PEG 3350, 11% (v/v) 2-methyl-2,4-pentanediol, 0.2 M lithium sulfate, 0.1 M imidazole pH 6.5). Single crystals were bathed in a cryoprotectant of 20% (w/v) PEG 3350, 0.2 M lithium sulfate, 0.1 M imidazole pH 6.5, and 15% (v/v) 2R,3R-butanediol followed by immersion in Paratone-N and flash frozen in liquid nitrogen. Data to 2.19 Å were collected at a wavelength of 1.00 Å at the SER-CAT beamline BM-22.

PG9/V1V2 Complex Structure Determination, Model Building and Refinement.

Diffraction data were processed with the HKL2000 suite (Otwinowski et al., Methods Enzymol, 276:307-326, 1997) and a molecular replacement solution for the 1FD6-ZM109 dataset consisting of two unbound PG9 Fab molecules per asymmetric unit was obtained using PHASER™ (McCoy et al., J. Appl. Crystallogr., 40:658-674, 2007). Model building was carried out using COOT™ (Emsley et al., Acta Crystallogr., Sect. D: Biol. Crystallogr., 60: 2126-2132, 2004) and refinement was performed with PHENIX™ (Adams et al., Acta Crystallogr., Sect. D: Biol. Crystallogr., 58:1948-1954, 2002). Electron density for the Man₅GlcNac₂ attached to Asn160 and the two disulfide bonds were used as landmarks to build the V1V2 strands. Final data collection and refinement statistics are presented in Supplementary Table 7 (shown in FIG. 33). The Ramachandran plot as determined by MOLPROBITY™ (Davis et al., Nucl. Acids Res., 35:W375-383, 2007) shows 98.0% of all residues in favored regions and 100% of all residues in allowed regions.

The PG9/1FD6-ZM109 structure was used as the search model for the 1FD6-CAP45 dataset. A molecular replacement solution consisting of two complexes per asymmetric unit was obtained using PHASER (McCoy et al., J. Appl. Crystallogr., 40:658-674, 2007), and COOT™ (Emsley et al., Acta Crystallogr., Sect. D: Biol. Crystallogr., 60: 2126-2132, 2004) and PHENIX™ (Adams et al., Acta Crystallogr., Sect. D: Biol. Crystallogr., 58:1948-1954, 2002) were used for model building and refinement, respectively. The Ramachandran plot for this complex as determined by MOLPROBITY™ (Davis et al., Nucl. Acids Res., 35:W375-383, 2007) shows 97.3% of all residues in favored regions and 100% of all residues in allowed regions.

Surface Plasmon Resonance.

The binding kinetics of different V1V2 scaffolds to antibodies PG9 and PG16 were determined on a Biacore T-200 (GE Healthcare) at 25° C. with buffer HBS-EP+ (10 mM HEPES, pH 7.4, 150 mM NaCl, 3 mM EDTA, and 0.05% surfactant P-20). For comparison, PG9 and PG16 binding to full length HIV-1 gp120s was performed in parallel. The effects of the gp120 V3 loop on antibody binding were also assessed with V3 loop-deleted gp120s. In total, five full length gp120 proteins (strains ZM109, 16055, AD244, CAP45, and TRJO), two V3 loop-deleted gp120 proteins (160554V3 and AD244ΔV3), and five V1V2 scaffolds (1FD6-ZM109, 1JO8-ZM109, 1FD6-16055, 1JO8-CAP45, and 1JO8-TRJO) were immobilized onto CM5 chips to 500 response units (RUs) with standard amine coupling. PG9 Fab and PG16 Fab were injected over the channels at 2-fold increasing concentrations with a flow rate of 30 μl/min for 3 minutes and allowed to dissociate for another 5 minutes. Regenerations were performed with one 25 μl injection of 3.0 M MgCl₂ at a flow rate of 50 μl/ml following the dissociation phase. T-200 Biacore Evaluation software was used to subtract appropriate blank references and fit sensorgrams globally using a 1:1 Langmuir model. In some cases, especially the binding to V1V2 scaffolds, the sensorgrams could not reasonably be fit to a 1:1 Langmuir model due to heterogeneity of the immobilized ligands, and thus a 1:1 model assuming heterogeneous ligands was used. The relative percentage of each component in the heterogeneous ligands was calculated by its contribution to the total Rₘₐₓ and the kinetic parameters are listed separately. Mass transfer effects were assessed by the t_{c} values given by the T-200 Biacore Evaluation software. No significant mass transport effects were detected in all measurements (t_{c}>10¹⁰).

Electron Microscopy and Image Processing.

Negative stained grids were prepared by applying 40 μg/ml of the purified T13-gp120 16055 (82-492)—PG9 ternary complex to a freshly glow discharged carbon coated 400 Cu mesh grid and stained with 2% Uranyl Formate. Grids were viewed using a FEI Tecnai TF20 electron microscope operating at a high tension of 120 kV at the National Resource for Automated Molecular Microscopy. Initial models were generated using the random conical tilt method through the Appion package (Lander et al., Journal of structural biology, 166:95-102, 2009 and Radermacher et al., Journal of microscopy, 146:113-136, 1987). Images were acquired at a magnification of 62,000 with a defocus range of 1.5 to 2.5 μm onto a Gatan 4k×4k CCD using the Leginon package (Subway et al., Journal of structural biology, 151: 41-60, 2005). The pixel size of the CCD was calibrated using a 2D catalase crystal with known cell parameters. The initial models were improved using a dataset collected at a magnification of 150,000× at 0, 15, 30, 45, and 55° tilts with a defocus range of 500 to 700 nm through a multi-model approach developed in-house with the SPIDER package (Frank et al., Ultramicroscopy, 6:343-358, 1987). The tilts provided additional particle orientations to improve the image reconstructions.

PG9 Fab Crystallization and Refinement.

PG9 Fab with an N23Q mutation in the light chain was obtained by cleaving the recombinant IgG described above with HRV3C protease, followed by gel filtration chromatography. PG9 Fab at a concentration of 13.7 mg/ml was screened against 576 crystallization conditions, and initial crystals were obtained using the sitting drop vapor diffusion method. Crystals were obtained from a reservoir containing (25% (w/v) PEG 3350, 15% (v/v) 2-methyl-2,4-pentanediol, 0.2 M lithium sulfate, 0.1 M imidazole pH 6.5). After cryo-protection with 15% 2R,3R-butanediol, crystals were mounted and flash frozen in liquid nitrogen. Data to 3.30 Å were collected at a wavelength of 1.00 Å at the SER-CAT beamline ID-22. Statistics for data collection and data processing in HKL2000 (Otwinowski et al., Methods Enzymol, 276:307-326, 1997) are summarized in Supplementary Table 19 (shown in FIG. 45). The structure in space group P1 was solved by molecular replacement using the program PHASER™ (McCoy et al., J. Appl. Crystallogr., 40:658-674, 2007) with the PG16 Fab structure (PDB ID 3LRS) (Pancera et al., J. Virol., 84:8098-8110, 2010) as a search model. Model building and refinement were performed using COOT™ (Emsley et al., Acta Crystallogr., Sect. D: Biol. Crystallogr., 60: 2126-2132, 2004) and PHENIX™ (Adams et al., Acta Crystallogr., Sect. D: Biol. Crystallogr., 58:1948-1954, 2002), respectively. Refinement statistics for the PG9 Fab model are reported in Supplementary Table 19 (shown in FIG. 45).

CH04 and CH04H/CH02L Fab Expression, Crystallization and Refinement.

A mammalian codon-optimized gene encoding the CH04 heavy chain with a stop codon inserted after Asp234 was synthesized and cloned into pVRC8400. Similarly, the CH04 and CH02 light chains were synthesized and cloned into the pVRC8400 vector. The CH04 heavy and light chain plasmids were transiently co-transfected into HEK293F cells (or CH04 heavy with CH02 light chain), and Fab was purified from the supernatant after five days using Kappa agarose column (CaptureSelect Fab ic; BAC). CH04 and CH04H/CH02L Fabs at a concentration of 16 mg/ml and 10 mg/ml, respectively, were screened against 576 crystallization conditions using a Cartesian Honeybee crystallization robot. CH04 Fab crystals were obtained in 20% (w/v) PEG 8000, 3% (v/v) 2-methyl-2,4-pentanediol, 70 mM imidazole pH 6.5. Single crystals were flash frozen in liquid nitrogen in 24% (w/v) PEG 8000, 3.4% (v/v) 2-methyl-2,4-pentanediol, 85 mM imidazole pH 6.5, and 15% (v/v) 2R,3R-butanediol. CH04H/CH02L Fabs crystals were obtained in 16% PEG 400, 8% PEG 8000, 0.1 M acetate pH 4.5 (orthorhombic forms) and 15% PEG 3350, 9% 2-methyl-2,4-pentanediol, 0.1 M lithium sulfate, 0.1 M imidazole pH 6.5 (tetragonal forms) Data to 1.90 Å (CH04 Fab) and 2.90 Å (CH04H/CH02L Fab) were collected at a wavelength of 1.00 Å at the SER-CAT beamline ID-22 and BM-22, respectively.

Diffraction data were processed with the HKL2000 suite (Otwinowski et al., Methods Enzymol, 276:307-326, 1997) and a molecular replacement solution for the CH04 data set consisting of two CH04 Fab molecules per asymmetric unit was obtained using PHASER (McCoy et al., J. Appl. Crystallogr., 40:658-674, 2007) and PDB ID codes 1DFB (heavy chain) (He et al., Natl. Acad. Sci., 89:7154-7158, 1992) and 1QLR (light chain) (Cauerhff et al., The Journal of Immunology, 156:6422-6428, 2000) as search models. CH04 Fab was used as the search model for CH04H/CH02L. Model building was carried out using COOT (Emsley et al., Acta Crystallogr., Sect. D: Biol. Crystallogr., 60: 2126-2132, 2004), and refinement was performed with PHENIX (Adams et al., Acta Crystallogr., Sect. D: Biol. Crystallogr., 58:1948-1954, 2002). Final data collection and refinement statistics are presented in Supplementary Table 19 (shown in FIG. 45).

PGT145 Fab Expression, Crystallization and Refinement.

Expression and purification of PGT145 was performed using a similar protocol to that previously described (Pejchal et al., Proc Natl Acad Sci USA, 107: 11483-11488, 2010). Briefly, the Fab was produced as a secreted protein by co-transfecting the heavy and light chain genes into HEK 293T cells. Three days after transfection, the media was recovered, concentrated and flowed over an anti-human kappa light chain affinity matrix (CaptureSelect Fab κ; BAC). The eluted fraction containing the Fab was further purified by cation exchange chromatography followed by size exclusion chromatography. PGT145 Fab at a concentration of 10 mg/ml was crystallized using the sitting drop vapor diffusion method. Crystals were obtained in a mother liquor containing 0.1 M HEPES, pH 7.5, 2 M ammonium sulfate and 20% PEG 400. After cryo-protection in 20% glycerol, crystals were mounted and flash frozen in liquid nitrogen. PGT145 Fab crystals were exposed to a monochromatic X-ray beam at the Advanced Photon Source Sector 23-ID (Argonne National Laboratory, Illinois). Statistics for data collection and data processing in HKL2000 (Otwinowski et al., Methods Enzymol, 276:307-326, 1997) are summarized in Supplementary Table 19 (shown in FIG. 45). The structure in space group P4₁2₁2 was solved by molecular replacement using the program PHASER (McCoy et al., J. Appl. Crystallogr., 40:658-674, 2007) with the PG16 Fab structure (PDB ID 3MUG) (Pejchal et al., Proc Natl Acad Sci USA, 107: 11483-11488, 2010) as a search model. Refinement of the structure was performed using a combination of CNS (Brunger et al., Acta Crystallogr D Biol Crystallogr, 54: 905-921, 1998), CCP4 (Winn et al., Acta Crystallogr D. Biol Crystallogr, 67:235-242, 2011) and COOT (Emsley et al., Acta Crystallogr., Sect. D: Biol. Crystallogr., 60: 2126-2132, 2004). The final statistics of the refined PGT145 Fab model are reported in Supplementary Table 19 (shown in FIG. 45).

STD Experiments by NMR.

All NMR experiments were carried out at 298 K on Bruker avance 600 or avance 500 instruments equipped with a triple resonance cryo-probe incorporating gradients in z-axis. 1D STD spectra were acquired by selectively irradiating at −1 ppm and +40 ppm as on- and off-resonance frequencies, respectively, using a train of 50 ms Gaussian-shaped radio frequency pulses separated by 1 ms delays and an optimized power level of 57 db. During NMR experiments water suppression was achieved by binomial 3-9-19 pulse sequence and protein resonances were suppressed by applying 10 ms T1ρ filter. Samples were prepared in 20 mM sodium phosphate buffer containing 50 mM sodium chloride at pH 6.8. The NMR data were processed and analyzed by using TOPSPIN 2.1. The STD amplification factor, A_{STD}, was obtained according to the equation, A_{STD}=(I₀−I_{SAT})I₀⁻¹([Lt]/[P]), where Lt and P are the total ligand and protein concentrations, respectively (Mayer et al., J. Am. Chem. Soc., 123: 6108-6117).

Surface Areas and Average Surface Electrostatic Potentials Calculations.

Surface area calculations were performed using PISA (Krissinel et al., J. Mol. Biol., 372: 774-797, 2007) and MS (Connolly, J. Appl. Cryst., 16:548-558, 1983). The interactive surfaces with PG9 for CAP45 and ZM109 were obtained using pymol and selecting atoms of V1V2 within 5.5 Å of PG9 residues. Electrostatic surface potentials for the CDR H3 and interacting surface for CAP45 and ZM109 were obtained using GRASP (Nicholls et al., Proteins, 11:281-296, 1991). The Poisson-Boltzmann (PB) potential grid map and surface points of each CDR H3 region and CAP45 and ZM109 interacting surfaces were determined using GRASP. The PB potential for each surface point was determined by trilinear interpolation from the values of the eight corners of the cube where the surface point resided in. The average surface PB potential is the linear average of the PB potentials of all surface points.

Figures.

Structure figures were prepared using PYMOL (The PyMOL Molecular Graphics System, Version 1.4, Schrödinger, LLC.).

### Example 2

### Minimal PG9 Epitope Synthesized as a Glycopeptide

This example illustrates isolated polypeptides including the minimal PG9 epitope from the V1/V2 domain of HIV-gp120. The minimal PG9 epitope includes gp120 positions 154-177. The isolated polypeptides are stabilized to maintain a PG9-bound conformation by introduction of a pair of cysteine residues at positions 155 and 176, and include an asparagine residue at positions 160 and 156, or at positions 160 and 173. The results show that the minimal PG9 epitope peptides specifically bind to PG9 antibody with a K_{D} as low as ˜5 μM.

General Procedure for Peptide Synthesis:

Peptides were synthesized on a Pioneer automatic

Peptide Synthesizer (Applied Biosystems) using Fmoc-protected amino acids as building blocks and 2-(1-H-azabenzotriazol-1-yl)-1,1,3,3-tetramethyluronium hexafluorophosphate (HATU) and diisopropylethyl amine (DIPEA) as coupling reagent following standard procedure on a CLEAR amide resin. GlcNAc-attached peptides were synthesized by using GlcNAc-Asn building block namely, N⁴-(2-Acetamido-3,4,6-tri-O-acetyl-2-deoxy-β-D-glucopyranosyl)-N²-(fluorenylmethoxycarbonyl)asparagine (see, e.g., Kirsch et al., Bioorg. Med. Chem. 1995, 3, 1631-1636.). A Biotin with six carbon spacer was installed at the N-terminal of peptides on resin by treatment with succinymidyl-6-(biotinamido)hexanoate in presence of DIPEA. The Peptides were cleaved from the resin by using Cocktail R (TFA/thioanisole/EDT/Anisole=90/5/3/2) followed by precipitation with cold ether. Removal of acetyl group from GlcNAc moiety and cyclization through two cysteine residue at two ends was achieved simultaneously by treatment with 2.5% aqueous hydrazine. The crude peptide was purified by reverse phase HPLC to afford peptides 25-36% yield (0.05-0.1 mmole scale).

General Procedure for Syntheses of Glycopeptides:

Glycopeptides including 154-177 of the indicated HIV-1 strains were synthesized by treating the 154-177 peptide with three different glycosynthase enzymes, namely EndoD-N223Q, EndoM-175Q and EndoA-N171A by using respective oxazoline donor and GlcNAc peptides as follows:

a) General transglycosylation procedure with EndoD-N223Q: A mixture of GlcNAc-peptide (acceptor) and M₅GlcNAc oxazoline (donor) (1:3=acceptor:donor) in 50 mM phosphate buffer pH 7.3 was incubated with EndoD-N223Q of a final concentration of 40 ng/μL for 0.5 hours. All transglycosylation reactions were stopped by diluting the solution with 0.1% TFA (aq.). The reaction was monitored by reverse phase HPLC and the yield was calculated from the absorbance at 280 nm from the ratio of acceptor peptide and newly formed glycosylated peptide peak.

b) General transglycosylation procedure with EndoM-N175Q: A mixture of GlcNAc-peptide (acceptor) and respective complex type oxazoline donor (SCT and CT) (1:3=acceptor:donor) in 50 mM phosphate buffer pH 7.2 was incubated with EndoM-N175Q of a final concentration of 0.4 μg/μL for 0.5 hours.

c) General transglycosylation procedure with EndoA-N171A: A mixture of GlcNAc-peptide (acceptor) and respective M₉GlcNAc oxazoline donor (1:3=acceptor:donor) in 50 mM phosphate buffer pH 7.3 containing 10% DMSO was incubated with EndoA-N175A of a final concentration of 2 μg/μL for 3.5 hours. EndoA wild type 0.1 μg/μL was utilized for transglycosylation reaction with M₃GlcNAc oxazoline.

Surface Plasmon Resonance (SPR) Measurements:

SPR measurements were performed on a Biacore T100 instrument (GE Healthcare). Bioinylated glycopeptides were immobilized on streptavidin-coated sensor chips (SA) in a solution of HBS-P buffer 1× (0.1M HEPES, 1.5M NaCl, 0.5% v/v surfactant P20, pH 7.4) by injecting manually until to achieve 20-30 RU or 300-330 RU. PG9 Fab and PG16 Fab were injected over four cells at 2-fold increasing concentrations with a flow rate of 50 μl/min for three minutes and allowed to dissociate for another five minutes. Regeneration were performed by injecting 3M MgCl₂ with a flow rate of 50 μL/min for three minutes followed by injection of HBS-P buffer 1× with a flow rate of 50 μl/min for five minutes. Three blanks were tested and same concentrations were duplicated. The temperature of the instrument was set at 25° C. and data were collected at the rate of 10 Hz. T-100 Biacore Evaluation software were utilized to subtract suitable blank reference and to fit the sensorgrams globally applying a 1:1 Langmuir model. Mass transfer effects were checked by the t, values displayed by the T-100 Biacore. No significant mass transportation was observed.

Results:

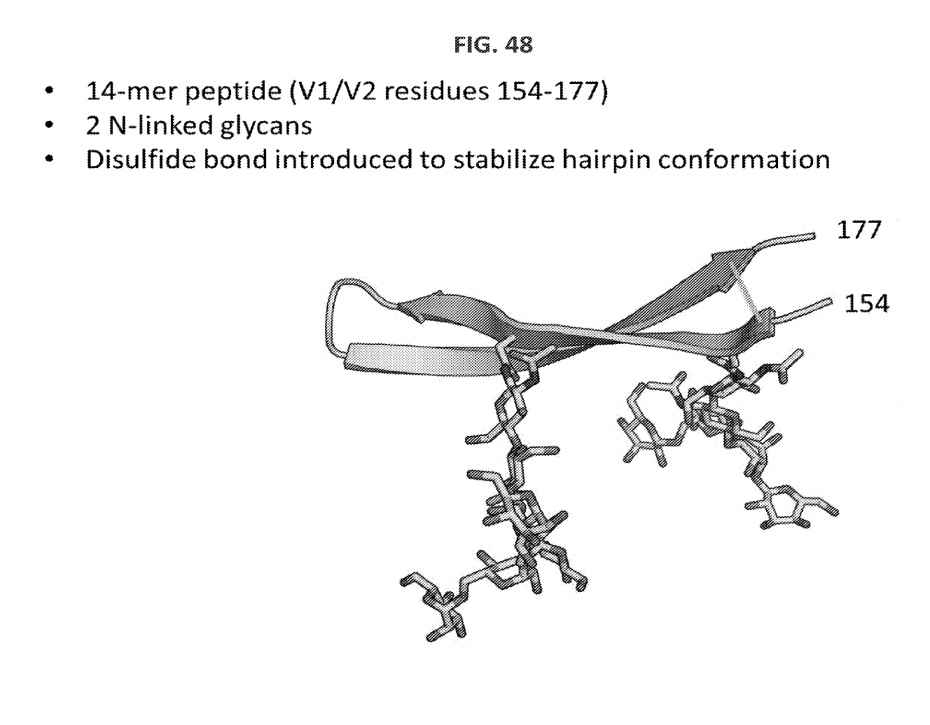

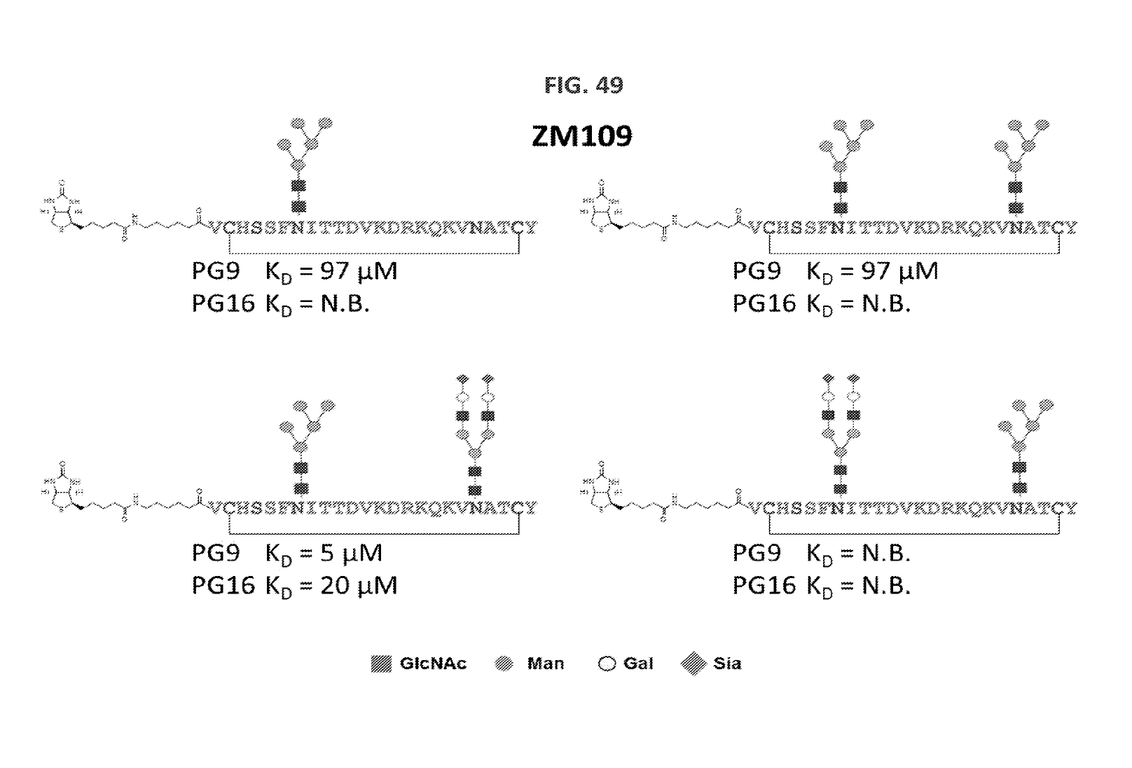

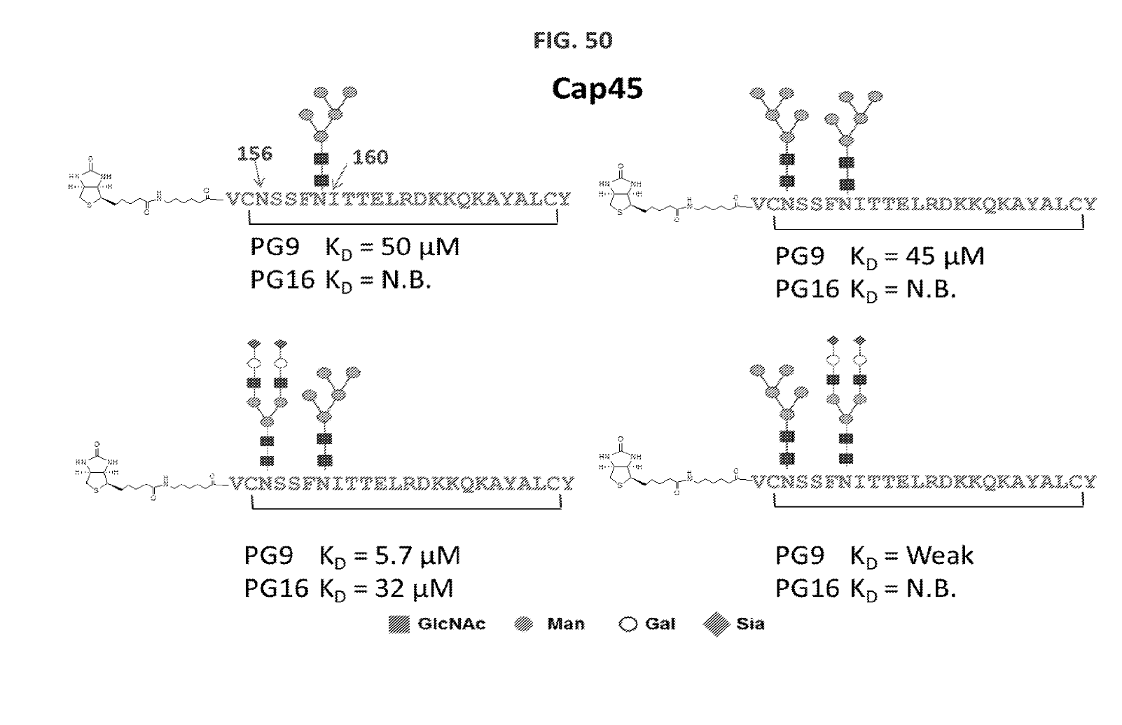

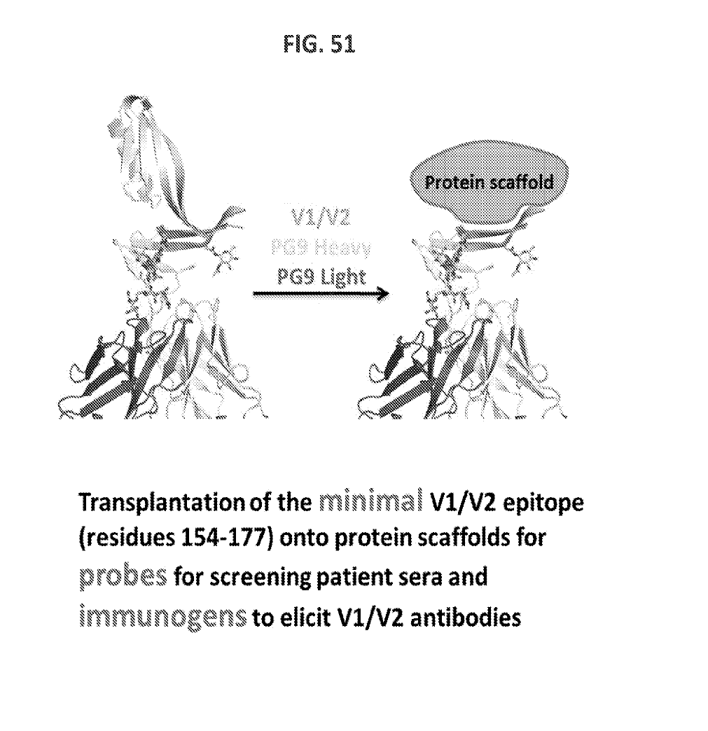

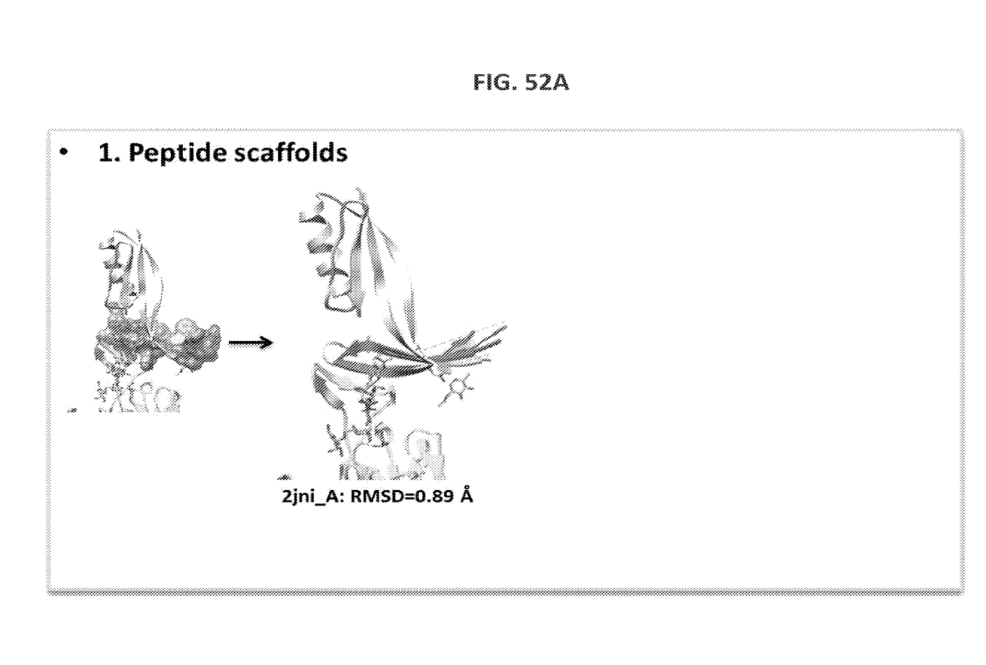

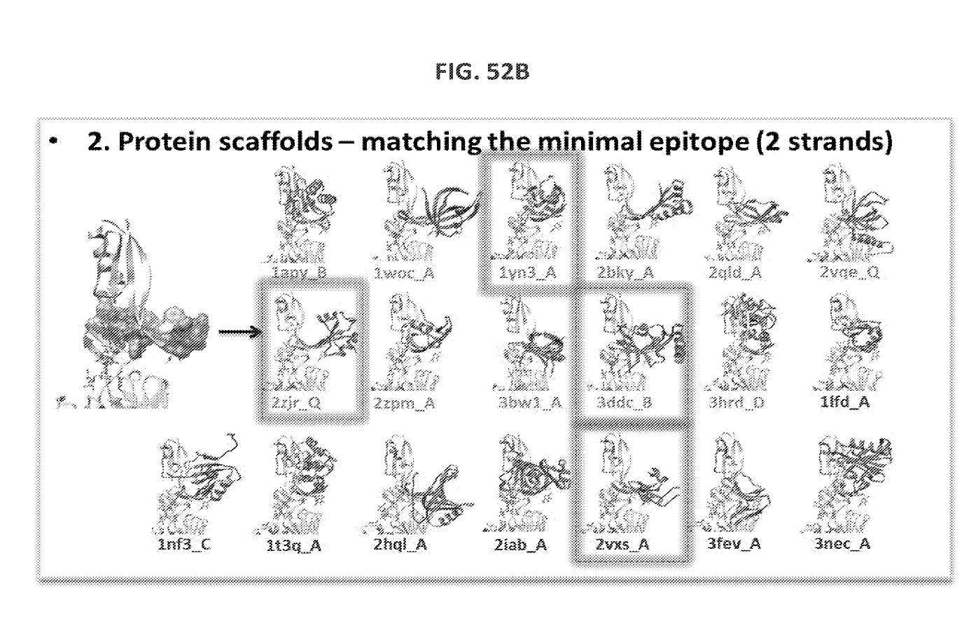

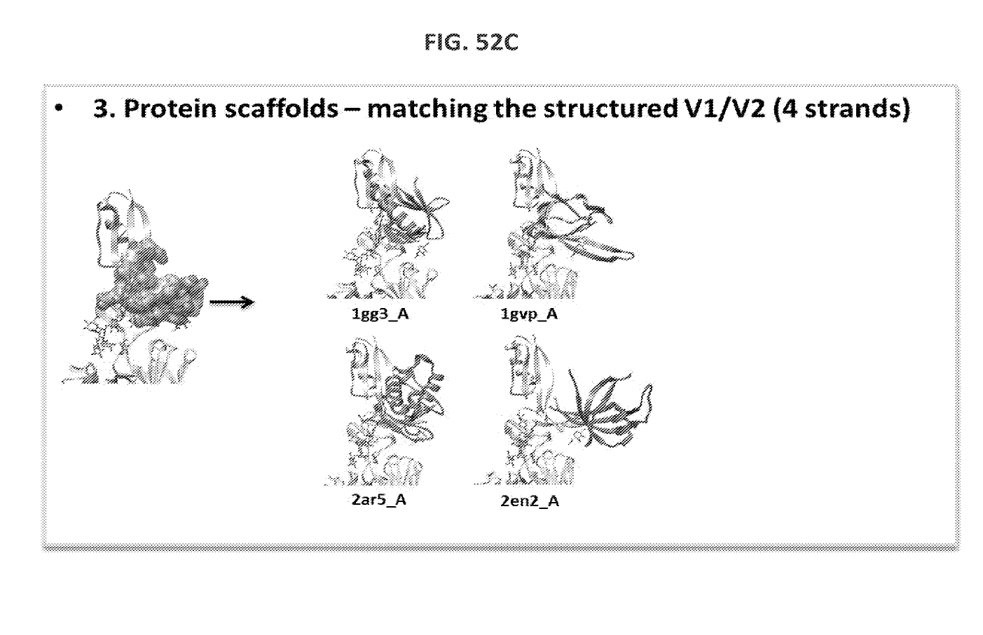

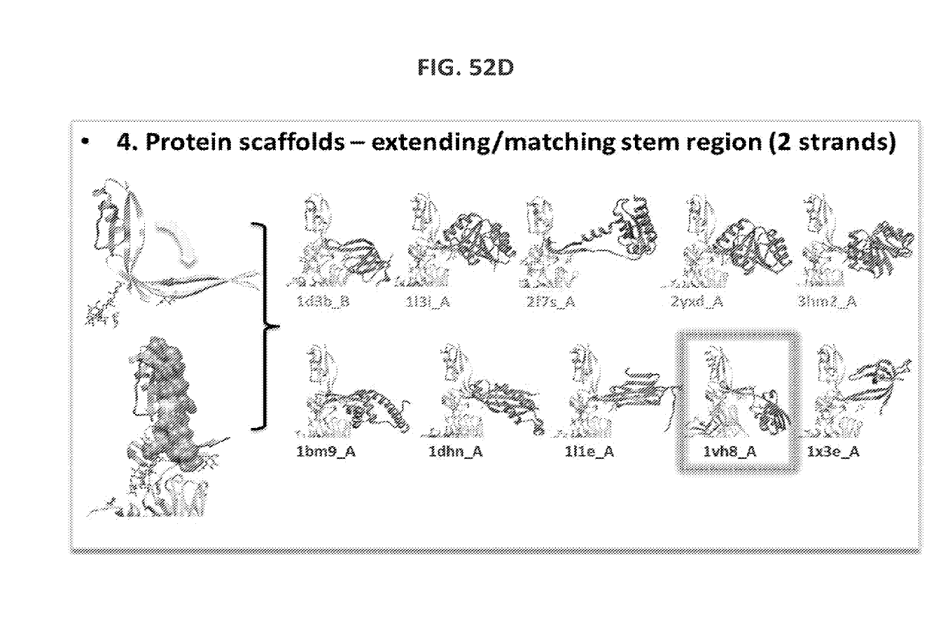

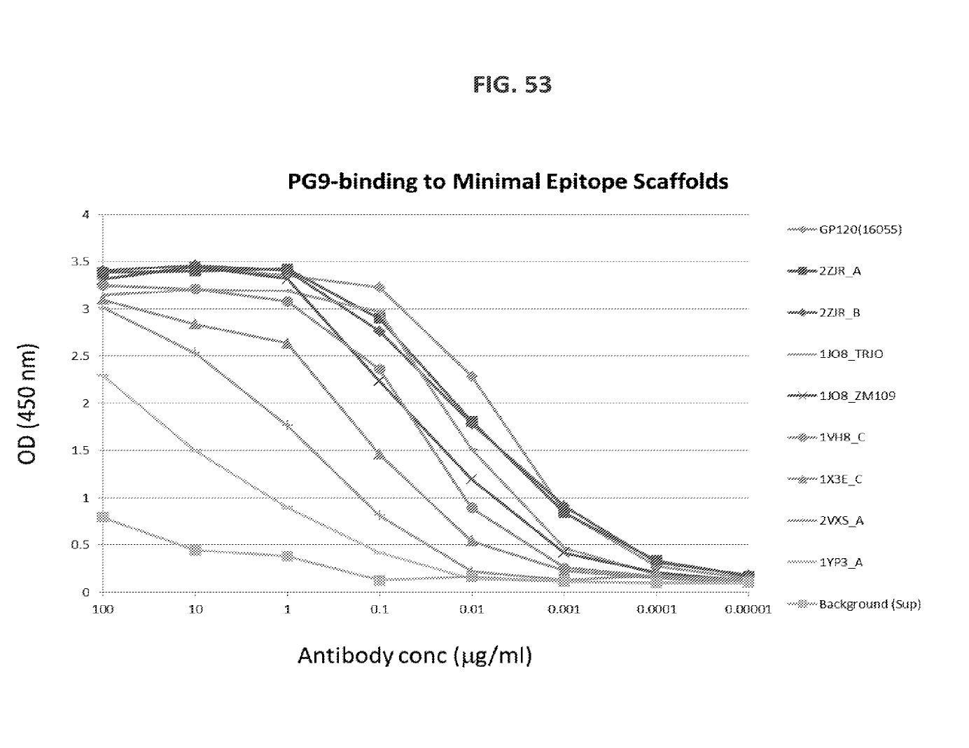

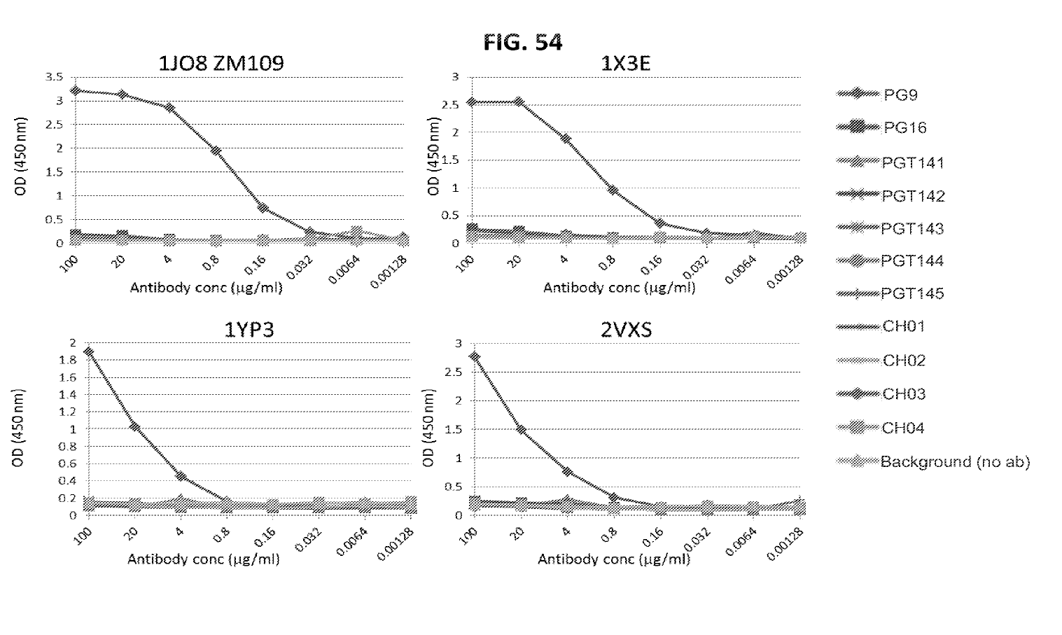

The results demonstrate that a Man5GlcNAc2 moiety at position N160 of the 154-177 gp120 peptide is sufficient for weak PG9 binding, whereas PG16 binding requires an additional complex glycan at position N156 (CAP45) or N173 (ZM109) of the gp120 peptide (see FIGS. 48-50). Both PG9 and PG16 have the highest affinity for glycopeptides containing a Man5GlcNAc2 at position N160 and a complex glycan at position N156 (CAP45) or N173 (ZM109). For both PG9 and PG16, a complex glycan at position N160 reduced binding.

### Example 3

### Minimal PG9 Epitope Polypeptides on an Epitope-Scaffold

This example illustrates isolated epitope-scaffolds including the minimal PG9 epitope from the V1/V2 domain of gp120 grafted onto scaffold proteins. The results show that several PG9-epitope scaffolds specifically bind to monoclonal antibody PG9.

Methods Used to Select Scaffolds.

Scaffolds were selected from all available PDB structures based on several search criteria including: structures which matched the stem region of the 154-177 sequence, structures which aligned best with the four V1V2 strands, structures which best aligned with only the two strands from 154-177 and peptide scaffolds. Candidate protein scaffolds were modeled and filtered to remove those with a root mean squared deviation over 1.5 angstroms, those that were over 150 residues and those that had surface exposure of the epitope below 40%. Finally, PG9 docking to the modeled scaffolds was performed to eliminate those that would cause clashing issues.

Methods Used to Produce Scaffolds.

A 96-well microplate-formatted transient transgene expression approach was used to initially screen V1/V2 minimal epitope scaffolds. 100 μl of physiologically growing GnTI⁻ cells was seeded in each well of a 96-well microplate at a density of 2.5×10⁵ cells/ml in Dulbecco's Modified Eagle Medium supplemented with 10% Ultra-Low IgG Fetal Bovine Serum and 1×-Non-Essential Amino Acids (Invitrogen, CA). Cells were transfected with 0.25 μg of plasmid DNA encoding the minimal epitope scaffolds and grown for 5 days. V1/V2 minimal epitope scaffolds, which all contain a poly-his tag, that were expressed in the 96 well format were then screened for expression using biolayer interferometry (Octet, ForteBio) with sensors coated with an anti-his antibody. A series of minimal-PG9-epitope scaffolds were designed and produced. The amino acid sequence of these epitope scaffolds is provided as SEQ ID NOs: 9-77 (see Table 2).

Methods Used to Test Binding.

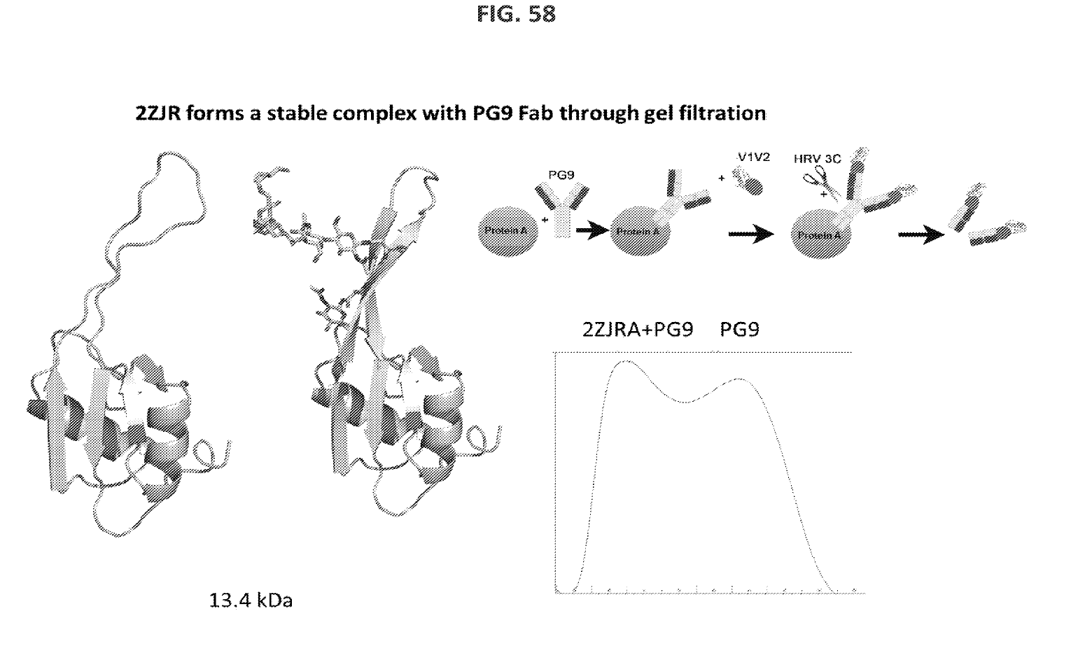

The supernatants of all wells expressing minimal epitope scaffolds were tested for binding to PG9, CH01, CH03, PGT145 and PGT142 antibodies through ELISA assays in which the supernatant was diluted 5-fold in PBS and incubated on nickel coated plates. Those scaffolds in wells that displayed high signal when screened with antibody were expressed at a larger scale (1 L), purified on Ni-NTA columns and tested for binding to PG9, PG16, CH01, CH02, CH03, CH04, PGT141, PGT142, PGT143, PGT144, and PGT145 antibodies by ELISA in a dilution series. Some, such as 2ZJR_A, were run over a protein A column coated in PG9 which was subsequently cleaved from the column resulting in an eluted complex consisting of the scaffolds and the PG9 Fab. This was run through gel filtration and displayed a shift in the elution profile indicating the intact complex (FIG. 58).

Results.

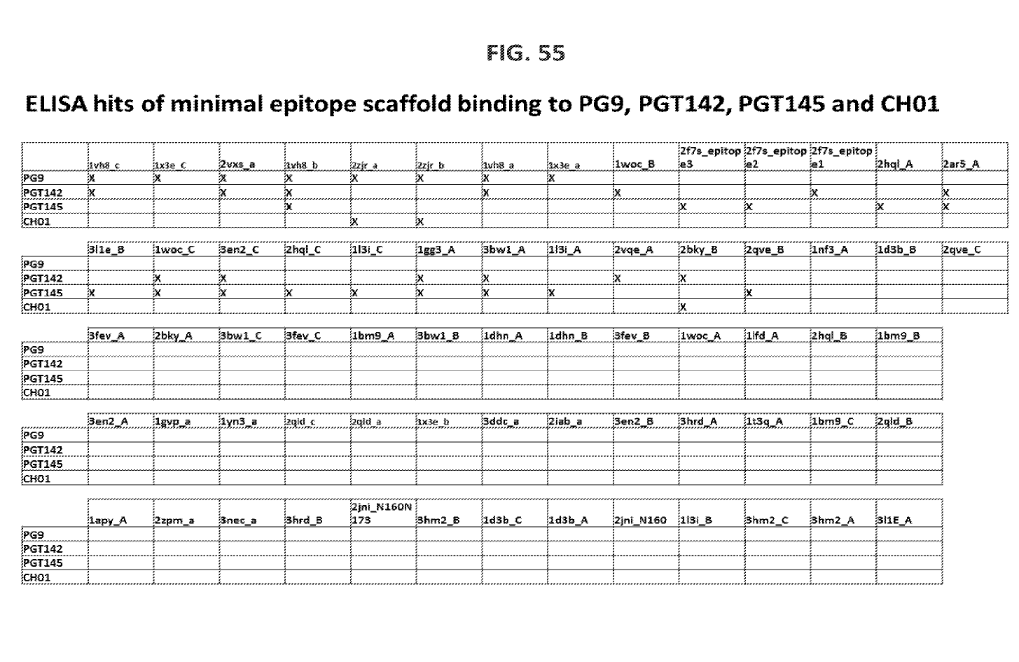

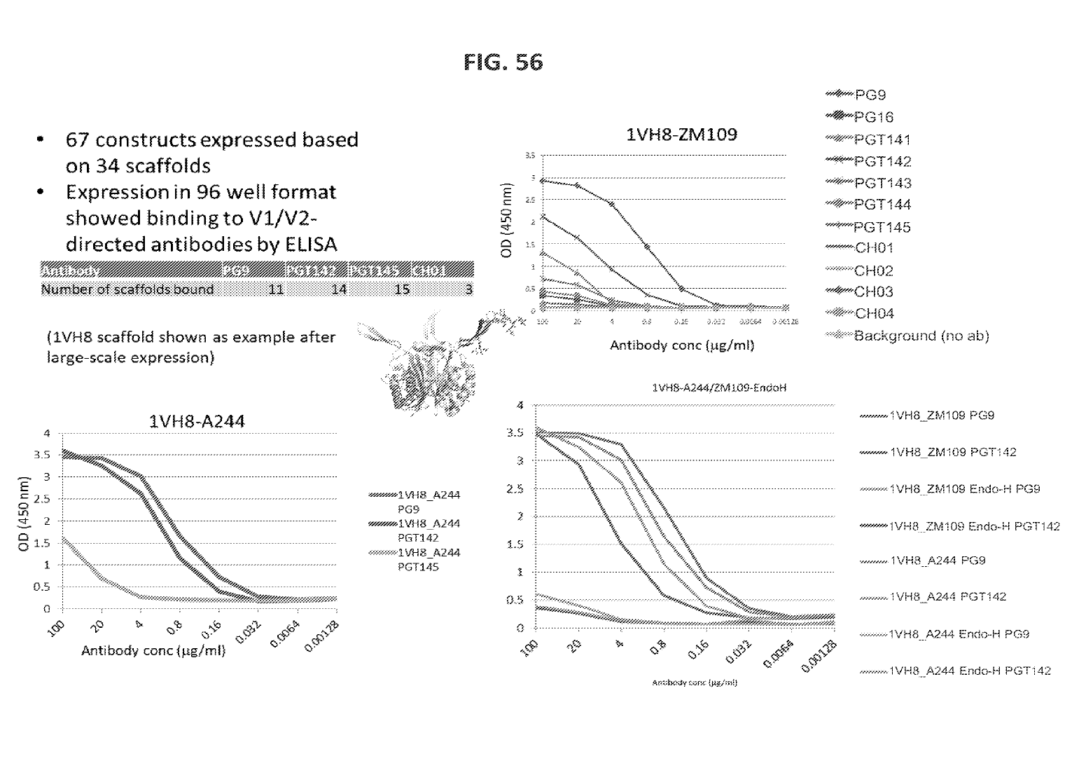

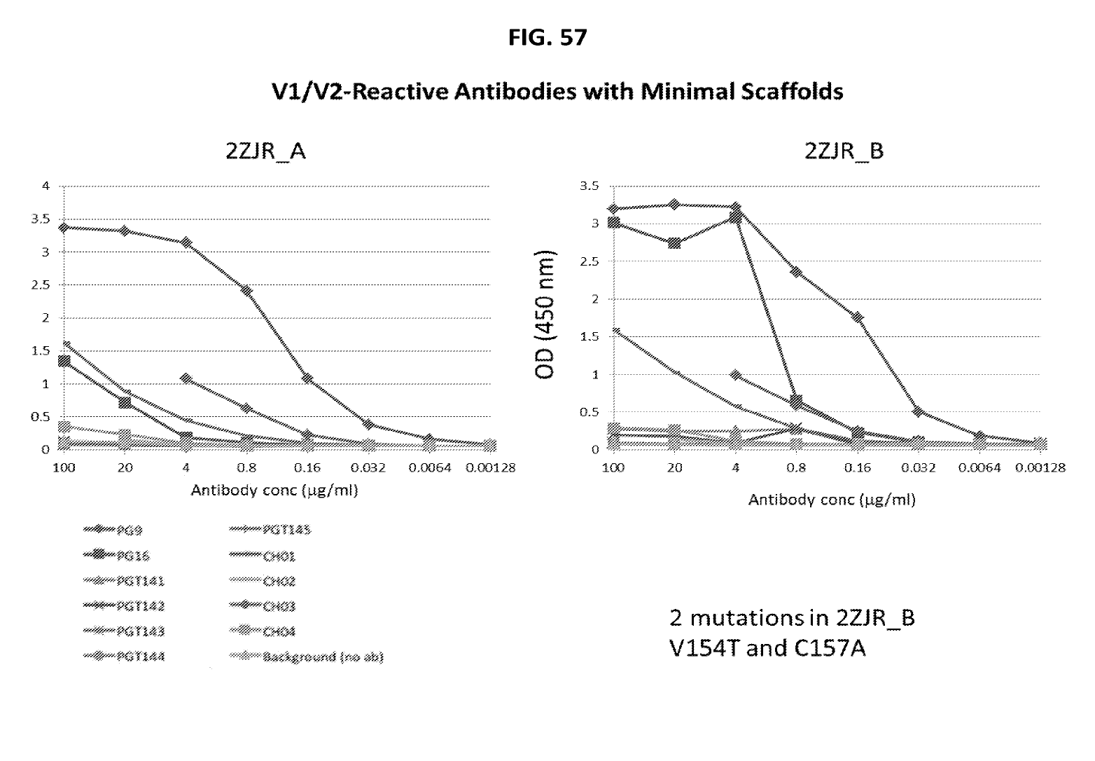

The minimal epitope scaffolds produced in the 96 well plate format reveal that many of the scaffolds express at least at low levels. Some of the scaffolds which do express are able bind PG9 and form stable complexes and many also show binding to various other types of V1V2 binding antibodies such as CH01, CH03, PGT142 and PGT145 indicating that the two strands comprising residues 154-177 are sufficient for a variety of broadly neutralizing antibodies that target the V1V2 region. The results show that the following epitope scaffolds bind to monoclonal antibody PG9: 1vh8_c, 1YN3_A, 1x3e_C, 2vxs_a, 1vh8 b, 2zjr_a, 2zjr_b, 1vh8_a, 1x3e_a, 3pyr_a, 1t0a_a, 2f7s_B, and 2f7s_C (see FIG. 55)

TABLE 2
Minimal PG9 Epitope-Scaffolds

| Epitope- | Epitope- | Native Scaffold | |
|---|---|---|---|
| Scaffold | Scaffold | PDB Acc. No. and | Substitutions/insertions/deletions in Epitope- |
| Name | Sequence | SEQ ID NO | Scaffold compared to Native Scaffold |
| 2JNI_A | SEQ ID NO: 9 | 2JNI (SEQ ID NO: 78) | Y7N + R9T) |
| 2JNI_B | SEQ ID NO: 10 | 2JNI (SEQ ID NO: 78) | Y7N + R9T, C3F + R18N + C20T, ins(V-Nterm and |
| | | | Cterm-Y)) |
| 3BW1_A | SEQ ID NO: 11 | 3BW1 (SEQ ID NO: 79) | 46-67−>154-177) |
| 3BW1_B | SEQ ID NO: 12 | 3BW1 (SEQ ID NO: 79) | 46-67−>154-177, V154D, C157A, Y177E) |
| 3BW1_C | SEQ ID NO: 13 | 3BW1 (SEQ ID NO: 79) | 46-67−>154-177, V154D, C157A, Y177E, |
| | | | S45C + M68C) |
| 2QLD_A | SEQ ID NO: 14 | 2QLD (SEQ ID NO: 80) | Del85-174, 21-44−>154-177) |
| 2QLD_B | SEQ ID NO: 15 | 2QLD (SEQ ID NO: 80) | Del85-174, 21-44−>154-177, L11T, C157A) |
| 2QLD_C | SEQ ID NO: 16 | 2QLD (SEQ ID NO: 80) | Del85-174, 21-44−>154-177, L11T, C157A, |
| | | | F159H, I161R) |
| 2ZJR_A | SEQ ID NO: 17 | 2ZJR (SEQ ID NO: 81) | 55-78−>154-177, K31G, Y177A) |
| 2ZJR_B | SEQ ID NO: 18 | 2ZJR (SEQ ID NO: 81) | 55-78−>154-177, K31G, V154T, Y177A, C157A) |
| 2BKY_A | SEQ ID NO: 19 | 2BKY (SEQ ID NO: 82) | 62-84−>154-177) |
| 2BKY_B | SEQ ID NO: 20 | 2BKY (SEQ ID NO: 82) | 62-84−>154-177, Y177I, C157A) |
| 2VQE_A | SEQ ID NO: 21 | 2VQE (SEQ ID NO: 83) | Del80-104, 19-42−>154-177, K155V) |
| 2VQE_B | SEQ ID NO: 22 | 2VQE (SEQ ID NO: 83) | Del80-104, 19-42−>154-177, K155V, C157A) |
| 2VQE_C | SEQ ID NO: 23 | 2VQE (SEQ ID NO: 83) | Del80-104, 19-42−>154-177, K155V, C157A, |
| | | | F159R) |
| 1APY_A | SEQ ID NO: 24 | 1APY (SEQ ID NO: 84) | 121-142−>156-177, C157F, F176C, Y177I) |
| 3DDC_A | SEQ ID NO: 25 | 3DDC (SEQ ID NO: 85) | 37-85−>154-177, K155V, C157L, F159L, I161R) |
| 3HRD_A | SEQ ID NO: 26 | 3HRD (SEQ ID NO: 86) | 1-20−>154-175, V154M, C157I) |
| 3HRD_B | SEQ ID NO: 27 | 3HRD (SEQ ID NO: 86) | 1-20−>154-175, V154M, C157I, Q170R, V172I, |
| | | | A174T) |
| 1YN3_A | SEQ ID NO: 28 | 1YN3 (SEQ ID NO: 87) | l-32−>154-177, V154G, K155S, C157V) |
| 1WOC_A | SEQ ID NO: 29 | 1WOC (SEQ ID NO: 88) | 32-49−>154-177, K155H) |
| 1WOC_B | SEQ ID NO: 30 | 1WOC (SEQ ID NO: 88) | 32-49−>154-177, K155H, C157A) |
| 1WOC_C | SEQ ID NO: 31 | 1WOC (SEQ ID NO: 88) | 32-49−>154-177, K155H, C157A, L31C + M50C) |
| 2ZPM_A | SEQ ID NO: 32 | 2ZPM (SEQ ID NO: 89) | 47-66−>155-176, C157L, F150L, F176P) |
| 1LFD_AA | SEQ ID NO: 33 | 1LFD (SEQ ID NO: 90) | l-26−>154-177, V154G, K155D, F159I, I161V, |
| | | | F176S, K39A, N41A) |
| 1T3Q_A | SEQ ID NO: 34 | 1T3Q (SEQ ID NO: 91) | l-23−>154-177, V154S, C157M, F176P, Y177R) |
| 2IAB_A | SEQ ID NO: 35 | 2IAB (SEQ ID NO: 92) | 24-43−>156-175, C157A) |
| 3NEC_A | SEQ ID NO: 36 | 3NEC (SEQ ID NO: 93) | 49-67−>157-175, C157H |
| 2VXS_A | SEQ ID NO: 37 | 2VXS (SEQ ID NO: 94) | 58-86−>157-175, C157I, F159Q) |
| 1NF3_A | SEQ ID NO: 38 | 1NF3 (SEQ ID NO: 95) | 44-65−>154-177, V154I, K155R, C157G, F159S, |
| | | | I161R, Y177I) |
| 2HQL_A | SEQ ID NO: 39 | 2HQL (SEQ ID NO: 96) | Del100-104, 28-41−>154-177, V154K) |
| 2HQL_B | SEQ ID NO: 40 | 2HQL (SEQ ID NO: 96) | Del100-104, 28-41−>154-177, V154K, C157A) |
| 2HQL_C | SEQ ID NO: 41 | 2HQL (SEQ ID NO: 96) | Del100-104, 28-41−>154-177, V154K, C157A, |
| | | | C15T, I27C + Y42C) |
| 3FEV_A_fit_epitope | SEQ ID NO: 42 | 3FEV (SEQ ID NO: 97) | 5-14−>154-177, V154T) |
| 3FEV_B | SEQ ID NO: 43 | 3FEV (SEQ ID NO: 97) | 5-14−>154-177, V154T, C157A) |
| 3FEV_C | SEQ ID NO: 44 | 3FEV (SEQ ID NO: 97) | 5-14−>154-177, V154T, C157A, K155C + F176C) |
| 1GVP_A | SEQ ID NO: 45 | 1GVP (SEQ ID NO: 98) | 28-50−>154-177, V154L, C157Q, A174I, F176L, Y177D) |
| 3EN2_A_fit_epitope | SEQ ID NO: 46 | 3EN2 (SEQ ID NO: 99) | 34-47−>154-177, ins(H81 + GSG + A86)) |
| 3EN2_B | SEQ ID NO: 47 | 3EN2 (SEQ ID NO: 99) | 34-47−>154-177, ins(H81 + GSG + A86), C157A) |
| 3EN2_C | SEQ ID NO: 48 | 3EN2 (SEQ ID NO: 99) | 34-47−>154-177, ins(H81 + GSG + A86), C157A, Y33C + F48C) |
| 1GG3_A | SEQ ID NO: 49 | 1GG3 (SEQ ID NO: 100) | Del1-185, 238-258−>156-175, C157F, F159I, |
| | | | D197G, L198G, E199G) |
| 2AR5_A | SEQ ID NO: 50 | 2AR5 (SEQ ID NO: 101) | Del115-118, 24-44−>156-175, C157Y) |
| 2F7S_A | SEQ ID NO: 51 | 2F7S (SEQ ID NO: 102) | 42-69−>154-177) |
| 2F7S_B | SEQ ID NO: 52 | 2F7S (SEQ ID NO: 102) | 42-69−>154-177, C157A) |
| 2F7S_C | SEQ ID NO: 53 | 2F7S (SEQ ID NO: 102) | 42-69−>154-177, C157A, D41C + D70C) |
| 3HM2_A | SEQ ID NO: 54 | 3HM2 (SEQ ID NO: 103) | 149-162−>154-177, K155H) |
| 3HM2_B | SEQ ID NO: 55 | 3HM2 (SEQ ID NO: 103) | 149-162−>154-177, K155H, C157A) |
| 3HM2_C | SEQ ID NO: 56 | 3HM2 (SEQ ID NO: 103) | 149-162−>154-177, K155H, C157A, |
| | | | I148C + A163C) |
| 1D3B_A | SEQ ID NO: 57 | 1D3B (SEQ ID NO: 104) | 45-57−>154-177) |
| 1D3B_B | SEQ ID NO: 58 | 1D3B (SEQ ID NO: 104) | 45-57−>154-177, C157A) |
| 1D3B_C | SEQ ID NO: 59 | 1D3B (SEQ ID NO: 104) | 45-57−>154-177, C157A, R44C + E58C) |
| lL3I_A_fit_epitope | SEQ ID NO: 60 | 1L3I (SEQ ID NO: 105) | 163-176−>154-177) |
| 1L3I_B | SEQ ID NO: 61 | 1L3I (SEQ ID NO: 105) | 163-176−>154-177, C157A) |
| 1L3I_C | SEQ ID NO: 62 | 1L3I (SEQ ID NO: 105) | 163-176−>154-177, C157A, I162C + R177C) |
| 1VH8_A | SEQ ID NO: 63 | 1VH8 (SEQ ID NO: 106) | 15-32−>154-177) |
| 1VH8_B | SEQ ID NO: 64 | 1VH8 (SEQ ID NO: 106) | 15-32−>154-177, C157A) |
| 1VH8_C | SEQ ID NO: 65 | 1VH8 (SEQ ID NO: 106) | 15-32−>154-177, C157A, V154G, Y177G) |
| 1X3E_A | SEQ ID NO: 66 | 1X3E (SEQ ID NO: 107) | 35-49−>GS, Del111-119, 83-98−>154-177) |
| 1X3E_B | SEQ ID NO: 67 | 1X3E (SEQ ID NO: 107) | 35-49−>GS, Del111-119, 83-98−>154-177, |
| | | | C157A) |
| 1X3E_C | SEQ ID NO: 68 | 1X3E (SEQ ID NO: 107) | 35-49−>GS, Del111-119, 83-98−>154-177, |
| | | | C157A, K82C + E99C) |
| 3L1E_A | SEQ ID NO: 69 | 3L1E (SEQ ID NO: 108) | Del88-105, 41-55−>154-177) |
| 3L1E_B | SEQ ID NO: 70 | 3L1E (SEQ ID NO: 108) | Del88-105, 41-55−>154-177, C157A) |
| 1DHN_A | SEQ ID NO: 71 | 1DHN (SEQ ID NO: 109) | 100-114−>154-177) |
| 1DHN_B | SEQ ID NO: 72 | 1DHN (SEQ ID NO: 109) | 100-114−>154-177, C157A) |
| 1BM9_A | SEQ ID NO: 73 | 1BM9 (SEQ ID NO: 110) | 68-89−>154-177) |
| 1BM9_B | SEQ ID NO: 74 | 1BM9 (SEQ ID NO: 110) | 68-89−>154-177, Y177F, C157A) |
| 1BM9_C | SEQ ID NO: 75 | 1BM9 (SEQ ID NO: 110) | 68-89−>154-177, Y177F, C157A, L33G) |
| 3PYR_A | SEQ ID NO: 76 | 3PYR (SEQ ID NO: 111) | |
| 1T0A_A | SEQ ID NO: 77 | 1T0A (SEQ ID NO: 112) | |

In Table 1, “Del” refers to deletion; “Ins” refers to insertion; “−>” refers to substitution, for example “68-89->154-177” indicates that residues 68-89 of the scaffold sequence were replaced with positions 154-177 of gp120.

### Example 4

### Protein Nanoparticles Including a Minimal PG9 Epitope

This example illustrates protein nanoparticles including minimal PG9 epitopes. Minimal PG9 epitope sequences with and without a pair of stabilizing cysteine residues at gp120 positions 155 and 176 were placed on the N-terminus, the C-terminus, or on an internal loop of the ferritin, encapsulin or SOR proteins. Minimal PG9 epitope sequences that do not include a pair of stabilizing cysteine residues at gp120 positions 155 and 176 were placed on an internal loop of the ferritin, encapsulin or SOR protein. Self-assembling protein nanoparticles including the minimal PG9 epitope were produced, and screened for binding to monoclonal antibody PG9.

Methods:

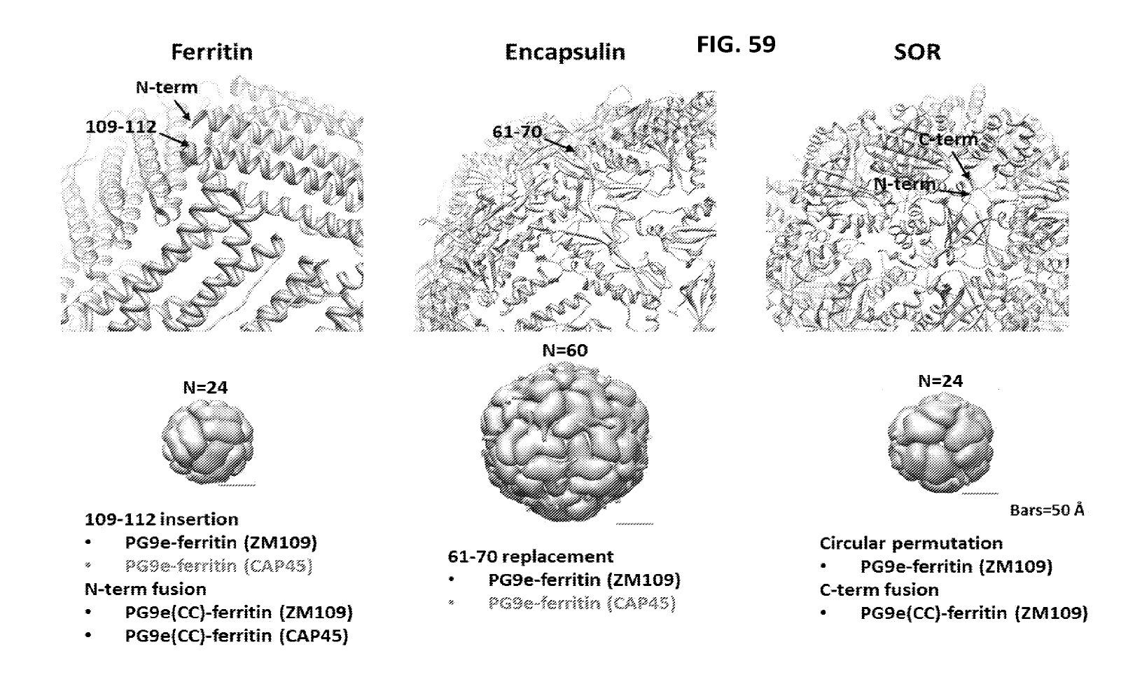

The minimal PG9 epitope (residues 154-177) or variations thereof, were inserted or fused to ferritin, encapsulin or SOR genes using the schemes shown in FIG. 59. The expression plasmids were transfected into HEK293 cells grown in the presence of swainsonine, or transfected into HEK293 GnTI^{-/-} cells. Particles were purified from the media using lectin affinity chromatography (snow drop lectin from Galanthus nivalis) followed by size-exclusion chromatography. Binding experiments were performed by incubating purified particles or particle-containing expression supernatant with the listed antibodies (PG9, PG16, VRC01) and Protein A agarose resin. After this incubation, the resin was pelleted and washed several times, and then incubated with SDS-containing buffer at 100 C. The solubilized and denatured proteins were separated by SDS-PAGE and visualized with Coomassie stain.

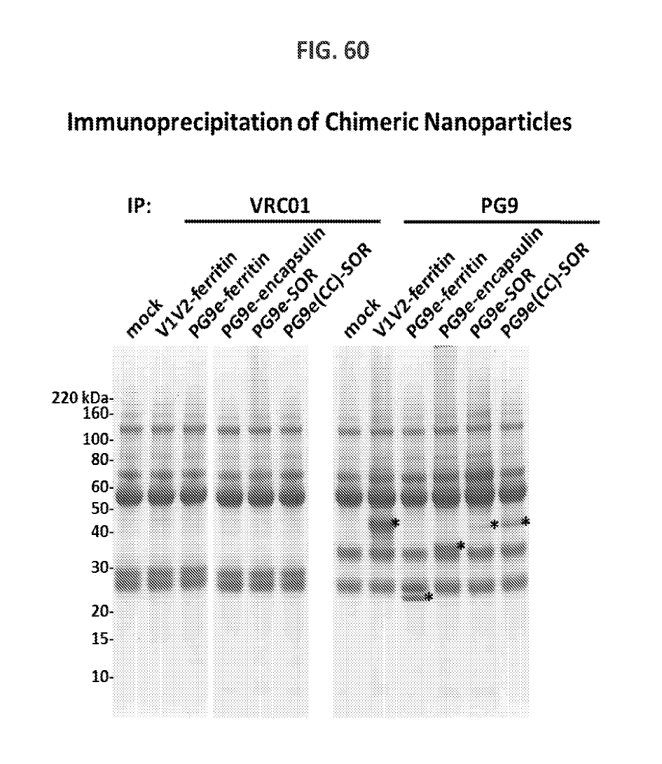

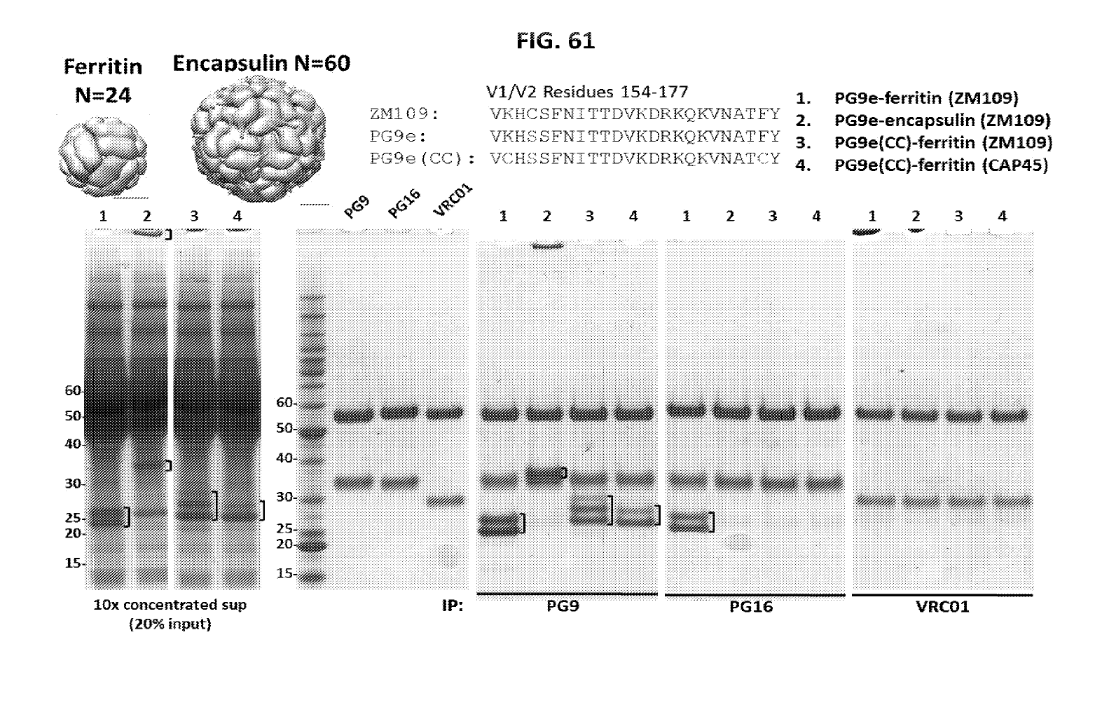

The results show that PG9 can immunoprecipitate ferritin, encapsulin, or SOR particles displaying the minimal PG9 epitope (FIG. 60). VRC01, a CD4-binding site-directed antibody, does not interact with the particles, as expected. PG9 can immunoprecipitate PG9e-ferritin (ZM109), PG9e-encapsulin (ZM109), PG9e(CC)-ferritin (ZM109) and PG9e(CC)-ferritin (CAP45), whereas PG16 only interacts with PG9e-ferritin (ZM109) (FIG. 61).

### Example 5

### PG9 Epitope Multimers

This example illustrates multimers of the gp120 V1/V2 domain covalently linked to form a dimer. The C-terminus of a first V1/V2 domain was linked to the N-terminus of a second V1/V2 domain via an eight amino acid linker. Additionally, V1/V2 domain multimers with truncated variable loops (V1 loop and V2 loop) were also generated and tested for binding to monoclonal antibody PG9. The results show that V1/V2 dimer (with and without the V1 and V2 variable loops) is specifically bound by monoclonal antibody PG9 with nanomolar affinity.

Method Used to Generate Multimers.

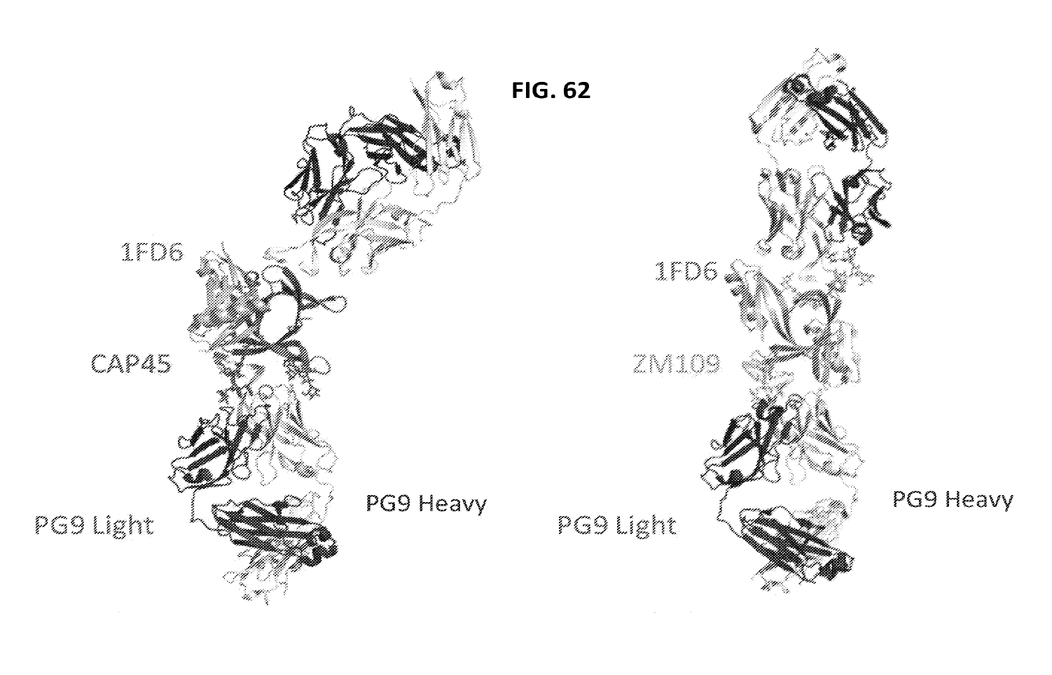

The crystal structures of PG9 in complex with the 1FD6A_V1V2 scaffold revealed that the scaffold formed dimers and the dimerization was mediated solely through the V1V2 region (see, for example, FIG. 62). Using the structures of PG9 with 1FD6_Cap45 and 1FD6_ZM109 as templates, a short peptide linker region was added connecting the C-terminal of one subunit to the N-terminal of the second. The linked dimers were expressed in GnTI-cells and subsequently purified on Ni-NTA columns. Initial binding was conducted using ELISA assays and followed up with quantitative surface plasmon resonance data. Linked dimers mixed at a 1:5 ratio with PG9 show a shift in the gel filtration peak corresponding to the complex.

Results.

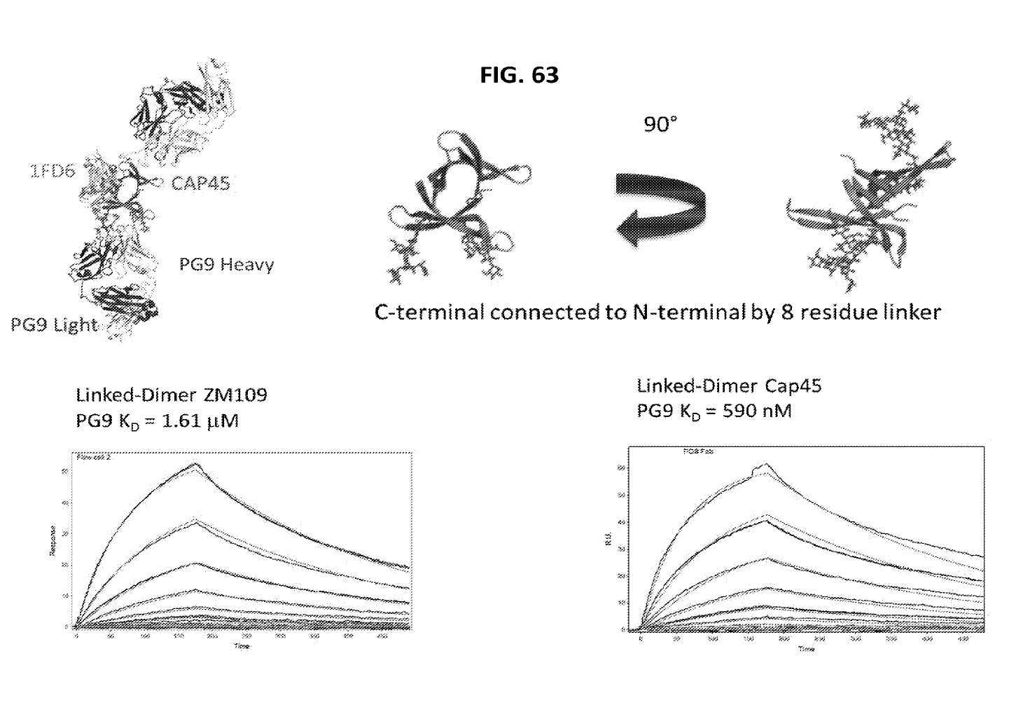

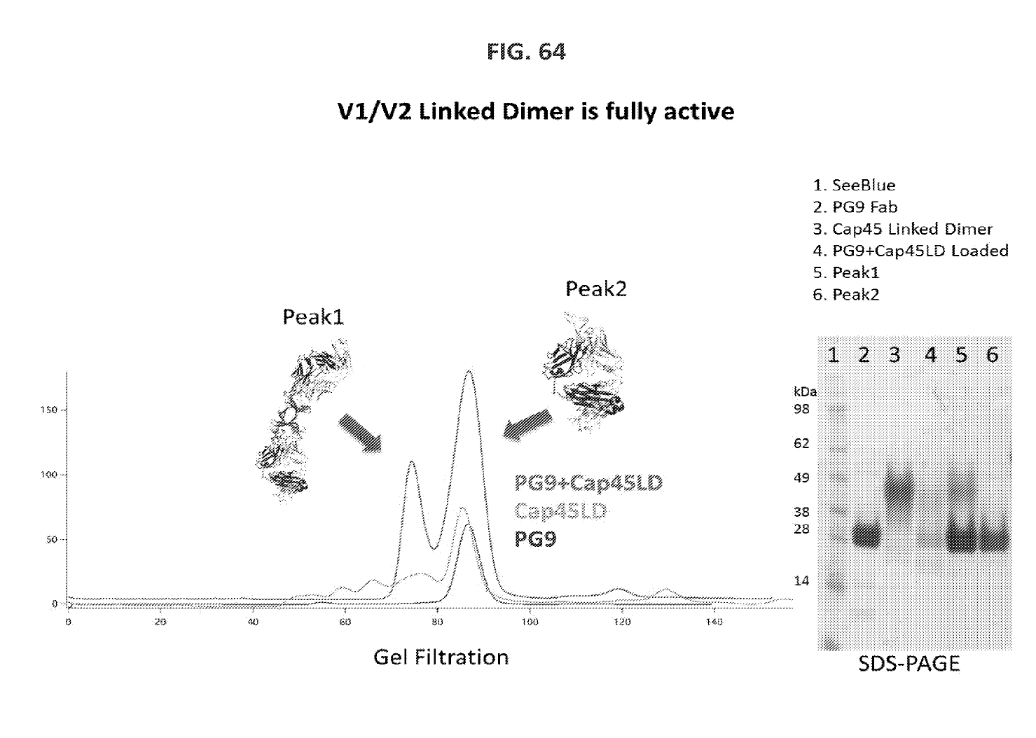

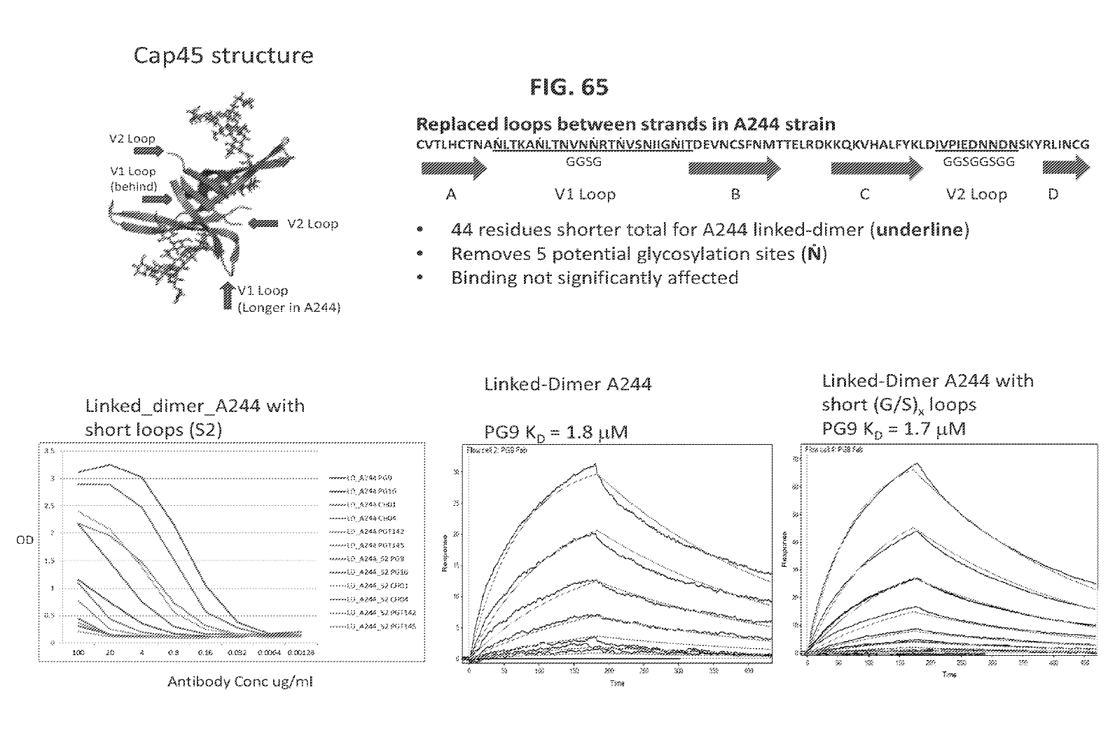

The linked dimers display good expression and binding to PG9 (k_{D}˜1 μM or below; see FIG. 63). Further, the linked dimer shifts fully when complexed with PG9 indicating that it is close to 100% active for PG9 binding (see FIG. 64). ELISA assays reveal that the linked dimers are also able to bind various other V1/V2 antibodies such as CH01, CH04, PGT142 and PGT145. The variable loops which exist between strands A and B and between C and D can be shortened in this context or replaced with (GS) linkers with no loss of binding to antibodies, potentially better exposing the epitope in an immunogen context (see FIG. 65).

### Example 6

### Protein Nanoparticles Including PG9 Epitope Multimers

This example illustrates exemplary protein nanoparticles including V1/V2 domain dimers. In some examples, the V1/V2 dimers are fused to ferritin, encapsulin or SOR protein sequences, respectively. The V1/V2 dimers are fused to the N- or the C-Terminus of the ferritin, encapsulin or SOR protein. Self-assembling protein nanoparticles including these fusion proteins are produced, and screened for binding to monoclonal antibody PG9, for example, using methods familiar to the person of ordinary skill in the art and/or described herein.

In one example, V1/V2 proteins from several different HIV-1 strains are fused to the N-terminus of ferritin and encapsulin using an amino acid linker (such as a 10 amino acid linker, e.g., GS₅) and are expressed to generate ferritin or encapsulin protein nanoparticles with the V1/V2 domain. The V1/V2 proteins include linked dimers with shortened V1 and V2 variable loops as well as dimers consisting of two different strains. The particles can be expressed and purified, for example, as described herein.

### Example 7

### Immunization of Animals

This example describes exemplary procedures for the production of immunogens including a disclosed antigen (such as a polypeptide including a PG9 epitope), as well as and immunization of animals with the disclosed immunogens (such as a polypeptide including a PG9 epitope).

In some examples nucleic acid molecules encoding the disclosed immunogens are cloned into expression vector CMV/R. Expression vectors are then transfected into 293F cells using 293Fectin (Invitrogen, Carlsbad, Calif.). Five days after transfection, cell culture supernatant is harvested and concentrated/buffer-exchanged to 500 mM NaCl/50 mM Tris pH8.0. The protein initially is purified using HiTrap IMAC HP Column (GE, Piscataway, N.J.), and subsequent gel-filtration using SUPERDEX™ 200 (GE). In some examples the 6×His tag is cleaved off using 3C protease (Novagen, Madison, Wis.).

For vaccinations with the disclosed immunogens 3-4 months old rabbits (NZW) (Covance, Princeton, N.J.) are immunized using the Sigma Adjuvant System (Sigma, St. Louis, Mo.) according to manufacture's protocol. Specifically, three rabbits in each group are vaccinated with 50 μg of protein in 300 μl PBS emulsified with 300 μl of adjuvant intramuscularly (both legs, 300 μl each leg) for example at week 0, 4, 8, 12, 16. Sera are collected for example at week 6 (Post-1), 10 (Post-2), 14 (Post-3), and 18 (Post-4), and subsequently analyzed for their neutralization activities against a panel of HIV-1 strains, and the profile of antibodies that mediate the neutralization.

The immunogens are also used to probe for rabbit anti-sera for existence of V1/V2 domain specific antibodies in the anti-sera.

### Example 8

### A Short Segment of the HIV-1 Gp120 V1/V2 Region is a Major Determinant of Resistance to V1/V2 Neutralizing Antibodies

This example illustrates that mutations in a short segment of V1/V2 resulted in gain of sensitivity to PG9 and related V1/V2 neutralizing antibodies. The results show both a common mechanism of HIV-1 resistance to and a common mode of recognition by this class of antibodies.

Antibody PG9 is a prototypical member of a class of V1/V2-directed antibodies that effectively neutralizes diverse strains of HIV-1. Antibody PG9 recognizes an epitope primarily in the VI/V2 region of HIV-1 gp120, requires an N-linked glycan at residue 160, and generally binds with much higher affinity to membrane-associated trimeric forms of Env than to monomeric forms of gp120. Members of this class of V1/V2-directed antibodies include PG9 and the somatically related PG16, as well as antibodies CH01-CH04 and PGT141-145 from two other donors (Bonsignori, et al., 2011. J Virol 85:9998-10009.; Walker et al. 2009. Science 326:285-9.; and Walker, et al. 2011. PNAS 108:20125-9). To gain a more complete understanding of the mechanism of naturally occurring viral resistance to PG9 and similar mAbs, a combination of sequence and structural analyses to predict gain-of-sensitivity mutations among PG9-resistant strains was performed. The effect of the mutations on resistance to PG9 and five other members of the VI/V2 antibody class were then assessed.

Antibody PG9 is one of the most broadly cross-reactive of the class and neutralizes 70-80% of diverse HIV-1 isolates. The structure of PG9 in complex with scaffolded forms of V1/V2 is disclosed herein: when bound by PG9, VI/V2 adopts a 4-stranded β-sheet structure, with PG9 interacting with two glycans (at residues 156 and 160) and with one β-strand (strand C, at the sheet edge). The free antibody structures of PG9 as well as other antibodies from this class (PG16, CH04, and PGT145) are also known, and suggest a common mode of Env recognition mediated primarily by the long anionic complementarity-determining region (CDR) H3 loops of these antibodies. Studies indicate that virus neutralization sensitivity to PG9 might correlate with V2 length, the number and positioning of potential N-linked glycosylation sites in V1, V2, and V3, and net charge of the PG9-interacting strand C. Additionally, residues outside of the structure-identified epitope—both in VI/V2, as well as in V3—were found to affect PG9 and PG16 neutralization. Resistance conferred by an N160K mutation was described as a defining attribute for this class, but this residue does not account for all instances of resistance.

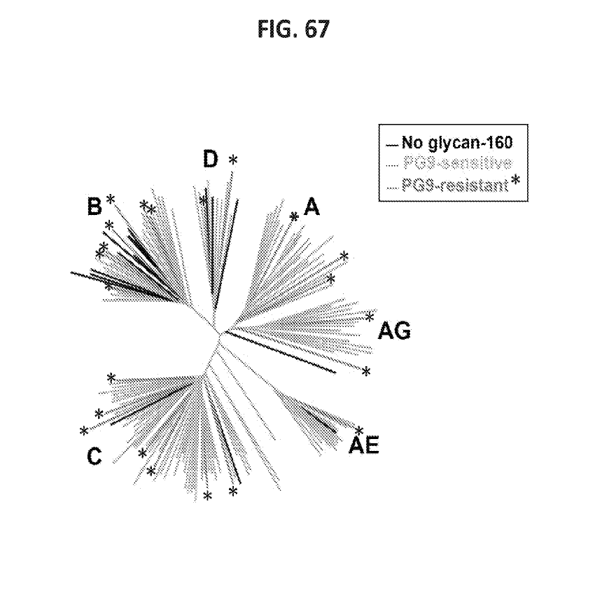

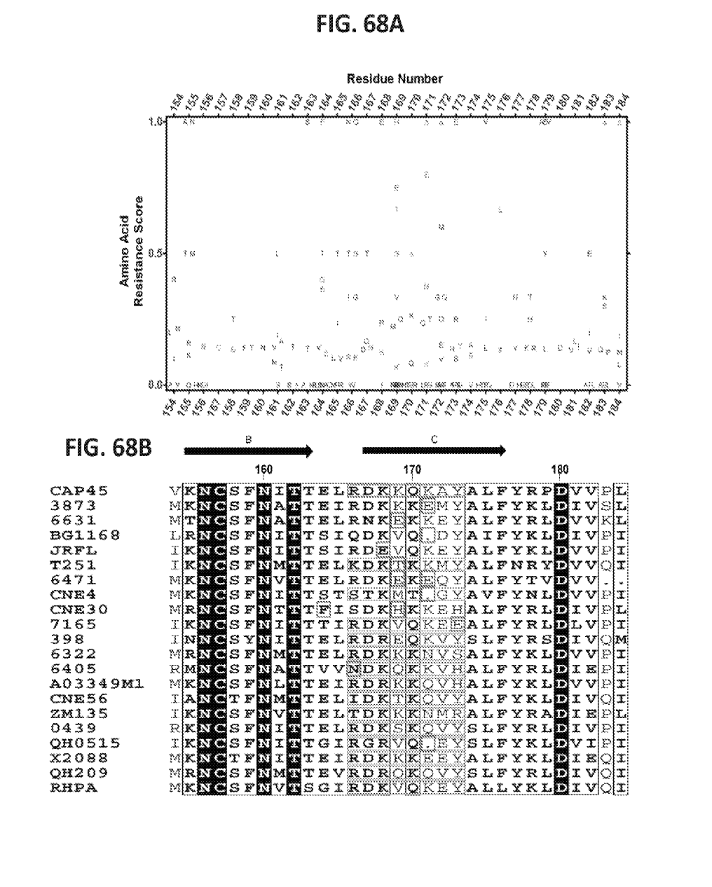

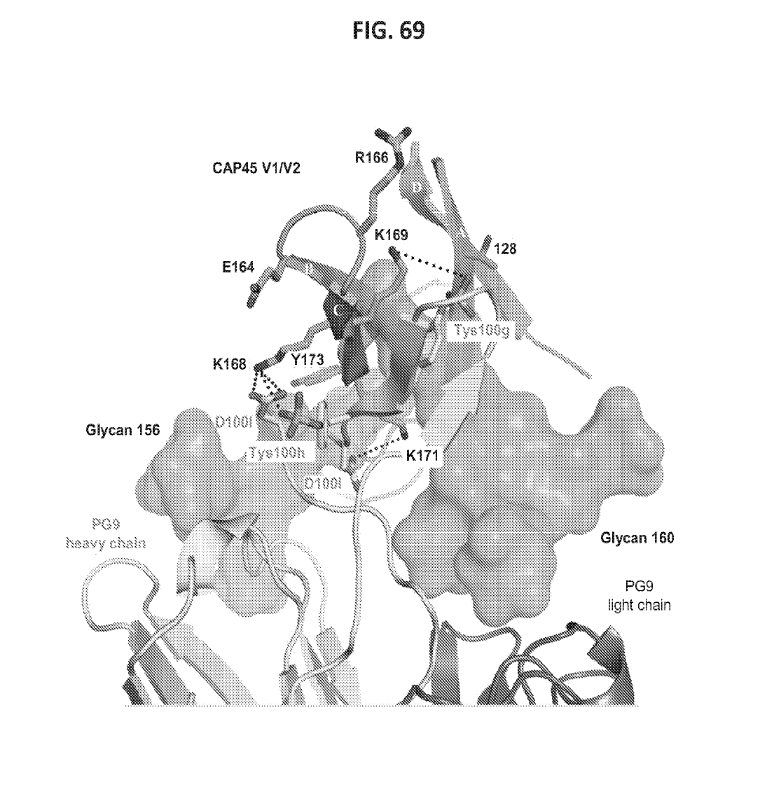

Among a panel of 172 HIV-1 Env-pseudoviruses, 38 strains (22%) were found to be resistant to PG9 (Doria-Rose et al., 2012. J Virol 86:3393-7; and Walker et al, 2009. Science 326:285-9). Examination of strain sequences indicated that 16 were missing the N-linked glycan at position 160, leaving a total of 134 sensitive and 22 resistant strains to be analyzed for protein sequence-based resistance signatures (FIG. 67). Initially, residues 154-184 of VI/V2 (HXB2-relative residue numbering) a region that spans β-strands B and C and is relatively conserved (with few insertions/deletions), and includes the entire PG9 epitope, was examined. Specifically, based on sequence alignments, we searched for amino acids that were preferentially found among PG9-resistant versus sensitive strains for a given residue position (FIG. 68A). A number of such amino acids at positions at or near the PG9 interface (as observed in the crystal structure of scaffolded V1/V2) were selected for gain-of-sensitivity mutations (FIG. 68B). Each of the selected residues was mutated to amino acids commonly observed among PG9-sensitive sequences (FIG. 68A). This sequence analysis was able to identify candidate mutations for 11 of the PG9-resistant strains. However, since the selected mutations were primarily in the short segment between residues 166-173, which overlaps strand C of V1/V2, we swapped that 8-residue segment in nine additional strains, as well as in five of the strains identified by the sequence analysis, with the corresponding segment from CAP45, a sensitive strain used for the PG9 crystal structure (FIG. 68B). Additionally, analysis of potential N-linked glycosylation sites (PNGS) revealed that residue 128 was the location of a PNGS in the PG9-resistant strain CNE4 but not in any of the other strains in the neutralization panel. Since glycans may create substantial steric hindrance, PNGS 128 in CNE4 was also selected for gain-of-sensitivity experiments, despite a more distal position with respect to the PG9 interface in the scaffolded V1/V2 structures (FIG. 69).

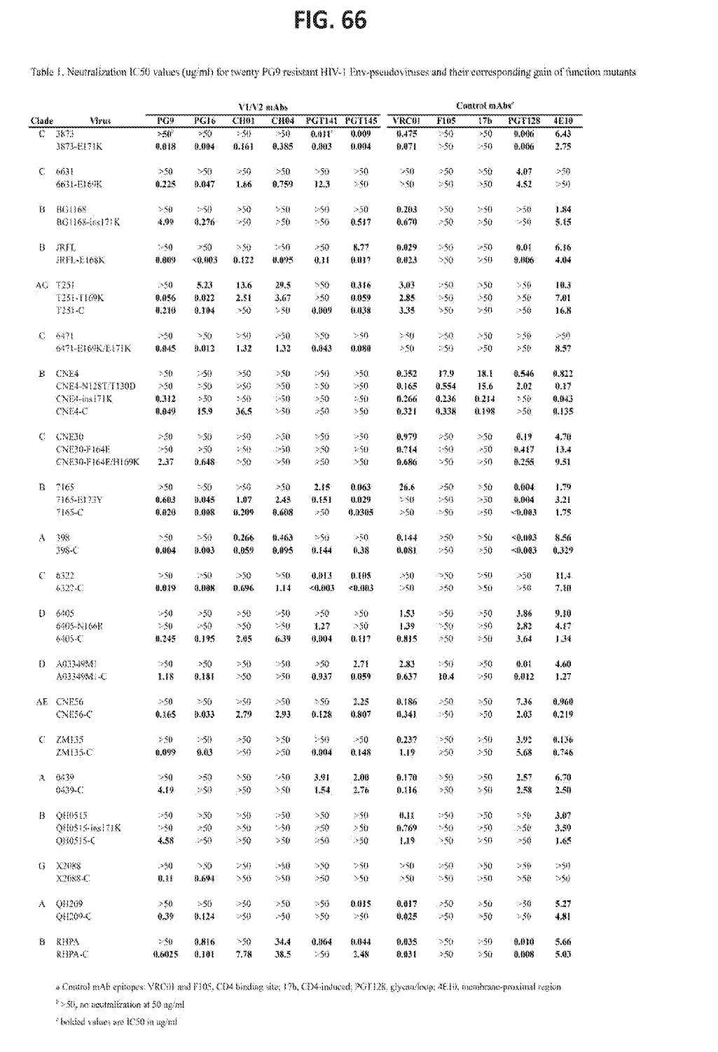

In total, 20 PG9-resistant HIV-1 isolates from six clades were analyzed by mutagenesis and neutralization assays (FIG. 66). The point mutations and strand C swaps were generated by site directed mutagenesis (GeneImmune LLC, New York, N.Y.) on Env expression plasmids. Parental and mutant Envs were used to construct pseudoviruses for the single round of infection neutralization assays using TZM-bl target cells as previously described (Shu et al., Vaccine, 25:1398-1408, 2007; and Wu et al., Science, 329:856-861, 2010). Each pair of parental/mutant viruses was tested against six members of the V1/V2-directed class of broadly neutralizing antibodies, isolated from three different donors: PG9 and PG 16, CH01 and CH04, and PGT141 and PGT145. In each case, the parental virus was resistant to PG9 at an IC50>50 ug/ml, although several were sensitive to other V1/V2 mAbs. mAbs to other epitopes (mAbs VRC01, F105, 17b, PGT128 and 4E10) were included as controls to assess the impact of the mutations on overall Env conformation and neutralization sensitivity.

Mutations that changed the glutamic acid (E) to lysine (K) at positions 168, 169, or 171 had the most dramatic effects on sensitivity to the V1/V2 mAbs (FIG. 66). For viral strains 3873, 6631, BG 1168, JRFL, and T251-18, a single point mutation at one of these three sites was sufficient to confer sensitivity to multiple V1/V2 mAbs. For resistant strain 6471, the double mutation E169K/E171K restored neutralization sensitivity to all six V1/V2 mAbs tested. Point mutations had a more modest effect on some viral strains: CNE4 with an inserted 171K gained sensitivity to just PG9, and CNE30-F164E/H169K gained sensitivity to both PG9 and PG 16 but no others.

These observations confirm and extend the information gained from the crystal structures of PG9 with scaffolded V1/V2 from strains ZM109 and CAP45. In these structures, V1/V2 residues 168, 169, and 171 are part of the cationic V1/V2 strand C that interacts directly with a number of negatively-charged residues in the CDRH3 of PG9: sulfated tyrosines Tys 100g and Tys 100h, and Asp 100i and Asp 1001 (Kabat residue numbering). Negatively charged residues and deletions at positions 168, 169, and 171 likely disturb interactions and/or create charge repulsion with PG9 CDRH3 (FIG. 69). Mutagenesis studies have found that K169E confers resistance to PG9 and PG16, while the less drastic K171A mutation had a more moderate effect on neutralization by these antibodies. Additional positions in strand C also affected sensitivity to V1/V2 antibodies. The E173Y mutation in 7165.18 effectively conferred sensitivity, in agreement with previous results showing loss of neutralization of Y173A in JR-CSF for both PG9 and PG 16 (14). E173Y could potentially stabilize the positioning of glycan-156 and may thus have an indirect effect on interactions with PG9 (FIG. 69).

Replacement of an 8-residue segment (residues 166-173, overlapping strand C) with the corresponding segment from CAP45 was also effective, conferring sensitivity to all mAbs resisted by the parental strains 398, 6322, 6405, A03349M1, CNE56, and ZM135. Sensitivity to PG9 (but not the other mAbs) was also observed for the CAP45 C-strand chimeras of 0439 and QH0515, and to PG9 and PG16 for QH209 and X2088. Among three strains for which both point mutants and CAP45 C-strand chimeras were tested, the strand C swap had the more dramatic effect. Strain CNE4 was resistant to all six mAbs; the PNG-removal mutant CNE4-NI28T.T130D had no effect; CNE4-insI71K gained sensitivity only to PG9; but the CAP45 strand-C chimera was sensitive to PG9, PG16, and CH01. Similarly, on strain 6405, the point mutant N166R only gained sensitivity to PGT141 (possibly indicating additional interactions with the longer PGT141 penetrating loop which may extend further toward the 166 region as compared to PG9, FIG. 69); in contrast, the CAP45 strand C provided sensitivity to all 6 mAbs. Finally, the point mutation in QH0515-ins171K had no effect on sensitivity, but the CAP45 strand-C chimera conferred PG9 neutralization.

Paradoxically, in four cases, while the CAP45 strand-C chimeras gained sensitivity to PG9 and PG16, a gain of resistance was noted for CH01 and CH04 (strain T251-18), PGT141 (RHPA and 7165), or PGT145 (QH209). This observation suggests that, despite overall similarity in the epitope recognized and the requirement for the N160 glycan, there is some variation in the mode of recognition by members of the V1/V2 class of neutralizing mAbs.

The mutations tested did not cause global alterations in the neutralization sensitivity as assessed by mAbs to non-V1/V2 epitopes (FIG. 66). The one exception was strain CNE4, for which the mutants increased accessibility to CD4 binding site (targeted by control mAb F105) and CD4-induced epitopes (targeted by 17b) while decreasing the potency of PGT128 (glycans). The other 19 strains showed little change in sensitivity to the control mAbs, indicating that the effects of the mutations were likely specific for V1/V2 recognition.

These gain-of-sensitivity mutational analyses support the conclusions drawn from the scaffolded V1/V2-PG9-crystal structures, suggesting that the conformations observed for these engineered/crystalline constructs are biologically and functionally relevant. For each of the PG9-resistant strains selected for gain-of-function experiments, at least one of the selected mutants gained sensitivity to one or more of the V1/V2 mAbs, thus validating the predictions based on structure and sequence. While correlations of PG9 resistance with other factors such as glycosylation and length of V2 have also been noted, our results suggest a general mechanism of resistance to V1/V2-directed broadly neutralizing antibodies that involves alteration of basic residues within strand C of the V1/V2 domain. Additionally, our observation that gain-of-sensitivity mutations generally affected not only PG9, but also antibodies PG 16, CH01, CH04, PGTI41, and PGTI45, provides further evidence that the members of this class recognize a similar epitope on the native HIV-1 envelope glycoprotein

### Example 9

### Treatment of HIV in a Subject

This example describes exemplary methods for treating or inhibiting an HIV infection in a subject, such as a human subject by administration of one or more of the antigens disclosed herein. Although particular methods, dosages and modes of administrations are provided, one skilled in the art will appreciate that variations can be made without substantially affecting the treatment.

HIV, such as HIV type 1 (HIV-1) or HIV type 2 (HIV-2), is treated by administering a therapeutically effective amount of a disclosed antigen including a PG9 epitope (such as a PG9 epitope stabilized in a PG9 bound conformation) that induces an immune response to HIV, for example by inducing an immune response, such as a neutralizing antibody response to gp120 polypeptide present on the surface of HIV.

Briefly, the method includes screening subjects to determine if they have HIV, such as HIV-1 or HIV-2. Subjects having HIV are selected for further treatment. In one example, subjects are selected who have increased levels of HIV antibodies in their blood, as detected with an enzyme-linked immunosorbent assay, Western blot, immunofluorescence assay or nucleic acid testing, including viral RNA or proviral DNA amplification methods. In one example, half of the subjects follow the established protocol for treatment of HIV (such as a highly active antiretroviral therapy). The other half follow the established protocol for treatment of HIV (such as treatment with highly active antiretroviral compounds) in combination with administration of the agents including a therapeutically effective amount of a disclosed antigen that induces an immune response to HIV. In another example, half of the subjects follow the established protocol for treatment of HIV (such as a highly active antiretroviral therapy). The other subjects receive a therapeutically effective amount of a disclosed PG9 antigen that induces an immune response to HIV, such as a neutralizing antibody response.

### Screening Subjects

In particular examples, the subject is first screened to determine if the subject has HIV. Examples of methods that can be used to screen for HIV include measuring a subject's CD4+ T cell count and the level of HIV in serum blood levels.

In some examples, HIV testing consists of initial screening with an enzyme-linked immunosorbent assay (ELISA) to detect antibodies to HIV, such as to HIV-1. Specimens with a nonreactive result from the initial ELISA are considered HIV-negative unless new exposure to an infected partner or partner of unknown HIV status has occurred. Specimens with a reactive ELISA result are retested in duplicate. If the result of either duplicate test is reactive, the specimen is reported as repeatedly reactive and undergoes confirmatory testing with a more specific supplemental test (for example, Western blot or an immunofluorescence assay (IFA)). Specimens that are repeatedly reactive by ELISA and positive by IFA or reactive by Western blot are considered HIV-positive and indicative of HIV infection. Specimens that are repeatedly ELISA-reactive occasionally provide an indeterminate Western blot result, which may be either an incomplete antibody response to HIV in an infected person or nonspecific reactions in an uninfected person. IFA can be used to confirm infection in these ambiguous cases. In some instances, a second specimen will be collected more than a month later and retested for subjects with indeterminate Western blot results. In additional examples, nucleic acid testing (for example, viral RNA or proviral DNA amplification method) can also help diagnosis in certain situations.

The detection of HIV in a subject's blood is indicative that the subject has HIV and is a candidate for receiving the therapeutic compositions disclosed herein. Moreover, detection of a CD4+ T cell count below 350 per microliter, such as 200 cells per microliter, is also indicative that the subject is likely to have HIV.

Pre-screening is not required prior to administration of the therapeutic compositions disclosed herein.

### Pre-Treatment of Subjects

In particular examples, the subject is treated prior to diagnosis of AIDS with the administration of a therapeutically effective amount of a disclosed antigen including a PG9 epitope (such as a PG9 epitope stabilized in a PG9 bound conformation) that induces an immune response to HIV. In some examples, the subject is treated with an established protocol for treatment of AIDS (such as a highly active antiretroviral therapy) prior to treatment with the administration of a therapeutic agent that includes one or more of the disclosed antigen that induces an immune response to HIV. However, such pre-treatment is not always required and can be determined by a skilled clinician.

### Administration of Therapeutic Compositions

Following selection, a therapeutic effective dose of a therapeutically effective amount of a disclosed antigen including a PG9 epitope (such as a PG9 epitope stabilized in a PG9 bound conformation) that induces an immune response to HIV is administered to the subject (such as an adult human or a newborn infant either at risk for contracting HIV or known to be infected with HIV). Additional agents, such as anti-viral agents, can also be administered to the subject simultaneously or prior to or following administration of the disclosed agents. Administration can be achieved by any method known in the art, such as oral administration, inhalation, intravenous, intramuscular, intraperitoneal or subcutaneous.

The amount of the immunogenic composition administered to prevent, reduce, inhibit, and/or treat HIV or a condition associated with it depends on the subject being treated, the severity of the disorder and the manner of administration of the immunogenic composition. Ideally, a therapeutically effective amount of the immunogenic composition is the amount sufficient to prevent, reduce, and/or inhibit, and/or treat the condition (for example, HIV) in a subject without causing a substantial cytotoxic effect in the subject. An effective amount can be readily determined by one skilled in the art, for example using routine trials establishing dose response curves. In addition, particular exemplary dosages are provided above. The therapeutic compositions can be administered in a single dose delivery, via continuous delivery over an extended time period, in a repeated administration protocol (for example, by a daily, weekly or monthly repeated administration protocol). In one example, a therapeutically effective amount of a disclosed antigen that induces an immune response to HIV is administered intravenously to a human. As such, these compositions may be formulated with an inert diluent or with a pharmaceutically acceptable carrier. Immunogenic compositions can be taken long term (for example over a period of months or years).

### Assessment

Following the administration of one or more therapies, subjects having HIV (for example, HIV-1 or HIV-2) can be monitored for reductions in HIV levels, increases in a subjects CD4+ T cell count or reductions in one or more clinical symptoms associated with HIV infection. In particular examples, subjects are analyzed one or more times, starting 7 days following treatment. Subjects can be monitored using any method known in the art. For example, biological samples from the subject, including blood, can be obtained and alterations in HIV or CD4+ T cell levels evaluated.

### Additional Treatments

In particular examples, if subjects are stable or have a minor, mixed or partial response to treatment, they can be re-treated after re-evaluation with the same schedule and preparation of agents that they previously received for the desired amount of time, including the duration of a subject's lifetime. A partial response is a reduction, such as at least a 10%, at least 20%, at least 30%, at least 40%, at least 50% or at least 70% reduction of HIV viral load, HIV replication or combination thereof. A partial response may also be an increase in CD4+ T cell count such as at least 350 T cells per microliter.

### Example 10

### Treatment of Subjects

This example describes methods that can be used to treat a subject that has or is at risk of having an infection from HIV that can be treated by eliciting an immune response, such as a neutralizing antibody response to HIV. In particular examples, the method includes screening a subject having, thought to have or at risk of having a HIV infection. Subjects of an unknown infection status can be examined to determine if they have an infection, for example using serological tests, physical examination, enzyme-linked immunosorbent assay (ELISA), radiological screening or other diagnostic technique known to those of skill in the art. In some examples, subjects are screened to identify a HIV infection, with a serological test, or with a nucleic acid probe specific for a HIV. Subjects found to (or known to) have a HIV infection can be administered a disclosed antigen including a PG9 epitope (such as a PG9 epitope stabilized in a PG9 bound conformation) that can elicit an antibody response to HIV. Subjects may also be selected who are at risk of developing HIV for example, subjects exposed to HIV.

Subjects selected for treatment can be administered a therapeutic amount of the disclosed antigen including a PG9 epitope (such as a PG9 epitope stabilized in a PG9 bound conformation). The antigen can be administered at doses of 1 μg/kg body weight to about 1 mg/kg body weight per dose, such as 1 μg/kg body weight-100 μg/kg body weight per dose, 100 μg/kg body weight-500 μg/kg body weight per dose, or 500 μg/kg body weight-1000 μg/kg body weight per dose. However, the particular dose can be determined by a skilled clinician. The antigen can be administered in one or several doses, for example continuously, daily, weekly, or monthly. When administered sequentially the time separating the administration of the antigen can be seconds, minutes, hours, days, or even weeks.

The mode of administration can be any used in the art. The amount of agent administered to the subject can be determined by a clinician, and may depend on the particular subject treated. Specific exemplary amounts are provided herein (but the disclosure is not limited to such doses).

It will be apparent that the precise details of the methods or compositions described may be varied or modified without departing from the spirit of the described embodiments. We claim all such modifications and variations that fall within the scope and spirit of the claims below.

1-53. (canceled)

54. An epitope-scaffold protein, comprising:
(A) a gp120 polypeptide, comprising:
gp120 positions 126-196 according to the HXB2 numbering system and corresponding to the amino acid positions in the amino acid sequence set forth as SEQ ID NO: 1;
a first pair of cross-linked cysteines at positions 126 and 196, and a second pair of crosslinked cysteines at positions 131 and 157;
a first N-linked glycosylation site comprising an asparagine residue at position 160 and a second N-linked glycosylation site comprising an asparagine residue at position 156 or position 173, wherein the first and second glycosylation sites are glycosylated; and
(B) a heterologous scaffold comprising a 1VH8 scaffold; wherein
the 1VH8 scaffold is linked to the gp120 polypeptide, and the epitope scaffold protein specifically binds to monoclonal antibody PG9.

55. The epitope scaffold protein of claim 54, wherein the 1VH8 scaffold comprises the amino acid sequence set forth as SEQ ID NO: 106.

56. The epitope scaffold protein of claim 54, wherein the gp120 polypeptide does not comprise any cysteine residues at gp120 positions 127-130, 132-156 and 158-195;

57. The epitope scaffold protein of claim 54, wherein the gp120 polypeptide comprises at most four amino acid substitutions compared to a wild-type HIV-1 gp120.

58. The epitope scaffold protein of claim 57, wherein the wild type HIV-1 gp120 comprises an amino acid sequence set forth as any one of SEQ ID NOs: 1-8 or 154-160.

59. The epitope scaffold protein of claim 54, wherein
the asparagine at position 160 is glycosylated with a Man₅GlcNAc₂ glycan moiety; and
the asparagine at position 156 or the asparagine at position 173 is glycosylated with a complex glycan.

60. The epitope scaffold protein of claim 54, wherein monoclonal antibody PG9 specifically binds to the antigen or protein nanoparticle with a K_{D} of 100 μM or less.

61. A multimer of the epitope scaffold protein of claim 54.

62. A protein nanoparticle comprising the epitope scaffold protein of claim 54.

63. The protein nanoparticle of claim 62, wherein the protein nanoparticle is a virus-like particle, a ferritin nanoparticle, an encapsulin nanoparticle or a Sulfur Oxygenase Reductase (SOR) nanoparticle.

64. An isolated nucleic acid molecule encoding the epitope scaffold protein of claim 54.

65. The nucleic acid molecule of claim 64 operably linked to a promoter.

66. A vector comprising the nucleic acid molecule of claim 65.

67. An immunogenic composition comprising an effective amount of the epitope scaffold protein of claim 54, and a pharmaceutically acceptable carrier.

68. A method for generating an immune response to HIV-1 gp120 in a subject, comprising administering to the subject an effective amount of the immunogenic composition of claim 67, thereby generating the immune response.

69. The method of claim 68, wherein the subject has a HIV-1 infection.

70. A method for treating or preventing an HIV-1 infection in a subject, comprising administering to the subject a therapeutically effective amount of the immunogenic composition of claim 67, thereby treating the subject or preventing HIV-1 infection of the subject.

71. The method of claim 70, wherein the subject has a HIV-1 infection.

72. A kit for inducing an immune response to HIV-1 gp120 in a subject, comprising the epitope scaffold protein of claim 54; and instructions for using the kit.
